(19) United States
(12) Patent Application Publication
(10) Pub. No.: US 2026/0327225 A1
(43) Pub. Date: Oct. 22, 2026

(54) HETEROCYCLIC COMPOUNDS AS TRIGGERING RECEPTOR EXPRESSED ON MYELOID CELLS 2 AGONISTS AND METHODS OF USE

(71) Applicant: VIGIL NEUROSCIENCE, INC., Morristown, NJ (US)

(72) Inventors: Jonathan B. HOUZE, Morristown, NJ (US); Bhaumik PANDYA, Morristown, NJ (US)

(21) Appl. No.: 19/703,779

(22) Filed: Jun. 10, 2026

Related U.S. Application Data
(63) Continuation of application No. PCT/US24/59814, filed on Dec. 12, 2024.
(60) Provisional application No. 63/609,320, filed on Dec. 12, 2023.

(51) Int. Cl. C07D 471/04; A61K 31/519; A61K 31/5377; C07D 487/04; C07D 519/00

(52) U.S. Cl. CPC C07D 471/04; A61K 31/519; A61K 31/5377; C07D 487/04; C07D 519/00

(57) ABSTRACT

The present disclosure provides compounds of Formula I, useful for the activation of Triggering Receptor Expressed on Myeloid Cells 2 (“TREM2”). This disclosure also provides pharmaceutical compositions comprising the compounds, uses of the compounds, and compositions for treatment of, for example, a neurodegenerative disorder. Further, the disclosure provides intermediates useful in the synthesis of compounds of Formula I.

### CROSS REFERENCE TO RELATED APPLICATION

This application is a continuation of International Application No. PCT/US2024/059814, filed Dec. 12, 2024, which claims the benefit under 35 U.S.C. § 119(e) to U.S. Provisional Application No. 63/609,320, filed Dec. 12, 2023, the entirety of each of which is incorporated herein by reference.

### FIELD

The present disclosure provides compounds useful for the activation of Triggering Receptor Expressed on Myeloid Cells 2 (“TREM2”). This disclosure also provides pharmaceutical compositions comprising the compounds, uses of the compounds, and compositions for treatment of, for example, a neurodegenerative disorder. Further, the disclosure provides intermediates useful in the synthesis of compounds of Formula I.

### BACKGROUND

Microglia are resident innate immune cells in the brain and are important for the maintenance of homeostatic conditions in the central nervous system (Hickman et al. Nat Neurosci 2018, Li and Barres, Nat Rev Immunol., 2018). These resident macrophages express a variety of receptors that allow them to sense changes in their microenvironment and alter their phenotypes to mediate responses to invading pathogens, proteotoxic stress, cellular injury, and other infarcts that can occur in health and disease. Id. Microglia reside in the parenchyma of the brain and spinal cord where they interact with neuronal cell bodies (Cserep et al. Science, 2019), neuronal processes (Paolicelli et al. Science, 2011, Ikegami et al. Neruopathology, 2019) in addition to other types of glial cells (Domingues et al. Front Cell Dev Biol, 2016; Liddelow et al. Nature, 2017, Shinozaki et al. Cell Rep., 2017), playing roles in a multitude of physiological processes. With the ability to rapidly proliferate in response to stimuli, microglia characteristically exhibit myeloid cell functions such as phagocytosis, cytokine/chemokine release, antigen presentation, and migration (Colonna and Butovsky, Annu Rev Immunol, 2017). More specialized functions of microglia include the ability to prune synapses from neurons and directly communicate with their highly arborized cellular processes that survey the area surrounding the neuronal cell bodies (Hong et al. Curr Opin Neurobiol, 2016; Sellgren et al. Nat Neurosci, 2019).

The plasticity of microglia and their diverse states as described through single-cells RNASeq profiling are thought to arise through the integration of signaling from a diverse array of cell surface receptors (Hickman et al. Nat Neurosci 2013). Collectively known as the microglial “sensome,” these receptors are responsible for transducing activating or activation-suppressing intracellular signaling and include protein families such as Sialic acid-binding immunoglobulin-type lectins (“SIGLEC”), Toll-like receptors (“TLR”), Fc receptors, nucleotide-binding oligomerization domain (“NOD”) and purinergic G protein-coupled receptors. Doens and Fernandez 2014, Madry and Attwell 2015, Hickman and El Khoury 2019. Similar to other cells of the myeloid lineage, the composition of microglial sensomes is dynamically regulated and acts to recognize molecular pattern that direct phenotypic responses to homeostatic changes in the central nervous system (“CNS”). Id. One of the receptors selectively expressed by brain microglia is TREM2, composed of a single-pass transmembrane domain, an extracellular stalk region, and extracellular immunoglobulin variable (“IgV”)-like domain responsible for ligand interaction (Kleinberger et al. Sci Transl Med, 2014). As TREM2 does not possess intracellular signal transduction-mediating domains, biochemical analysis has illustrated that interaction with adaptor proteins DAP10 and DAP12 mediate downstream signal transduction following ligand recognition (Peng et al. Sci Signal 2010; Jay et al. Mol Neurodegener, 2017). TREM2/DAP12 complexes in particular act as a signaling unit that can be characterized as pro-activation on microglial phenotypes in addition to peripheral macrophages and osteoclasts (Otero et al. J Immunol, 2012; Kobayashi et al. J Neurosci, 2016; Jaitin et al., Cell, 2019. In the CNS, signaling through TREM2 has been studied in the context of ligands such as phospholipids, cellular debris, apolipoproteins, and myelin (Wang et al. Cell, 2015; Kober and Brett, J Mol Biol, 2017; Shirotani et al., Sci Rep, 2019). In mice lacking functional TREM2 expression or expressing a mutated form of the receptor, a core observation is blunted microglial responses to insults such as oligodendrocyte demyelination, stroke-induced tissue damage in the brain, and proteotoxic inclusions in vivo (Cantoni et al., Acta Neuropathol, 2015, Wu et al., Mol Brain, 2017).

Coding variants in the TREM2 locus has been associated with late onset Alzheimer's disease (“LOAD”) in human genome-wide association studies, linking a loss-of-receptor function to a gain in disease risk (Jonsson et al. N Engl J Med 2013, Sims et al. Nat Genet 2017). Genetic variation of other genes selectively expressed by microglia in the CNS, for example, CD33, PLCg2 and MS4A4A/6A have reached genome-wide significance for their association with LOAD risk (Hollingworth et al. Nat Genet 2011, Sims et al. Nat Genet 2017, Deming et al. Sci Transl Med 2019). Together, these genetic findings link together in a putative biochemical circuit that highlights the importance of microglial innate immune function in LOAD. Additionally, increase or elevation in the soluble form of TREM2 (“sTREM2”) in the cerebrospinal fluid (CSF) of human subjects is associated with disease progression and emergence of pathological hallmarks of LOAD including phosphorylated Tau (Suarez-Calvet et al. Mol Neurodegener 2019). Furthermore, natural history and human biology studies indicate that baseline sTREM2 levels in the CSF can stratify the rate of temporal lobe volume loss and episodic memory decline in longitudinally monitored cohorts (Ewers et al. Sci Transl Med 2019).

In addition to human genetic evidence supporting a role of TREM12 in LOAD, homozygous loss-of-function mutations in TREM2 are causal for an early onset dementia syndrome known as Polycystic lipomembranous osteodysplasia with sclerosing leukoencephalopathy (“PLOSL”) or Nasu-Hakola disease (“NHD”) (Golde et al. Alzheimers Res Ther 2013, Dardiotis et al. Neurobiol Aging 2017). This progressive neurodegenerative disease typically manifests in the 3^{rd} decade of life and is pathologically characterized by loss of myelin in the brain concomitant with gliosis, unresolved neuroinflammation, and cerebral atrophy. Typical neuropsychiatric presentations are often preceded by osseous abnormalities, such as bone cysts and loss of peripheral bone density (Bianchin et al. Cell Mol Neurobiol 2004; Madry et al. Clin Orthop Relat Res 2007, Bianchin et al. Nat Rev Neurol 2010). Given that osteoclasts of the myeloid lineage are also known to express TREM2, the PLOSL-related symptoms of wrist and ankle pain, swelling, and fractures indicate that TREM2 may act to regulate bone homeostasis through defined signaling pathways that parallel the microglia in the CNS (Paloneva et al. J Exp Med 2003, Otero et al. J Immunol 2012). The link between TREM2 function and PLOSL has illustrated the importance of the receptor in sustaining key physiological aspects of myeloid cell function in the human body.

Efforts have been made to model the biology of TREM2 in mice prompting the creation of TREM2 knock out (“KO”) mice in addition to the LOAD-relevant TREM2 R47H loss-of-function mutant transgenic mice (Ulland et al. Cell, 2017, Kang et al. Hum Mol Genet 2018). Although unable to recapitulate the neurological manifestations of PLOSL, TREM12 KO mice show abnormalities in bone ultrastructure (Otero et al. J Immunol 2012). When the TREM2 KO or mutant mice have been crossed onto familial Alzheimer's disease transgenic mouse background such as the 5XFAD amyloidogenic mutation lines, marked phenotypes have been observed (Ulrich et al. Neuron, 2017). These in vivo phenotypes of TREM2 loss-of-function in the CNS include elevated the plaque burden and lower levels of secreted microglial factors SPP1 and Osteopontin that are characteristic of the microglial response to amyloid pathology (Ulland et al. Cell, 2017). Other rodent studies have demonstrated that loss of TREM2 leads to decreased microglial clustering around plaques and emergence of less compact plaque morphology in familial AD amyloid models (Parhizkar et al. Nat Neurosci 2019). With regards to the Tau protein pathology that is observed in LOAD, familial tauopathy models in mice demonstrated an enhanced spreading of pathological human Tau aggregates from point of injection into mouse brain in TREM2 KO mice (Leyns et al. Nat Neurosci 2019). Furthermore, single-cell RNASeq studies with the TREM2 KO mice in aged scenarios, 5XFAD familial Alzheimer's disease model mice, and Amyotrophic Lateral Sclerosis SOD1 mutant mouse backgrounds indicate that TREM2 receptor function is critical for a conserved set of phenotypic transformations within microglial populations in response to CNS pathology (Keren-Shaul et al. Cell 2017).

In rodent models where TREM2 expression levels are elevated, brain amyloid pathology in the 5XFAD transgenic mice displayed reduced plaque volume and altered morphology (Lee et al. Neuron, 2018). The changes in immunohistological markers relating to brain amyloid pathology were also accompanied by an attenuated presence of dystrophic neurites when TREM12 was overexpressed. Id. Therefore, the pharmacological activation of TREM2 is a target of interest for treating or preventing neurological, neurodegenerative and other diseases. Despite many attempts to alter disease progression by targeting the pathological hallmarks of LOAD through anti-amyloid and anti-Tau therapeutics, there is a need for activators of TREM2 to address the genetics-implicated neuroimmune aspects of, for example, LOAD. Such TREM2 activators may be suitable for use as therapeutic agents and remain in view of the significant continuing societal burden that remains unmitigated for diseases.

### SUMMARY

First, provided herein is a compound of Formula (I):

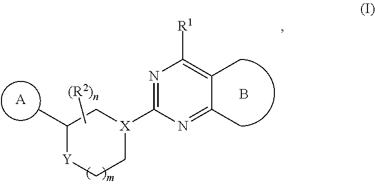

or a pharmaceutically acceptable salt, solvate, stereoisomer, or tautomer thereof, or a pharmaceutically acceptable salt and/or solvate of said compound, stereoisomer, or tautomer, wherein:
Ring A is selected from cycloalkyl, heterocyclyl, aryl, or 5- to 9-membered heteroaryl, wherein each cycloalkyl, heterocyclyl, aryl, and heteroaryl is optionally substituted with one or more R³; wherein when Ring A is a 6-membered heteroaryl, the 6-membered heteroaryl is

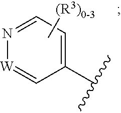

Ring B together with the 6-membered ring system to which it is fused forms a bicyclic ring system selected from one of:

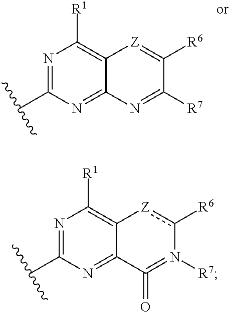

W is C(R⁵) or N;
X is C(R⁵) or N;
Y is C(R¹¹)(R¹²) or O;
Z is selected from C(R¹³), C(R¹³)(R¹⁴), or N, wherein the dashed bond comprises a single or double bond depending on valency;
R¹ is selected from cycloalkyl, heterocyclyl, aryl, or heteroaryl, wherein each cycloalkyl, heterocyclyl, aryl, and heteroaryl is optionally substituted with one or more R⁴;
each R² is independently C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), —N(R^{B})(R^{C}), wherein each alkyl, alkenyl, alkynyl, heteroalkyl, haloalkyl, cycloalkyl, and heterocyclyl is optionally substituted with one or more R⁵; or
two R², taken together with the carbon atom to which they are attached, form an oxo group;
each R³ and R⁴ is independently deuterium, C₁₋₆ alkyl, C₂₋₆ alkenyl, C₂₋₆ alkynyl, C₁₋₆ heteroalkyl, C₁₋₆haloalkyl, cycloalkyl, heterocyclyl, halogen, cyano, oxo, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R⁸;
R⁵ is hydrogen, C₁₋₆ alkyl, C₂₋₆ alkenyl, C₂₋₆ alkynyl, C₁₋₆ heteroalkyl, C₁₋₆ haloalkyl, halogen, cyano, —O(R^{A}), or —N(R^{B})(R^{C}), wherein each alkyl, alkenyl, alkynyl, heteroalkyl, and haloalkyl is optionally substituted with one or more R⁸;
R⁶ and R⁹ are each independently hydrogen, C₁₋₆ alkyl, C₂₋₆ alkenyl, C₂₋₆ alkynyl, C₁₋₆ heteroalkyl, C₁₋₆haloalkyl, cycloalkyl, heterocyclyl, aryl, heteroaryl, halogen, cyano, —O(R^{A}), —C(O)(R^{A}), —C(O)O(R^{A}), —C(O)N(R^{B})(R^{C}), —C(═N—OR^{A})(R^{C}), —N(R^{B})(R^{C}), —N(O)(R^{A}), or —S(O)ₓ(R^{C}), wherein alkyl, alkenyl, alkynyl, heteroalkyl, haloalkyl, cycloalkyl, heterocyclyl, aryl, and heteroaryl is optionally substituted with one or more R⁹;
each R⁸ and R⁹ is independently deuterium, C₁₋₆ alkyl, C₂₋₆ alkenyl, C₂₋₆ alkynyl, C₁₋₆ heteroalkyl, C₁₋₆haloalkyl, cycloalkyl, heterocyclyl, halogen, cyano, —C(O)O(R^{A}), —O(R^{A}), or —N(R^{B})(R^{C}), wherein each alkyl, alkenyl, alkynyl, heteroalkyl, haloalkyl, cycloalkyl, and heterocyclyl is optionally substituted with one or more R¹⁰;
each R^{A} is independently hydrogen, deuterium, C₁₋₆ alkyl, C₁₋₆ heteroalkyl, C₁₋₆haloalkyl, cycloalkyl, heterocyclyl, or —N(R^{B})(R^{C}), wherein each alkyl, heteroalkyl, haloalkyl, cycloalkyl, and heterocyclyl is optionally substituted with one or more R¹⁷;
each R^{B} and R^{C} is independently hydrogen, C₁₋₆ alkyl, C₁₋₆ heteroalkyl, C₁₋₆haloalkyl, cycloalkyl, heterocyclyl, or —C(O)-alkyl, wherein each alkyl, heteroalkyl, haloalkyl, cycloalkyl, and heterocyclyl is optionally substituted with one or more R¹⁷;
each R¹⁰ is deuterium, C₁₋₆ alkyl, C₁₋₆ heteroalkyl, C₁₋₆ haloalkyl, cycloalkyl, halogen, or cyano;
each R¹¹ and R¹² is selected from hydrogen, C₁₋₆ alkyl, C₂₋₆ alkenyl, C₂₋₆ alkynyl, C₁₋₆ heteroalkyl, C₁₋₆ haloalkyl, halogen, cyano, —O(R^{A}), or —N(R^{B})(R^{C}), wherein each alkyl, alkenyl, alkynyl, heteroalkyl, and haloalkyl is optionally substituted with one or more R¹⁷;
each R¹⁷ is selected from deuterium, C₁₋₆ alkyl, C₁₋₆ heteroalkyl, C₁₋₆ haloalkyl, cycloalkyl, halogen, or cyano;
m is 0, 1, or 2;
n is 0, 1, 2, 3, 4, 5, or 6; and
x is 0, 1, or 2.

Second, provided herein is a pharmaceutical composition comprising a compound of Formula (I), or a tautomer thereof, or a pharmaceutically acceptable salt of said compound or said tautomer, and a pharmaceutically acceptable excipient.

Third, provided herein is a compound of Formula (I), or a tautomer thereof, or a pharmaceutically acceptable salt of said compound or said tautomer, or a pharmaceutical composition as described hereinabove, for use in treating or preventing a condition associated with a loss of function of human TREM2.

Fourth, provided herein is a compound of Formula (I), or a tautomer thereof, or a pharmaceutically acceptable salt of said compound or said tautomer, or a pharmaceutical composition described hereinabove, for use in treating or preventing Parkinson's disease, rheumatoid arthritis, Alzheimer's disease, Nasu-Hakola disease, frontotemporal dementia, multiple sclerosis, prion disease, or stroke.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### DETAILED DESCRIPTION

Provided herein is a compound of Formula (I):

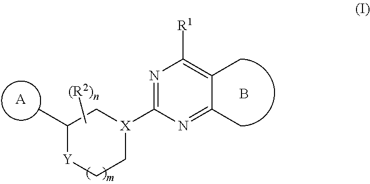

or a pharmaceutically acceptable salt, solvate, stereoisomer, or tautomer thereof, or a pharmaceutically acceptable salt and/or solvate of said compound, stereoisomer, or tautomer, wherein:
Ring A is selected from cycloalkyl, heterocyclyl, aryl, or 5- to 9-membered heteroaryl, wherein each cycloalkyl, heterocyclyl, aryl, and heteroaryl is optionally substituted with one or more R³;
wherein when Ring A is a 6-membered heteroaryl, the 6-membered heteroaryl is

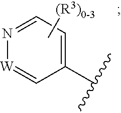

Ring B together with the 6-membered ring system to which it is fused forms a bicyclic ring system selected from one of:

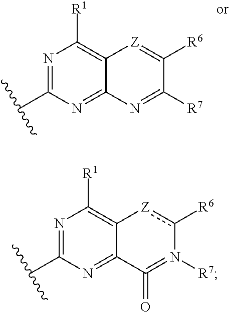

W is C(R⁵) or N;
X is C(R⁵) or N;
Y is C(R¹¹)(R¹²) or O;
Z is selected from C(R¹³), C(R¹³)(R¹⁴), or N, wherein the dashed bond comprises a single or double bond depending on valency;
R¹ is selected from cycloalkyl, heterocyclyl, aryl, or heteroaryl, wherein each cycloalkyl, heterocyclyl, aryl, and heteroaryl is optionally substituted with one or more R⁵;
each R² is independently C₁₋₆ alkyl, C₂₋₆ alkenyl, C₂₋₆ alkynyl, C₁₋₆ heteroalkyl, C₁₋₆haloalkyl, cycloalkyl, heterocyclyl, halogen, cyano, —O(R^{A}), —N(R^{B})(R^{C}), wherein each alkyl, alkenyl, alkynyl, heteroalkyl, haloalkyl, cycloalkyl, and heterocyclyl is optionally substituted with one or more R⁵; or
two R², taken together with the carbon atom to which they are attached, form an oxo group;
each R³ and R⁴ is independently deuterium, C₁₋₆ alkyl, C₂₋₆ alkenyl, C₂₋₆ alkynyl, C₁₋₆ heteroalkyl, C₁₋₆haloalkyl, cycloalkyl, heterocyclyl, halogen, cyano, oxo, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R⁸;
R⁵ is hydrogen, C₁₋₆ alkyl, C₂₋₆ alkenyl, C₂₋₆ alkynyl, C₁₋₆ heteroalkyl, C₁₋₆ haloalkyl, halogen, cyano, —O(R^{A}), or —N(R^{B})(R^{C}), wherein each alkyl, alkenyl, alkynyl, heteroalkyl, and haloalkyl is optionally substituted with one or more R⁸;
R⁶ and R⁹ are each independently hydrogen, C₁₋₆ alkyl, C₂₋₆ alkenyl, C₂₋₆ alkynyl, C₁₋₆ heteroalkyl, C₁₋₆haloalkyl, cycloalkyl, heterocyclyl, aryl, heteroaryl, halogen, cyano, —O(R^{A}), —C(O)(R^{A}), —C(O)O(R^{A}), —C(O)N(R^{B})(R^{C}), —C(═N-OR^{A})(R^{C}), —N(R^{B})(R^{C}), —N(O)(R^{A}), or —S(O)ₓ(R^{C}), wherein alkyl, alkenyl, alkynyl, heteroalkyl, haloalkyl, cycloalkyl, heterocyclyl, aryl, and heteroaryl is optionally substituted with one or more R⁹;
each R⁸ and R⁹ is independently deuterium, C₁₋₆ alkyl, C₂₋₆ alkenyl, C₂₋₆ alkynyl, C₁₋₆ heteroalkyl, C₁₋₆haloalkyl, cycloalkyl, heterocyclyl, halogen, cyano, —C(O)O(R^{A}), —O(R^{A}), or —N(R^{B})(R^{C}), wherein each alkyl, alkenyl, alkynyl, heteroalkyl, haloalkyl, cycloalkyl, and heterocyclyl is optionally substituted with one or more R¹⁰;
each R^{A} is independently hydrogen, deuterium, C₁₋₆ alkyl, C₁₋₆ heteroalkyl, C₁₋₆haloalkyl, cycloalkyl, heterocyclyl, or —N(R^{B})(R^{C}), wherein each alkyl, heteroalkyl, haloalkyl, cycloalkyl, and heterocyclyl is optionally substituted with one or more R¹¹;
each R^{B} and Re is independently hydrogen, C₁₋₆ alkyl, C₁₋₆ heteroalkyl, C₁₋₆ haloalkyl, cycloalkyl, heterocyclyl, or —C(O)-alkyl, wherein each alkyl, heteroalkyl, haloalkyl, cycloalkyl, and heterocyclyl is optionally substituted with one or more R¹⁷;
each R¹⁰ is deuterium, C₁₋₆ alkyl, C₁₋₆ heteroalkyl, C₁₋₆ haloalkyl, cycloalkyl, halogen, or cyano;
each R¹¹ and R¹² is selected from hydrogen, C₁₋₆ alkyl, C₂₋₆ alkenyl, C₂₋₆ alkynyl, C₁₋₆ heteroalkyl, C₁₋₆ haloalkyl, halogen, cyano, —O(R^{A}), or —N(R^{B})(R^{C}), wherein each alkyl, alkenyl, alkynyl, heteroalkyl, and haloalkyl is optionally substituted with one or more R¹⁷;
each R¹⁷ is selected from deuterium, C₁₋₆ alkyl, C₁₋₆ heteroalkyl, C₁₋₆ haloalkyl, cycloalkyl, halogen, or cyano;
m is 0, 1, or 2;
n is 0, 1, 2, 3, 4, 5, or 6; and
x is 0, 1, or 2.

In some embodiments, the compound of Formula (I) is a compound of Formula (I-a):

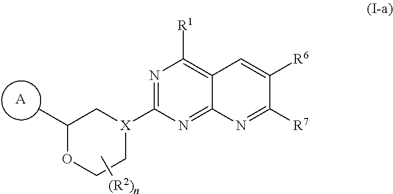

or a pharmaceutically acceptable salt, solvate, stereoisomer, or tautomer thereof, or a pharmaceutically acceptable salt and/or solvate of said compound, stereoisomer, or tautomer, wherein:
Ring A is cycloalkyl, heterocyclyl, aryl, heteroaryl, each of which is optionally substituted with one or more R³;
X is C(R⁵) or N;
R¹ is cycloalkyl, heterocyclyl, aryl, or heteroaryl, each of which is optionally substituted with one or more R⁵;
each R² is independently C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), —N(R^{B})(R^{C}), wherein each alkyl, alkenyl, alkynyl, heteroalkyl, haloalkyl, cycloalkyl, and heterocyclyl is optionally substituted with one or more R⁵; or
two R², taken together with the carbon atom to which they are attached, form an oxo group;
each R³ and R⁴ is independently deuterium, C₁₋₆ alkyl, C₂₋₆ alkenyl, C₂₋₆ alkynyl, C₁₋₆ heteroalkyl, C₁₋₆haloalkyl, cycloalkyl, heterocyclyl, halogen, cyano, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R⁸;
R⁵ is hydrogen, C₁₋₆ alkyl, C₂₋₆ alkenyl, C₂₋₆ alkynyl, C₁₋₆ heteroalkyl, C₁₋₆ haloalkyl, halogen, cyano, —O(R^{A}), or —N(R^{B})(R^{C}), wherein each alkyl, alkenyl, alkynyl, heteroalkyl, and haloalkyl is optionally substituted with one or more R⁸;
R⁶ and R⁹ are each independently hydrogen, C₁₋₆ alkyl, C₂₋₆ alkenyl, C₂₋₆ alkynyl, C₁₋₆ heteroalkyl, C₁₋₆haloalkyl, cycloalkyl, heterocyclyl, aryl, heteroaryl, halogen, cyano, —O(R^{A}), —C(O)(R^{A}), —C(O)O(R^{A}), —N(R^{B})(R^{C}), —N(O)(R^{A}), or —S(O)ₓ(R^{C}), wherein alkyl, alkenyl, alkynyl, heteroalkyl, haloalkyl, cycloalkyl, heterocyclyl, aryl, and heteroaryl is optionally substituted with one or more R⁹;
each R⁸ and R⁹ is independently deuterium, C₁₋₆ alkyl, C₂₋₆ alkenyl, C₂₋₆ alkynyl, C₁₋₆ heteroalkyl, C₁₋₆haloalkyl, cycloalkyl, heterocyclyl, halogen, cyano, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R¹⁰;
each R^{A} is independently hydrogen, deuterium, C₁₋₆ alkyl, C₁₋₆ heteroalkyl, C₁₋₆haloalkyl, cycloalkyl, heterocyclyl, or —N(R^{B})(R^{C}), wherein each alkyl, heteroalkyl, haloalkyl, cycloalkyl, and heterocyclyl is optionally substituted with one or more R¹⁷;
each R^{B} and R^{C} is independently hydrogen, C₁₋₆ alkyl, C₁₋₆ heteroalkyl, C₁₋₆ haloalkyl, cycloalkyl, heterocyclyl, or —C(O)-alkyl, wherein each alkyl, heteroalkyl, haloalkyl, cycloalkyl, and heterocyclyl is optionally substituted with one or more R¹⁷;
each R¹⁰ is deuterium, C₁₋₆ alkyl, C₁₋₆ heteroalkyl, C₁₋₆ haloalkyl, cycloalkyl, halogen, or cyano; n is 0, 1, 2, 3, 4, 5, or 6; and
x is 0, 1, or 2.

As generally described herein, Ring A is selected from cycloalkyl, heterocyclyl, aryl, or heteroaryl, each of which is optionally substituted with one or more R³. In some embodiments, Ring A is heteroaryl, optionally substituted with one or more R³. In some embodiments, Ring A is selected from:

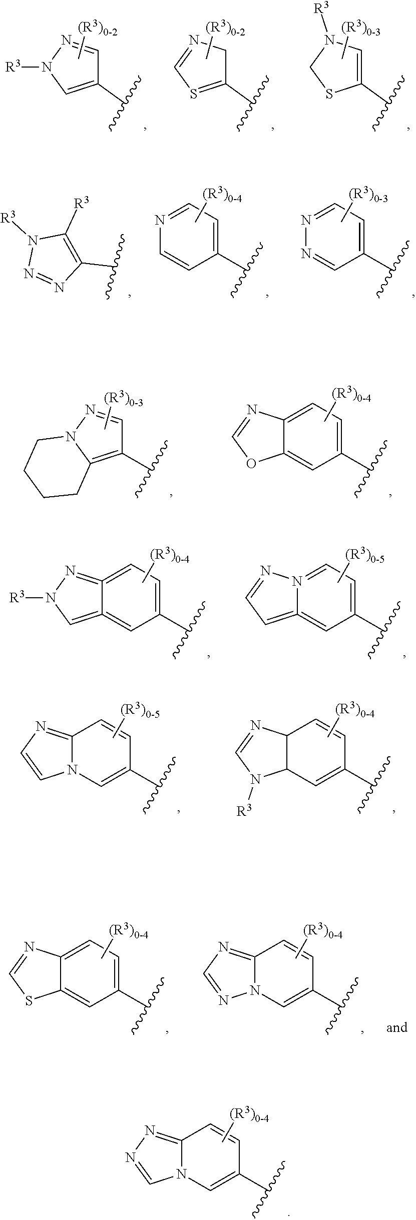

and In some embodiments, Ring A is selected

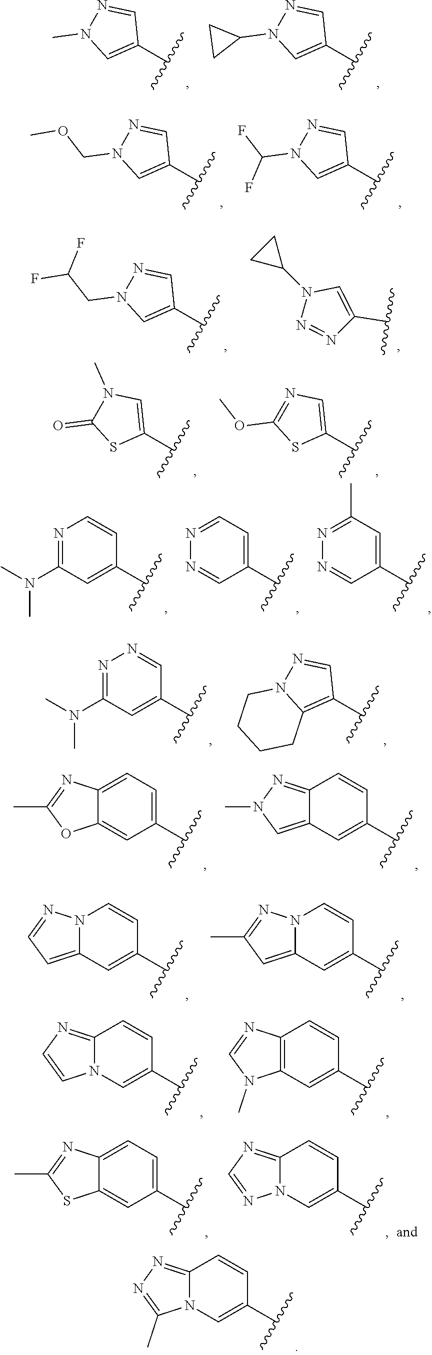

In some embodiments, R¹ is a 4-membered, 5-membered, or 6-membered cycloalkyl, each of which is optionally substituted with one or more R⁴. In some embodiments, R¹ is selected from cyclopropyl, cyclopentyl, or cyclohexyl, each of which is optionally substituted with one or more R⁴.

In some embodiments, R¹ is selected from: cyclohexyl, cyclopentenyl, cyclobutyl, 3-(trifluoromethyl)-1-bicyclo[1.1.1]pentanyl, 3-(difluoromethyl)bicyclo[1.1.1]pentan-1-yl), bicyclo[1.1.1]pentane-1-carbonitrile, 4-(trifluoromethyl)-cuban-1-yl, cubanyl, 1-bicyclo[1.1.1]pentanyl, or 3-(difluoromethyl)bicyclo[1.1.1]pentan-1-yl.

In some embodiments, R¹ is selected from cyclohexyl, 4,4-difluoro-cyclohexyl, 4-fluoro-cyclohexyl, 4-fluoromethyl-cyclohexyl, (spiro)-cyclopropyl-cyclopropyl-F2, bicyclo[1.1.1]-trifluoromethyl, bicyclo[1.1.1]-difluoromethyl, bicyclo[1.1.1]—CF—(CH₃)₂, bicyclo[1.1.1]—CH₂—CF₃, bicyclo[1.1.1]—CH₂—CHF₂, bicyclo[1.1.1]—CF₂—CH₃, bicyclo[1.1.1]-chloro, or bicyclo[2.2.1]—CF₂—CH₃.

In some embodiments, R¹ is a substituent selected from those shown below:

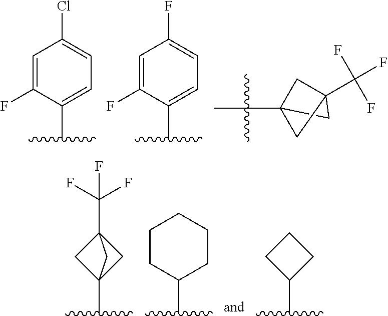

In some embodiments, R¹ is

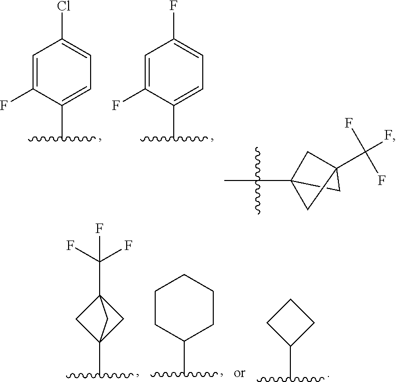

cycloalkyl, optionally substituted with one or more R⁴. In some embodiments, R¹ is selected from

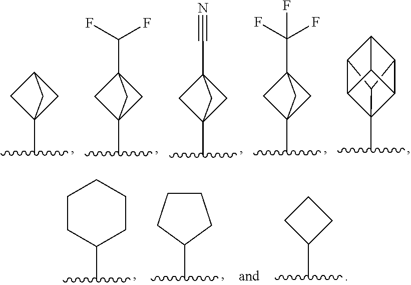

In some embodiments, R¹ is aryl or heteroaryl, optionally substituted with one or more R⁴. In some embodiments, R¹ is selected from

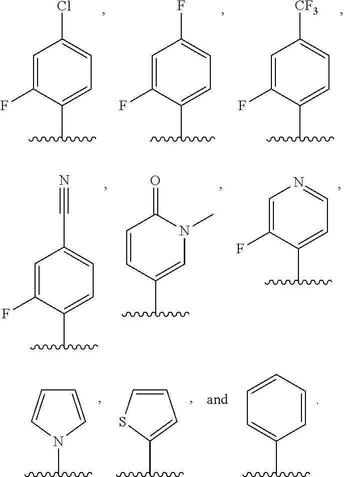

In some embodiments, R⁴ is deuterium, C₁₋₆ alkyl, C₂₋₆ alkenyl, C₂₋₆ alkynyl, C₁₋₆ heteroalkyl, C₁₋₆haloalkyl, cycloalkyl, heterocyclyl, halogen, cyano, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R⁸. In some embodiments, R⁴ is deuterium, C₁₋₆ alkyl, C₁₋₆ heteroalkyl, C₁₋₆ haloalkyl, or halogen.

In some embodiments, at least one hydrogen atom of the compound is a deuterium atom. In some embodiments, at least one C₁-C₆alkyl group of the compound is substituted with at least one deuterium atom. In some embodiments, R⁴ is substituted with C₁₋₃alkyl, comprising one or more deuteriums. In some embodiments, R⁴ is substituted with 1 to 3 substitutents selected from —CD₃, —CHD₂, and —CH₂D.

In some embodiments, R⁴ is selected from those depicted in in Table A below.

In some embodiments, R² is independently C₁₋₆ alkyl, C₂₋₆ alkenyl, C₂₋₆ alkynyl, C₁₋₆ heteroalkyl, C₁₋₆haloalkyl, C₃₋₁₂ cycloalkyl, heterocyclyl, halogen, cyano, —O(R^{A}), —N(R^{B})(R^{C}), wherein each alkyl, alkenyl, alkynyl, heteroalkyl, haloalkyl, cycloalkyl, and heterocyclyl is optionally substituted with one or more R⁵; or two R², taken together with the carbon atom to which they are attached, form an oxo group.

In some embodiments, at least one hydrogen atom of the compound is a deuterium atom. In some embodiments, at least one C₁-C₆alkyl group of the compound is substituted with at least one deuterium atom. In some embodiments, R² is deuterium. In some embodiments, the hydrogen atom attached to the same carbon as R² is deuterium.

In some embodiments, R² is selected from those depicted in in Table A below.

In some embodiments, Ring A is heteroaryl substituted with one or more R³. In some embodiments, Ring A is pyrazolyl substituted with one or more R³. In some embodiments, Ring A is heterocyclyl substituted with one or more R³. In some embodiments, Ring A is pyridonyl substituted with one or more R³. In some embodiments, Ring A is selected from those depicted in in Table A below.

In some embodiments, R³ is deuterium, C₁₋₆ alkyl, C₂₋₆ alkenyl, C₂₋₆ alkynyl, C₁₋₆ heteroalkyl, C₁₋₆haloalkyl, cycloalkyl, heterocyclyl, halogen, cyano, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R⁸. In some embodiments, R³ is deuterium, C₁₋₆ alkyl, C₁₋₆ heteroalkyl, C₁₋₆ haloalkyl, cycloalkyl, halogen. In some embodiments, R³ is cycloalkyl (e.g., cyclopropyl).

In some embodiments, R³ is methyl,

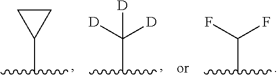

In some embodiments, R³ is substituted with C₁₋₃alkyl, comprising one or more deuteriums. In some embodiments, R³ is substituted with 1 to 3 substitutents selected from —CD₃, —CHD₂, and —CH₂D.

In some embodiments, R³ is selected from those depicted in Table A below.

In some embodiments, R⁶ and R⁹ are each independently hydrogen, C₁₋₆ alkyl, C₁₋₆ heteroalkyl, C₁₋₆haloalkyl, cycloalkyl, heterocyclyl, halogen, cyano, —O(R^{A}), —C(O)(R^{A}), —C(O)O(R^{A}), or —S(O)ₓ(R^{C}), wherein alkyl, heteroalkyl, haloalkyl, cycloalkyl, and heterocyclyl is optionally substituted with one or more R⁹.

In some embodiments, R⁶ is heterocyclyl, heteroaryl, halogen, or cyano, —O(R^{A}), —C(O)(R^{A}), —C(O)O(R^{A}), —N(R^{B})(R^{C}), —N(O)(R^{A}), or —S(O)ₓ(R^{C}), and R⁹ is hydrogen or C₁₋₆alkyl (e.g., CH₃). In some embodiments, R⁶ is —O(R^{A}), —C(O)(R^{A}), —C(O)O(R^{A}), —N(O)(R^{A}), or —S(O)ₓ(R^{C}), and R⁹ is hydrogen or C₁₋₆ alkyl (e.g., CH₃).

In some embodiments R⁶ is a substituent selected from those shown below:

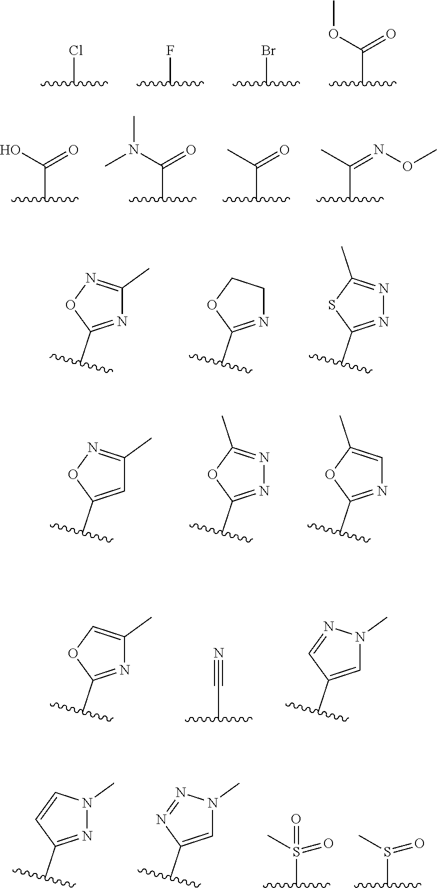

and R⁷ is hydrogen or C₁₋₆ alkyl.

In some embodiments, R⁶ is heterocyclyl, heteroaryl, halogen, or cyano and R⁷ is hydrogen or C₁₋₆ alkyl (e.g., CH₃).

In some embodiments, R⁶ is halogen or cyano and R⁹ is hydrogen or C₁₋₆ alkyl (e.g., CH₃).

In some embodiments, R⁶ is selected from

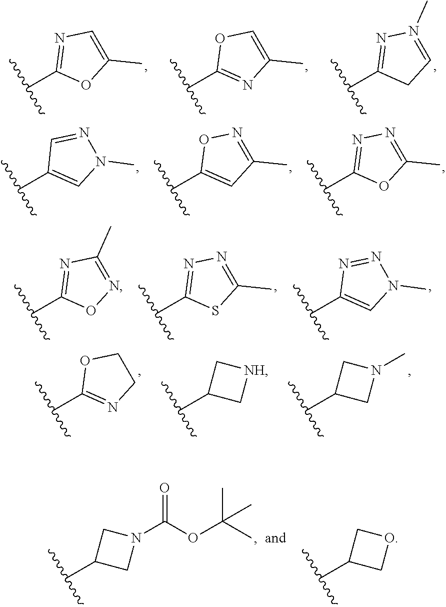

In some embodiments, R⁹ is C₁₋₆ alkyl (e.g., CH₃).

In some embodiments, at least one hydrogen atom of the compound is a deuterium atom. In some embodiments, at least one C₁-C₆alkyl group of the compound is substituted with at least one deuterium atom. In some embodiments, R⁶ is —CD₃. In some embodiments, R⁹ is —CD₃. In some embodiments, R⁶ and R⁹ are both —CD₃. In some embodiments, R⁶ and R⁹ are each independently selected from H, D, —CH₃, —CD₃, —CHD₂, and —CH₂D. In some embodiments, R⁶ and R⁹ are each independently selected from —CH₃, —CD₃, —CHD₂, and —CH₂D. In some embodiments, R² is deuterium. In some embodiments, the hydrogen atom attached to the same carbon as R² is deuterium. In some embodiments, R⁴ is substituted with C₁₋₃alkyl, comprising one or more deuteriums. In some embodiments, R⁴ is substituted with 1 to 3 substitutents selected from —CD₃, —CHD₂, and —CH₂D.

In some embodiments, R⁶ and R⁹ is selected from those depicted in in Table A below.

In some embodiments, X is C(R⁵) (e.g., CH₃). In some embodiments, X is N. In some embodiments, X is selected from those depicted in in Table A below.

In some embodiments, Y is O. In some embodiments, Y is C(R¹¹)(R¹²) (e.g., CH₂). In some embodiments, Y is CH₂.

In some embodiments, W is N. In some embodiments, W is C(R⁵) (e.g., CH).

In some embodiments, Z is selected from C(R¹³) (e.g., CH) or N. In some embodiments, Z is C(R¹³). In some embodiments, Z is N.

In some embodiments, m is 0 or 1. In some embodiments, m is 1.

In some embodiments, n is 0 or 1.

In some embodiments, Ring B together with the 6-membered ring system to which it is fused forms a bicyclic ring system selected from

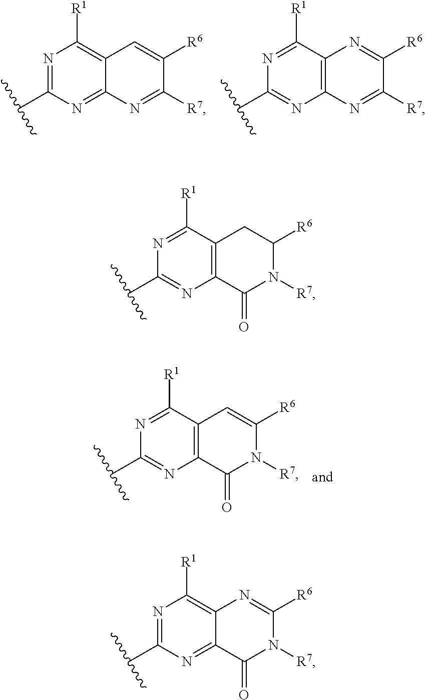

In some embodiments, the compound of

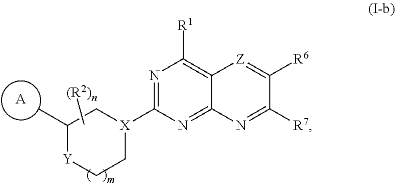

or a pharmaceutically acceptable salt, solvate, stereoisomer, or tautomer thereof, or a pharmaceutically acceptable salt and/or solvate of said compound, stereoisomer, or tautomer, wherein each of Ring A, R¹, R², R⁶, R⁷, X, Y, Z, m, n, and subvariables thereof are defined as for Formula (I).

In some embodiments, the compound of Formula (I) is a compound of Formula (I-c):

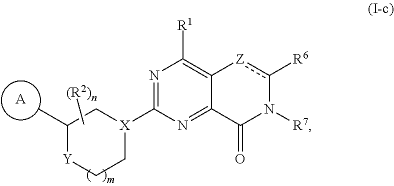

or a pharmaceutically acceptable salt, solvate, stereoisomer, or tautomer thereof, or a pharmaceutically acceptable salt and/or solvate of said compound, stereoisomer, or tautomer, wherein each of Ring A, R¹, R², R⁶, R⁷, X, Y, Z, m, n, and subvariables thereof are defined as for Formula (I).

In some embodiments, the compound of Formula (I) is a compound of Formula (I-d)

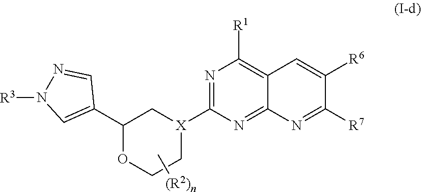

or a pharmaceutically acceptable salt, solvate, stereoisomer, or tautomer thereof, or a pharmaceutically acceptable salt and/or solvate of said compound, stereoisomer, or tautomer, wherein each of R¹, R², R³, R⁶, R⁷, n and subvariables thereof are defined as for Formula (I).

In some embodiments, the compound of Formula (I) is a compound of Formula (I-e)

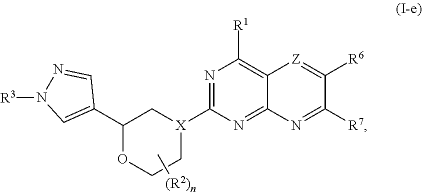

or a pharmaceutically acceptable salt, solvate, stereoisomer, or tautomer thereof, or a pharmaceutically acceptable salt and/or solvate of said compound, stereoisomer, or tautomer, wherein each of R¹, R², R³, R⁶, R⁷, Z, n and subvariables thereof are as defined for Formula (I).

In some embodiments, the compound of Formula (I) is a compound of Formula (I-f)

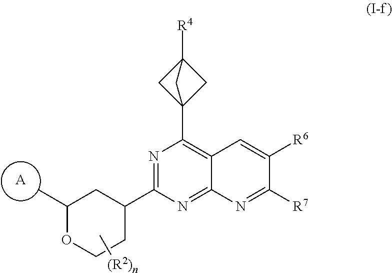

or a pharmaceutically acceptable salt, solvate, stereoisomer, or tautomer thereof, or a pharmaceutically acceptable salt and/or solvate of said compound, stereoisomer, or tautomer, wherein each of R², R⁴, R⁶, R⁷, n and subvariables thereof are defined as for Formula (I).

In some embodiments, the compound of Formula (I) is a compound of Formula (I-g)

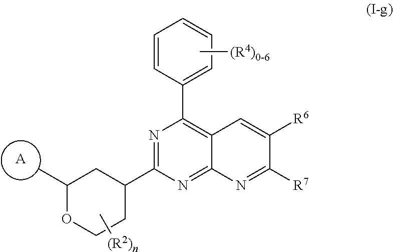

or a pharmaceutically acceptable salt, solvate, stereoisomer, or tautomer thereof, or a pharmaceutically acceptable salt and/or solvate of said compound, stereoisomer, or tautomer, wherein each of R², R⁴, R⁶, R⁷, n and subvariables thereof are defined as for Formula (I).

In some embodiments, the compound of Formula (I) is a compound of Formula (I-h)

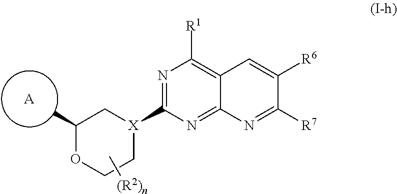

or a pharmaceutically acceptable salt, solvate, stereoisomer, or tautomer thereof, or a pharmaceutically acceptable salt and/or solvate of said compound, stereoisomer, or tautomer, wherein each of R¹, R², R⁶, R⁷, n and subvariables thereof are defined as for Formula (I).

In some embodiments, the compound of Formula (I) is a compound of Formula (I-i)

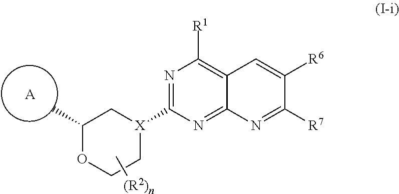

or a pharmaceutically acceptable salt, solvate, stereoisomer, or tautomer thereof, or a pharmaceutically acceptable salt and/or solvate of said compound, stereoisomer, or tautomer, wherein each of R², R¹³, n and subvariables thereof are defined as for Formula (I).

In some embodiments, the compound of Formula (I) is a compound of Formula (I-j)

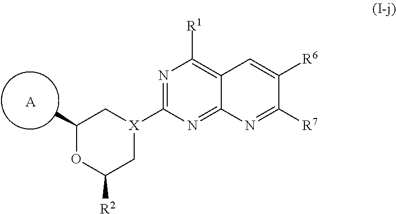

or a pharmaceutically acceptable salt, solvate, stereoisomer, or tautomer thereof, or a pharmaceutically acceptable salt and/or solvate of said compound, stereoisomer, or tautomer, wherein each of R¹, R², R⁶, R⁷, n and subvariables thereof are defined as for Formula (I).

In some embodiments, the compound is a compound provided in Table A.

Further provided herein is a pharmaceutical composition comprising one or more of the compounds disclosed herein and a pharmaceutically acceptable excipient.

In some embodiments, the compound is a compound of any of the previous embodiments, or a pharmaceutical composition of the previous embodiments, for use in treating or preventing a condition associated with a loss of function of human TREM2.

Further provided herein is a method of treating or preventing a condition associated with a loss of function of human TREM2 in a subject in need thereof, the method comprising administering to the subject a therapeutically effective amount of the compound according to any one of the previous embodiments, or a pharmaceutical composition of the previous embodiments.

TABLE A
Exemplary Compounds

| Ex # | Structure |
|---|---|
| | 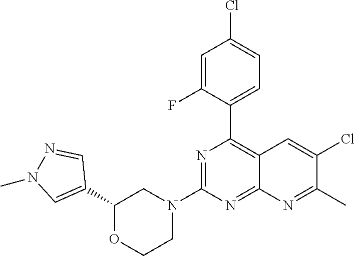 [SMILES: Cc1nc2nc(N3CCO[C@H](c4cnn(C)c4)C3)nc(-c3ccc(Cl)cc3F)c2cc1Cl] |
| 1 | 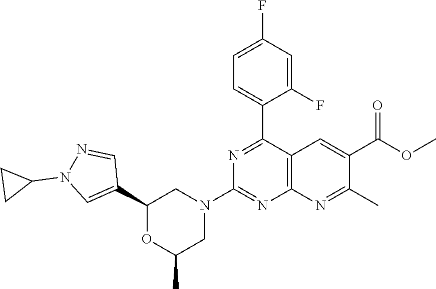 [SMILES: COC(=O)c1cc2c(-c3ccc(F)cc3F)nc(N3C[C@@H](C)O[C@@H](c4cnn(C5CC5)c4)C3)nc2nc1C] |
| 2 | 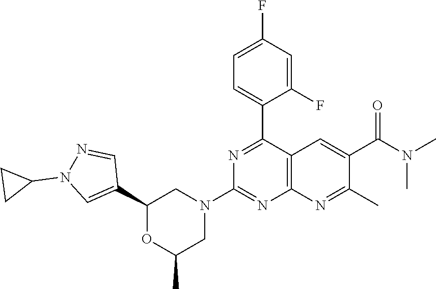 [SMILES: Cc1nc2nc(N3C[C@@H](C)O[C@@H](c4cnn(C5CC5)c4)C3)nc(-c3ccc(F)cc3F)c2cc1C(=O)N(C)C] |
| 3 | 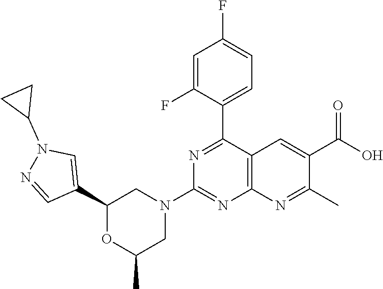 [SMILES: Cc1nc2nc(N3C[C@@H](C)O[C@@H](c4cnn(C5CC5)c4)C3)nc(-c3ccc(F)cc3F)c2cc1C(=O)O] |
| 4 | 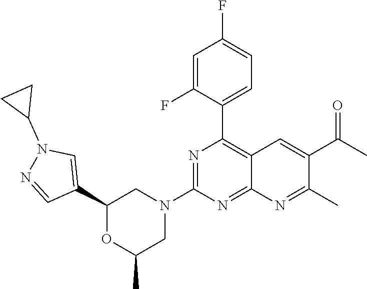 [SMILES: CC(=O)c1cc2c(-c3ccc(F)cc3F)nc(N3C[C@@H](C)O[C@@H](c4cnn(C5CC5)c4)C3)nc2nc1C] |
| 5 | 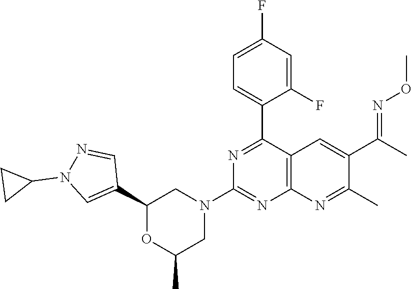 [SMILES: CO/N=C(\C)c1cc2c(-c3ccc(F)cc3F)nc(N3C[C@@H](C)O[C@@H](c4cnn(C5CC5)c4)C3)nc2nc1C] |
| 6 | 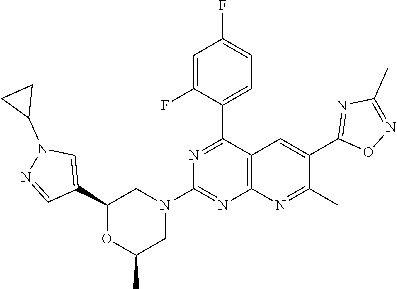 [SMILES: Cc1noc(-c2cc3c(-c4ccc(F)cc4F)nc(N4C[C@@H](C)O[C@@H](c5cnn(C6CC6)c5)C4)nc3nc2C)n1] |
| 7 | 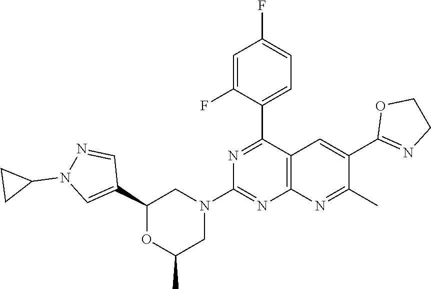 [SMILES: Cc1nc2nc(N3C[C@@H](C)O[C@@H](c4cnn(C5CC5)c4)C3)nc(-c3ccc(F)cc3F)c2cc1C1=NCCO1] |
| 8 | 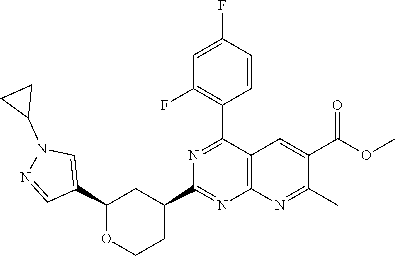 [SMILES: COC(=O)c1cc2c(-c3ccc(F)cc3F)nc([C@H]3CCO[C@@H](c4cnn(C5CC5)c4)C3)nc2nc1C] |
| 9 | 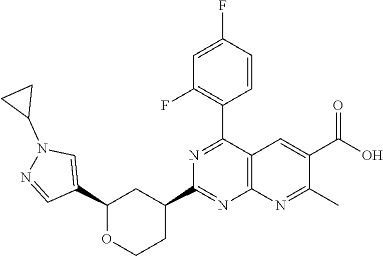 [SMILES: Cc1nc2nc([C@H]3CCO[C@@H](c4cnn(C5CC5)c4)C3)nc(-c3ccc(F)cc3F)c2cc1C(=O)O] |
| 10 | 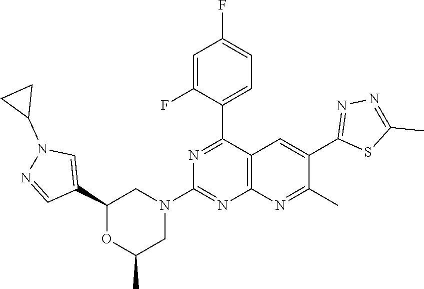 [SMILES: Cc1nnc(-c2cc3c(-c4ccc(F)cc4F)nc(N4C[C@@H](C)O[C@@H](c5cnn(C6CC6)c5)C4)nc3nc2C)s1] |
| 11 | 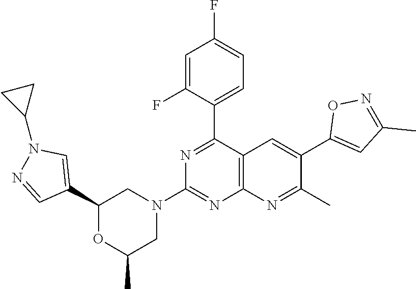 [SMILES: Cc1cc(-c2cc3c(-c4ccc(F)cc4F)nc(N4C[C@@H](C)O[C@@H](c5cnn(C6CC6)c5)C4)nc3nc2C)on1] |
| 12 | 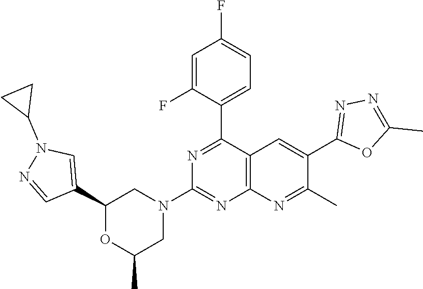 [SMILES: Cc1nnc(-c2cc3c(-c4ccc(F)cc4F)nc(N4C[C@@H](C)O[C@@H](c5cnn(C6CC6)c5)C4)nc3nc2C)o1] |
| 13 | 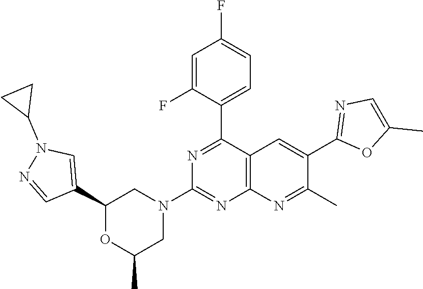 [SMILES: Cc1cnc(-c2cc3c(-c4ccc(F)cc4F)nc(N4C[C@@H](C)O[C@@H](c5cnn(C6CC6)c5)C4)nc3nc2C)o1] |
| 15 | 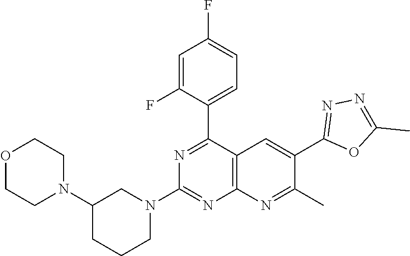 [SMILES: Cc1nnc(-c2cc3c(-c4ccc(F)cc4F)nc(N4CCCC(N5CCOCC5)C4)nc3nc2C)o1] |
| 16 | 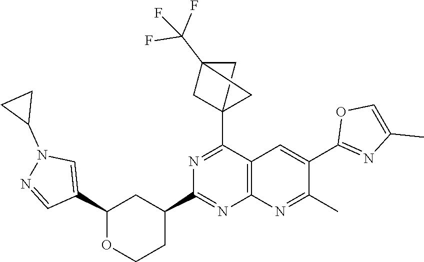 [SMILES: Cc1coc(-c2cc3c(C45CC(C(F)(F)F)(C4)C5)nc([C@H]4CCO[C@@H](c5cnn(C6CC6)c5)C4)nc3nc2C)n1] |
| 18 | 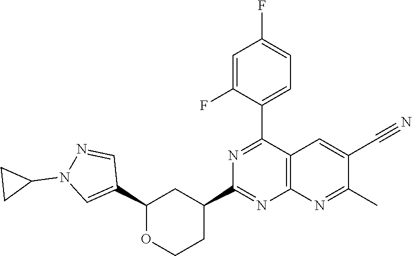 [SMILES: Cc1nc2nc([C@H]3CCO[C@@H](c4cnn(C5CC5)c4)C3)nc(-c3ccc(F)cc3F)c2cc1C#N] |
| 19 | 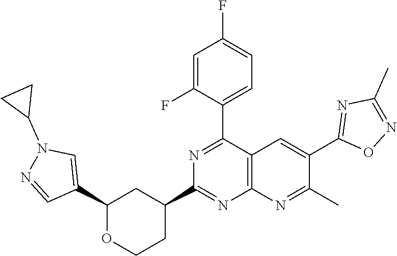 [SMILES: Cc1noc(-c2cc3c(-c4ccc(F)cc4F)nc([C@H]4CCO[C@@H](c5cnn(C6CC6)c5)C4)nc3nc2C)n1] |
| 21 | 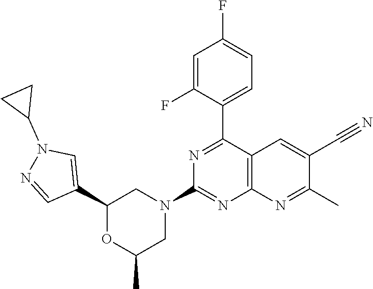 |
| 22 | 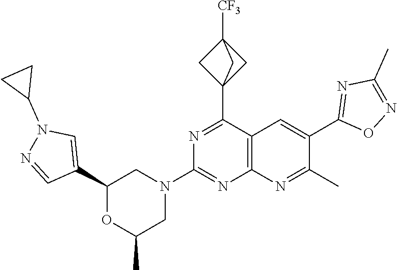 [SMILES: Cc1noc(-c2cc3c(C45CC(C(F)(F)F)(C4)C5)nc(N4C[C@@H](C)O[C@@H](c5cnn(C6CC6)c5)C4)nc3nc2C)n1] |
| 23 | 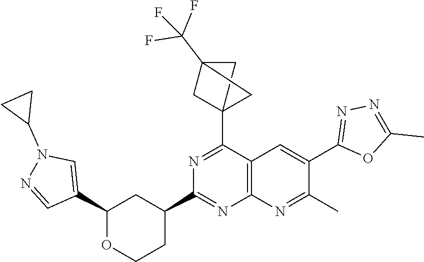 [SMILES: Cc1nnc(-c2cc3c(C45CC(C(F)(F)F)(C4)C5)nc([C@H]4CCO[C@@H](c5cnn(C6CC6)c5)C4)nc3nc2C)o1] |
| 24 | 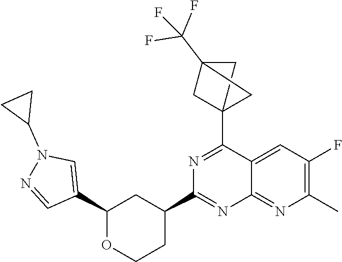 [SMILES: Cc1nc2nc([C@H]3CCO[C@@H](c4cnn(C5CC5)c4)C3)nc(C34CC(C(F)(F)F)(C3)C4)c2cc1F] |
| 25 | 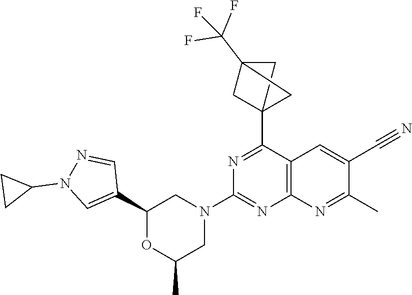 [SMILES: Cc1nc2nc(N3C[C@@H](C)O[C@@H](c4cnn(C5CC5)c4)C3)nc(C34CC(C(F)(F)F)(C3)C4)c2cc1C#N] |
| 26 | 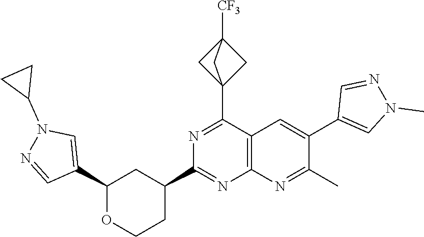 [SMILES: Cc1nc2nc([C@H]3CCO[C@@H](c4cnn(C5CC5)c4)C3)nc(C34CC(C(F)(F)F)(C3)C4)c2cc1-c1cnn(C)c1] |
| 27 | 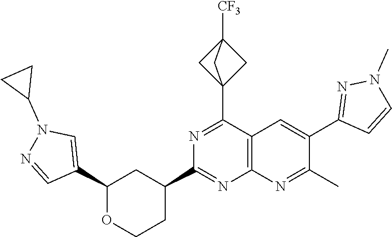 [SMILES: Cc1nc2nc([C@H]3CCO[C@@H](c4cnn(C5CC5)c4)C3)nc(C34CC(C(F)(F)F)(C3)C4)c2cc1-c1ccn(C)n1] |
| 28 | 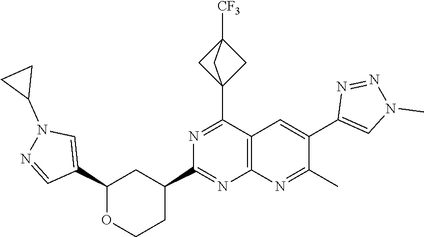 [SMILES: Cc1nc2nc([C@H]3CCO[C@@H](c4cnn(C5CC5)c4)C3)nc(C34CC(C(F)(F)F)(C3)C4)c2cc1-c1cn(C)nn1] |
| 29 | 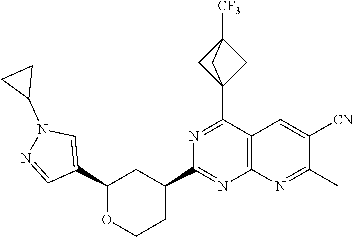 [SMILES: Cc1nc2nc([C@H]3CCO[C@@H](c4cnn(C5CC5)c4)C3)nc(C34CC(C(F)(F)F)(C3)C4)c2cc1C#N] |
| 31 | 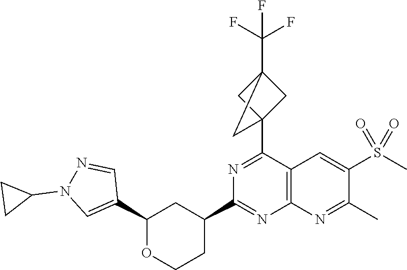 [SMILES: Cc1nc2nc([C@H]3CCO[C@@H](c4cnn(C5CC5)c4)C3)nc(C34CC(C(F)(F)F)(C3)C4)c2cc1S(C)(=O)=O] |
| 32 | 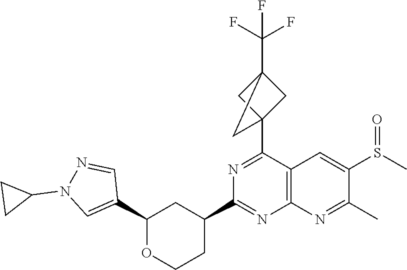 [SMILES: Cc1nc2nc([C@H]3CCO[C@@H](c4cnn(C5CC5)c4)C3)nc(C34CC(C(F)(F)F)(C3)C4)c2cc1S(C)=O] |
| 33 | 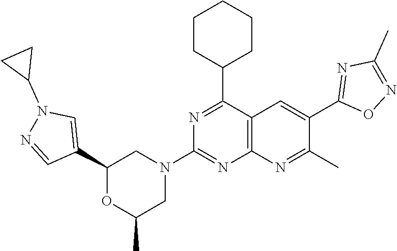 [SMILES: Cc1noc(-c2cc3c(C4CCCCC4)nc(N4C[C@@H](C)O[C@@H](c5cnn(C6CC6)c5)C4)nc3nc2C)n1] |
| 35 | 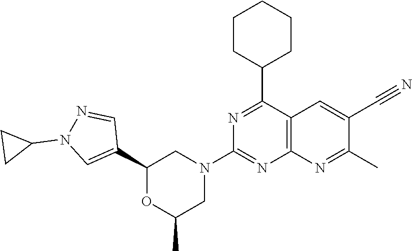 [SMILES: Cc1nc2nc(N3C[C@@H](C)O[C@@H](c4cnn(C5CC5)c4)C3)nc(C3CCCCC3)c2cc1C#N] |
| 42 | 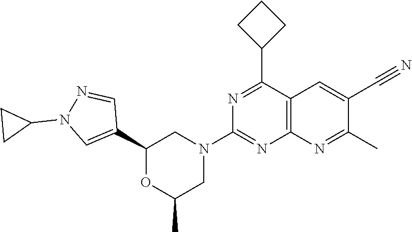 [SMILES: Cc1nc2nc(N3C[C@@H](C)O[C@@H](c4cnn(C5CC5)c4)C3)nc(C3CCC3)c2cc1C#N] |
| 44 | 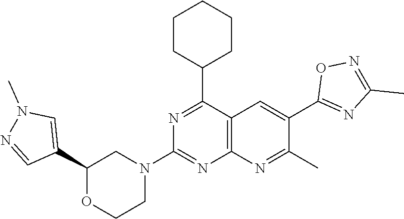 [SMILES: Cc1noc(-c2cc3c(C4CCCCC4)nc(N4CCO[C@@H](c5cnn(C)c5)C4)nc3nc2C)n1] |
| 45 | 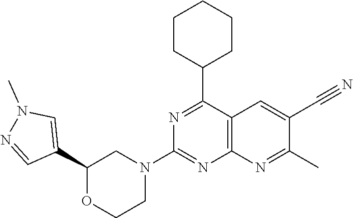 [SMILES: Cc1nc2nc(N3CCO[C@@H](c4cnn(C)c4)C3)nc(C3CCCCC3)c2cc1C#N] |
| 46 | 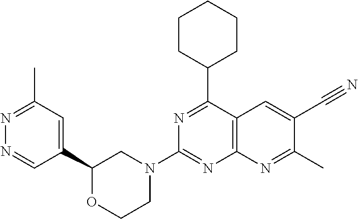 [SMILES: Cc1cc([C@H]2CN(c3nc(C4CCCCC4)c4cc(C#N)c(C)nc4n3)CCO2)cnn1] |
| 65 | 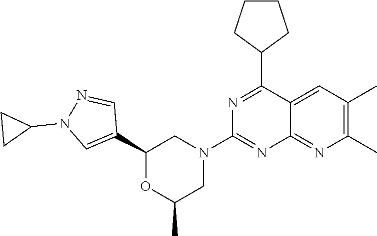 [SMILES: Cc1cc2c(C3CCCC3)nc(N3C[C@@H](C)O[C@@H](c4cnn(C5CC5)c4)C3)nc2nc1C] |
| 66 | 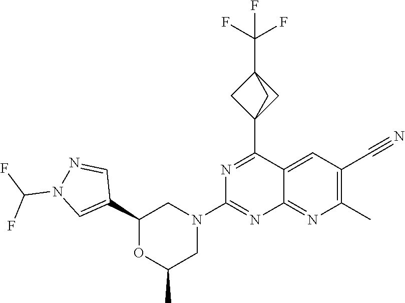 [SMILES: Cc1nc2nc(N3C[C@@H](C)O[C@@H](c4cnn(C(F)F)c4)C3)nc(C34CC(C(F)(F)F)(C3)C4)c2cc1C#N] |
| 67 | 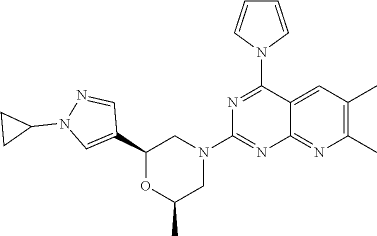 [SMILES: Cc1cc2c(-n3cccc3)nc(N3C[C@@H](C)O[C@@H](c4cnn(C5CC5)c4)C3)nc2nc1C] |
| 68 | 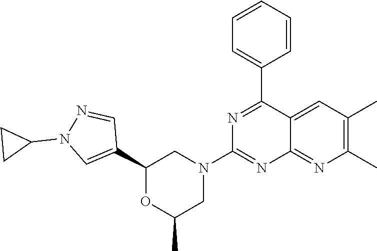 [SMILES: Cc1cc2c(-c3ccccc3)nc(N3C[C@@H](C)O[C@@H](c4cnn(C5CC5)c4)C3)nc2nc1C] |
| 69 | 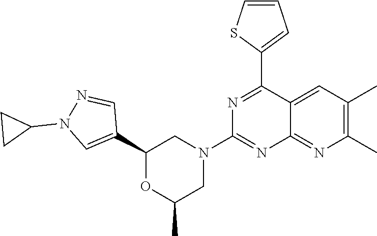 [SMILES: Cc1cc2c(-c3cccs3)nc(N3C[C@@H](C)O[C@@H](c4cnn(C5CC5)c4)C3)nc2nc1C] |
| 70 | 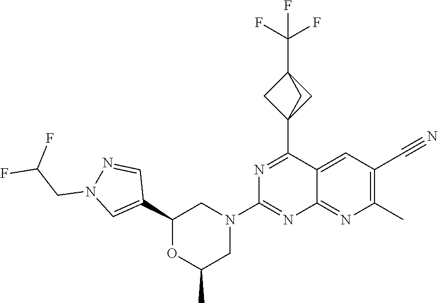 [SMILES: Cc1nc2nc(N3C[C@@H](C)O[C@@H](c4cnn(CC(F)F)c4)C3)nc(C34CC(C(F)(F)F)(C3)C4)c2cc1C#N] |
| 71 | 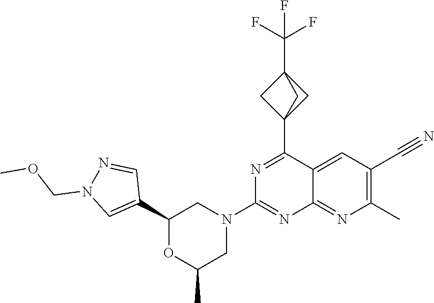 [SMILES: COCn1cc([C@H]2CN(c3nc(C45CC(C(F)(F)F)(C4)C5)c4cc(C#N)c(C)nc4n3)C[C@@H](C)O2)cn1] |
| 72 | 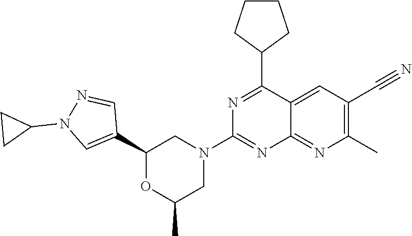 [SMILES: Cc1nc2nc(N3C[C@@H](C)O[C@@H](c4cnn(C5CC5)c4)C3)nc(C3CCCC3)c2cc1C#N] |
| 73 | 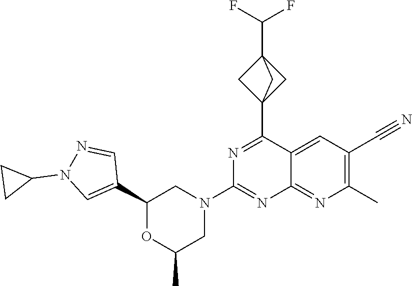 [SMILES: Cc1nc2nc(N3C[C@@H](C)O[C@@H](c4cnn(C5CC5)c4)C3)nc(C34CC(C(F)F)(C3)C4)c2cc1C#N] |
| 74 | 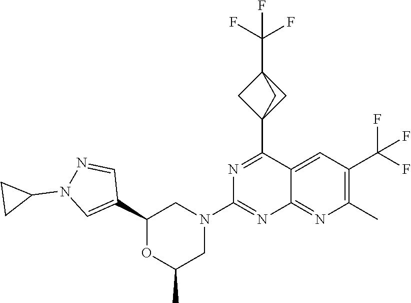 [SMILES: Cc1nc2nc(N3C[C@@H](C)O[C@@H](c4cnn(C5CC5)c4)C3)nc(C34CC(C(F)(F)F)(C3)C4)c2cc1C(F)(F)F] |
| 81 | 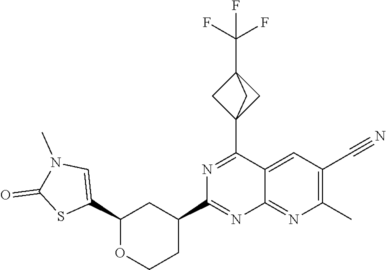 [SMILES: Cc1nc2nc([C@H]3CCO[C@@H](c4cn(C)c(=O)s4)C3)nc(C34CC(C(F)(F)F)(C3)C4)c2cc1C#N] |
| 84 | 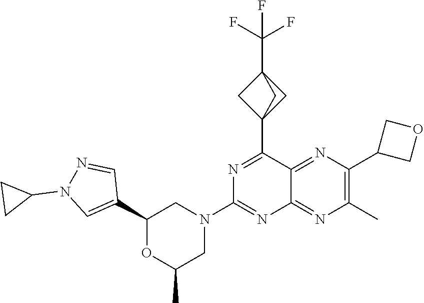 [SMILES: Cc1nc2nc(N3C[C@@H](C)O[C@@H](c4cnn(C5CC5)c4)C3)nc(C34CC(C(F)(F)F)(C3)C4)c2nc1C1COC1] |
| 86 | 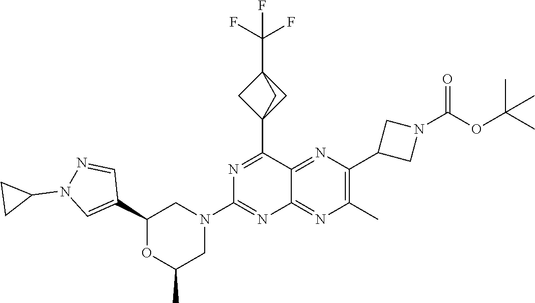 [SMILES: Cc1nc2nc(N3C[C@@H](C)O[C@@H](c4cnn(C5CC5)c4)C3)nc(C34CC(C(F)(F)F)(C3)C4)c2nc1C1CN(C(=O)OC(C)(C)C)C1] |
| 87 | 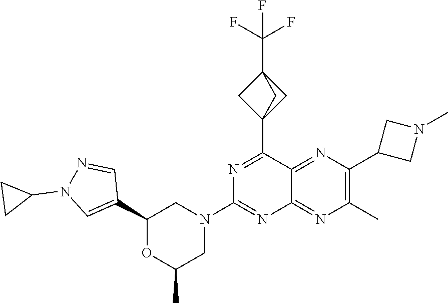 [SMILES: Cc1nc2nc(N3C[C@@H](C)O[C@@H](c4cnn(C5CC5)c4)C3)nc(C34CC(C(F)(F)F)(C3)C4)c2nc1C1CN(C)C1] |
| 88 | 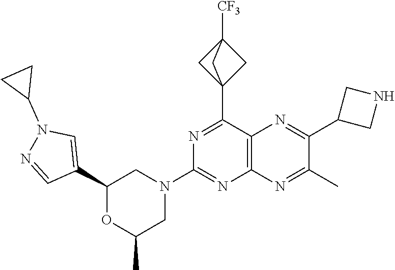 [SMILES: Cc1nc2nc(N3C[C@@H](C)O[C@@H](c4cnn(C5CC5)c4)C3)nc(C34CC(C(F)(F)F)(C3)C4)c2nc1C1CNC1] |
| 90 | 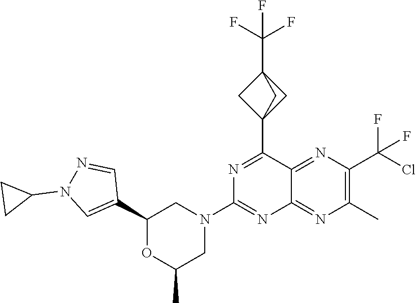 [SMILES: Cc1nc2nc(N3C[C@@H](C)O[C@@H](c4cnn(C5CC5)c4)C3)nc(C34CC(C(F)(F)F)(C3)C4)c2nc1C(F)(F)Cl] |
| 91 | 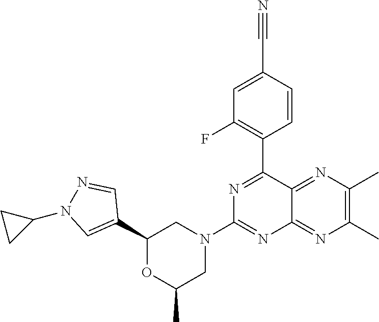 [SMILES: Cc1nc2nc(N3C[C@@H](C)O[C@@H](c4cnn(C5CC5)c4)C3)nc(-c3ccc(C#N)cc3F)c2nc1C] |
| 92 | 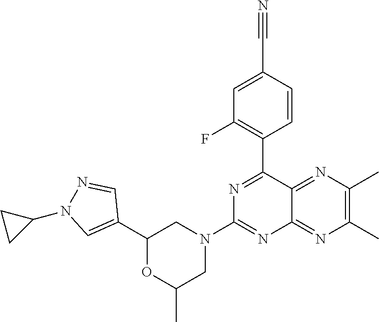 [SMILES: Cc1nc2nc(N3CC(C)OC(c4cnn(C5CC5)c4)C3)nc(-c3ccc(C#N)cc3F)c2nc1C] |
| 93 | 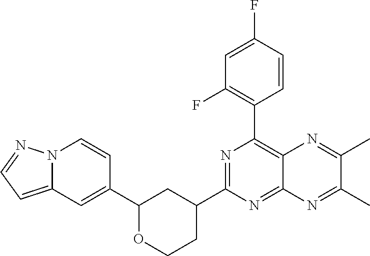 [SMILES: Cc1nc2nc(C3CCOC(c4ccn5nccc5c4)C3)nc(-c3ccc(F)cc3F)c2nc1C] |
| 94 | 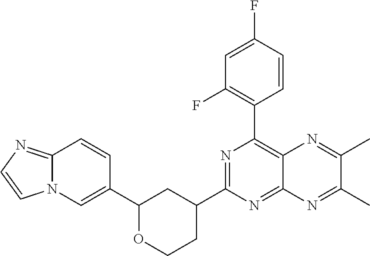 [SMILES: Cc1nc2nc(C3CCOC(c4ccc5nccn5c4)C3)nc(-c3ccc(F)cc3F)c2nc1C] |
| 95 | 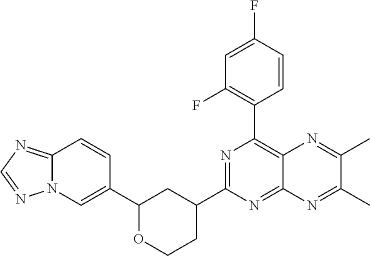 [SMILES: Cc1nc2nc(C3CCOC(c4ccc5ncnn5c4)C3)nc(-c3ccc(F)cc3F)c2nc1C] |
| 96 | 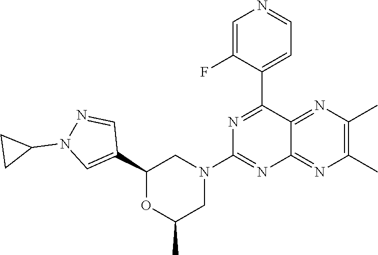 [SMILES: Cc1nc2nc(N3C[C@@H](C)O[C@@H](c4cnn(C5CC5)c4)C3)nc(-c3ccncc3F)c2nc1C] |
| 97 | 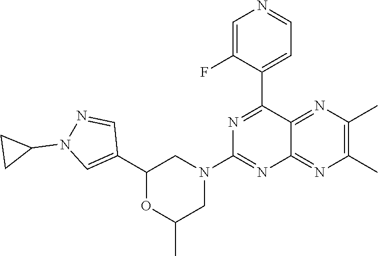 [SMILES: Cc1nc2nc(N3CC(C)OC(c4cnn(C5CC5)c4)C3)nc(-c3ccncc3F)c2nc1C] |
| 98 | 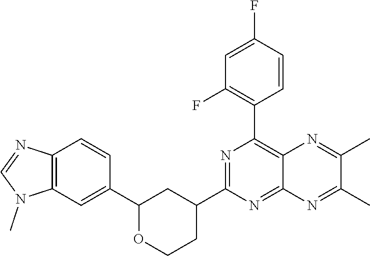 [SMILES: Cc1nc2nc(C3CCOC(c4ccc5ncn(C)c5c4)C3)nc(-c3ccc(F)cc3F)c2nc1C] |
| 99 | 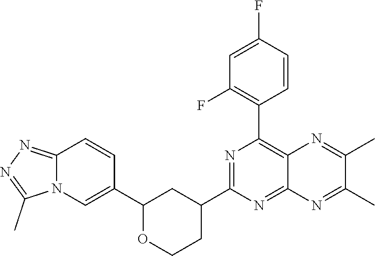 [SMILES: Cc1nc2nc(C3CCOC(c4ccc5nnc(C)n5c4)C3)nc(-c3ccc(F)cc3F)c2nc1C] |
| 100 | 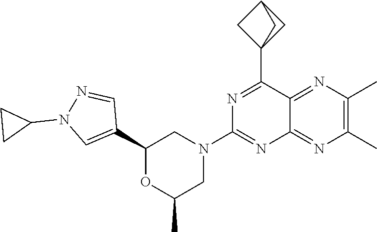 [SMILES: Cc1nc2nc(N3C[C@@H](C)O[C@@H](c4cnn(C5CC5)c4)C3)nc(C34CC(C3)C4)c2nc1C] |
| 101 | 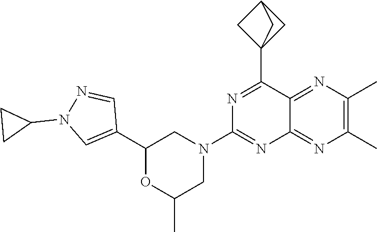 [SMILES: Cc1nc2nc(N3CC(C)OC(c4cnn(C5CC5)c4)C3)nc(C34CC(C3)C4)c2nc1C] |
| 102 | 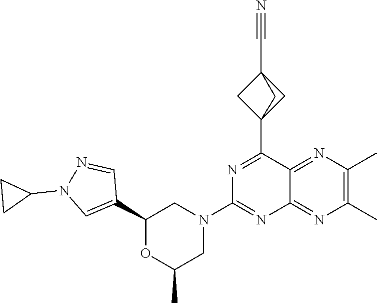 [SMILES: Cc1nc2nc(N3C[C@@H](C)O[C@@H](c4cnn(C5CC5)c4)C3)nc(C34CC(C#N)(C3)C4)c2nc1C] |
| 103 | 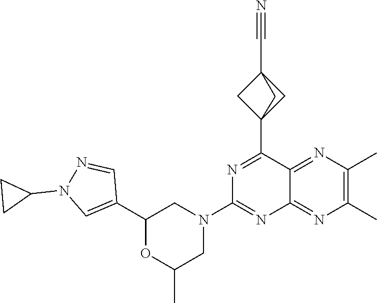 [SMILES: Cc1nc2nc(N3CC(C)OC(c4cnn(C5CC5)c4)C3)nc(C34CC(C#N)(C3)C4)c2nc1C] |
| 104 | 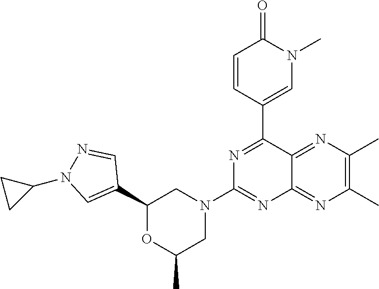 [SMILES: Cc1nc2nc(N3C[C@@H](C)O[C@@H](c4cnn(C5CC5)c4)C3)nc(-c3ccc(=O)n(C)c3)c2nc1C] |
| 105 | 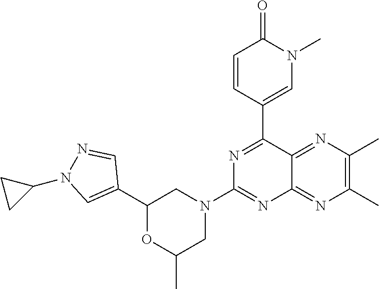 [SMILES: Cc1nc2nc(N3CC(C)OC(c4cnn(C5CC5)c4)C3)nc(-c3ccc(=O)n(C)c3)c2nc1C] |
| 106 | 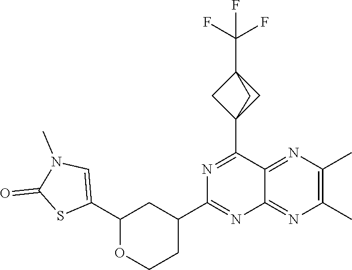 [SMILES: Cc1nc2nc(C3CCOC(c4cn(C)c(=O)s4)C3)nc(C34CC(C(F)(F)F)(C3)C4)c2nc1C] |
| 107 | 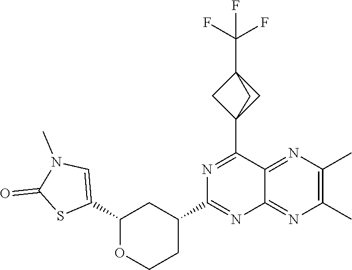 [SMILES: Cc1nc2nc([C@@H]3CCO[C@H](c4cn(C)c(=O)s4)C3)nc(C34CC(C(F)(F)F)(C3)C4)c2nc1C] |
| 108 | 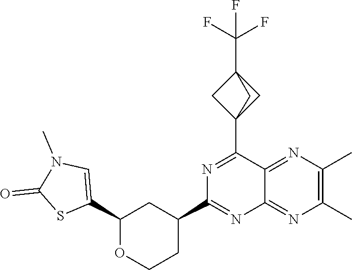 [SMILES: Cc1nc2nc([C@H]3CCO[C@@H](c4cn(C)c(=O)s4)C3)nc(C34CC(C(F)(F)F)(C3)C4)c2nc1C] |
| 109 | 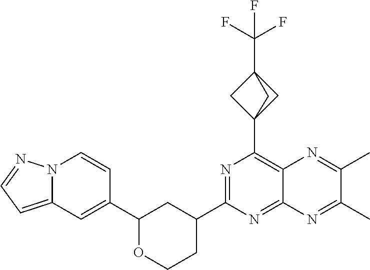 [SMILES: Cc1nc2nc(C3CCOC(c4ccn5nccc5c4)C3)nc(C34CC(C(F)(F)F)(C3)C4)c2nc1C] |
| 110 | 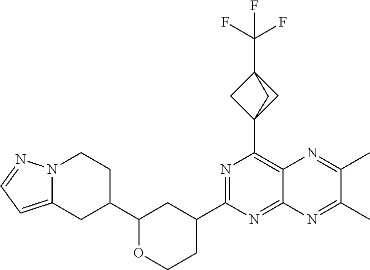 [SMILES: Cc1nc2nc(C3CCOC(C4CCn5nccc5C4)C3)nc(C34CC(C(F)(F)F)(C3)C4)c2nc1C] |
| 111 | 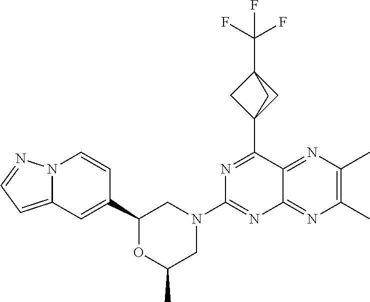 [SMILES: Cc1nc2nc(N3C[C@@H](C)O[C@@H](c4ccn5nccc5c4)C3)nc(C34CC(C(F)(F)F)(C3)C4)c2nc1C] |
| 112 | 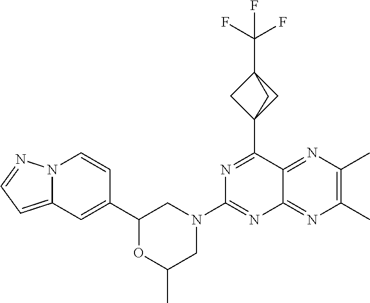 [SMILES: Cc1nc2nc(N3CC(C)OC(c4ccn5nccc5c4)C3)nc(C34CC(C(F)(F)F)(C3)C4)c2nc1C] |
| 113 | 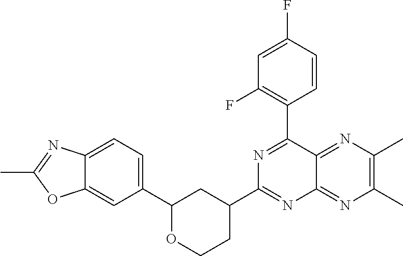 |
| 114 | 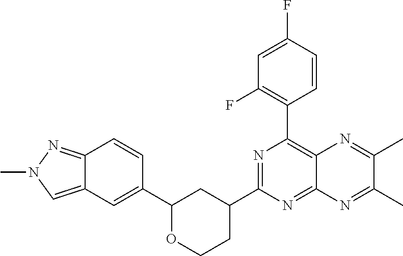 |
| 115 | 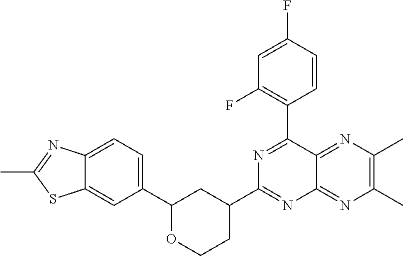 |
| 116 | 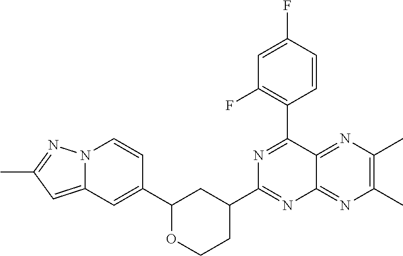 |
| 117 | 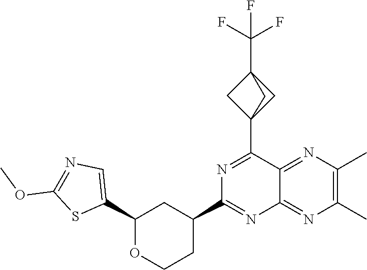 |
| 118 | 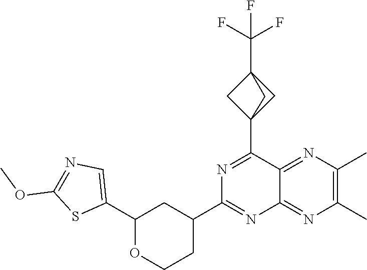 |
| 119 | 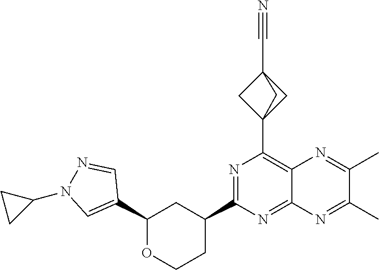 [SMILES: Cc1nc2nc([C@H]3CCO[C@@H](c4cnn(C5CC5)c4)C3)nc(C34CC(C#N)(C3)C4)c2nc1C] |
| 120 | 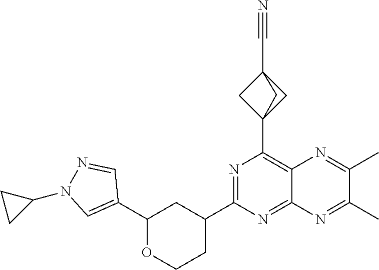 [SMILES: Cc1nc2nc(C3CCOC(c4cnn(C5CC5)c4)C3)nc(C34CC(C#N)(C3)C4)c2nc1C] |
| 121 | 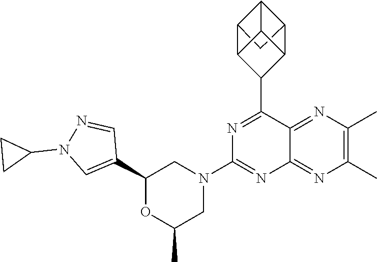 [SMILES: Cc1nc2nc(N3C[C@@H](C)O[C@@H](c4cnn(C5CC5)c4)C3)nc(C3C4C5CC6C5C4C63)c2nc1C] |
| 122 | 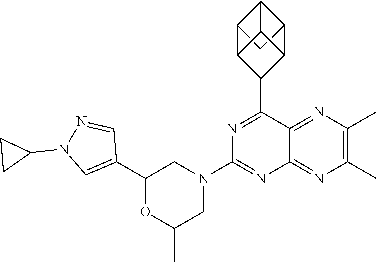 [SMILES: Cc1nc2nc(N3CC(C)OC(c4cnn(C5CC5)c4)C3)nc(C3C4C5CC6C5C4C63)c2nc1C] |
| 123 | 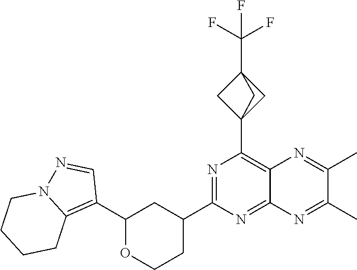 [SMILES: Cc1nc2nc(C3CCOC(c4cnn5c4CCCC5)C3)nc(C34CC(C(F)(F)F)(C3)C4)c2nc1C] |
| 124 | 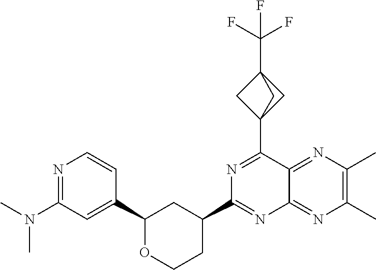 |
| 125 | 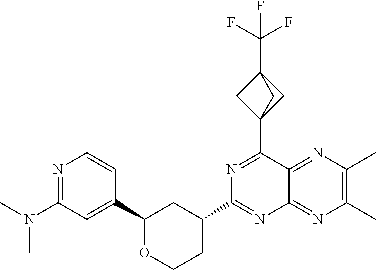 |
| 126 | 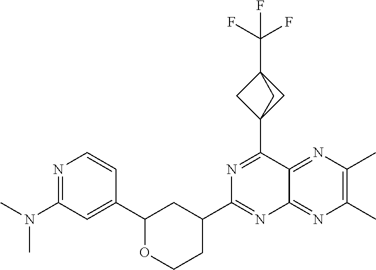 |
| 127 | 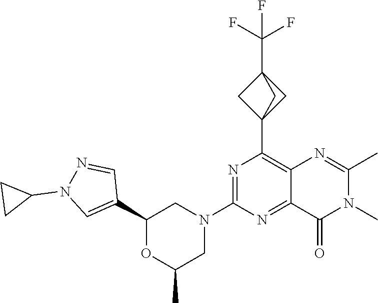 [SMILES: Cc1nc2c(C34CC(C(F)(F)F)(C3)C4)nc(N3C[C@@H](C)O[C@@H](c4cnn(C5CC5)c4)C3)nc2c(=O)n1C] |
| 128 | 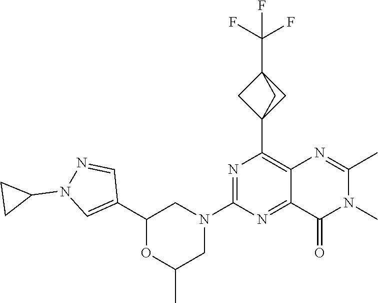 [SMILES: Cc1nc2c(C34CC(C(F)(F)F)(C3)C4)nc(N3CC(C)OC(c4cnn(C5CC5)c4)C3)nc2c(=O)n1C] |
| 129 | 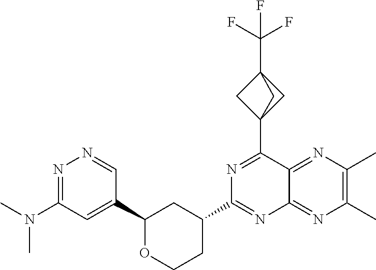 |
| 130 | 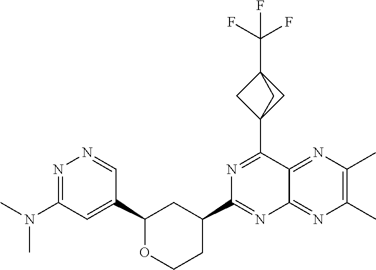 |
| 131 | 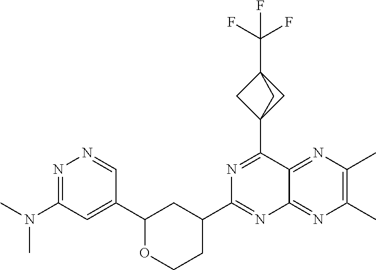 |
| 132 | 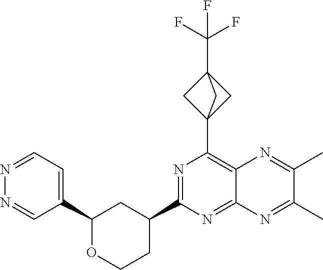 [SMILES: Cc1nc2nc([C@H]3CCO[C@@H](c4ccnnc4)C3)nc(C34CC(C(F)(F)F)(C3)C4)c2nc1C] |
| 133 | 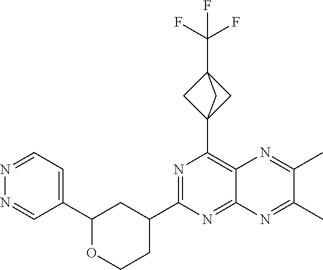 [SMILES: Cc1nc2nc(C3CCOC(c4ccnnc4)C3)nc(C34CC(C(F)(F)F)(C3)C4)c2nc1C] |
| 134 | 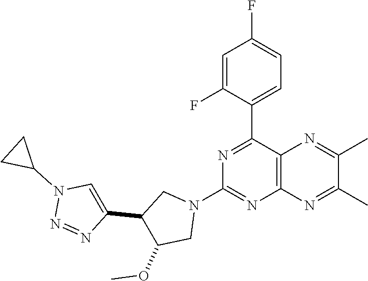 [SMILES: CO[C@H]1CN(c2nc(-c3ccc(F)cc3F)c3nc(C)c(C)nc3n2)C[C@@H]1c1cn(C2CC2)nn1] |
| 135 | 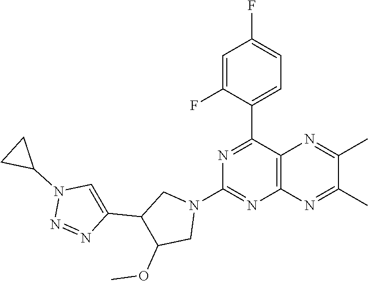 [SMILES: COC1CN(c2nc(-c3ccc(F)cc3F)c3nc(C)c(C)nc3n2)CC1c1cn(C2CC2)nn1] |
| 136 | 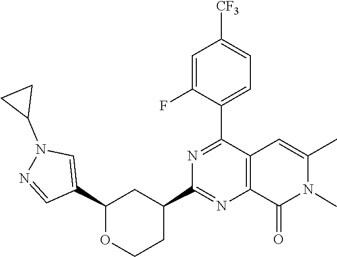 [SMILES: Cc1cc2c(-c3ccc(C(F)(F)F)cc3F)nc([C@H]3CCO[C@@H](c4cnn(C5CC5)c4)C3)nc2c(=O)n1C] |
| 137 | 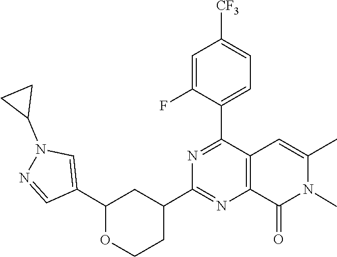 [SMILES: Cc1cc2c(-c3ccc(C(F)(F)F)cc3F)nc(C3CCOC(c4cnn(C5CC5)c4)C3)nc2c(=O)n1C] |
| 138 | 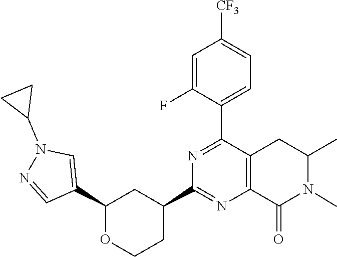 [SMILES: CC1Cc2c(nc([C@H]3CCO[C@@H](c4cnn(C5CC5)c4)C3)nc2-c2ccc(C(F)(F)F)cc2F)C(=O)N1C] |
| 139 | 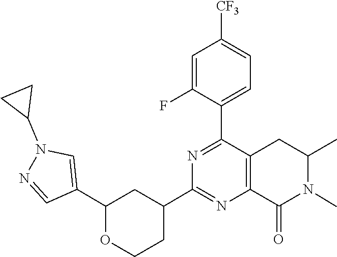 [SMILES: CC1Cc2c(nc(C3CCOC(c4cnn(C5CC5)c4)C3)nc2-c2ccc(C(F)(F)F)cc2F)C(=O)N1C] |
| 140 | 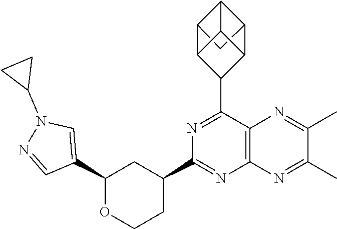 [SMILES: Cc1nc2nc([C@H]3CCO[C@@H](c4cnn(C5CC5)c4)C3)nc(C3C4C5CC6C5C4C63)c2nc1C] |
| 142 | 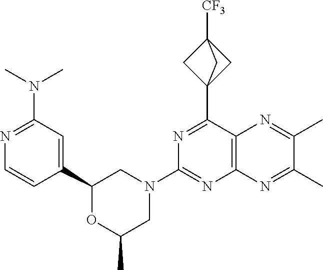 [SMILES: Cc1nc2nc(N3C[C@@H](C)O[C@@H](c4ccnc(N(C)C)c4)C3)nc(C34CC(C(F)(F)F)(C3)C4)c2nc1C] |
| 143 | 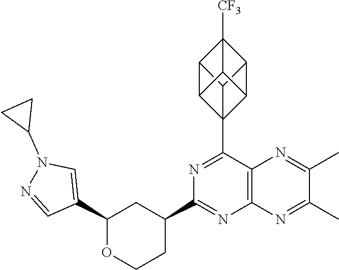 [SMILES: Cc1nc2nc([C@H]3CCO[C@@H](c4cnn(C5CC5)c4)C3)nc(C34C5C6C3C3C4C5C63C(F)(F)F)c2nc1C] |
| 145 | 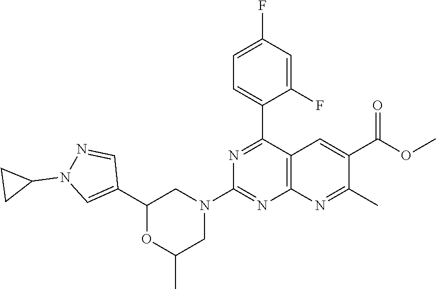 [SMILES: COC(=O)c1cc2c(-c3ccc(F)cc3F)nc(N3CC(C)OC(c4cnn(C5CC5)c4)C3)nc2nc1C] |
| 146 | 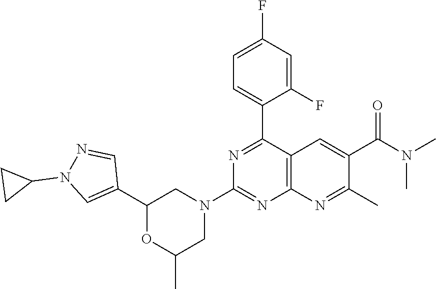 [SMILES: Cc1nc2nc(N3CC(C)OC(c4cnn(C5CC5)c4)C3)nc(-c3ccc(F)cc3F)c2cc1C(=O)N(C)C] |
| 147 | 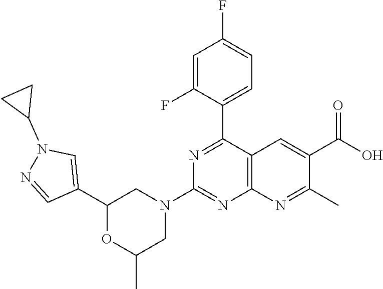 [SMILES: Cc1nc2nc(N3CC(C)OC(c4cnn(C5CC5)c4)C3)nc(-c3ccc(F)cc3F)c2cc1C(=O)O] |
| 148 | 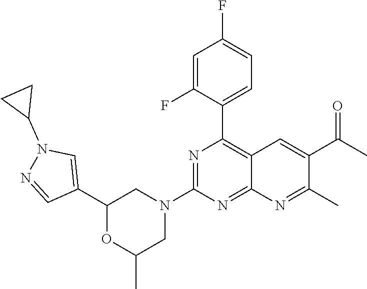 [SMILES: CC(=O)c1cc2c(-c3ccc(F)cc3F)nc(N3CC(C)OC(c4cnn(C5CC5)c4)C3)nc2nc1C] |
| 149 | 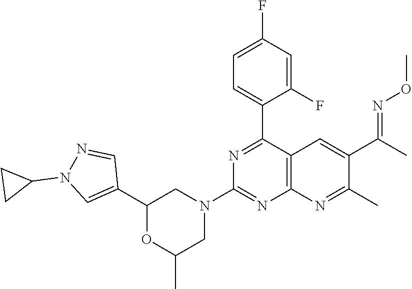 [SMILES: CO/N=C(\C)c1cc2c(-c3ccc(F)cc3F)nc(N3CC(C)OC(c4cnn(C5CC5)c4)C3)nc2nc1C] |
| 150 | 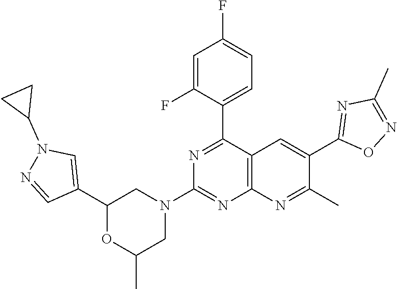 [SMILES: Cc1noc(-c2cc3c(-c4ccc(F)cc4F)nc(N4CC(C)OC(c5cnn(C6CC6)c5)C4)nc3nc2C)n1] |
| 151 | 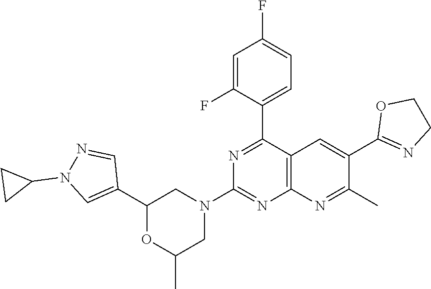 [SMILES: Cc1nc2nc(N3CC(C)OC(c4cnn(C5CC5)c4)C3)nc(-c3ccc(F)cc3F)c2cc1C1=NCCO1] |
| 152 | 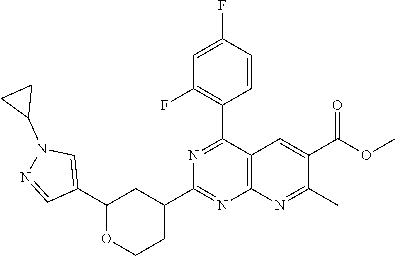 [SMILES: COC(=O)c1cc2c(-c3ccc(F)cc3F)nc(C3CCOC(c4cnn(C5CC5)c4)C3)nc2nc1C] |
| 153 | 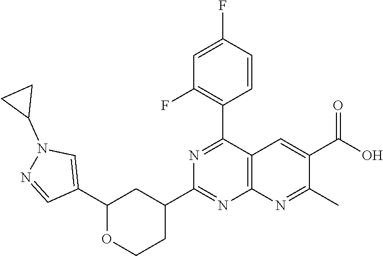 [SMILES: Cc1nc2nc(C3CCOC(c4cnn(C5CC5)c4)C3)nc(-c3ccc(F)cc3F)c2cc1C(=O)O] |
| 154 | 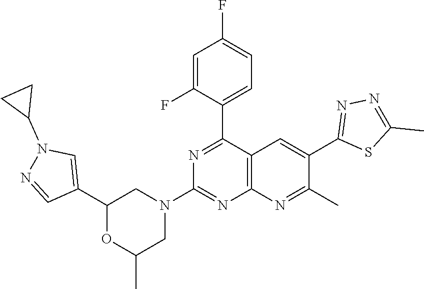 [SMILES: Cc1nnc(-c2cc3c(-c4ccc(F)cc4F)nc(N4CC(C)OC(c5cnn(C6CC6)c5)C4)nc3nc2C)s1] |
| 155 | 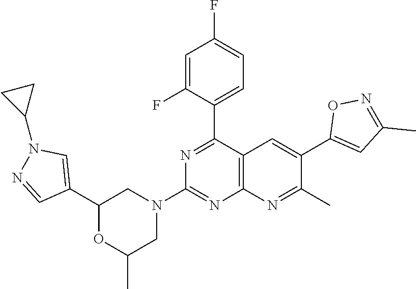 [SMILES: Cc1cc(-c2cc3c(-c4ccc(F)cc4F)nc(N4CC(C)OC(c5cnn(C6CC6)c5)C4)nc3nc2C)on1] |
| 156 | 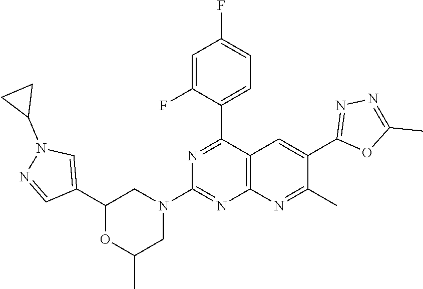 [SMILES: Cc1nnc(-c2cc3c(-c4ccc(F)cc4F)nc(N4CC(C)OC(c5cnn(C6CC6)c5)C4)nc3nc2C)o1] |
| 157 | 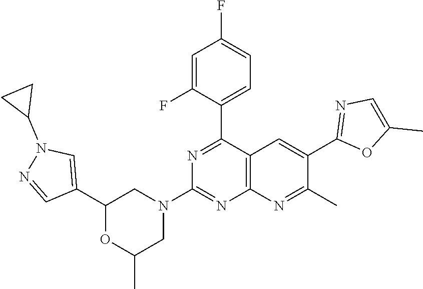 [SMILES: Cc1cnc(-c2cc3c(-c4ccc(F)cc4F)nc(N4CC(C)OC(c5cnn(C6CC6)c5)C4)nc3nc2C)o1] |
| 158 | 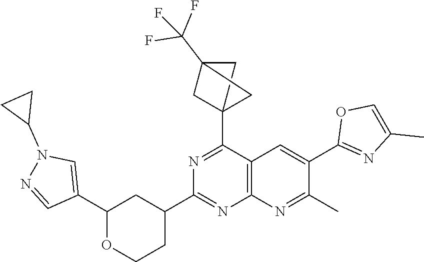 [SMILES: Cc1coc(-c2cc3c(C45CC(C(F)(F)F)(C4)C5)nc(C4CCOC(c5cnn(C6CC6)c5)C4)nc3nc2C)n1] |
| 159 | 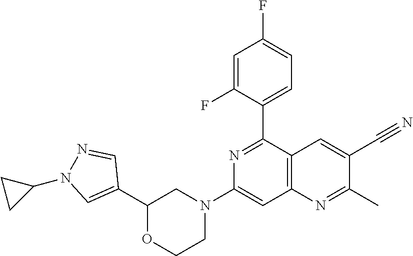 [SMILES: Cc1nc2cc(N3CCOC(c4cnn(C5CC5)c4)C3)nc(-c3ccc(F)cc3F)c2cc1C#N] |
| 160 | 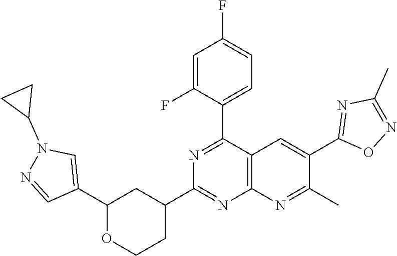 [SMILES: Cc1noc(-c2cc3c(-c4ccc(F)cc4F)nc(C4CCOC(c5cnn(C6CC6)c5)C4)nc3nc2C)n1] |
| 161 | 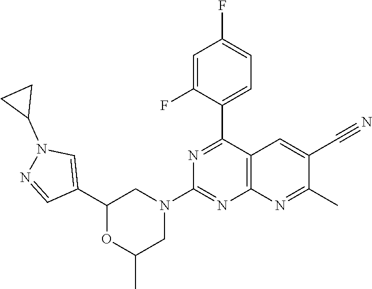 [SMILES: Cc1nc2nc(N3CC(C)OC(c4cnn(C5CC5)c4)C3)nc(-c3ccc(F)cc3F)c2cc1C#N] |
| 162 | 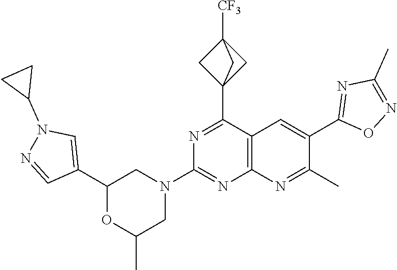 [SMILES: Cc1noc(-c2cc3c(C45CC(C(F)(F)F)(C4)C5)nc(N4CC(C)OC(c5cnn(C6CC6)c5)C4)nc3nc2C)n1] |
| 163 | 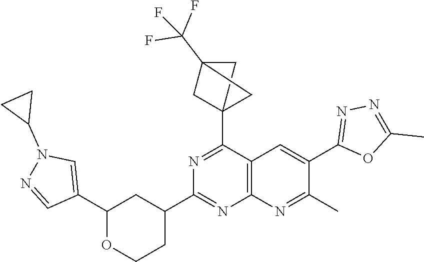 [SMILES: Cc1nnc(-c2cc3c(C45CC(C(F)(F)F)(C4)C5)nc(C4CCOC(c5cnn(C6CC6)c5)C4)nc3nc2C)o1] |
| 164 | 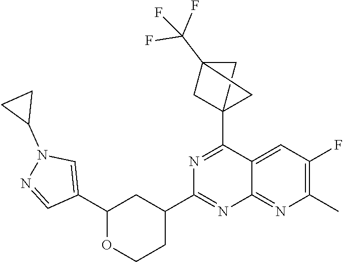 [SMILES: Cc1nc2nc(C3CCOC(c4cnn(C5CC5)c4)C3)nc(C34CC(C(F)(F)F)(C3)C4)c2cc1F] |
| 165 | 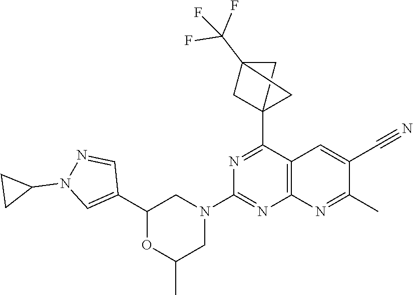 [SMILES: Cc1nc2nc(N3CC(C)OC(c4cnn(C5CC5)c4)C3)nc(C34CC(C(F)(F)F)(C3)C4)c2cc1C#N] |
| 166 | 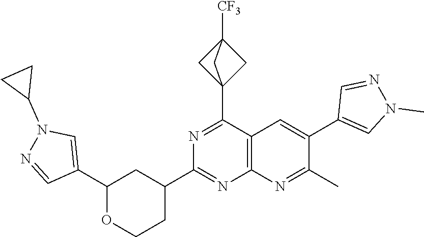 [SMILES: Cc1nc2nc(C3CCOC(c4cnn(C5CC5)c4)C3)nc(C34CC(C(F)(F)F)(C3)C4)c2cc1-c1cnn(C)c1] |
| 167 | 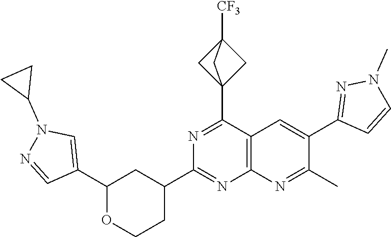 [SMILES: Cc1nc2nc(C3CCOC(c4cnn(C5CC5)c4)C3)nc(C34CC(C(F)(F)F)(C3)C4)c2cc1-c1ccn(C)n1] |
| 168 | 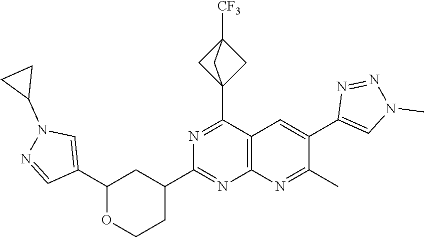 [SMILES: Cc1nc2nc(C3CCOC(c4cnn(C5CC5)c4)C3)nc(C34CC(C(F)(F)F)(C3)C4)c2cc1-c1cn(C)nn1] |
| 169 | 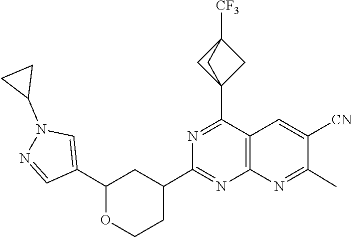 [SMILES: Cc1nc2nc(C3CCOC(c4cnn(C5CC5)c4)C3)nc(C34CC(C(F)(F)F)(C3)C4)c2cc1C#N] |
| 170 | 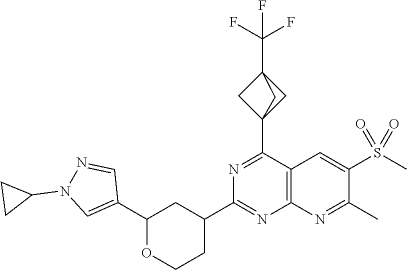 [SMILES: Cc1nc2nc(C3CCOC(c4cnn(C5CC5)c4)C3)nc(C34CC(C(F)(F)F)(C3)C4)c2cc1S(C)(=O)=O] |
| 171 | 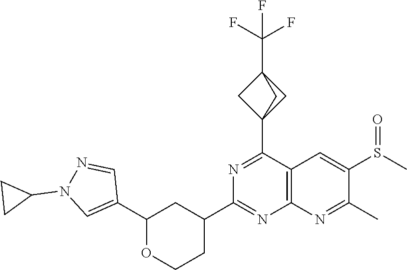 [SMILES: Cc1nc2nc(C3CCOC(c4cnn(C5CC5)c4)C3)nc(C34CC(C(F)(F)F)(C3)C4)c2cc1S(C)=O] |
| 172 | 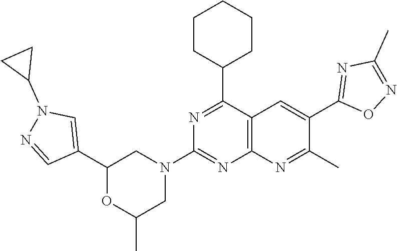 [SMILES: Cc1noc(-c2cc3c(C4CCCCC4)nc(N4CC(C)OC(c5cnn(C6CC6)c5)C4)nc3nc2C)n1] |
| 173 | 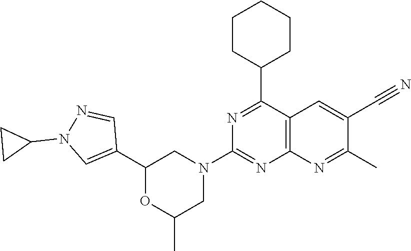 [SMILES: Cc1nc2nc(N3CC(C)OC(c4cnn(C5CC5)c4)C3)nc(C3CCCCC3)c2cc1C#N] |
| 174 | 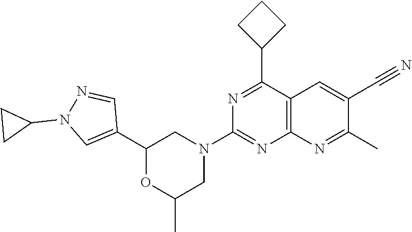 [SMILES: Cc1nc2nc(N3CC(C)OC(c4cnn(C5CC5)c4)C3)nc(C3CCC3)c2cc1C#N] |
| 175 | 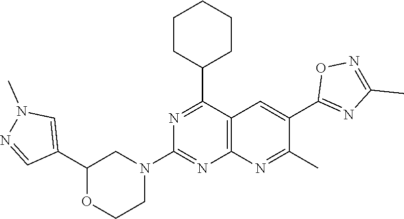 [SMILES: Cc1noc(-c2cc3c(C4CCCCC4)nc(N4CCOC(c5cnn(C)c5)C4)nc3nc2C)n1] |
| 176 | 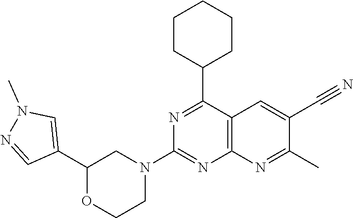 [SMILES: Cc1nc2nc(N3CCOC(c4cnn(C)c4)C3)nc(C3CCCCC3)c2cc1C#N] |
| 177 | 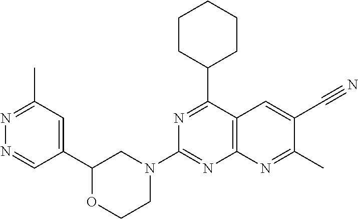 [SMILES: Cc1cc(C2CN(c3nc(C4CCCCC4)c4cc(C#N)c(C)nc4n3)CCO2)cnn1] |
| 178 | 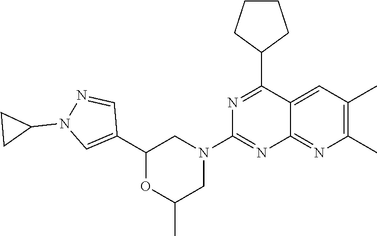 [SMILES: Cc1cc2c(C3CCCC3)nc(N3CC(C)OC(c4cnn(C5CC5)c4)C3)nc2nc1C] |
| 179 | 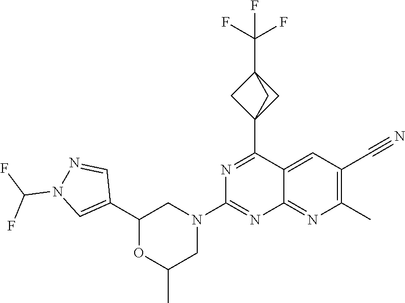 [SMILES: Cc1nc2nc(N3CC(C)OC(c4cnn(C(F)F)c4)C3)nc(C34CC(C(F)(F)F)(C3)C4)c2cc1C#N] |
| 180 | 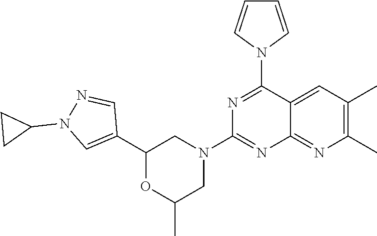 [SMILES: Cc1cc2c(-n3cccc3)nc(N3CC(C)OC(c4cnn(C5CC5)c4)C3)nc2nc1C] |
| 181 | 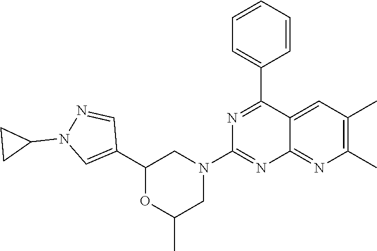 [SMILES: Cc1cc2c(-c3ccccc3)nc(N3CC(C)OC(c4cnn(C5CC5)c4)C3)nc2nc1C] |
| 182 | 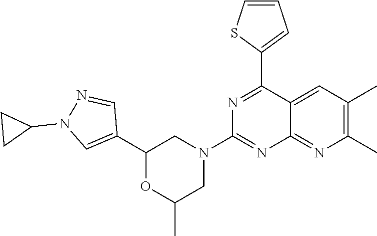 [SMILES: Cc1cc2c(-c3cccs3)nc(N3CC(C)OC(c4cnn(C5CC5)c4)C3)nc2nc1C] |
| 183 | 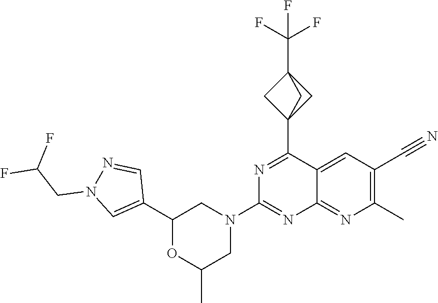 [SMILES: Cc1nc2nc(N3CC(C)OC(c4cnn(CC(F)F)c4)C3)nc(C34CC(C(F)(F)F)(C3)C4)c2cc1C#N] |
| 184 | 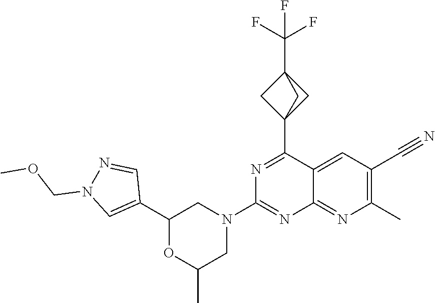 [SMILES: COCn1cc(C2CN(c3nc(C45CC(C(F)(F)F)(C4)C5)c4cc(C#N)c(C)nc4n3)CC(C)O2)cn1] |
| 185 | 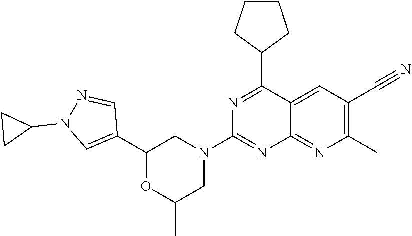 [SMILES: Cc1nc2nc(N3CC(C)OC(c4cnn(C5CC5)c4)C3)nc(C3CCCC3)c2cc1C#N] |
| 186 | 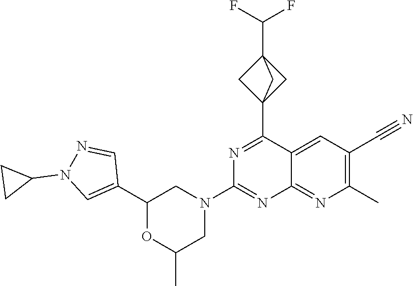 [SMILES: Cc1nc2nc(N3CC(C)OC(c4cnn(C5CC5)c4)C3)nc(C34CC(C(F)F)(C3)C4)c2cc1C#N] |
| 187 | 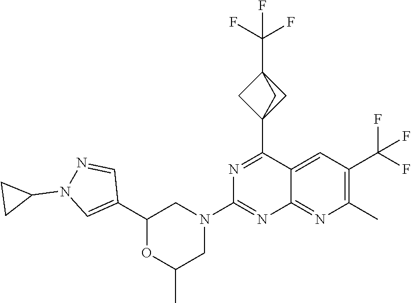 [SMILES: Cc1nc2nc(N3CC(C)OC(c4cnn(C5CC5)c4)C3)nc(C34CC(C(F)(F)F)(C3)C4)c2cc1C(F)(F)F] |
| 188 | 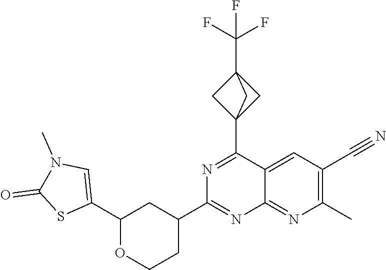 [SMILES: Cc1nc2nc(C3CCOC(c4cn(C)c(=O)s4)C3)nc(C34CC(C(F)(F)F)(C3)C4)c2cc1C#N] |
| 189 | 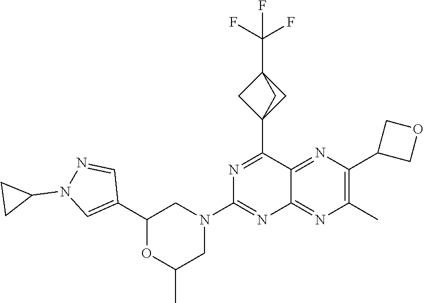 [SMILES: Cc1nc2nc(N3CC(C)OC(c4cnn(C5CC5)c4)C3)nc(C34CC(C(F)(F)F)(C3)C4)c2nc1C1COC1] |
| 190 | 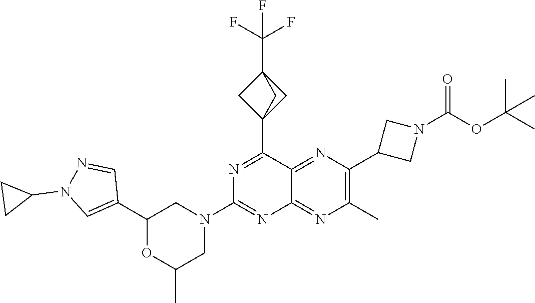 [SMILES: Cc1nc2nc(N3CC(C)OC(c4cnn(C5CC5)c4)C3)nc(C34CC(C(F)(F)F)(C3)C4)c2nc1C1CN(C(=O)OC(C)(C)C)C1] |
| 191 | 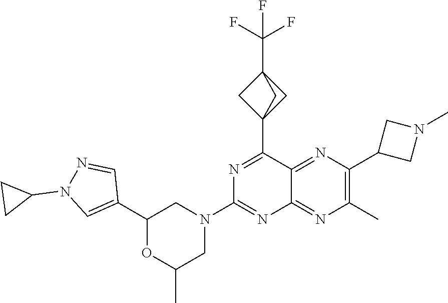 [SMILES: Cc1nc2nc(N3CC(C)OC(c4cnn(C5CC5)c4)C3)nc(C34CC(C(F)(F)F)(C3)C4)c2nc1C1CN(C)C1] |
| 192 | 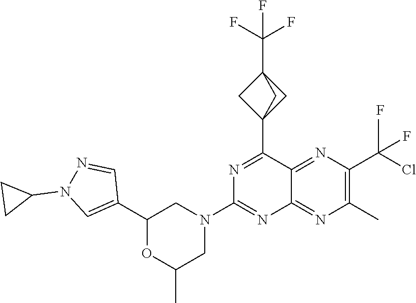 |

The foregoing merely summarizes certain aspects of this disclosure and is not intended, nor should it be construed, as limiting the disclosure in any way.

### Formulation and Route of Administration

While it may be possible to administer a compound disclosed herein alone in the uses described, the compound administered normally will be present as an active ingredient in a pharmaceutical composition. Thus, in one embodiment, provided herein is a pharmaceutical composition comprising a compound disclosed herein in combination with one or more pharmaceutically acceptable excipients, such as diluents, carriers, adjuvants and the like, and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018. In one embodiment, a pharmaceutical composition comprises a therapeutically effective amount of a compound disclosed herein.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

In one aspect, the disclosure provides a pharmaceutical composition comprising a compound of Formula (I), or a tautomer thereof, or a pharmaceutically acceptable salt of said compound or said tautomer, and a pharmaceutically acceptable excipient.

In another aspect, the disclosure provides a compound of Formula (I), or a tautomer thereof, or a pharmaceutically acceptable salt of said compound or said tautomer, or a pharmaceutical composition comprising said compound, or said tautomer, or said salt, for use as a medicament.

### Pharmaceutically Acceptable Compositions

According to some embodiments, the present disclosure provides a composition comprising a compound of this disclosure or a pharmaceutically acceptable derivative thereof and a pharmaceutically acceptable carrier, adjuvant, or vehicle. The amount of compound in compositions of this disclosure is such that it is effective to measurably activate a TREM2 protein, or a mutant thereof, in a biological sample or in a patient. In certain embodiments, the amount of compound in compositions of this disclosure is such that it is effective to measurably activate a TREM2 protein, or a mutant thereof, in a biological sample or in a patient. In certain embodiments, a composition of this disclosure is formulated for administration to a patient in need of such composition. In some embodiments, a composition of this disclosure is formulated for oral administration to a patient.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Methods of Use

As discussed herein (see, section entitled “Definitions”), the compounds described herein are to be understood to include all stereoisomers, tautomers, or pharmaceutically acceptable salts of any of the foregoing or solvates of any of the foregoing. Accordingly, the scope of the methods and uses provided in the instant disclosure is to be understood to encompass also methods and uses employing all such forms.

Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

Without wishing to be bound by any particular theory, the following is noted: TREM2 has been implicated in several myeloid cell processes, including phagocytosis, proliferation, survival, and regulation of inflammatory cytokine production. Ulrich and Holtzman 2016. In the last few years, TREM2 has been linked to several diseases. For instance, mutations in both TREM2 and DAP12 have been linked to the autosomal recessive disorder Nasu-Hakola Disease, which is characterized by bone cysts, muscle wasting and demyelination phenotypes. Guerreiro et al. 2013. More recently, variants in the TREM2 gene have been linked to increased risk for Alzheimer's disease (AD) and other forms of dementia including frontotemporal dementia. Jonsson et al. 2013, Guerreiro, Lohmann et al. 2013, and Jay, Miller et al. 2015. In particular, the R47H variant has been identified in genome-wide studies as being associated with increased risk for late-onset AD with an overall adjusted odds ratio (for populations of all ages) of 2.3, second only to the strong genetic association of ApoE to Alzheimer's. The R47H mutation resides on the extracellular lg V-set domain of the TREM2 protein and has been shown to impact lipid binding and uptake of apoptotic cells and Abeta (Wang et al. 2015; Yeh et al. 2016), suggestive of a loss-of-function linked to disease. Further, postmortem comparison of AD patients' brains with and without the R47H mutation are supportive of a novel loss-of-microglial barrier function for the carriers of the mutation, with the R47H carrier microglia putatively demonstrating a reduced ability to compact plaques and limit their spread. Yuan et al. 2016. Impairment in microgliosis has been reported in animal models of prion disease, multiple sclerosis, and stroke, suggesting that TREM2 may play an important role in supporting microgliosis in response to pathology or damage in the central nervous system. Ulrich and Holtzman 2016. In addition, knockdown of TREM2 has been shown to aggravate a-syn-induced inflammatory responses in vitro and exacerbate dopaminergic neuron loss in response to AAV-SYN in vivo (a model of Parkinson's disease), suggesting that impaired microglial TREM2 signaling exacerbates neurodegeneration by modulating microglial activation states. Guo et. al. 2019. A variety of animal models also suggest that Toll-Like Receptor (TLR) signaling is important in the pathogenesis of Rheumatoid Arthritis (R^{A}) via persistent expression of pro-inflammatory cytokines by macrophages. Signaling through TREM2/DAP12 inhibits TLR responses by reducing MAPK (Erkl/2) activation, suggesting that TREM2 activation may act as a negative regulator of TLR driven RA pathogenesis. Huang and Pope 2009.

In view of the data indicating that deficits in TREM2 activity affect macrophage and microglia function, the compounds disclosed herein are of particular use in disorders, such as those described above and in the embodiments that follow and in neurodegenerative disorders more generally.

In one aspect, the disclosure provides a compound of Formula (I), or a tautomer thereof, or a pharmaceutically acceptable salt of said compound or said tautomer, or a pharmaceutical composition thereof for use in treating or preventing a condition associated with a loss of function of human TREM2.

In another aspect, the present disclosure features a compound of Formula (I), a pharmaceutically acceptable salt, solvate, stereoisomer, or tautomer thereof, or a pharmaceutically acceptable salt and/or solvate of said compound, stereoisomer, or tautomer, or a pharmaceutical composition thereof for use in treating a disease, disorder, or condition. Exemplary diseases, disorders, or conditions include proliferative diseases, cardiovascular diseases, metabolic diseases, inflammatory diseases, autoimmune disorders, neurodegenerative disorders, infectious diseases, and tissues injuries.

In some embodiments, the proliferative disease is a benign condition, e.g., a benign neoplasm. In some embodiments, the proliferative disease is a cancer. The cancer may be a cancer of any cell or tissue in the body, for example, a cancer of the brain, eye, thyroid, breast, lung, stomach, kidney, pancreas, bladder, colon, rectum, uterus, ovaries, prostate, skin, fibrous tissues, lympathic system, bone marrow, blood, or immune system. The cancer may comprise a tumor or solid cancer (e.g., a carcinoma) or a non-mass cancer. Exemplary cancers include glioblastoma, retinoblastoma, skin cancer, ocular cancer, gastrointestinal cancer, breast cancer, lung cancer, ductal carcinoma, lungadenocarcinoma, lymphoma, endometrial cancer, liver cancer, pancreatic cancer, renal cell cancer, ovarian cancer, fibrosarcoma, leukemia, myeloma, and polycythemia vera.

In some embodiments, the disease, disorder, or condition is cardiovascular. Exemplary cardiovascular diseases, disorders, and conditions include stroke, coronary heart disease, cardiomyopathy, arrhythmia (e.g. atrial fibrillation), aortic aneurysms, and venous thrombosis.

In some embodiments, the disease, disorder, or condition is metabolic disease. Exemplary metabolic diseases include diabetes (e.g., Type 1 diabetes or Type 2 diabetes), metabolic dysfunction-associated steatohepatitis (MASH), non-alcoholic steatohepatitis (NASH), and Gaucher's disease.

In some embodiments, the disease, disorder, or condition is an inflammatory disease. Exemplary inflammatory diseases include arthritis, acute and chronic colitis, ulcerative colitis, inflammatory bowel disease, Behcet's disease, and granulomatous disorders.

In some embodiments, the disease, disorder, or condition is an autoimmune disease. Exemplary autoimmune diseases include diabetes (e.g., Type 1 diabetes), lupus, sarcoidosis, and multiple sclerosis.

In some embodiments, the disease, disorder, or condition is a neurological disease. In some embodiments, the neurological disease is a neurodegenerative disease. Exemplary neurodegernative diseases include dementia, Alzheimer's disease, Creutzfeldt-Jakob disease, Parkinson's disease, demential with Lewy bodies, amyotrophic lateral sclerosis (ALS), Huntington's disease, taupathy disease, Nasu-Hakola disease, dry age-related macular degeneration (dry AMD), multiple system atrophy (MSA), Shy-Drager syndrome, progressive supranuclear palsy, cortical basal ganglionic degeneration, glaucoma, retinitis pigmentosa, and retinal degeneration, In other embodiments, the neurological condition is acute trauma, chronic trauma, acute disseminated encephalomyelitis, cognitive deficit and memory loss, essential tremor, central nervous system (CNS) lupus, normal pressure hydrocephalus, and seizures.

In some embodiments, the disease, disorder, or condition is an infectious disease. An infectious disease may be local or systemic. Exemplary infectious diseases include eye infections, malaria, respiratory tract infections, sepsis, herpes (e.g., CNS herpes), parasitic infections, Trypanosome infection, Cruzi infection, Pseudomonas aeruginosa infection, Leishmania donovani infection, group B Streptococcus infection, Campylobacter jejuni infection, Neisseria meningitidis infection, type I HIV, and Haemophilus influenza.

In some embodiments, the disease, disorder, or condition is a tissue injury. Any tissues of the body may be injured. Exemplary injuries include ocular (e.g. hyphoma), spinal cord injury, traumatic brain injury, hepatocellular, and wound repair in diabetes.

In one aspect, the disclosure provides a compound of Formula (I), a pharmaceutically acceptable salt, solvate, stereoisomer, or tautomer thereof, or a pharmaceutically acceptable salt and/or solvate of said compound, stereoisomer, or tautomer, or a pharmaceutical composition thereof for treating or preventing a disease, disorder or condition. In one aspect, the disclosure provides a compound of Formula (I), or a tautomer thereof, or a pharmaceutically acceptable salt of said compound or said tautomer, or a pharmaceutical composition thereof for use in treating or preventing Parkinson's disease, rheumatoid arthritis, Alzheimer's disease, Nasu-Hakola disease, frontotemporal dementia, multiple sclerosis, prion disease, or stroke.

In one aspect, the disclosure provides a compound of Formula (I), or a tautomer thereof, or a pharmaceutically acceptable salt of said compound or said tautomer, or a pharmaceutical composition thereof for use in the preparation of a medicament for treating or preventing a condition associated with a loss of function of human TREM2.

In one aspect, the disclosure provides a compound of Formula (I), or a tautomer thereof, or a pharmaceutically acceptable salt of said compound or said tautomer, or a pharmaceutical composition thereof for use in the preparation of a medicament for treating or preventing Parkinson's disease, rheumatoid arthritis, Alzheimer's disease, Nasu-Hakola disease, frontotemporal dementia, multiple sclerosis, prion disease, or stroke.

In another aspect, the disclosure provides a method of treating or preventing a condition associated with a loss of function of human TREM2 in a subject in need thereof, the method comprising administering to the subject a therapeutically effective amount of the compound of the present disclosure, or a tautomer thereof, or a pharmaceutically acceptable salt of said compound or said tautomer, or a pharmaceutical composition thereof.

In another aspect, the disclosure provides a method of treating or preventing Parkinson's disease, rheumatoid arthritis, Alzheimer's disease, Nasu-Hakola disease, frontotemporal dementia, multiple sclerosis, prion disease, or stroke in a subject in need thereof, the method comprising administering to the subject a therapeutically effective amount of the compound of the present disclosure, or a tautomer thereof, or a pharmaceutically acceptable salt of said compound or said tautomer, or a pharmaceutical composition thereof.

### CSFIR

CSF1R is a cell-surface receptor primarily for the cytokine colony stimulating factor 1 (CSF-1), also known until recently as macrophage colony-stimulating factor (M-CSF), which regulates the survival, proliferation, differentiation and function of mononuclear phagocytic cells, including microglia of the central nervous system. CSF1R is composed of a highly glycosylated extracellular ligand-binding domain, a trans-membrane domain and an intracellular tyrosine-kinase domain. Binding of CSF-1 to CSF1R results in the formation of receptor homodimers and subsequent auto-phosphorylation of several tyrosine residues in the cytoplasmic domain, notably Syk. In the brain, CSF1R is predominantly expressed in microglial cells. It has been found that microglia in CSF1R+/−patients are depleted and show increased apoptosis (Oosterhof et al., 2018).

The present disclosure relates to the unexpected discovery that administration of a TREM2 agonist can rescue the loss of microglia in cells having mutations in CSF1R. It has been previously shown that TREM2 agonist antibody 4D9 increases ATP luminescence (a measure of cell number and activity) in a dose dependent manner when the levels of M-CSF in media are reduced to 5 ng/mL (Schlepckow et al, EMBO Mol Med., 2020) and that TREM2 agonist AL002c increases ATP luminescence when M-CSF is completely removed from the media (Wang et al, J. Exp. Med.; 2020, 217(9): e20200785). This finding suggests that TREM2 agonism can compensate for deficiency in CSF1R signaling caused by a decrease in the concentration of its ligand. In a 5xFAD murine Alzheimer's disease model of amyloid pathology, doses of a CSF1R inhibitor that almost completely eliminate microglia in the brains of wild-type animals show surviving microglia clustered around the amyloid plaques (Spangenberg et al, Nature Communications 2019). Plaque amyloid has been demonstrated in the past to be a ligand for TREM2, and it has been shown that microglial engagement with amyloid is dependent on TREM2 (Condello et al, Nat Comm., 2015). The present dsiclosure relates to the unexpected discovery that it is activation of TREM2 that rescued the microglia in the presence of the CSF1R inhibitor, and that this effect is also observed in patients suffering from loss of microglia due to CSF1R mutation. This discovery has not been previously taught or suggested in the available art.

To date, no prior study has shown that TREM2 agonism can rescue the loss of microglia in cells where mutations in the CSF1R kinase domain reduce CSF1R activity, rather than the presence of a CSF1R inhibitor or a deficiency in CSF1R ligand. Furthermore, no prior study has taught or suggested that reversal of the loss of microglia due to a CSF1R mutation through TREM2 agonism can be used to treat a disease or disorder caused by and/or associated with a CSF1R mutation.

Adult-onset leukoencephalopathy with axonal spheroids and pigmented glia (ALSP), previously recognized as hereditary diffuse leukoencephalopathy with axonal spheroids (HDLS) or pigmentary orthochromatic leukodystrophy (POLD), is an autosomal-dominant central nervous system disease that manifests in the form of variable behavioral, cognitive and motor function changes in patients suffering from the disease. ALSP is characterized by patchy cerebral white matter abnormalities visible by magnetic resonance imaging. However, the clinical symptoms and MRI changes are not specific to ALSP and are common for other neurological conditions, including Nasu-Hakola disease (NHD) and AD, making diagnosis and treatment of ALSP very difficult.

Recent studies have discovered that ALSP is a Mendelian disorder in which patients carry a heterozygous loss of function mutation in the kinase domain of CSF1R, suggesting a reduced level of signaling on the macrophage colony-stimulating factor (M-CSF)/CSF1R axis (Rademakers et al, Nat Genet 2012; Konno et al, Neurology 2018). In one aspect, the present disclosure relates to the surprising discovery that activation of the TREM2 pathway can rescue the loss of microglia in CSF1R+/−ALSP patients, preventing microglia apoptosis, thereby treating the ALSP condition.

In one aspect, the disclosure provides a compound of Formula (I), or a tautomer thereof, or a pharmaceutically acceptable salt of said compound or said tautomer, or a pharmaceutical composition thereof for use in treating or preventing a condition associated with dysfunction of Colony stimulating factor 1 receptor (CSF1R, also known as macrophage colony-stimulating factor receptor/M-CSFR, or cluster of differentiation 115/CD 115).

In one aspect, the disclosure provides a compound of Formula (I), or a tautomer thereof, or a pharmaceutically acceptable salt of said compound or said tautomer, or a pharmaceutical composition thereof for use in treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS).

In one aspect, the disclosure provides a compound of Formula (I), or a tautomer thereof, or a pharmaceutically acceptable salt of said compound or said tautomer, or a pharmaceutical composition thereof for use in the preparation of a medicament for treating or preventing a condition associated with dysfunction of CSF1R.

In one aspect, the disclosure provides a compound of Formula (I), or a tautomer thereof, or a pharmaceutically acceptable salt of said compound or said tautomer, or a pharmaceutical composition thereof for use in the preparation of a medicament for treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS).

In another aspect, the disclosure provides a method of treating or preventing a disease or disorder associated with dysfunction of CSF1R in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the subject is selected for treatment based on a diagnosis that includes the presence of a mutation in a CSF1R gene affecting the function of CSF1R. In some embodiments, the mutation in the CSF1R gene is a mutation that causes a decrease in CSF1R activity or a cessation of CSF1R activity. In some embodiments, the disease or disorder is caused by a heterozygous CSF1R mutation. In some embodiments, the disease or disorder is caused by a homozygous CSF1R mutation. In some embodiments, the disease or disorder is caused by a splice mutation in the csf1r gene. In some embodiments, the disease or disorder is caused by a missense mutation in the csf1r gene. In some embodiments, the disease or disorder is caused by a mutation in the catalytic kinase domain of CSF1R. In some embodiments, the disease or disorder is caused by a mutation in an immunoglobulin domain of CSF1R. In some embodiments, the disease or disorder is caused by a mutation in the ectodomain of CSF1R. In some embodiments, the disease or disorder is a disease or disorder resulting from a change (e.g. increase, decrease or cessation) in the activity of CSF1R. In some embodiments, the disease or disorder is a disease or disorder resulting from a decrease or cessation in the activity of CSF1R. CSF1R related activities that are changed in the disease or disorder include, but are not limited to: decrease or loss of microglia function; increased microglia apoptosis; decrease in Src signaling; decrease in Syk signaling; decreased microglial proliferation; decreased microglial response to cellular debris; decreased phagocytosis; and decreased release of cytokines in response to stimuli. In some embodiments, the disease or disorder is caused by a loss-of-function mutation in CSF1R. In some embodiments, the loss-of-function mutation results in a complete cessation of CSF1R function. In some embodiments, the loss-of-function mutation results in a partial loss of CSF1R function, or a decrease in CSF1R activity.

In another aspect, the disclosure provides a method of treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS) in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the method treats or prevents ALSP, which is an encompassing and superseding name for both HDLS and POLD. In some embodiments, the disease or disorder is a homozygous mutation in CSF1R. In some embodiments, the method treats or prevents pediatric-onset leukoencephalopathy. In some embodiments, the method treats or prevents congenital absence of microglia. In some embodiments, the method treats or prevents brain abnormalities neurodegeneration and dysosteosclerosis (BANDDOS).

In yet another aspect, the disclosure provides a method of treating or preventing Nasu-Hakola disease, Alzheimer's disease, frontotemporal dementia, multiple sclerosis, Guillain-Barre syndrome, amyotrophic lateral sclerosis (ALS), Parkinson's disease, traumatic brain injury, spinal cord injury, systemic lupus erythematosus, rheumatoid arthritis, prion disease, stroke, osteoporosis, osteopetrosis, osteosclerosis, skeletal dysplasia, dysosteoplasia, Pyle disease, cerebral autosomal dominant arteriopathy with subcortical infarcts and leukoencephalopathy, cerebral autosomal recessive arteriopathy with subcortical infarcts and leukoencephalopathy, cerebroretinal vasculopathy, or metachromatic leukodystrophy wherein any of the aforementioned diseases or disorders are present in a patient exhibiting CSF1R dysfunction, or having a mutation in a gene affecting the function of CSF1R, the method comprising administering to the subject a therapeutically effective amount of a compound of the present disclosure, or a tautomer thereof, or a pharmaceutically acceptable salt of said compound or said tautomer, or a pharmaceutical composition thereof.

### ABCD1

The ABCD1 gene provides instructions for producing the adrenoleukodystrophy protein (ALDP). ABCD1 (ALDP) maps to Xq28. ABCD1 is a member of the ATP-binding cassette (ABC) transporter superfamily. The superfamily contains membrane proteins that translocate a wide variety of substrates across extra- and intracellular membranes, including metabolic products, lipids and sterols, and drugs. ALDP is located in the membranes of cell structures called peroxisomes. Peroxisomes are small sacs within cells that process many types of molecules. ALDP brings a group of fats called very long-chain fatty acids (VLCFAs) into peroxisomes, where they are broken down. As ABCD1 is highly expressed in microglia, it is possible that microglial dysfunction and their close interaction with other cell types actively participates in neurodegenerative processes (Gong et al., Annals of Neurology. 2017; 82(5):813-827.). It has been shown that severe microglia loss and damage is an early feature in patients with cerebral form of x-linked ALD (cALD) carrying ABCD1 mutations (Bergner et al., Glia. 2019; 67: 1196-1209). It has also been shown that ABCD1-deficiency leads to an impaired plasticity of myeloid lineage cells that is reflected in incomplete establishment of anti-inflammatory responses, thus possibly contributing to the devastating rapidly progressive demyelination in cerebral adrenoleukodystrophy (Weinhor et al., BRAIN 2018: 141; 2329-2342). These findings emphasize microglia/monocytes/macrophages as crucial therapeutic targets for preventing or stopping myelin destruction in patients with X-linked adrenoleukodystrophy.

The present disclosure relates to the unexpected discovery that administration of a TREM2 agonist can rescue the loss of microglia in cells having mutations in the ABCD1 gene. It has been previously shown that TREM2 agonist antibody 4D9 increases ATP luminescence (a measure of cell number and activity) in a dose dependent manner when the levels of M-CSF in media are reduced to 5 ng/mL (Schlepckow et al, EMBO Mol Med., 2020) and that TREM2 agonist AL002c increases ATP luminescence when M-CSF is completely removed from the media (Wang et al, J. Exp. Med.; 2020, 217(9): e20200785). This finding suggests that TREM2 agonism can compensate for deficiency in ABCD1 function leading to sustained activation, proliferation, chemotaxis of microglia, maintenance of anti-inflammatory environment and reduced astrocytosis caused by a decrease in ABCD1 and accumulation of VLCFAs. The present disclosure relates to the unexpected discovery that activation of TREM2 can rescue the microglia in the presence of the ABCD1 mutation and an increase in VLCFA, and that this effect may be also observed in patients suffering from loss of microglia due to ABCD1 mutation. This discovery has not been previously taught or suggested in the available art.

To date, no prior study has shown that TREM2 agonism can rescue the loss of microglia in cells where mutations in the ABCD1 and a VLCFA increase is present. No prior study has taught or suggested that reversal of the loss of microglia due to an ABCD1 mutation through TREM2 agonism can be used to treat a disease or disorder caused by and/or associated with an ABCD1 mutation.

In one aspect, the disclosure provides a compound of Formula (I), or a tautomer thereof, or a pharmaceutically acceptable salt of said compound or said tautomer, or a pharmaceutical composition thereof for use in treating or preventing a condition associated with dysfunction of ATP-binding cassette transporter 1 (ABCD1).

In one aspect, the disclosure provides a compound of Formula (I), or a tautomer thereof, or a pharmaceutically acceptable salt of said compound or said tautomer, or a pharmaceutical composition thereof for use in treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX).

In one aspect, the disclosure provides a compound of Formula (I), or a tautomer thereof, or a pharmaceutically acceptable salt of said compound or said tautomer, or a pharmaceutical composition thereof for use in the preparation of a medicament for treating or preventing a condition associated with dysfunction of ABCD1.

In one aspect, the disclosure provides a compound of Formula (I), or a tautomer thereof, or a pharmaceutically acceptable salt of said compound or said tautomer, or a pharmaceutical composition thereof for use in the preparation of a medicament for treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX).

In yet another aspect, the disclosure provides a method of treating or preventing a disease or disorder associated with dysfunction of ABCD1 in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the patient is selected for treatment based on a diagnosis that includes the presence of a mutation in an ABCD 1 gene affecting the function of ABCD 1. In some embodiments, the mutation in the ABCD1 gene is a mutation that causes a decrease in ABCD1 activity or a cessation of ABCD1 activity. In some embodiments, the disease or disorder is caused by a heterozygous ABCD1 mutation. In some embodiments, the disease or disorder is caused by a homozygous ABCD1 mutation. In some embodiments, the disease or disorder is caused by a splice mutation in the ABCD1 gene. In some embodiments, the disease or disorder is caused by a missense mutation in the ABCD1 gene. In some embodiments, the disease or disorder is a disease or disorder resulting from a change (e.g. increase, decrease or cessation) in the activity of ABCD1. In some embodiments, the disease or disorder is a disease or disorder resulting from a decrease or cessation in the activity of ABCD1. ABCD1 related activities that are changed in the disease or disorder include, but are not limited to peroxisomal import of fatty acids and/or fatty acyl-CoAs and production of adrenoleukodystrophy protein (ALDP). In some embodiments, the disease or disorder is caused by a loss-of-function mutation in ABCD1. In some embodiments, the loss-of-function mutation results in a complete cessation of ABCD1 function. In some embodiments, the loss-of-function mutation results in a partial loss of ABCD1 function, or a decrease in ABCD1 activity. In some embodiments, the disease or disorder is caused by a homozygous mutation in ABCD1. In some embodiments, the disease or disorder is a neurodegenerative disorder. In some embodiments, the disease or disorder is a neurodegenerative disorder caused by and/or associated with an ABCD1 dysfunction. In some embodiments, the disease or disorder is an immunological disorder. In some embodiments, the disease or disorder is an immunological disorder caused by and/or associated with an ABCD1 dysfunction.

In yet another aspect, the disclosure provides a method of treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX) in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, any of the aforementioned diseases are present in a patient exhibiting ABCD1 dysfunction or having a mutation in a gene affecting the function of ABCD1. In some embodiments, the method treats or prevents X-linked adrenoleukodystrophy (x-ALD). In some embodiments, the x-ALD is a cerebral form of x-linked ALD (cALD). In some embodiments, the method treats or prevents Addison disease wherein the patient has been found to have a mutation in one or more ABCD1 genes affecting ABCD1 function. In some embodiments, the method treats or prevents Addison disease, wherein the patient has a loss-of-function mutation in ABCD1.

In yet another aspect, the disclosure provides a method of treating or preventing Nasu-Hakola disease, Alzheimer's disease, frontotemporal dementia, multiple sclerosis, Guillain-Barre syndrome, amyotrophic lateral sclerosis (ALS), or Parkinson's disease, wherein any of the aforementioned diseases or disorders are present in a patient exhibiting ABCD1 dysfunction, or having a mutation in a gene affecting the function of ABCD1, the method comprising administering to the subject a therapeutically effective amount of a compound of the present disclosure, or a tautomer thereof, or a pharmaceutically acceptable salt of said compound or said tautomer, or a pharmaceutical composition thereof. Autism Spectrum Disorders

It has been found that TREM2 deficient mice exhibit symptoms reminiscent of autism spectrum disorders (ASDs) (Filipello et al., Immunity, 2018, 48, 979-991). It has also been found that microglia depletion of the autophagy Aatg7 gene results in defective synaptic pruning and results in increased dendritic spine density, and abnormal social interaction and repetitive behaviors indicative of ASDs (Kim, et al., Molecular Psychiatry, 2017, 22, 1576-1584.). Further studies have shown that increased dendritic spin density detected in post-mortem ASD brains, likely caused by defective synaptic pruning, results in circuit hypoconnectivity and behavioral defects and are a potential origin of a number of neurodevelopmental diseases (Tang, et al., Neuron, 2014, 83, 1131-1143). Without intending to be limited to any particular theory, these findings suggest that TREM2 activation can reverse microglia depletion, and therefore correct the defective synaptic pruning that is central to neurodevelopmental diseases such as ASDs. The present disclosure relates to the unexpected discovery that activation of TREM2, using a compound of Formula (I), can rescue microglia in subjects suffering from an ASD. This discovery has not been previously taught or suggested in the available art.

In one aspect, the disclosure provides a compound of Formula (I), or a tautomer thereof, or a pharmaceutically acceptable salt of said compound or said tautomer, or a pharmaceutical composition thereof for use in treating autism or autism spectrum disorders.

In yet another aspect, the present disclosure provides a compound of the present disclosure, or a tautomer thereof, or a pharmaceutically acceptable salt of said compound or said tautomer, or a pharmaceutical composition thereof for use in the preparation of a medicament for treating autism or autism spectrum disorders.

In yet another aspect, the present disclosure provides a method of treating autism or autism spectrum disorders in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the method treats autism. In some embodiments, the method treats Asperger syndrome.

In some embodiments, the disclosure provides a method of increasing the activity of TREM2, the method comprising contacting a compound of the present disclosure, or a pharmaceutically acceptable salt thereof with the TREM2. In some embodiments, the contacting takes place in vitro. In some embodiments, the contacting takes place in vivo. In some embodiments, the TREM2 is human TREM2.

### Combination Therapies

Depending upon the particular condition, or disease, to be treated, additional therapeutic agents, which are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this disclosure may also be combined with include, without limitation: treatments for Parkinson's disease, rheumatoid arthritis, Alzheimer's disease, Nasu-Hakola disease, frontotemporal dementia, multiple sclerosis, prion disease, or stroke.

As used herein, the term “combination,” “combined,” and related terms refers to the simultaneous or sequential administration of therapeutic agents in accordance with this disclosure. For example, a combination of the present disclosure may be administered with another therapeutic agent simultaneously or sequentially in separate unit dosage forms or together in a single unit dosage form.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the present disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the present disclosure may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the present disclosure are administered as a multiple dosage regimen within greater than 24 hours a parts.

In one embodiment, the present disclosure provides a composition comprising a provided compound or a pharmaceutically acceptable salt thereof and one or more additional therapeutic agents. The therapeutic agent may be administered together with a provided compound or a pharmaceutically acceptable salt thereof, or may be administered prior to or following administration of a provided compound or a pharmaceutically acceptable salt thereof. Suitable therapeutic agents are described in further detail below. In certain embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

### Definitions

The following definitions are provided to assist in understanding the scope of this disclosure.

Unless otherwise indicated, all numbers expressing quantities of ingredients, reaction conditions, and so forth used in the specification or claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound.

As used herein, the following definitions shall apply unless otherwise indicated. For purposes of this disclosure, the chemical elements are identified in accordance with the Periodic Table of the Elements, CAS version, Handbook of Chemistry and Physics, 101^{st} Ed. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the instant disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated. For example, (1R)-1-methyl-2-(trifluoromethyl)cyclohexane is meant to encompass (1R,2R)-1-methyl-2-(trifluoromethyl)cyclohexane and (1R,2S)-1-methyl-2-(trifluoromethyl)cyclohexane. A bond drawn with a wavy line indicates that both stereoisomers are encompassed. This is not to be confused with a wavy line drawn perpendicular to a bond which indicates the point of attachment of a group to the rest of the molecule.

The term “stereoisomer” or “stereoisomerically pure” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of the other enantiomer and diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. For example, the following is illustrative of tautomers of the compounds of Formula I, wherein Ring Ais a ring system of formula

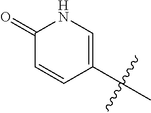 [SMILES: *c1ccc(=O)[nH]c1]

### and R³ is H:

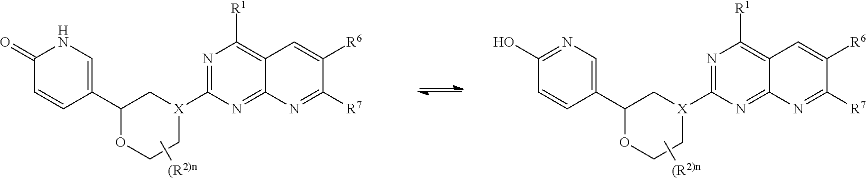

Accordingly, the scope of the instant disclosure is to be understood to encompass all tautomeric forms of the compounds disclosed herein.

### Isotopically-Labelled Compounds

Further, the scope of the present disclosure includes all pharmaceutically acceptable isotopically-labelled compounds of the compounds disclosed herein, such as the compounds of Formula I, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as 2H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula I, for example, those incorporating a radioactive isotope, are useful in drug and/or substrate tissue distribution studies. The radioactive isotopes tritium (H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### Solvates

As discussed above, the compounds disclosed herein and the stereoisomers, tautomers, and isotopically-labelled forms thereof or a pharmaceutically acceptable salt of any of the foregoing may exist in solvated or unsolvated forms.

The term “solvate” as used herein refers to a molecular complex comprising a compound or a pharmaceutically acceptable salt thereof as described herein and a stoichiometric or non-stoichiometric amount of one or more pharmaceutically acceptable solvent molecules. If the solvent is water, the solvate is referred to as a “hydrate.”

Accordingly, the scope of the instant disclosure is to be understood to encompass all solvents of the compounds disclosed herein and the stereoisomers, tautomers and isotopically-labelled forms thereof or a pharmaceutically acceptable salt of any of the foregoing.

### Miscellaneous Definitions

This section will define additional terms used to describe the scope of the compounds, compositions and uses disclosed herein.

The term “alkyl”, as used herein, means a straight-chain (i.e., unbranched) or branched, substituted or unsubstituted hydrocarbon chain that is completely saturated or that contains one or more units of unsaturation, that has a single point of attachment to the rest of the molecule. Unless otherwise specified, alkyl groups contain 1 to 6 alkyl carbon atoms. In some embodiments, alkyl groups contain 1 to 5 alkyl carbon atoms. In other embodiments, alkyl groups contain 1 to 4 alkyl carbon atoms. In still other embodiments, alkyl groups contain 1 to 3 alkyl carbon atoms, and in yet other embodiments, alkyl groups contain 1 to 2 alkyl carbon atoms.

The term “cycloalkyl” or “carbocyclic” as used herein, means a hydrocarbon ring, substituted or unsubstituted that is completely saturated or that contains one or more units of unsaturation but which is not aromatic, that has a single point of attachment to the rest of the molecule. In some embodiments “cycloalkyl,” refers to a monocyclic or bicyclic, bridged bicyclic, or spirocyclic ring, C₃₋₁₂ hydrocarbon that is completely saturated or that contains one or more units of unsaturation, but which is not aromatic, that has a single point of attachment to the rest of the molecule. Suitable cycloalkyl groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, and sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for alkyl groups. Additionally or alternatively, any substitutable nitrogen of a bridged bicyclic group is optionally substituted. Exemplary bicyclic rings include:

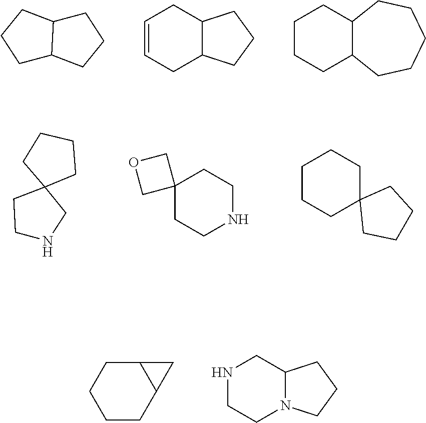

Exemplary bridged bicyclics include:

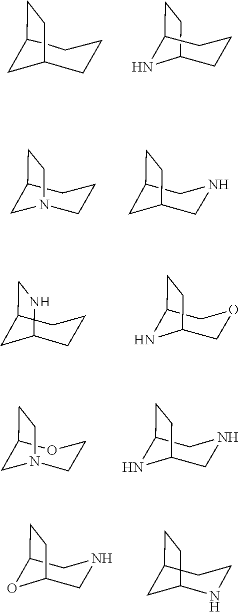

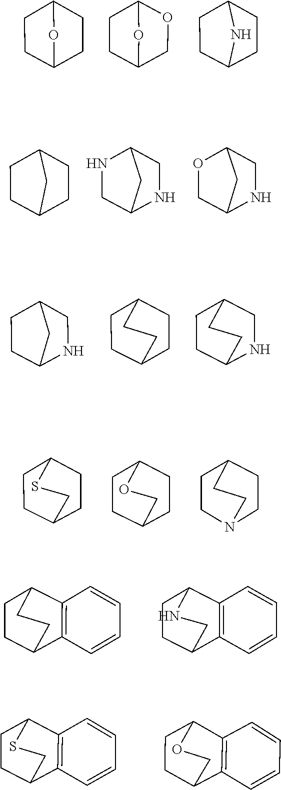

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “C₁₋₆ haloalkyl” refers to a C₁₋₆ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen; or an oxygen, sulfur, nitrogen, phosphorus, or silicon atom in a heterocyclic ring.

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted alkyl group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted alkyl group.

As used herein, the terms “heterocycle,” “heterocyclyl,” “heterocyclic radical,” and “heterocyclic ring” are used interchangeably and refer to a stable 5- to 7-membered monocyclic or 7 to 10-membered bicyclic heterocyclic moiety that is either saturated or partially unsaturated, and having, in addition to carbon atoms, one or more, preferably 1 to 4, heteroatoms, as defined above. When used in reference to a ring atom of a heterocycle, the term “nitrogen” includes a substituted nitrogen. As an example, in a saturated or partially unsaturated ring (having 0 to 3 heteroatoms selected from oxygen, sulfur and nitrogen.

A heterocyclic ring can be attached to a provided compound at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloalkyl rings, such as indolinyl, 3H-indolyl, chromanyl, phenanthridinyl, or tetrahydroquinolinyl. A heterocyclyl group may be monocyclic or bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The terms “C₁₋₃alkyl,” “C₁₋₈ alkyl,” and “C₁₋₆alkyl” as used herein refer to a straight or branched chain hydrocarbon containing from 1 to 3, 1 to 5, and 1 to 6 carbon atoms, respectively. Representative examples of C₁₋₃alkyl, C₁₋₈ alky, or C₁₋₆alkyl include, but are not limited to, methyl, ethyl, n-propyl, iso-propyl, n-butyl, sec-butyl, iso-butyl, tert-butyl, pentyl and hexyl.

The term “C₂₋₄alkenyl” as used herein refers to a saturated hydrocarbon containing 2 to 4 carbon atoms having at least one carbon-carbon double bond. Alkenyl groups include both straight and branched moieties. Representative examples of C₂₋₄alkenyl include, but are not limited to, 1-propenyl, 2-propenyl, 2-methyl-2-propenyl, and butenyl.

The term “C₃₋₆-cycloalkyl” as used herein refers to a saturated carbocyclic molecule wherein the cyclic framework has 3 to 6 carbon atoms. Representative examples of C₃₋₈ cycloalkyl include, but are not limited to, cyclopropyl, cyclobutyl, cyclopentyl, and cyclohexyl.

The terms “diC₁₋₃alkylamino” as used herein refer to —NR*R**, wherein R* and R** independently represent a C₁₋₃alkyl as defined herein. Representative examples of diC₁₋₃alkylamino include, but are not limited to, —N(CH₃)₂, —N(CH₂CH₃)₂, —N(CH₃)(CH₂CH₃), —N(CH₂CH₂CH₃)₂, and —N(CH(CH₃)₂)₂.

The term “C₁₋₃alkoxy” and “C₁₋₆alkoxy” as used herein refer to —OR, wherein R #represents a C₁₋₃alkyl and C₁₋₆alkyl group, respectively, as defined herein. Representative examples of C₁₋₃alkoxy or C₁₋₆alkoxy include, but are not limited to, methoxy, ethoxy, propoxy, iso-propoxy, and butoxy.

The term “halogen” as used herein refers to —F, —CI, —Br, or —I.

The term “halo” as used herein as a prefix to another term for a chemical group refers to a modification of the chemical group, wherein one or more hydrogen atoms are substituted with a halogen as defined herein. The halogen is independently selected at each occurrence. For example, the term “C₁₋₆haloalkyl” refers to a C₁₋₆alkyl as defined herein, wherein one or more hydrogen atoms are substituted with a halogen. Representative examples of C₁₋₆haloalkyl include, but are not limited to, —CH₂F, —CHF₂, —CF₃, —CHFCl, —CH₂CF₃, —CFHCF₃, —CF₂CF₃, —CH(CF₃)₂, —CF(CHF₂)₂, and —CH(CH₂F)(CF₃). Further, the term “C₁₋₆haloalkoxy” for example refers to a C₁₋₆alkoxy as defined herein, wherein one or more hydrogen atoms are substituted with a halogen. Representative examples of C₁₋₆haloalkoxy include, but are not limited to, —OCH₂F, —OCHF₂, —OCF₃, —OCHFCl, —OCH₂CF₃, —OCFHCF₃, —OCF₂CF₃, —OCH(CF₃)₂, —OCF(CHF₂)₂, and —OCH(CH₂F)(CF₃).

The term “5-membered heteroaryl” or “6-membered heteroaryl” as used herein refers to a 5 or 6-membered carbon ring with two or three double bonds containing one ring heteroatom selected from N, S, and O and optionally one or two further ring N atoms instead of the one or more ring carbon atom(s). Representative examples of a 5-membered heteroaryl include, but are not limited to, furyl, imidazolyl, pyrazolyl, isoxazolyl, isothiazolyl, oxadiazolyl, and oxazolyl. Representative examples of a 6-membered heteroaryl include, but are not limited to, pyridyl, pyrimidyl, pyrazyl, and pyridazyl.

The term “C₃₋₆heterocycloalkyl” as used herein refers to a saturated carbocyclic molecule wherein the cyclic framework has 3 to 6 carbons and wherein one carbon atom is substituted with a heteroatom selected from N, O, and S. If the C₃₋₆heterocycloalkyl group is a C₆heterocycloalkyl, one or two carbon atoms are substituted with a heteroatom independently selected from N, O, and S. Representative examples of C₃₋₆heterocycloalkyl include, but are not limited to, aziridinyl, azetidinyl, oxetanyl, pyrrolidinyl, piperazinyl, morpholinyl, and thiomorpholinyl.

The term “C₅₋₈ piroalkyl” as used herein refers a bicyclic ring system, wherein the two rings are connected through a single common carbon atom. Representative examples of C₅₋₈spiroalkyl include, but are not limited to, spiro[2.2]pentanyl, spiro[3.2]hexanyl, spiro[3.3]heptanyl, spiro[3.4]octanyl, and spiro[2.5]octanyl.

The term “C₅₋₈ tricycloalkyl” as used herein refers a tricyclic ring system, wherein all three cycloalkyl rings share the same two ring atoms. Representative examples of C₅₋₈tricycloalkyl include, but are not limited to, tricyclo[1.1.1.0^{1,3}]pentanyl,

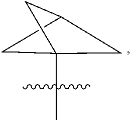 [SMILES: *C12CC(C1)C2]

tricyclo[2.1.1.0^{1,4}]hexanyl, tricyclo[3.1.1.0^{1,5}]hexanyl, and tricyclo[3.2.1.0^{1,5}]octanyl.

The term “aryl” used alone or as part of a larger moiety as in “aralkyl,” “aralkoxy,” or “aryloxyalkyl,” refers to monocyclic or bicyclic ring systems having a total of 4 to 14 ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As described herein, compounds of the present disclosure may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

The term “pharmaceutically acceptable” as used herein refers to generally recognized for use in subjects, particularly in humans.

The term “pharmaceutically acceptable salt” as used herein refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like; or (2) salts formed when an acidic proton present in the parent compound either is replaced by a metal ion, for example, an alkali metal ion, an alkaline earth ion, or an aluminum ion; or coordinates with an organic base such as ethanolamine, diethanolamine, triethanolamine, N-methylglucamine, dicyclohexylamine, and the like. Additional examples of such salts can be found in Berge et al., J. Pharm. Sci. 66(1):1-19 (1977). See also Stahl et al., Pharmaceutical Salts: Properties, Selection, and Use, 2^{nd} Revised Edition (2011).

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherants, glidants, disintegrants, flavoring agents, coatings, binders, sweeteners, lubricants, sorbents, preservatives, and the like.

The term “subject” as used herein refers to humans and mammals, including, but not limited to, primates, cows, sheep, goats, horses, dogs, cats, rabbits, rats, and mice. In one embodiment the subject is a human.

The term “therapeutically effective amount” as used herein refers to that amount of a compound disclosed herein that will elicit the biological or medical response of a tissue, a system, or subject that is being sought by a researcher, veterinarian, medical doctor or other clinician.

### General Synthetic Procedures

In order that the disclosure described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

All temperatures are in degrees Celsius (° C.) and are uncorrected. Reagent grade chemicals and anhydrous solvent were purchased from commercial sources and unless otherwise mentioned, were used without further purification. The names of the products were determined using the naming software included in ChemDraw (PerkinElmer).

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

The compounds provided herein can be synthesized according to the procedures described in the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

All starting materials are either commercially available, for example, from Merck Sigma-Aldrich Inc. and Enamine Ltd. or known in the art and may be synthesized by employing known procedures using ordinary skill. Starting material may also be synthesized via the procedures disclosed herein. Suitable reaction conditions, such as, solvent, reaction temperature, and reagents, may be found in the examples provided herein. Suitable leaving groups, can include but are not limited to, halogens (e.g. fluoride, chloride, bromide, iodide), sulfonates (e.g. mesylate, tosylate, benzenesulfonate, brosylate, nosylate, triflate), diazonium, and the like. Suitable organometal coupling reagent groups, can include but are not limited to, boronic acids and esters, organotin and organozinc reagents.

As can be appreciated by the skilled artisan, the representative examples are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds.

Purification methods for the compounds described herein are known in the art and include, for example, crystallization, chromatography (for example, liquid and gas phase), extraction, distillation, trituration, and reverse phase HPLC.

The disclosure further encompasses “intermediate” compounds, including structures produced from the synthetic procedures described, whether isolated or generated in-situ and not isolated, priorto obtaining the finally desired compound. These intermediates are included in the scope of this disclosure. Exemplary embodiments ofsuch intermediate compounds are set forth in the Examples below.

### EXAMPLES

This section provides specific examples of compounds of Formula I and methods of making the same.

List of Abbreviations

| | |
|---|---|
| AcOH | Acetic acid |
| aq or aq. | Aqueous |
| B₂pin₂ | Bis(pinacolato)diboron |
| DCM | Dichloromethane |
| DMAP | 4-dimethylaminopyridine |
| DMF | N,N-dimethylformamide |
| DMSO | dimethyl sulfoxide |
| Dppf, DPPF or dppf | 1,1′-bis(diphenylphosphino)ferrocene |
| eq or eq. or equiv. | Equivalent |
| ESI or ES | electrospray ionization |
| Et | Ethyl |
| EtOAc or EA | ethyl acetate |
| EtOH | Ethanol |
| G | gram(s) |
| h or hr | hour(s) |
| HPLC | high pressure liquid chromatography |
| iPr | isopropyl |
| iPr₂NEt, DIEA, or | N-ethyl diisopropylamine (Hunig′s base) |
| DIPEA | |
| LC MS, LCMS, LC-MS | liquid chromatography mass spectroscopy |
| or LC/MS | |
| m/z | mass divided by charge |
| Me | methyl |
| CH₃CN | acetonitrile |
| MeOH | methanol |
| Mg | milligrams |
| Min | minutes |
| mL | milliliters |
| MS | mass spectra |
| n-BuLi | n-butyllithium |
| NMR | nuclear magnetic resonance |
| Pd(amphos)Cl₂ | Bis(di-tert-butyl(4-dimethylaminophenyl)- |
| | phosphine)dichloropalladium(II) |
| PE | Petroleum ether |
| PEPPSI-SIPr | (1,3-Bis(2,6-diisopropylphenyl)imidazolidene) (3-chloropyridyl) |
| | palladium(II) dichloride |
| Ph | phenyl |
| RT or rt or r.t. | room temperature |
| RuPhos Pd G3 | (2-Dicyclohexylphosphino-2′,6′-diisopropoxy-1,1′-biphenyl)[2- |
| | (2′-amino-1,1′-biphenyl)]palladium(II) methanesulfonate |
| sat. | Saturated |
| SFC | supercritical fluid chromatography |
| TEA or Et₃N | triethylamine |
| THF | tetrahydrofuran |
| Xantphos Pd G3 | [(4,5-Bis(diphenylphosphino)-9,9-dimethylxanthene)-2-(2′- |
| | amino-1,1′-biphenyl)]palladium(II) methanesulfonate |

### General Analytical and Purification Methods

Provided in this section are descriptions of the general analytical and purification methods used to prepare the specific compounds provided herein.

### Column Chromatography:

Unless otherwise indicated, crude product-containing residues were purified by passing the crude material or concentrate through either a Biotage brand silica gel column pre-packed with flash silica (SiO₂) or reverse phase flash silica (C18) and eluting the product off the column with a solvent gradient as indicated. For example, a description of silica gel (0-40%0 EtOAc/hexane) means the product was obtained by elution from the column packed with silica using a solvent gradient of 0% to 40% o EtOAc in hexanes.

Flash chromatography was performed on Teledyne Isco instruments using pre-packaged disposable SiO_{z} stationary phase columns with eluent flow rate range of 15 to 200 mL/min, UV detection (254 and 220 nm).

### Preparative HPLC Method:

prep-HPLC purification was performed using one of the following HPLC conditions:

Where so indicated, the compounds described herein were purified via reverse phase HPLC using Waters Fractionlynx semi-preparative HPLC-MS system utilizing one of the following two HPLC columns: (a) Phenominex Gemini column (5 micron, C18, 150x30 mm) or (b) Waters X-select CSH column (5 micron, C18, 100x30 mm).

A typical run through the instrument included: eluting at 45 mL/min with a linear gradient of 10% (v/v) to 100% MeCN (0.1% v/v formic acid) in water (0.1% formic acid) over 10 minutes; conditions can be varied to achieve optimal separations.

Condition 1: Column: Gemini® 5 um NX-C18 110 Å, 100×30 mm; Mobile Phase A: water (0.1% ammonium bicarbonate additive); Mobile Phase B: MeCN; Gradient 1: 30% B to 50% B;

Condition 2: Column: Gemini® 5 um NX-C18 110 Å, 100×30 mm; Mobile Phase A: water (10 mM ammonium formate additive); Mobile Phase B: MeCN; Flow Rate: 45 mL/min; Gradient 1: 40% B to 60% B in 10.5 min; Gradient 2: 50% B to 70% B in 10.5 min; Gradient 3: 55% B to 75% B in 10.5 min; Gradient 4: 30% B to 50% B; Gradient 5: 50% B to 100% B;

Condition 3: Column: Phenomenex luna C18 150×25 mm×10 um; Mobile Phase A: water (0.1% FA); Mobile Phase B: MeCN; Flow Rate: 25 mL/min; Gradient 1: 45% B to 75% B in 9 min; Gradient 2: 45% B to 75% B in 15 min;

Condition 4: Column: Phenomenex luna C18 150×25 mm×10 um; Mobile Phase A: water (0.1% FA); Mobile Phase B: MeCN; Flow Rate: 120 mL/min; 0% B to 75% B in 20 min;

Condition 5: Column: Phenomenex luna C18 250×50 mm×10 um; Mobile Phase A: water (0.225% FA); Mobile Phase B: MeCN; Gradient 1: 45% B to 60% B;

Condition 6: C18 150×40 mm×15 um; Mobile Phase A: water (0.3% TFA); Mobile Phase B: MeCN;

Condition 7: Column: Waters Xbridge 150×25 mm×5 um; Mobile Phase A: water (ammonia hydroxide v/v); Mobile Phase B: MeCN;

Condition 8: C18 150×40 mm×15 um; Mobile Phase A: water (0.3% FA); Mobile Phase B: MeCN;

### Chiral HPLC Method:

Chiral HPLC purification was performed using one of the following Chiral HPLC conditions:

Condition 1: Column: Lux i-Amy-1 4.6×150 mm; Mobile Phase A: 10 mM ammonium formate (aq); Mobile Phase B: IPA; Gradient 1: 5% B to 60% B;

### Analytical HPLC Method:

Where so indicated, the compounds described herein were analyzed using an Aglilent 1100 series instrument with DAD detector (190 nm to 300 nm).

### Flash Chromatography Method:

Where so indicated, flash chromatography was performed on Teledyne Isco instruments using pre-packaged disposable SiO₂ stationary phase columns with eluent flow rate range of 15 to 200 mL/min, UV detection (254 and 220 nm).

### Preparative Chiral Supercritical Fluid Chromatography (SFC) Method:

Where so indicated, the compounds described herein were purified via chiral SFC using one of the two following chiral SFC columns: (a) Chiralpak IG 2x25 cm, 5 μm or (b) Chiralpak AD-H 2x15 cm, 5 m.

Some CP Analytical-SFC experiments were run on SFC Method Station (Thar, Waters) with the following conditions: Column temperature: 40° C., Mobile phase: CO₂/Methanol (0.2% Methanol Ammonia)=Flow: 4.0 ml/min, Back Pressure: 120 Bar, Detection wavelength: 214 nm.

Some CP Analytical-SFC experiments were run on SFC-80 (Thar, Waters) with the following conditions: Column temperature: 35° C., Mobile phase (example): CO₂/Methanol (0.2% Methanol Ammonia)=Flow rate: 80 g/min, Back pressure: 100 bar, Detection wavelength: 214 nm.

Preparative CP Method: Acidic reversed phase MPLC: Instrument type: Reveleris™ prep MPLC; Column: Phenomenex LUNA C18(3) (150x25 mm, 10); Flow: 40 mL/min; Column temp: room temperature; Eluent A: 0.1% (v/v) Formic acid in water, Eluent B: 0.1% (v/v) Formic acid in acetonitrile; using the indicated gradient and wavelength.

### Proton NMR Spectra:

Unless otherwise indicated, all ¹H NMR spectra were collected on a Bruker NMR Instrument at 300, 400 or 500 Mhz or a Varian NMR Instrument at 400 Mhz. Where so characterized, all observed protons are reported as parts-per-million (ppm) downfield from tetramethylsilane (TMS) using the internal solvent peak as reference. All NMR were collected at about 25° C.

¹H NMR spectra were recorded in CDCl₃ solution in 5-mm o.d. tubes at 24° C. and were collected on a Varian NMR (AS 400) at 400 MHz for ¹H. The chemical shifts (6) are reported relative to tetramethylsilane (TMS=0.00 ppm) and expressed in ppm.

### LC/MS, Mass Spectra

The mass spectra were recorded with a Waters Micromass ZQ detector at 130° C. The mass spectrometer was equipped with an electrospray ion source (ESI) operated in a positive ion mode and was set to scan between m/z 150-800 with a scan time of 0.3 s. Products and intermediates were analyzed by UPLC/MS on a Gemini-NX (5 M, 2.0×30 mm) using a low pH buffer gradient of 10% to 95% of ACN in H₂O (0.1% HCOOH) over 5 min at 1.0 mL/min for a 3.5 min run.

### Mass Spectra (MS)

Unless otherwise indicated, all mass spectral data for starting materials, intermediates and/or exemplary compounds are reported as mass/charge (m/z), having an [M+H]+ molecular ion. The molecular ion reported was obtained by electrospray detection method (commonly referred to as an ESI MS) utilizing a Waters Acquity UPLC/MS system or a Gemini-NX UPLC/MS system. Compounds having an isotopic atom, such as bromine and the like, are generally reported according to the detected isotopic pattern, as appreciated by those skilled in the art.

### SPECIFIC EXAMPLES

Provided in this section are the procedures to synthesize specific examples of the compounds provided herein. All starting materials are either commercially available from Sigma-Aldrich Inc., unless otherwise noted, or known in the art and may be synthesized by employing known procedures using ordinary skill.

### Example A1: Synthesis of Intermediates

### Method Int-1: Synthesis of A1

### Intermediate A1: 4-amino-2-chloro-6-(2,4-difluorophenyl)pyrimidine-5-carbaldehyde

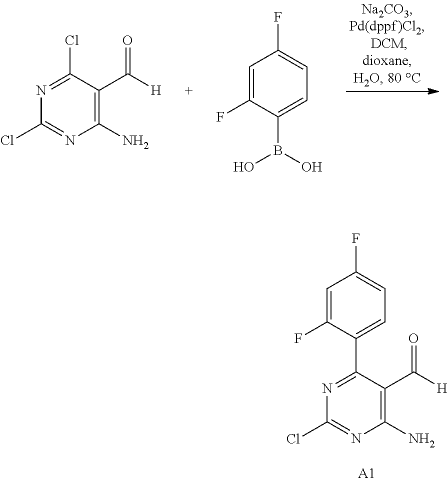

To a solution of 4-amino-2,6-dichloro-pyrimidine-5-carbaldehyde (1 eq, 3 g, 15.6 mmol) and (2,4-difluorophenyl)boronic acid (1 eq, 2.5 g, 15.6 mmol) and 1,4-dioxane (105 mL) at 23° C. was added Pd(dppf)Cl₂ dichloromethane complex (0.05 eq, 638 mg, 0.78 mmol) and Na₂CO₃ (2 M in water) (3 eq, 23 mL, 47 mmol). The mixture was purged under nitrogen atmosphere for 3 min and stirred at 80° C. for 2 h. The reaction mixture was cooled to room temperature, washed with water (200 mL) and extracted with EtOAc (3×200 mL). The organic layers were combined, washed with brine, dried over Na₂SO₄, and filtered. The filtrate was concentrated under reduced pressure and purified by reverse phase flash chromatography (30-70% MeCN in water (0.1% formic acid)) to afford 4-amino-2-chloro-6-(2,4-difluorophenyl)pyrimidine-5-carbaldehyde (A1, 2.9 g, 69% yield) as a solid. LC/MS (ESI, m/z⁺): 270.1 [M+H]⁺. ¹H NMR (CHCl₃-d, 400 MHz): δ_{H} 90.77 (1H, d, J=5.4 Hz), 8.70-8.77 (1H, m), 7.63 (1H, td, J=8.3, 6.3 Hz), 7.04-7.09 (1H, m), 6.92-6.97 (1H, m), 6.04-6.12 (1H, m).

### Method Int-2: Synthesis of A2

### Intermediate A2: 4-amino-6-(2,4-difluorophenyl)-2-[(2S,6R)-2-(1-cyclopropylpyrazol-4-yl)-6-methyl-morpholin-4-yl]pyrimidine-5-carbaldehyde

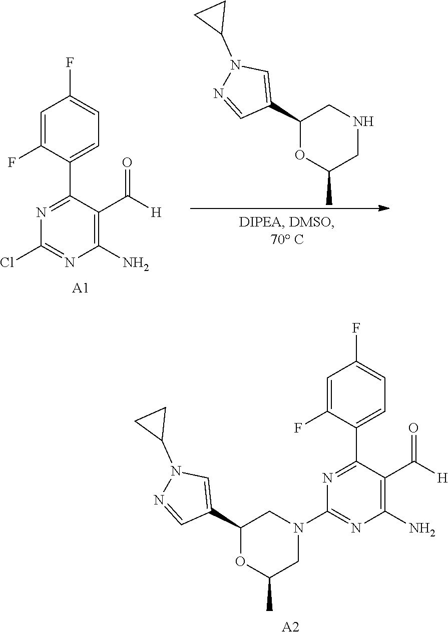

(2S,6R)-2-(1-cyclopropylpyrazol-4-yl)-6-methyl-morpholine (1 eq, 1.7 g, 8.2 mmol), 4-amino-2-chloro-6-(2,4-difluorophenyl)pyrimidine-5-carbaldehyde (A1, 1 eq, 2.2 g, 8.2 mmol) and diisopropylethylamine (5 eq, 7.1 mL, 40.8 mmol) were stirred at 90° C. in DMSO (74 mL) for 30 min. The reaction mixture was cooled to 23° C., washed with water (200 mL) and extracted with EtOAc (3×200 mL). The organic layers were combined, washed with brine, dried over Na₂SO₄, and filtered. The filtrate was concentrated under reduced pressure and purified by silica gel column chromatography (10-60% EtOAc in Hexanes) to afford 4-amino-6-(2,4-difluorophenyl)-2-[(2S,6R)-2-(1-cyclopropylpyrazol-4-yl)-6-methyl-morpholin-4-yl]pyrimidine-5-carbaldehyde (A2, 3.1 g, 86% yield) as a solid. LC/MS (ESI, m/z⁺): 441.2 [M+H]⁺. ¹H NMR (CHCl₃-d, 400 MHz): δ_{H} 90.49 (1H, d, J=4.4 Hz), 8.56-8.64 (1H, m), 7.44-7.51 (3H, m), 6.97-7.03 (1H, m), 6.87-6.92 (1H, m), 5.44-5.51 (1H, m), 4.73-4.92 (1H, m), 4.50 (1H, dd, J=10.9, 2.6 Hz), 3.70-3.75 (1H, m), 3.52-3.58 (1H, m), 2.94 (1H, dd, J=13.3, 11.0 Hz), 2.70 (1H, dd, J=13.3, 10.7 Hz), 1.51-1.54 (1H, m), 1.24-1.31 (3H, m), 1.10 (2H, t, J=3.8 Hz), 0.96-1.03 (2H, m).

### Method Int-4: Synthesis of A6-A9

### Intermediate A6: 4-amino-2-chloro-6-(2,4-difluorophenyl)pyrimidine-5-carbaldehyde

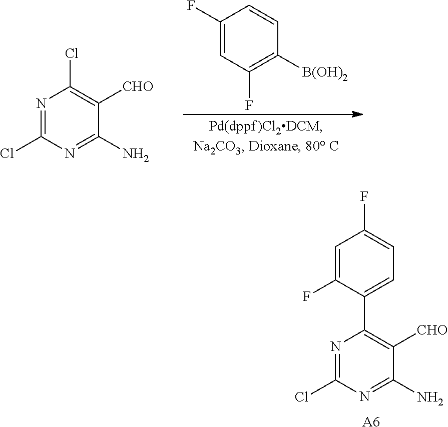

To a flame-dried round-bottom flask equipped with a Teflon-coated magnetic stirring bar was added, under an argon atmosphere, 2,4-dichloro-6-methylpyrimidine-5-carbaldehyde (1 eq, 5 g, 26 mmol) and 2,4-Difluorophenylboronic acid (1.05 eq, 4.32 g, 27.3 mmol), followed by anhydrous 1,4-dioxane (199 mL). The solution was degassed with argon for 5 minutes and then Pd(dppf)Cl₂ dichloromethane complex (0.05 eq, 1.06 g, 1.3 mmol) and Na₂CO₃ (2 M in water) (39.1 mL, 78.1 mmol) were successively added to the reaction mixture. After degassing with argon for 5 more minutes, the reaction was heated at 80° C. with stirring. After 5 h, the reaction was cooled to RT, diluted with EtOAc and washed with water and brine. The organic layer was dried over Na₂SO₄, filtered, and concentrated under reduced pressure. The crude mixture was purified by silica gel column chromatography (0%-10% EtOAc in DCM) to afford 4-amino-2-chloro-6-(2,4-difluorophenyl)pyrimidine-5-carbaldehyde (A6, 2.52 g, 9.36 mmol, 36% yield) as a solid. ¹H-NMR (CHCl₃-d, 400 MHz): δ_{H} 9.78 (d, J=5.44 Hz, 1H), 8.75 (s, 1H), 7.61-7.67 (m, 1H), 7.09 (t, J=8.32 Hz, 1H), 6.93-6.98 (m, 1H), 6.12 (s, 1H). LC/MS; [M+H]⁺ 270.1.

### Intermediate A7: (R⁵)-4-amino-2-(6-(1-cyclopropyl-1H-pyrazol-4-yl)-3,6-dihydro-2H-pyran-4-yl)-6-(2,4-difluorophenyl)-pyrimidine-5-carbaldehyde

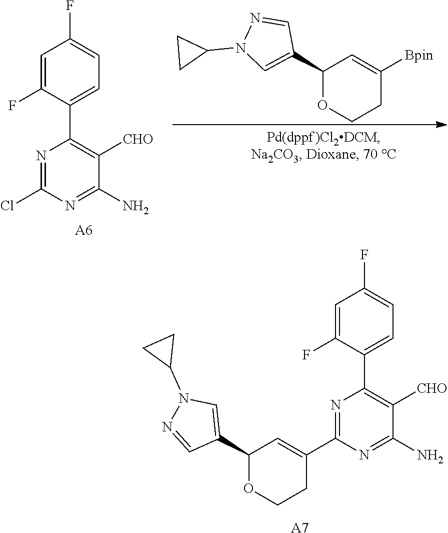

To a flame-dried round-bottom flask equipped with a Teflon-coated magnetic stirring bar was added, under argon atmosphere, 4-amino-2-chloro-6-(2,4-difluorophenyl)pyrimidine-5-carbaldehyde (A6, 1 eq, 1.11 g, 4.12 mmol) and (R⁵)-1-cyclopropyl-4-(4-(4,4,5,5-tetramethyl-1,3,2-dioxaborolan-2-yl)-5,6-dihydro-2H-pyran-2-yl)-1H-pyrazole (1.05 eq, 1.43 g, 4.53 mmol), followed by anhydrous 1,4-dioxane (44.3 mL). The solution was degassed with argon for 5 min and then Pd(dppf)Cl₂ dichloromethane complex (0.05 eq, 168 mg, 206 μmol) and sodium carbonate (2 M in water) (3 eq, 6.18 mL, 12.4 mmol) were successively added to the reaction mixture. After degassing with argon for 5 more minutes, the reaction was heated at 70° C. with stirring. After 15 h, the reaction was cooled to RT, diluted with EtOAc and washed successively with water and brine. The organic layer was dried over Na₂SO₄, filtered, and concentrated under reduced pressure. The crude mixture was purified by silica gel column chromatography (0%-40% EtOAc in DCM) to afford (R⁵)-4-amino-2-(6-(1-cyclopropyl-1H-pyrazol-4-yl)-3,6-dihydro-2H-pyran-4-yl)-6-(2,4-difluorophenyl)-pyrimidine-5-carbaldehyde (A7, 1.32 g, 3.13 mmol, 76% yield) as a solid. ¹H-NMR (CHCl₃-d, 400 MHz): δ_{H} 9.78 (d, J=5.25 Hz, 1H), 8.58 (s, 1H), 7.61 (td, J=8.35, 6.33 Hz, 1H), 7.49 (s, 1H), 7.46 (s, 1H), 7.39-7.40 (m, 1H), 7.03-7.08 (m, 1H), 6.94 (ddd, J=9.93, 8.69, 2.43 Hz, 1H), 5.81 (s, 1H), 5.37-5.39 (m, 1H), 4.04 (dt, J=11.46, 4.98 Hz, 1H), 3.84 (ddd, J=11.45, 7.40, 4.54 Hz, 1H), 3.55 (tt, J=7.22, 3.82 Hz, 1H), 2.70-2.77 (m, 1H), 2.63-2.69 (m, 1H), 1.08-1.12 (m, 2H), 0.96-1.01 (m, 2H). LC/MS; [M+H]⁺ 424.3.

### Intermediate A8: N-(6-methyl-3-(3-(trifluoromethyl)-bicyclo[1.1.1]pentane-1-carbonyl)pyridin-2-yl)pivalamide

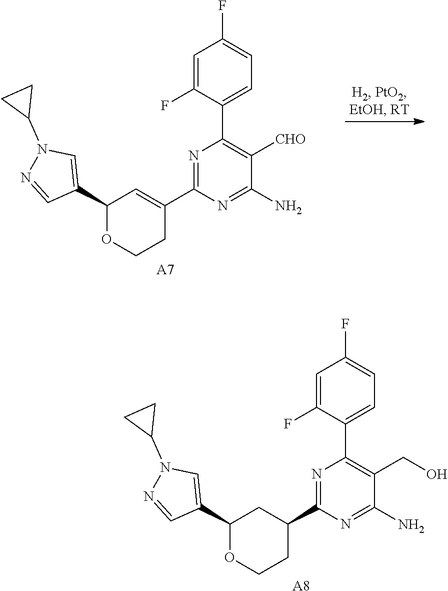

To a flame-dried glass vial, was added (R⁵)-4-amino-2-(6-(1-cyclopropyl-1H-pyrazol-4-yl)-3,6-dihydro-2H-pyran-4-yl)-6-(2,4-difluorophenyl)-pyrimidine-5-carbaldehyde (A7, 1 eq, 300 mg, 709 μmol) and platinum oxide (80.4 mg, 354 μmol), under an argon atmosphere. Anhydrous EtOH (7.1 mL) was added, the vial was capped, and the reaction vessel underwent vac/hydrogen cycles (×3) to purge the reaction vessel under an atmosphere of hydrogen. The vessel was fitted with a hydrogen balloon and the reaction mixture was stirred at RT. After 3 h, the reaction vessel was purged under argon and the reaction solution was filtered over a Celite plug, washing with 9:1 DCM:MeOH (100 mL). The filtrate was concentrated under reduced pressure and purified by silica gel column chromatography (5% MeOH in DCM) to afford N-(6-methyl-3-(3-(trifluoromethyl)-bicyclo[1.1.1]pentane-1-carbonyl)pyridin-2-yl)pivalamide (A8, 180 mg, 418 μmol, 59% yield, d.r.=9:1) as a solid. ¹H-NMR (CHCl₃-d 400 MHz): δ_{H} 7.49-7.51 (m, 1H), 7.45 (s, 2H), 7.00-7.05 (m, 1H), 6.88-6.94 (m, 1H), 5.58 (s, 1H), 4.50 (s, 2H), 4.44 (d, J=11.63 Hz, 1H), 4.18 (d, J=10.99 Hz, 1H), 3.70 (t, J=11.81 Hz, 1H), 3.51-3.54 (m, 1H), 3.00-3.09 (m, 1H), 2.13-2.17 (m, 1H), 1.86-2.00 (m, 3H), 1.06-1.07 (m, 2H), 0.93-0.95 (m, 2H). LC/MS: [M+H]⁺ 428.4.

### Intermediate A9: 4-amino-2-((2R,4S)-2-(1-cyclopropyl-1H-pyrazol-4-yl)tetrahydro-2H-pyran-4-yl)-6-(2,4-difluorophenyl)pyrimidine-5-carbaldehyde

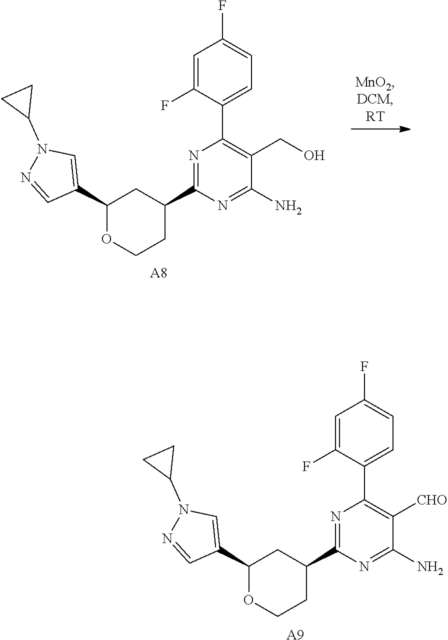

To a flame-dried glass vial equipped with a Teflon-coated magnetic stirring bar, was added (R⁵)-(4-amino-2-(2-(1-cyclopropyl-1H-pyrazol-4-yl)tetrahydro-2H-pyran-4-yl)-6-(2,4-difluorophenyl)-pyrimidin-5-yl)methanol (A8, 1 eq, 180 mg, 421 μmol) and Manganese(IV) oxide (10 eq, 370 mg, 4.21 mmol), and anhydrous DCM (1.7 mL). The vial was sealed, purged under Ar and the reaction mixture was stirred at room temperature for 22 h. The reaction solution was filtered over a Celite plug, washing with 9:1 DCM:MeOH (100 mL). The filtrate was concentrated under reduced pressure and the crude mixture was purified by silica gel column chromatography (60% EtOAc in DCM) to afford 4-amino-2-((2R,4S)-2-(1-cyclopropyl-1H-pyrazol-4-yl)tetrahydro-2H-pyran-4-yl)-6-(2,4-difluorophenyl)-pyrimidine-5-carbaldehyde (A9, 152 mg, 358 μmol, 85% yield, d.r.=95:05) as a solid. ¹H NMR (CHCl₃-d, 400 MHz): δ_{H} 9.78 (d, J=5.00 Hz, 1H), 8.57 (s, 1H), 7.58 (td, J=8.26, 6.39 Hz, 1H), 7.46 (d, J=2.46 Hz, 2H), 7.07 (td, J=8.26, 2.21 Hz, 1H), 6.92-6.97 (m, 1H), 5.81 (s, 1H), 4.45 (d, J=11.40 Hz, 1H), 4.18-4.22 (m, 1H), 3.67-3.74 (m, 1H), 3.51-3.57 (m, 1H), 3.08-3.16 (m, 1H), 2.20-2.23 (m, 1H), 1.94-2.04 (m, 3H), 1.06-1.09 (m, 2H), 0.96-0.99 (m, 2H). LC/MS: [M+H]⁺ 426.4.

Method Int-5: Synthesis of A1

### Intermediate A10: N′-acetyl-2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carbohydrazide

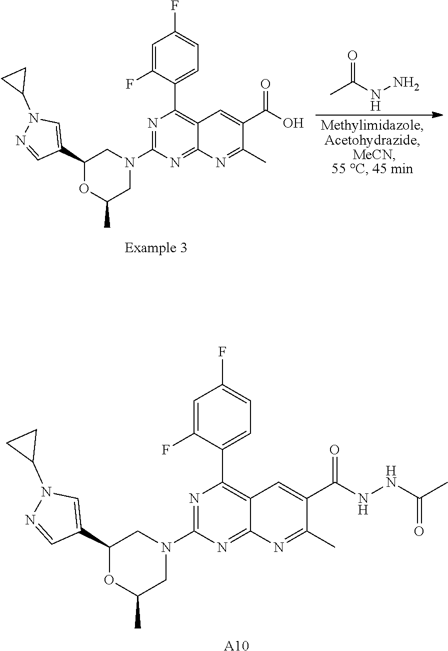

2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxylic acid (Example 3, 1 eq, 100 mg, 0.2 mmol) and N-methylimidazole (1.1 eq, 17 μL, 0.22 eq.) were added to a solution of acetohydrazide (2 eq, 27 μL, 0.4 mmol), and the mixture was stirred at room temperature for 45 min. Brine (30 mL) was added, and the mixture was extracted with EtOAc (2×30 mL). The combined organic layers were dried over Na₂SO₄, filtered, and concentrated under reduced pressure. The crude was purified by reverse phase chromatography (30-70% MeCN in water (0.1% ammonium bicarbonate)) to afford N-acetyl-2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carbohydrazide (A10, 45 mg, 0.08 mmol, 41% yield) as a solid. LC/MS (ESI, m/z⁺): 563.3 [M+H]⁺.

### Method Int-6: Synthesis of A12-13

### Intermediate A12: 2-[(2S,6R)-2-(1-cyclopropylpyrazol-4-yl)-6-methyl-morpholin-4-yl]-4-(2,4-difluorophenyl)-N-methoxy-N,7-dimethyl-pyrido[2,3-d]-pyrimidine-6-carboxamide

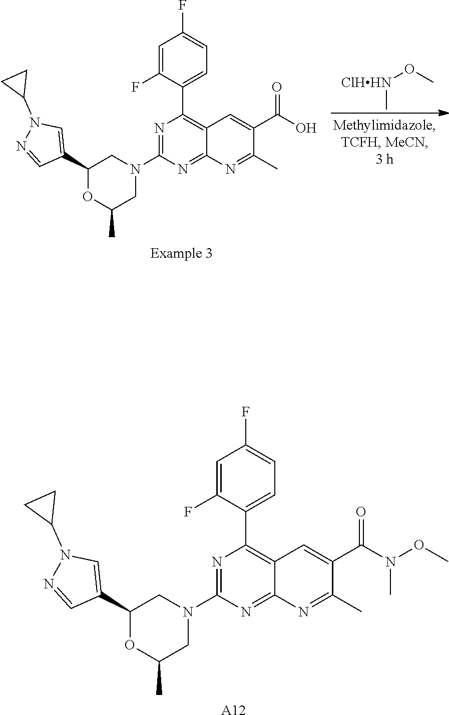

2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxylic acid (Example 3, 1 eq, 100 mg, 0.2 mmol), N,O-dimethylhydroxylamine hydrochloride (2 eq, 39 mg, 0.4 mmol), N-methylimidazole (3.5 eq, 55 μL, 0.7 mmol) and chloro-N,N,N′N′-tetramethylformamidinium hexafluorophosphate (1.5 eq, 83 mg, 0.3 mmol) were dissolved in MeCN (2 mL). The reaction was stirred at room temperature for 3 h. Brine (25 mL) was added, and the reaction was extracted with EtOAc (3×25 mL). The combined organic layers were dried over Na₂SO₄, filtered, concentrated under reduced pressure, and purified by normal phase chromatography (0-5% MeOH in DCM) to afford 2-[(2S,6R)-2-(1-cyclopropylpyrazol-4-yl)-6-methyl-morpholin-4-yl]-4-(2,4-difluorophenyl)-N-methoxy-N,7-dimethyl-pyrido[2,3-d]-pyrimidine-6-carboxamide (A12, 96 mg, 0.18 mmol, 88% yield). LC/MS (ESI, m/z⁺): 550.4 [M+H]⁺.

### Intermediate A13: 1-[2-[(2S,6R)-2-(1-cyclopropylpyrazol-4-yl)-6-methyl-morpholin-4-yl]-4-(2,4-difluorophenyl)-7-methyl-pyrido[2,3-d]pyrimidin-6-yl]but-2-yn-1-one

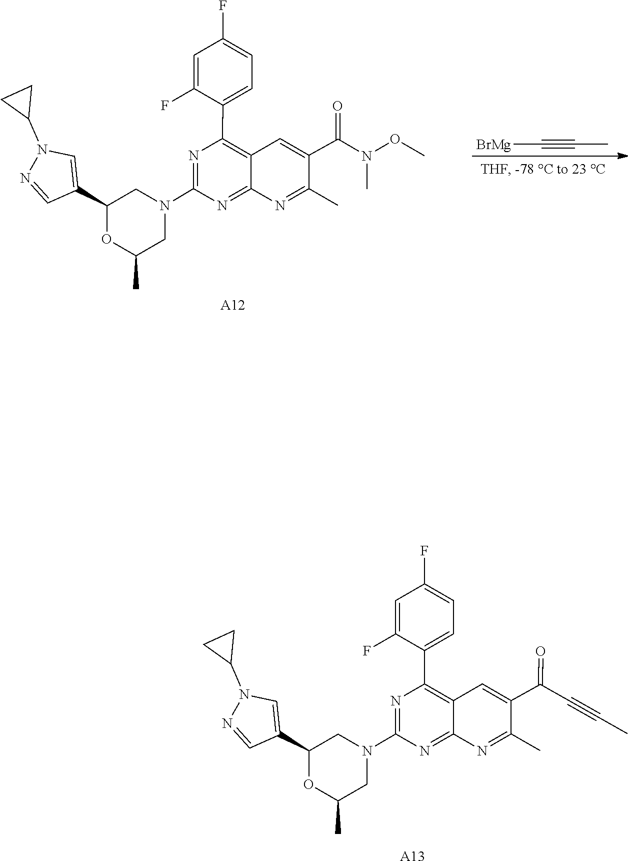

2-[(2S,6R)-2-(1-cyclopropylpyrazol-4-yl)-6-methyl-morpholin-4-yl]-4-(2,4-difluorophenyl)-N-methoxy-N,7-dimethyl-pyrido[2,3-d]pyrimidine-6-carboxamide (A12, 1 eq, 50 mg, 0.09 mmol) was dissolved in THF (1 mL). Bromo(prop-1-ynyl)magnesium (2 eq, 0.36 mL, 0.18 mmol) was added dropwise to the mixture at −78° C. The reaction was stirred 5 min at the same temperature and was warmed up to room temperature to be stirred for 2 h. The mixture was cooled at −78° C. and bromo(prop-1-ynyl)magnesium (1 eq, 0.18 mL, 0.09 mmol) was added. The mixture was warmed at room temperature and was stirred for 1 h. NH₄Cl (10 mL) was added, and the organic phase was extracted with EtOAc (20 mL). The organic layer was washed with brine, dried over Na₂SO₄, filtered, and concentrated under reduced pressure. The crude was purified by normal phase chromatography (0-5% MeOH in DCM) to afford 1-[2-[(2S,6R)-2-(1-cyclopropylpyrazol-4-yl)-6-methyl-morpholin-4-yl]-4-(2,4-difluorophenyl)-7-methyl-pyrido[2,3-d]pyrimidin-6-yl]but-2-yn-1-one (A13, 34 mg, 0.064 mmol, 70% yield). LC/MS (ESI, m/z⁺): 529.2 [M+H]⁺. ¹H NMR (CHCl₃-d, 400 MHz): δ_{H} 8.68 (1H, d, J=3.9 Hz), 7.64 (1H, d, J=8.9 Hz), 7.53 (2H, s), 7.13 (1H, s), 7.04 (1H, s), 4.92-5.14 (2H, m), 4.58 (1H, d, J=10.7 Hz), 3.81 (1H, s), 3.56 (1H, s), 3.10 (1H, dd, J=13.3, 11.0 Hz), 2.98 (3H, s), 2.87 (1H, dd, J=13.4, 10.6 Hz), 2.03-2.04 (3H, m), 1.32 (3H, d, J=6.1 Hz), 1.09-1.11 (2H, m), 1.01 (3H, d, J=7.2 Hz). ¹⁹F NMR (CHCl₃-d, 376 MHz): δ_{F}−105.2 (1F, m), −108.2 (1F, m).

### Method Int-7: Synthesis of A14

### Intermediate A14: N-acetonyl-2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxamide

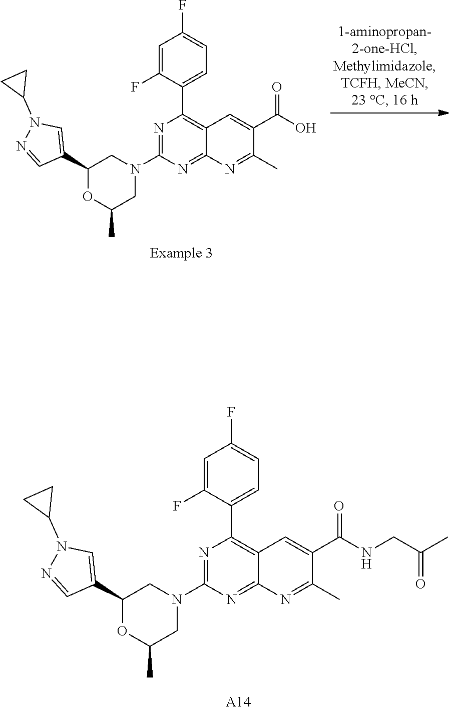 [SMILES: Cc1nc2nc(N3C[C@@H](C)O[C@@H](c4cnn(C5CC5)c4)C3)nc(-c3ccc(F)cc3F)c2cc1C(=O)O ; CC(=O)CNC(=O)c1cc2c(-c3ccc(F)cc3F)nc(N3C[C@@H](C)O[C@@H](c4cnn(C5CC5)c4)C3)nc2nc1C ; "A14"]

To a solution of 1-aminopropan-2-one hydrochloride (2 eq, 0.024 mL, 0.24 mmol) and 2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxylic acid (Example 3, 1 eq, 60 mg, 0.12 mmol) in MeCN (2 mL) was added methylimidazole (1.1 eq, 10 μL, 0.13 mmol) and chloro-N,N,N′N-tetramethylformamidinium hexafluorophosphate (1.1 eq, 37 mg, 0.13 mmol), and the reaction was stirred at room temperature for 16 h. The resulting mixture was concentrated under reduced pressure and purified by reverse phase chromatography (30-70% MeCN in water (0.1% ammonium bicarbonate)) to afford N-acetonyl-2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxamide (A14, 12 mg, 0.021 mmol, 18% yield) as a solid. LC/MS (ESI, m/z⁺): 562.3 [M+H]⁺.

### Method Int-8: Synthesis of A15-A16

### Intermediate A15: 4-amino-6-(2,4-difluorophenyl)-2-(3-morpholino-1-piperidyl)pyrimidine-5-carbaldehyde

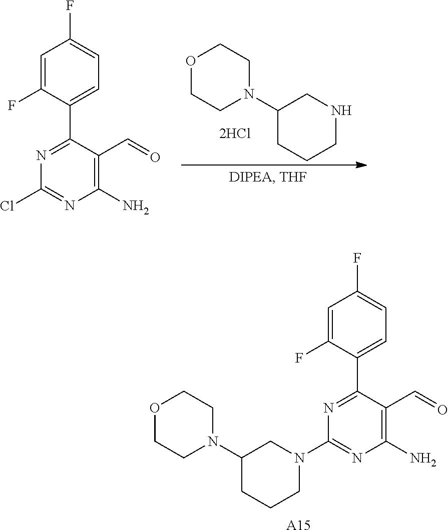

To a rt solution of 4-(3-piperidyl)morpholine-dihydrochloride (1.05 eq, 284 mg, 1.17 mmol) in THF (10 mL) was added 4-amino-2-chloro-6-(2,4-difluorophenyl)pyrimidine-5-carbaldehyde (1 eq, 300 mg, 1.11 mmol) and DIPEA (4 eq, 0.78 mL, 4.45 mmol), and the reaction was stirred at 70° C. for 3 h. The mixture was cooled down, diluted with EtOAc and washed with water, brine, dried over anhydrous Na₂SO₄, and concentrated under reduced pressure. The residue was purified by silica gel column chromatography (35-100% EtOAc in DCM) to afford 4-amino-6-(2,4-difluorophenyl)-2-(3-morpholino-1-piperidyl)pyrimidine-5-carbaldehyde (A15, 430 mg, 1.07 mmol, 96% yield) as a solid. ¹H NMR (CHCl₃-d, 400 MHz): δ_{H} 9.41 (1H, d, J=4.4 Hz), 8.53 (1H, s), 7.42 (1H, d, J=8.4 Hz), 6.95 (1H, td, J=8.3, 2.4 Hz), 6.85 (1H, td, J=9.3, 2.4 Hz), 5.85 (1H, s), 4.46-4.80 (2H, br m), 3.66 (4H, br s), 2.92 (2H, m), 2.57 (4H, br s), 2.25 (1H, m), 1.97 (1H, s), 1.77 (1H, s), 1.45 (2H, m). LC/MS (ESI, m/z⁺): 404.3 [M+1]⁺.

### Intermediate A16: methyl-4-(2,4-difluorophenyl)-7-methyl-2-(3-morpholino-1-piperidyl)pyrido[2,3-d]pyrimidine-6-carboxylate

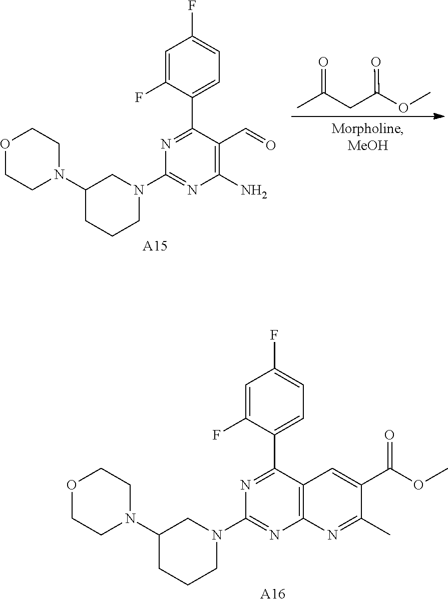

To a solution of methyl 3-oxobutanoate (2 eq, 248 mg, 2.13 mmol) in MeOH was added morpholine (1 eq, 0.09 mL, 1.07 mmol), and the reaction mixture was stirred at rt for 30 min. The reaction mixture was added to a solution of 4-amino-6-(2,4-difluorophenyl)-2-[rac-(1S)-3-morpholino-1-piperidyl]pyrimidine-5-carbaldehyde (1 eq, 430 mg, 1.07 mmol) in MeOH (10 mL), and the reaction was stirred at 90° C. for 4 h in a sealed vessel. The reaction mixture was cooled down, diluted with EtOAc, and washed with saturated aqueous NaHCO₃ and brine. The organic layer as dried over anhydrous Na₂SO₄, filtered, concentrated under reduced pressure, and purified by silica gel column chromatography (0-20% MeOH in DCM) to afford methyl-4-(2,4-difluorophenyl)-7-methyl-2-(3-morpholino-1-piperidyl)-pyrido[2,3-d]pyrimidine-6-carboxylate (A16, 90 mg, 0.19 mmol, 17% yield) as a crude. LC/MS (ESI, m/z⁺): 484.4 [M+H]⁺.

### Method Int-9: Synthesis of A17-A24

### Intermediate A17: N-methoxy-N-methyl-3-(trifluoromethyl)bicyclo[1.1.1]pentane-1-carboxamide

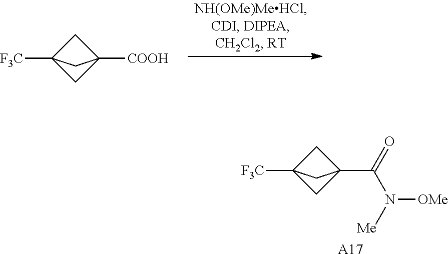

To a flame-dried round-bottomed flask equipped with a teflon-coated magnetic stirring bar was added 3-(trifluoromethyl)bicyclo[1.1.1]pentane-1-carboxylic acid (1 eq, 2.70 g, 15.0 mmol) and anhydrous DCM (40 mL). The vessel was sealed, purged under Ar and stirred at RT. CDI (1.2 eq, 2.92 g, 18 mmol) was added in portions. A vigorous evolution of CO₂ was observed. The reaction mixture was stirred for 1 h under Ar at RT. N,O-Dimethylhydroxylamine hydrochloride (1.1 eq, 1.61 g, 16.5 mmol) and N,N-diisopropylethylamine (1.2 eq, 3.1 mL, 18 mmol) were added and the reaction mixture was continued to stir at RT overnight. After 24 h, the reaction mixture was concentrated under reduced pressure and diluted with EtOAc (100 mL). The crude reaction solution was washed with 1.0 M HCl (aq) (×2), sat. NaHCO₃(aq) (×2) and brine. The organic layer was collected, dried over Na₂SO₄, filtered, and concentrated under reduced pressure to approx. 50 mL in volume. The crude solution was passed through a short plug of SiO₂, washing with (50% EtOAc in Hexanes, 500 mL). The filtrate was concentrated under reduced pressure to afford N-methoxy-N-methyl-3-(trifluoromethyl)bicyclo[1.1.1]pentane-1-carboxamide (A17, 3.09 g, 13.8 mmol, 92% yield) as an oil, which slowly crystallizes into a solid. ¹H NMR (CHCl₃-d, 400 MHz): δ_{H} 3.68 (3H, s), 3.19 (3H, s), 2.29 (6H, s). ¹⁹F NMR (CHCl₃-d, 376 MHz): δ_{F}−73.2 (3F, s). LC/MS: [M+H]⁺ 224.2.

### Intermediate A18: 2,2-dimethyl-N-(6-methyl-2-pyridyl)propanamide

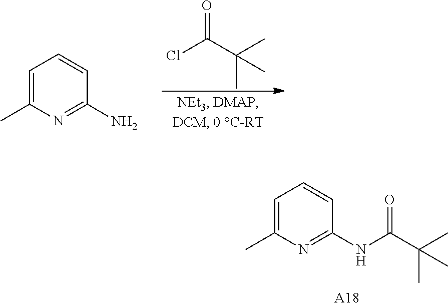

To a flame-dried, round-bottomed flask equipped with a Teflon-coated magnetic stirring bar was added 6-methylpyridin-2-amine (1 eq, 2.70 g, 25 mmol) and 4-dimethylaminopyridine (0.1 eq, 0.31 g, 2.5 mmol). The flask was capped, placed under 1 atm of Ar and dissolved in anhydrous DCM (20 mL) with stirring at RT. NEt₃ (1.5 eq, 5.2 mL, 37.5 mmol) was added and the reaction mixture was cooled to 0° C. in an ice bath with stirring. Pivaloyl chloride (1.2 eq, 3.7 mL, 30.0 mmol) was added and the reaction mixture was allowed to warm to RT and stirred overnight. The reaction mixture was diluted with DCM (50 mL) and washed with sat. NH₄Cl (aq). The aqueous layer was extracted with DCM (×2). The combined organic extracts were dried with Na₂SO₄, filtered, and concentrated under reduced pressure onto Celite (15 g). The crude reaction mixture was purified by silica gel column chromatography (2%-30% EtOAc in Hexanes) to afford 2,2-dimethyl-N-(6-methyl-2-pyridyl)propanamide (A18, 4.8 g, 25.0 mmol, 99% yield) as a syrup. ¹H NMR (CHCl₃-d, 400 MHz): δ_{H} 7.96 (1H, d, J=8.3 Hz), 7.87 (1H, s), 7.40 (1H, d, J=8.3 Hz), 2.39 (3H, s), 2.23 (3H, s), 1.32 (8H, s). LC/MS: [M+H]⁺ 193.2.

### Intermediate A19: N-(6-methyl-3-(3-(trifluoromethyl)bicyclo[1.1.1]pentane-1-carbonyl)pyridin-2-yl)pivalamide

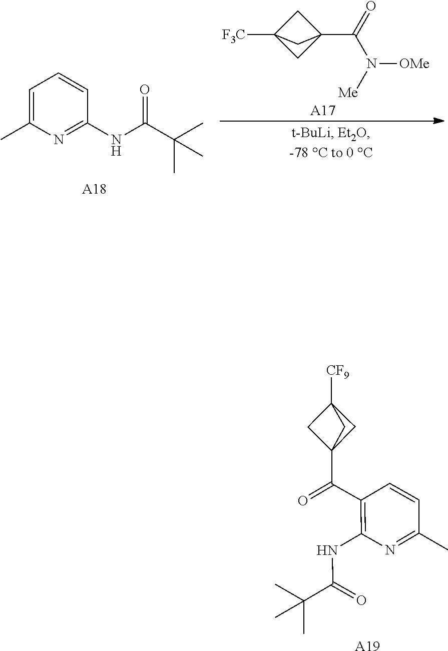

To a flame-dried, round-bottomed flask equipped with a Teflon-coated magnetic stirring bar was added 2,2-dimethyl-N-(6-methyl-2-pyridyl)propenamide (A18, 1 eq, 385 mg, 2 mmol). The flask was sealed and placed under 1 atmosphere of argon. Anhydrous Et₂O (10 mL) was added, and the resulting solution was stirred at −78° C. in an acetone-dry ice bath. tert-BuLi (2.3 eq, 2.7 mL, 4.6 mmol) was added dropwise over 3 min and the reaction mixture was continued to stir at −78° C. for 2 h. The reaction mixture was stirred for 45 min at 0° C. in an ice bath and then cooled back down to −78° C. N-methoxy-N-methyl-3-(trifluoromethyl)bicyclo[1.1.1]pentane-1-carboxamide (A17, 1.2 eq, 536 mg, 2.4 mmol) dissolved in anhydrous Et₂O (5 mL) was added to the reaction mixture dropwise. The reaction mixture was continued to stir at −78° C., gradually warming to RT. After 16 h, the reaction mixture was cooled to 0° C. in an ice bath and quenched with saturated NH₄Cl (aq) (3 mL) and diluted with EtOAc (100 mL) and saturated NH₄Cl (aq). The layers were separated, and the organic layer was washed with brine. The organic layer was collected, dried over MgSO₄, filtered, and concentrated under reduced pressure. The crude was purified by silica gel column chromatography (10-80% EtOAc in Hexanes) to afford N-(6-methyl-3-(3-(trifluoromethyl)bicyclo[1.1.1]pentane-1-carbonyl)pyridin-2-yl)pivalamide (A19, 400 mg, 56% yield) as a solid. ¹H NMR (CHCl₃-d, 400 MHz): δ_{H} 10.92 (1H, s), 8.08 (1H, d, J=8.0 Hz), 6.93 (1H, d, J=8.0 Hz), 2.60 (3H, s), 2.47 (6H, s), 1.34 (9H, s). LC/MS: [M+H]⁺ 355.2.

### Intermediate A20: (2-amino-6-methylpyridin-3-yl)(3-(trifluoromethyl)bicyclo[1.1.1]pentan-1-yl)methanone

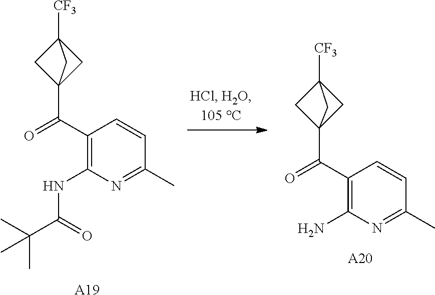

To a round-bottomed flask equipped with a teflon-coated magnetic stirring bar was added N-(6-methyl-3-(3-(trifluoromethyl)bicyclo[1.1.1]pentane-1-carbonyl)pyridin-2-yl)pivalamide (A19, 1 eq, 208 mg, 0.59 mmol), water (5 mL) and 6 M HCl (aq) (20 eq, 2 mL, 11.7 mmol). The flask was fitted with a reflux condenser and heated to 105° C. with stirring for 18 h. The reaction mixture was cooled to room temperature and then further to 0° C. in an ice bath with stirring. 1 M NaOH (aq) was added until the pH was adjusted to 10. The resulting precipitate was collected via vacuum filtration, rinsed with H₂O and dried under reduced pressure to afford (2-amino-6-methylpyridin-3-yl)(3-(trifluoromethyl)-bicyclo[1.1.1]pentan-1-yl)methanone (A20, 142 mg, 90% yield) as a solid. ¹H NMR (CHCl₃-d, 400 MHz): δ_{H} 8.01 (1H, d, J=8.0 Hz), 6.49 (1H, d, J=8.0 Hz), 2.45 (6H, s), 2.41 (3H, s). LC/MS: [M+H]⁺ 271.2.

### Intermediate A21: (2-amino-5-bromo-6-methylpyridin-3-yl)(3-(trifluoromethyl)-bicyclo[1.1.1]pentan-1-yl)methanone

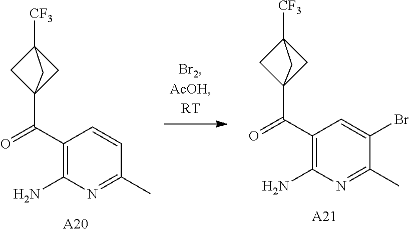

To a vial equipped with a Teflon-coated magnetic stirring bar was added (2-amino-6-methyl-3-pyridyl)-[3-(trifluoromethyl)-1-bicyclo[1.1.1]pentanyl]methanone (A20, 1 eq, 203 mg, 0.75 mmol) and acetic acid (2 mL), and the resulting solution was stirred at RT. Bromine (1.1 eq, 43 μL, 0.83 mmol) was added, and the reaction mixture was stirred at RT for 10 min. The reaction mixture was diluted with H₂O (2 mL) and basified to pH 10 with 1 M NaOH (aq). The resulting precipitate was collected via vacuum filtration, rinsed with H₂O, and dried under reduced pressure. The crude precipitate was further purified by silica gel column chromatography (5-20% EtOAc in Hexanes) to afford (2-amino-5-bromo-6-methyl-pyridin-3-yl)(3-(trifluoromethyl)-bicyclo[1.1.1]pentan-1-yl)methanone (A21, 217 mg, 83% yield) as a solid. LC/MS: [M+H]⁺ 351.1. ¹H NMR (CHCl₃-d, 400 MHz): δ_{H} 8.15 (1H, s), 2.52 (3H, s), 2.46 (6H, s).

### Intermediate A22: (2R,4S)-2-(1-cyclopropylpyrazol-4-yl)tetrahydropyran-2H-4-carbonyl chloride

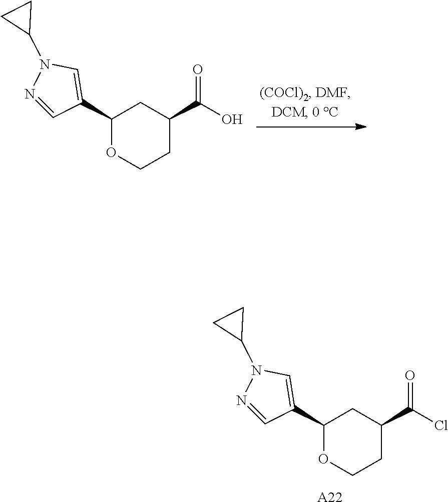

To a flame-dried round bottomed flask equipped with a Teflon-coated magnetic stirring bar was added (2R,4S)-2-(1-cyclopropylpyrazol-4-yl)tetrahydropyran-4-carboxylic acid (1 eq, 248 mg, 1.05 mmol), anhydrous DCM (5 mL) and N,N-dimethylformamide (0.05 eq, 4.1 μL, 0.052 mmol). The flask was sealed, purged under Ar and cooled to 0° C. in an ice bath with stirring. Oxalyl dichloride (1.2 eq, 0.11 mL, 1.26 mmol) was added and the reaction mixture was stirred at RT. The reaction mixture was concentrated under reduced pressure to afford (2R,4S)-2-(1-cyclopropylpyrazol-4-yl)tetrahydropyran-2H-4-carbonyl chloride (A22, 266 mg, 1.04 mmol, 99% yield) as a syrup. The product was used directly in the next step.

### Intermediate A23: (2R,4S)-N-[5-bromo-6-methyl-3-[3-(trifluoromethyl)bicyclo[1.1.1]pentane-1-carbonyl]-2-pyridyl]-2-(1-cyclopropylpyrazol-4-yl)tetrahydropyran-4-carboxamide

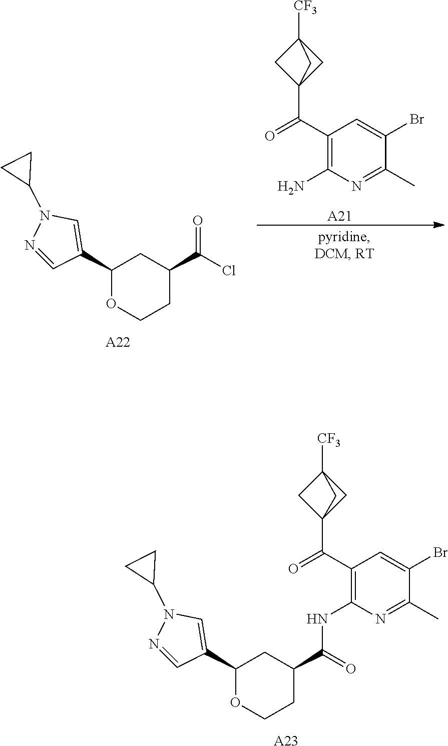

To a flame-dried, round-bottomed flask, was charged (2R,4S)-2-(1-cyclopropyl-pyrazol-4-yl)tetrahydropyran-2H-4-carbonyl chloride (A22, 1.35 eq, 268 mg, 1.05 mmol) and anhydrous DCM (2 mL). The resulting solution was purged under Ar and cooled to 0° C. with stirring. To a separate vial was charged (2-amino-5-bromo-6-methyl-3-pyridyl)-[3-(trifluoromethyl)-1-bicyclo[1.1.1]pentanyl]methanone (A21, 1 eq, 272 mg, 0.78 mmol), anhydrous DCM (4 mL) and pyridine (3 eq, 0.19 mL, 2.34 mmol). This solution was added dropwise to the flask containing acyl chloride solution. The resulting mixture was stirred at 0° C. under Ar, warming to RT overnight. After 16 h, the reaction mixture was concentrated under reduced pressure and dissolved in EtOAc (100 mL). The solution was washed with 5% citric acid (aq) and brine. The organic layer was collected, dried over Na₂SO₄, filtered, and concentrated under reduced pressure. The crude mixture was purified by silica gel column chromatography (20-70% EtOAc in DCM) to afford (2R,4S)-N-[5-bromo-6-methyl-3-[3-(trifluoromethyl)bicyclo[1.1.1]pentane-1-carbonyl]-2-pyridyl]-2-(1-cyclopropylpyrazol-4-yl)tetrahydropyran-4-carboxamide (A23, 297 mg, 0.52 mmol, 67% yield) as a foam. ¹H NMR (CHCl₃-d, 400 MHz): δ_{H} 10.55 (1H, s), 8.23 (1H, s), 7.45 (2H, s), 4.39 (1H, dd, J=11.4, 2.1 Hz), 4.18 (1H, dt, J=11.4, 3.1 Hz), 3.62-3.68 (1H, m), 3.55 (1H, tt, J=7.2, 3.8 Hz), 2.89-2.96 (1H, m), 2.68 (3H, s), 2.48 (6H, s), 2.18 (1H, d, J=13.3 Hz), 1.86-1.95 (3H, m), 1.06-1.09 (2H, m), 0.97-1.00 (2H, m). LC/MS: [M+H]⁺569.1.

### Intermediate A24: 6-bromo-2-[(2R,4S)-2-(1-cyclopropylpyrazol-4-yl)tetrahydropyran-4-yl]-7-methyl-4-[3-(trifluoromethyl)-1-bicyclo[1.1.1]pentanyl]pyrido[2,3-d]pyrimidine

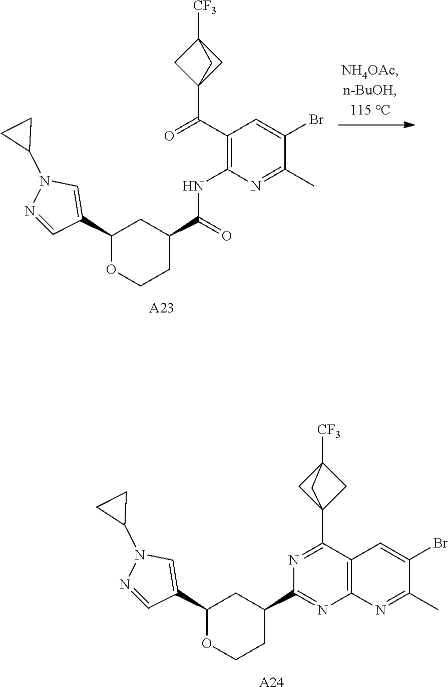

To a glass vial equipped with a Teflon-coated magnetic stirring bar was added (2R,4S)-N-[5-bromo-6-methyl-3-[3-(trifluoromethyl)bicyclo[1.1.1]pentane-1-carbonyl]-2-pyridyl]-2-(1-cyclopropyl-pyrazol-4-yl)tetrahydropyran-4-carboxamide (A23, 1 eq, 232 mg, 0.41 mmol), ammonium acetate (20 eq, 630 mg, 8.2 mmol) and 1-Butanol (5 mL). The vial was sealed and heated to 115° C. with stirring. After 1 h, the reaction mixture was cooled to RT and diluted with EtOAc (50 mL). The crude solution was washed with 5% citric acid (aq), sat, NaHCO₃(aq) and brine. The organic later was collected, dried over Na₂SO₄, filtered, and concentrated under reduced pressure. The crude mixture was purified by silica gel column chromatography (50-100% EtOAc in hexanes) to afford 6-bromo-2-[(2R,4S)-2-(1-cyclopropyl-pyrazol-4-yl)tetrahydropyran-4-yl]-7-methyl-4-[3-(trifluoromethyl)-1-bicyclo[1.1.1]pentanyl]-pyrido[2,3-d]pyrimidine (A24, 209 mg, 0.38 mmol, 93% yield) as a foam. ¹H NMR (CHCl₃-d, 400 MHz): δ_{H} 8.63 (1H, s), 7.49 (1H, s), 7.48 (1H, s), 4.54 (1H, dd, J=11.4, 2.0 Hz), 4.23-4.27 (1H, m), 3.75-3.82 (1H, m), 3.55 (1H, tt, J=7.2, 3.8 Hz), 3.38-3.46 (1H, m), 2.93 (3H, s), 2.63 (6H, s), 2.36 (1H, d, J=13.4 Hz), 2.07-2.16 (3H, m), 1.07-1.10 (2H, m), 0.95-1.00 (2H, m). LC/MS: Tr=1.81 min; [M+H]⁺ 550.1.

### Method Int-10: Synthesis of A25

### Intermediate A25: tributyl-(4-methyloxazol-2-yl)stannane

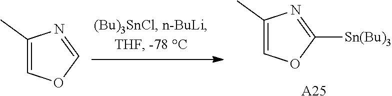

To a flame-dried round-bottomed flask equipped with a teflon-coated magnetic stirring bar was added 4-methyloxazole (1 eq, 1.04 g, 12.5 mmol) and anhydrous THF (25 mL). The flask was sealed and purged under Ar. The resulting solution was cooled to −78° C. with stirring. n-Butyllithium (1 eq, 5.0 mL, 12.5 mmol) was added dropwise over 15 min. After 30 min tri-n-butyltin chloride (1 eq, 3.4 mL, 12.5 mmol) was added dropwise. After another 15 min at −78° C., the reaction mixture was warmed to RT and stirred for 1 h. The reaction mixture was concentrated under reduced pressure. The crude residue was suspended in hexanes (50 mL) and filtered over a short bed of celite, washing with hexanes (15 mL). The filtrate was concentrated under reduced pressure to afford crude tributyl-(4-methyloxazol-2-yl)stannane (A25, 4.7 mg, 12.5 mmol, 99.9% yield) as an oil. The product was used directly in the next step without further purification.

### Method Int-11: Synthesis of A26 Intermediate of A26: 4-(2,4-difluorophenyl)-7-methyl-2-[rac-(2R,4S)-2-(1-cyclopropylpyrazol-4-yl)tetrahydropyran-4-yl]pyrido[2,3-d]pyrimidine-6-carboxamide

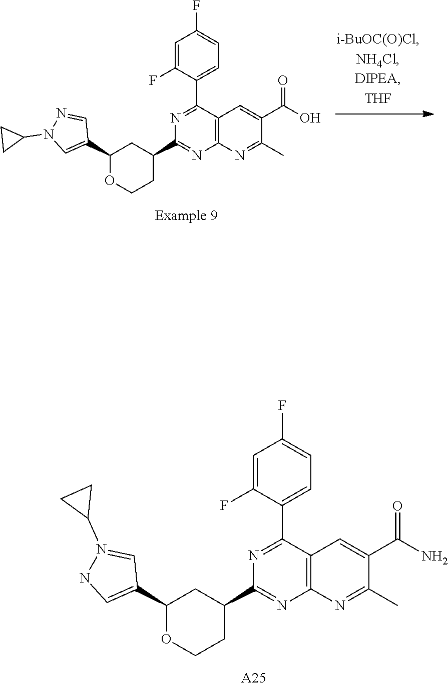

To a flame-dried, flask equipped with a Teflon-coated magnetic stirring bar was added 4-(2,4-difluorophenyl)-7-methyl-2-[(2R,4S)-2-(1-cyclopropylpyrazol-4-yl)tetrahydropyran-4-yl]pyrido[2,3-d]pyrimidine-6-carboxylic acid (Example 9, 1 eq, 50 mg, 0.102 mmol), THF (1 mL), DIPEA (5 eq, 0.09 mL, 0.51 mmol) and isobutyl chloroformate (1.2 eq, 0.016 mL, 0.12 mmol), and the reaction was stirred at rt for 2 h. Ammonium chloride (2 eq, 11 mg, 0.20 mmol) was added and the reaction was stirred to completion. The mixture was diluted with EtOAc, and the organic phase was washed with water, brine, dried over anhydrous Na₂SO₄, and filtered. The filtrate was concentrated under reduced pressure and purified by silica gel column chromatography (0-20% MeOH in DCM) to afford 4-(2,4-difluorophenyl)-7-methyl-2-[rac-(2R,4S)-2-(1-cyclopropylpyrazol-4-yl)tetrahydropyran-4-yl]pyrido[2,3-d]pyrimidine-6-carboxamide (A25, 44 mg, 0.09 mmol, 88% yield) as a solid.

### Method Int-12: Synthesis of A27

### Intermediate A27: 2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxamide

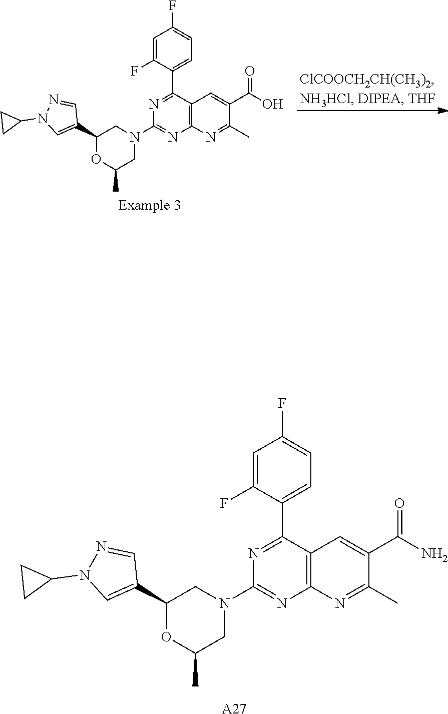

DIPEA (1.1 eq, 19 μL, 0.11 mmol) and isobutyl chloroformate (1.1 eq, 14 μL, 0.11 mmol) were added to a solution of 2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxylic acid (1 eq, 50 mg, 0.10 mmol) in dry THF (1 mL) at 0° C. The reaction was stirred for 1 h, before bubbling ammonia (balloon) through the mixture for 16 h while allowing the reaction mixture to reach 23° C. The mixture was washed with water (20 mL), extracted with EtOAc (2×20 mL). The combined organic layers were washed with brine (20 mL), dried over Na₂SO₄, filtered, and concentrated under reduced pressure. The crude was purified by reverse phase chromatography (10-40% MeCN in water (0.1% formic acid)) to afford 2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxamide (A27, 42 mg, 84% yield) as a solid. LC/MS (ESI, m/z⁺): 506.3 [M+H]⁺. ¹H NMR (CHCl₃-d, 400 MHz): δ_{H} 7.91 (1H, d, J=3.9 Hz), 7.54-7.60 (1H, m), 7.51-7.53 (2H, m), 7.06-7.12 (1H, m), 6.98-7.04 (1H, m), 5.63-5.72 (2H, m), 4.86-5.26 (2H, m), 4.55-4.61 (1H, m), 3.77-3.84 (1H, m), 3.53-3.59 (1H, m), 3.04-3.10 (1H, m), 2.87 (3H, s), 2.81-2.85 (1H, m), 1.31 (3H, d, J=6.2 Hz), 1.08-1.12 (2H, m), 0.98-1.02 (2H, m).

### Method Int-13: Synthesis of A28-A32

### Intermediate A28: N-(5-fluoro-6-methyl-2-pyridyl)-2,2-dimethyl-propanamide

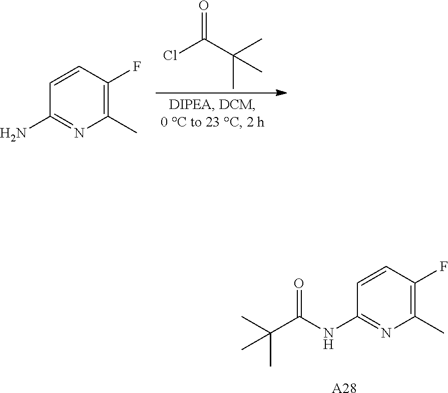

Pivaloyl chloride (1.2 eq, 3.5 mL, 28.5 mmol) was added dropwise over 4 minutes to a solution of 5-fluoro-6-methyl-pyridin-2-amine (1 eq, 3 g, 23.8 mmol), DCM (119 mL) and triethylamine (1.3 eq, 4.3 mL, 31.0 mmol) at 0° C. The mixture was stirred at 23° C. for 3 h. The crude was washed with water (100 mL) and extracted with EtOAc (2×100 mL). The combined organic layers were washed with brine (100 mL), dried over Na₂SO₄, filtered, and concentrated under reduced pressure. The crude was purified by normal phase chromatography (10-50% EtOAc in Hexanes) to afford N-(5-fluoro-6-methyl-2-pyridyl)-2,2-dimethyl-propanamide (A28, 4.8 g, 96% yield) as a solid. LC/MS (ESI, m/z⁺): 211.1 [M+H]⁺. ¹H NMR (CHCl₃-d, 400 MHz): δ_{H} 8.05 (1H, dd, J=8.9, 3.5 Hz), 7.96 (1H, s), 7.31 (1H, t, J=8.7 Hz), 2.39 (3H, d, J=2.9 Hz), 1.30 (9H, s).

### Intermediate A29: N-[5-fluoro-6-methyl-3-[3-(trifluoromethyl)bicyclo[1.1.1]pentane-1-carbonyl]-2-pyridyl]-2,2-dimethyl-propanamide

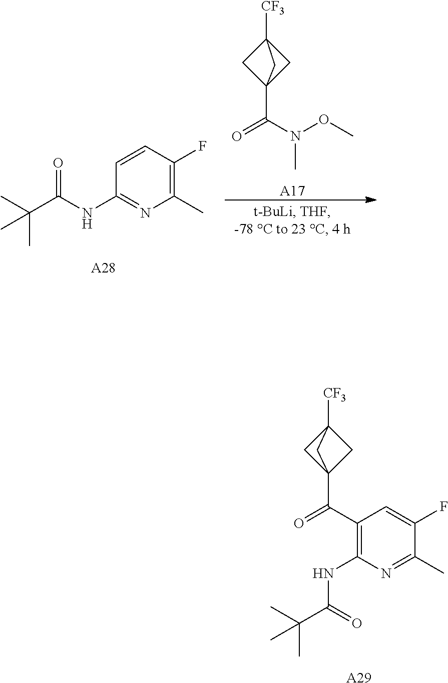

Tert-butyllithium (2.3 eq, 3.0 mL, 5.15 mmol) was added dropwise over 3 minutes to a solution of N-(5-fluoro-6-methyl-2-pyridyl)-2,2-dimethyl-propanamide (A28, 1.2 eq, 0.6 g, 2.7 mmol) and anhydrous Et₂O (5.6 mL) at −78° C. The reaction mixture was continued to stir at −78° C. for 2 h and at 0° C. for 45 min. The solution was cooled down to −78° C. and a solution of N-methoxy-N-[3-(trifluoromethyl)-1-bicyclo[1.1.1]pentanyl]acetamide (A17, 1 eq, 500 mg, 2.24 mmol), in anhydrous Et₂O (2 mL) was added to the reaction mixture dropwise. The resulting mixture was continued to stir at −78° C. for 10 min and gradually warming to 23° C. for 4 h. NH₄Cl (aq., 20 mL) was added and the organic phase was extracted with EtOAc (3×20 mL), washed with brine (30 mL), dried over Na₂SO₄ and concentrated under reduced pressure. The crude was purified by normal phase chromatography (10-40% EtOAc in hexanes) to afford N-[5-fluoro-6-methyl-3-[3-(trifluoromethyl)bicyclo[1.1.1]pentane-1-carbonyl]-2-pyridyl]-2,2-dimethyl-propanamide (A29, 310 mg, 37% yield) as a solid. LC/MS (ESI, m/z⁺): 373.2 [M+H]⁺. ¹H NMR (CHCl₃-d, 400 MHz): δ_{H} 10.37 (1H, s), 7.72 (1H, d, J=8.9 Hz), 2.53 (3H, d, J=3.0 Hz), 2.44 (6H, s), 1.30 (9H, s). ¹⁹F NMR (CHCl₃-d, 376 MHz): δ_{F}−73.3 (3F, s), −130.1 (1F, s).

### Intermediate A30: (2-amino-5-fluoro-6-methyl-3-pyridyl)-[3-(trifluoromethyl)-1-bicyclo[11.1.1]pentanyl]methanone

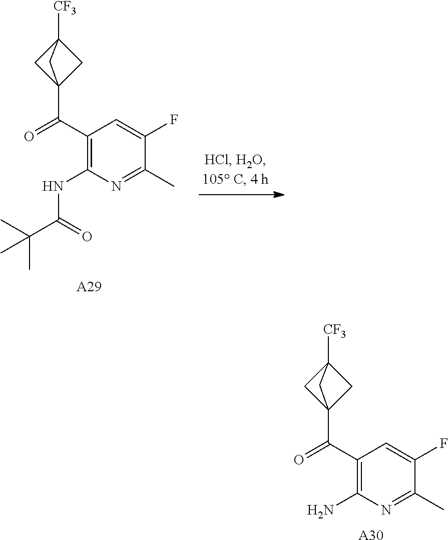

N-[5-fluoro-6-methyl-3-[3-(trifluoromethyl)bicyclo[1.1.1]pentane-1-carbonyl]-2-pyridyl]-2,2-dimethyl-propanamide (A29, 1 eq, 310 mg, 0.83 mmol) and HCl (20 eq, 2.8 mL, 16.7 mmol) were heated in water (7 mL) at 105° C. for 4 h. The reaction mixture was cooled to 23° C. and the pH was adjusted to 10 with NaOH (aq., 1.0 M). The formed precipitate was filtered, rinsed with water (10 mL) and concentrated under reduced pressure. The crude was purified by normal phase chromatography (30-60% EtOAc in hexanes) to afford (2-amino-5-fluoro-6-methyl-3-pyridyl)-[3-(trifluoromethyl)-1-bicyclo[1.1.1]pentanyl]methanone (A30, 230 mg, 96% yield) as a solid. LC/MS (ESI, m/z⁺): 289.2 [M+H]⁺.

### Intermediate A31: (2R,4S)-2-(1-cyclopropylpyrazol-4-yl)tetrahydropyran-4-carbonyl chloride

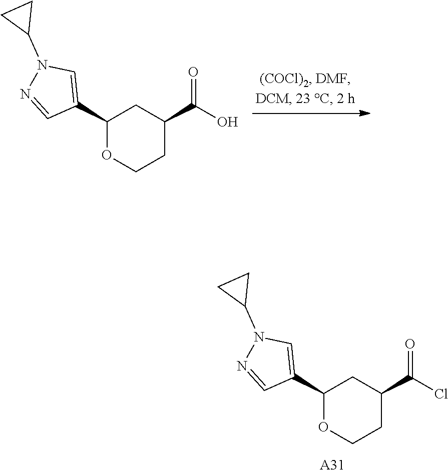

To a flame-dried round bottom flask equipped with a Teflon-coated magnetic stirring bar was added (2R,4S)-2-(1-cyclopropylpyrazol-4-yl)tetrahydropyran-4-carboxylic acid (1 eq, 100 mg, 0.42 mmol), anhydrous DCM (2.2 mL) and N,N-dimethylformamide (0.05 eq, 1.6 μL, 0.02 mmol). The flask was sealed, purged under Ar and cooled to 0° C. in an ice bath with stirring. Oxalyl dichloride (1.2 eq, 44 μL, 0.51 mmol) was added and the ice bath was removed with stirring at 23° C. for 2 h. The reaction mixture was concentrated under reduced pressure to afford (2R,4S)-2-(1-cyclopropylpyrazol-4-yl)tetrahydropyran-4-carbonyl chloride (A31, 95 mg, 88% yield) as a syrup. The compound was used as a crude in the subsequent step.

### Intermediate A32: (2R,4S)-2-(1-cyclopropylpyrazol-4-yl)-N-[5-fluoro-6-methyl-3-[3-(trifluoromethyl)bicyclo[1.1.1]pentane-1-carbonyl]-2-pyridyl]-tetrahydropyran-4-carboxamide

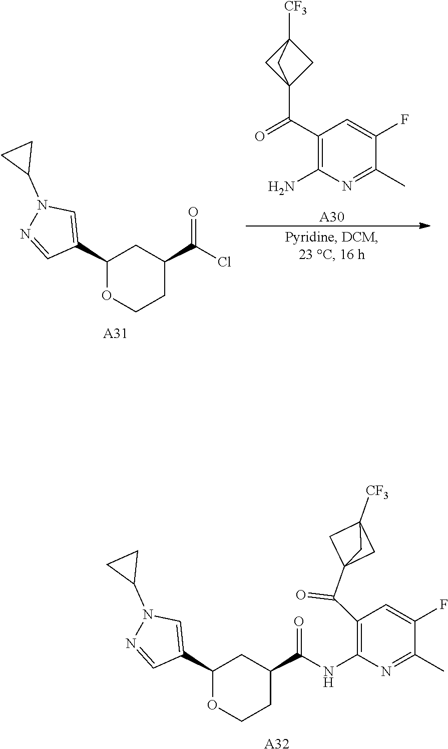

(2R,4S)-2-(1-cyclopropylpyrazol-4-yl)tetrahydropyran-4-carbonyl chloride (A31, 1 eq, 98 mg, 0.39 mmol) dissolved in anhydrous DCM (1 mL) was added over 5 minutes a solution of (2-amino-5-fluoro-6-methyl-3-pyridyl)-[3-(trifluoromethyl)-1-bicyclo[1.1.1]pentanyl]methanone (A30, 1 eq, 111 mg, 0.39 mmol) and pyridine (3 eq, 93 μL, 1.15 mmol) in anhydrous DCM (1 mL) at 0° C. The resulting solution was stirred at 23° C. for 16 h. Water (20 mL) was added and the organic phase was extracted with EtOAc (3×20 mL), washed with brine (30 mL), dried over Na₂SO₄, filtered and concentrated under reduced pressure. The crude was purified by normal phase chromatography (50-100% EtOAc in DCM) to afford (2R,4S)-2-(1-cyclopropylpyrazol-4-yl)-N-[5-fluoro-6-methyl-3-[3-(trifluoromethyl)-bicyclo[1.1.1]pentane-1-carbonyl]-2-pyridyl]-tetrahydropyran-4-carboxamide (A32, 88 mg, 45% yield) as a foam. LC/MS (ESI, m/z⁺): 507.3 [M+H]⁺. ¹H NMR (CHCl₃-d, 400 MHz): δ_{H} 10.40 (1H, s), 7.75 (1H, d, J=8.9 Hz), 7.43 (2H, s), 4.37 (1H, dd, J=11.4, 2.1 Hz), 4.16 (1H, dt, J=11.4, 3.1 Hz), 3.58-3.65 (1H, m), 3.53 (1H, tt, J=7.2, 3.8 Hz), 2.80-2.84 (1H, m), 2.53 (3H, d, J=2.9 Hz), 2.46 (6H, s), 2.16 (1H, d, J=13.4 Hz), 2.02 (1H, s), 1.87-1.93 (3H, m), 1.04-1.08 (2H, m), 0.93-0.98 (2H, m). ¹⁹F NMR (CHCl₃-d, 376 MHz): δ_{F}−73.3 (3F, s), −129.7 (1F, s).

### Method Int-14: Synthesis of A33-A37

### Intermediate A33: 4-amino-2-chloro-6-[3-(trifluoromethyl)-1-bicyclo[1.1.1]pentanyl]pyrimidine-5-carboxylate

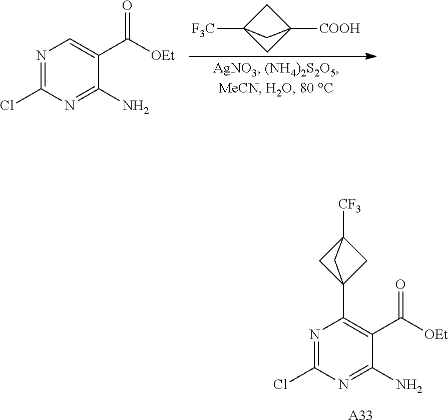

A solution of ethyl 4-amino-2-chloro-pyrimidine-5-carboxylate (1 eq, 0.40 g, 2 mmol), 3-(trifluoromethyl)-bicyclo[1.1.1]pentane-1-carboxylic acid (1.24 eq, 0.47 g, 2.6 mmol), and AgNO₃ (1.3 eq, 0.44 g, 2.6 mmol), in MeCN (8 mL) and H₂O (4 mL), was heated to 80° C. with stirring. After 5 min, Ammonium persulfate (3.3 eq, 1.51 g, 3.3 mmol) was added. The reaction mixture was fitted with a reflux condenser and stirred at 80° C. for 1 h. The reaction mixture was cooled to RT and diluted with EtOAc (100 mL). The resulting suspension was washed with sat. NaHCO₃(aq) and brine. The organic layer was dried over Na₂SO₄, filtered, concentrated under reduced pressure onto Celite, and purified by silica gel column chromatography (5-25% EtOAc in Hexanes) to afford ethyl 4-amino-2-chloro-6-[3-(trifluoromethyl)-1-bicyclo[1.1.1]pentanyl]pyrimidine-5-carboxylate (A33, 120 mg, 0.36 mmol, 36% yield) as a solid. ¹H NMR (CHCl₃-d, 400 MHz): δ_{H} 6.50 (1H, br s), 4.43 (2H, q, J=7.2 Hz), 2.38 (6H, s), 1.45 (3H, t, J=7.2 Hz). LC/MS (ESI, m/z⁺): 336.1 [M+H]⁺.

### Intermediate A34: ethyl 4-amino-2-((2S,6R)-2-(1-cyclopropyl-1H-pyrazol-4-yl)-6-methylmorpholino)-6-(3-(trifluoromethyl)bicyclo[1.1.1]pentan-1-yl)pyrimidine-5-carboxylate

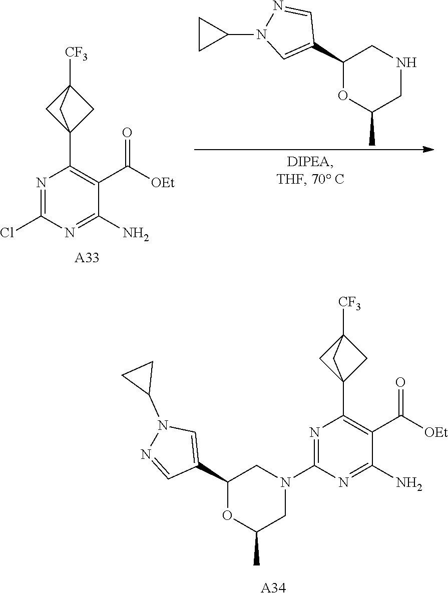

To a flame-dried, round-bottomed flask equipped with a Teflon-coated magnetic stirring bar was added (2S,6R)-2-(1-cyclopropyl-1H-pyrazol-4-yl)-6-methylmorpholine (1.07 eq, 32.4 mg, 0.16 mmol), ethyl 4-amino-2-chloro-6-(3-(trifluoromethyl)bicyclo[1.1.1]pentan-1-yl)pyrimidine-5-carboxylate (A33, 1 eq, 50 mg, 0.15 mmol), and anhydrous THF (0.8 mL). The flask was sealed, purged under Ar and the resulting solution was stirred at RT. DIPEA (3.94 eq, 0.1 mL, 0.58 mmol) was added dropwise. The reaction vessel was fitted with a reflux condenser and was heated to 70° C. with stirring under Ar. The reaction was cooled down to RT, diluted with EtOAc (25 mL) and washed successively with water and brine. The organic layer was collected, dried over Na₂SO₄, filtered, and concentrated under reduced pressure onto Celite. The crude reaction mixture was purified by silica gel column chromatography (0-20% EtOAc in DCM) to afford ethyl 4-amino-2-((2S,6R)-2-(1-cyclopropyl-1H-pyrazol-4-yl)-6-methylmorpholino)-6-(3-(trifluoromethyl)-bicyclo[1.1.1]pentan-1-yl)pyrimidine-5-carboxylate (A34, 57 mg, 76% yield) as a solid. ¹H-NMR (CHCl₃-d, 400 MHz): δ 7.49 (s, 2H), 4.73-4.82 (m, 1H), 4.62-4.72 (m, 1H), 4.46 (dd, J=10.89, 2.66 Hz, 1H), 4.34 (q, J=7.16 Hz, 2H), 3.66-3.72 (m, 1H), 3.52-3.58 (m, 1H), 2.85 (dd, J=13.29, 10.93 Hz, 1H), 2.62 (dd, J=13.29, 10.65 Hz, 1H), 2.31 (s, 6H), 1.40 (t, J=7.16 Hz, 3H), 1.26 (d, J=6.21 Hz, 3H), 1.08-1.12 (m, 2H), 0.96-1.01 (m, 2H). LC/MS (m/z⁺): 507.3 [M+1]⁺, RT: 1.76 min.

### Intermediate A35: 4-amino-2-((2S,6R)-2-(1-cyclopropyl-1H-pyrazol-4-yl)-6-methylmorpholino)-6-(3-(trifluoromethyl)bicyclo[1.1.1]pentan-1-yl)pyrimidine-5-carbaldehyde

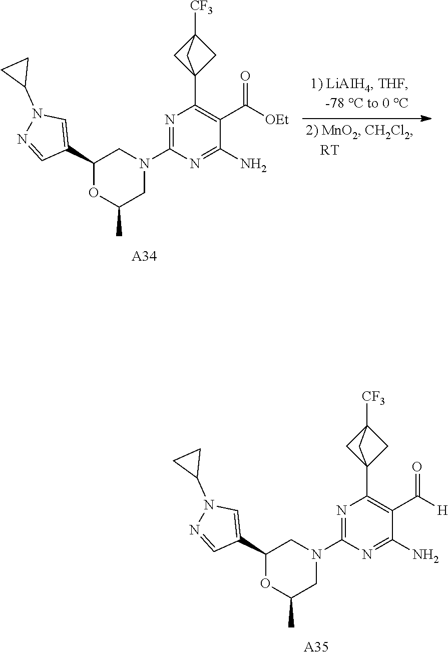

A solution of ethyl 4-amino-2-((2S,6R)-2-(1-cyclopropyl-1H-pyrazol-4-yl)-6-methyl-morpholino)-6-(3-(trifluoromethyl)bicyclo[1.1.1]pentan-1-yl)pyrimidine-5-carboxylate (A34, 0.39 g, 0.78 mmol) in anhydrous THF (8 mL) was sealed, purged under Ar and cooled to −78° C. with stirring. LiAlH₄ (2.0 M in THF) (4 eq, 1.56 mL, 3.11 mmol) was added dropwise and the reaction mixture was stirred for 3 h, gradually warming to RT. The reaction mixture was cooled back down to −78° C. and quenched with ice water and 1.0 M NaOH (aq) (3 drops). The reaction mixture was warmed to RT and diluted with EtOAc (50 mL). The layers were separated, and the aqueous layer was extracted with EtOAc (×3). The combined organic extracts were dried over Na₂SO₄, filtered, and concentrated under reduced pressure. Manganese (IV) oxide (15 eq, 1.02 g, 11.6 mmol) was added and the flask was purged under Ar. Anhydrous DCM (8 mL) was added and the resulting suspension was stirred at RT overnight. After 17 h, the reaction mixture was filtered over a Celite plus, which was washed with DCM and MeOH. The filtrate was concentrated under reduced pressure onto Celite (3 g) and purified by silica gel column chromatography (0-35% EtOAc in DCM) to afford 4-amino-2-((2S,6R)-2-(1-cyclopropyl-1H-pyrazol-4-yl)-6-methylmorpholino)-6-(3-(trifluoromethyl)bicyclo[1.1.1]pentan-1-yl)pyrimidine-5-carbaldehyde (A35, 326 mg, 91% yield) as a solid. ¹H-NMR (CHCl₃-d, 400 MHz): δ 10.18 (s, 1H), 8.65 (s, 1H), 7.50 (d, J=2.61 Hz, 2H), 5.43 (s, 1H), 4.67-4.97 (m, 2H), 4.47 (dd, J=10.89, 2.65 Hz, 1H), 3.66-3.71 (m, 1H), 3.53-3.58 (m, 1H), 2.85-2.94 (m, 1H), 2.63-2.69 (m, 1H), 2.39 (s, 6H), 1.27 (d, J=6.18 Hz, 3H), 1.10 (s, 2H), 0.98-1.01 (m, 2H). LC/MS (m/z⁺): 463.4 [M+1]⁺, RT: 1.71 min.

### Intermediate A36: methyl 2-((2S,6R)-2-(1-cyclopropyl-1H-pyrazol-4-yl)-6-methylmorpholino)-7-methyl-4-(3-(trifluoromethyl)bicyclo[1.1.1]pentan-1-yl)pyrido[2,3-d]pyrimidine-6-carboxylate

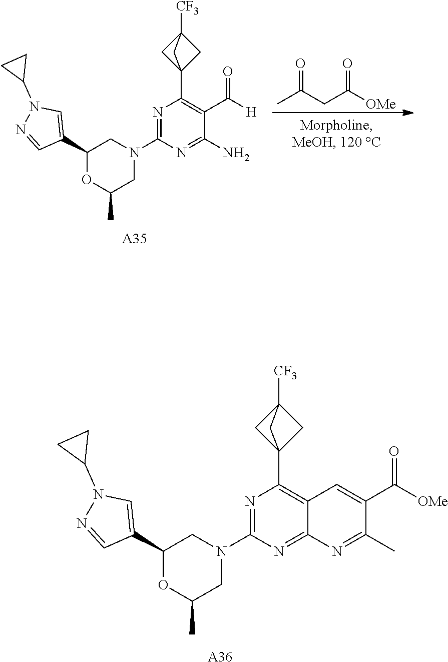

To a vial equipped with a Teflon-coated magnetic stirring bar, was charged methyl acetoacetate (2 eq, 0.15 mL, 141 mmol) and morpholine (1 eq, 62.3 μL, 0.71 mmol), and the vial was sealed and stirred at RT overnight. The resulting solution was taken up into a syringe and added to a separate flame-dried vial charged with 4-amino-2-((2S,6R)-2-(1-cyclopropyl-1H-pyrazol-4-yl)-6-methyl-morpholino)-6-(3-(trifluoromethyl)bicyclo[1.1.1]pentan-1-yl)pyrimidine-5-carbaldehyde (A35, 1 eq, 326 mg, 0.71 mmol) dissolved in anhydrous MeOH (1.1 mL). The reaction mixture was sealed and heated to 120° C. with stirring. After 72 h, the reaction mixture was cooled to RT and concentrated under reduced pressure onto Celite (5 g). The crude mixture was purified by silica gel column chromatography (0-40% EtOAc in DCM) to afford methyl 2-((2S,6R)-2-(1-cyclopropyl-1H-pyrazol-4-yl)-6-methylmorpholino)-7-methyl-4-(3-(trifluoromethyl)bicyclo[1.1.1]pentan-1-yl)pyrido[2,3-d]pyrimidine-6-carboxylate (A36, 227 mg, 59% yield) as a solid. ¹H NMR (CHCl₃-d, 400 MHz): δ 8.86 (s, 1H), 7.51-7.53 (m, 2H), 4.88-5.20 (m, 2H), 4.53-4.56 (m, 1H), 3.97 (s, 3H), 3.75-3.81 (m, 1H), 3.55-3.60 (m, 1H), 2.99-3.08 (m, 1H), 2.95 (s, 3H), 2.81 (t, J=11.97 Hz, 1H), 2.57-2.59 (m, 6H), 1.32 (s, 3H), 1.10-1.13 (m, 2H), 1.00-1.03 (m, 2H). LC/MS (m/z⁺): 543.4 [M+1]⁺, RT: 1.68 min.

### Intermediate A37: 2-[(2S,6R)-2-(1-cyclopropylpyrazol-4-yl)-6-methyl-morpholin-4-yl]-7-methyl-4-[3-(trifluoromethyl)-1-bicyclo[11.1.1]pentanyl]pyrido[2,3-d]pyrimidine-6-carboxamide

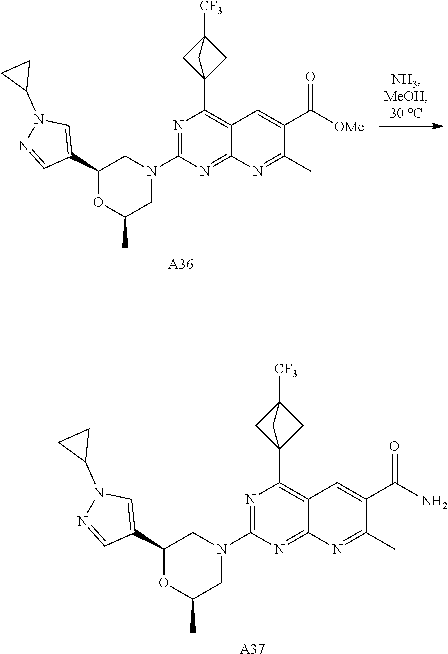 [SMILES: COC(=O)c1cc2c(C34CC(C(F)(F)F)(C3)C4)nc(N3C[C@@H](C)O[C@@H](c4cnn(C5CC5)c4)C3)nc2nc1C ; "A36" ; Cc1nc2nc(N3C[C@@H](C)O[C@@H](c4cnn(C5CC5)c4)C3)nc(C34CC(C(F)(F)F)(C3)C4)c2cc1C(N)=O ; "A37"]

To a vial was added methyl 2-((2S,6R)-2-(1-cyclopropyl-1H-pyrazol-4-yl)-6-methylmorpholino)-7-methyl-4-(3-(trifluoromethyl)bicyclo[1.1.1]pentan-1-yl)pyrido[2,3-d]-pyrimidine-6-carboxylate (A36, 1 eq, 60 mg, 0.11 mmol) and NH₃ (7.0 N in MeOH) (95 eq, 1.5 mL, 10.5 mmol), and the vial was sealed and heated to 30° C. for 3 days with stirring. The vial was further heated to 50° C. for another 6 days. The reaction mixture was cooled to RT and concentrated under reduced pressure to yield 2-[(2S,6R)-2-(1-cyclopropylpyrazol-4-yl)-6-methyl-morpholin-4-yl]-7-methyl-4-[3-(trifluoromethyl)-1-bicyclo[1.1.1]pentanyl]pyrido[2,3-d]pyrimidine-6-carboxamide (A37, 58 mg, 0.11 mmol, 99% yield) as a solid. The product was used directly in the next step without further purification. ¹H NMR (CHCl₃-d, 400 MHz): δ_{H} 8.39 (1H, s), 7.54 (2H, s), 5.79 (2H, s), 4.88-5.18 (1H, m), 4.57 (1H, d, J=10.8 Hz), 3.75-3.81 (1H, m), 3.56-3.60 (1H, m), 3.05 (1H, t, J=12.3 Hz), 2.77-2.84 (4H, m), 2.57 (6H, s), 1.32 (3H, s), 1.25 (1H, s), 1.12 (2H, s), 1.02 (2H, d, J=7.1 Hz). LC/MS (m/z⁺): 528.3 [M+H]⁺, RT: 1.33 min.

### Method Int-15: Synthesis of A38-40 and A43

### Intermediate A38: 2,2-dimethyl-N-[6-methyl-3-[3-(trifluoromethyl)bicyclo[1.1.1]pentane-1-carbonyl]-2-pyridyl]-propanamide

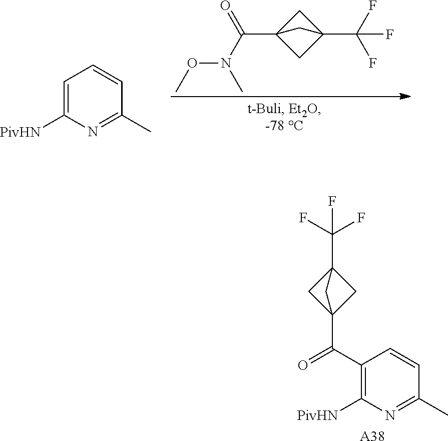

To a flame-dried, round-bottom flask equipped with a teflon-coated magnetic stirring bar was added 2,2-dimethyl-N-(6-methyl-2-pyridyl)propanamide (1 eq, 7.78 g, 40.5 mmol) and anhydrous Et₂O (250 mL), and the reaction was stirred at −74° C. (internal temperature) in an acetone-dry ice bath. t-Butyllithium (2.1 eq, 50 mL, 85 mmol) was added dropwise at a rate such as the internal temperature did not exceed −69° C. and the reaction mixture was left to stir at −78° C. for 90 min. The reaction mixture was stirred for 45 min at 0° C. in an ice bath and then cooled back down to −78° C. N-methoxy-N-methyl-3-(trifluoromethyl)bicyclo[1.1.1]pentane-1-carboxamide (A17, 1.01 eq, 9.15 g, 41 mmol), dissolved separately in anhydrous Et₂O (75 mL) was added to the reaction mixture dropwise. The reaction mixture was left to stir at −78° C., gradually warming to rt over 5 h. The reaction mixture was cooled to 0° C. in an ice bath and quenched with sat. NH₄Cl (aq.) and diluted with EtOAc and water. The layers were separated, and the organic layer was washed with brine. The organic layer was dried over Na₂SO₄, filtered, and concentrated under reduced pressure. The crude reaction mixture was purified by silica gel column chromatography (10-50% EtOAc in Hexanes) to afford 2,2-dimethyl-N-[6-methyl-3-[3-(trifluoromethyl)bicyclo[1.1.1]pentane-1-carbonyl]-2-pyridyl]propanamide (A38, 9.35 g, 26.4 mmol, 65% yield) as a solid.

### Intermediate A39: (2-amino-5-bromo-6-methyl-3-pyridyl)-[3-(trifluoromethyl)-1-bicyclo[11.1.1]pentanyl]-methanone

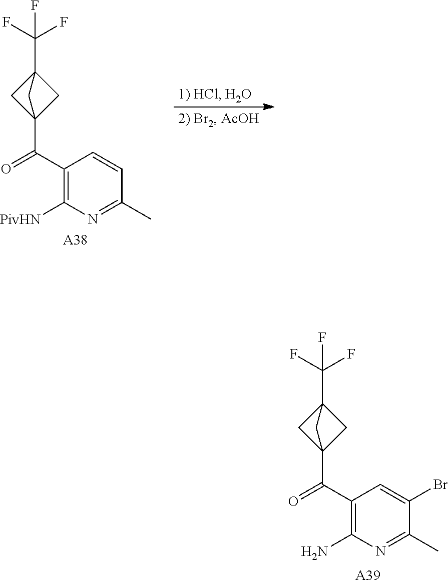

Step 1. To a suspension of 2,2-dimethyl-N-[6-methyl-3-[3-(trifluoromethyl)-bicyclo[1.1.1]pentane-1-carbonyl]-2-pyridyl]propanamide (1 eq, 9.35 g, 26.4 mmol) in water (200 mL), was added HCl (20 eq, 88 mL, 528 mmol), and the reaction was stirred at 105° C. overnight. The reaction mixture was then cooled to 0° C. and basified slowly with aq. NaOH (50% wt) to pH 10. A precipitate resulted which was diluted with EtOAc, extracted, washed with brine, dried over Na₂SO₄ and concentrated. Purified by filtration on a plug of silica to afford (2-amino-6-methyl-3-pyridyl)-[3-(trifluoromethyl)-1-bicyclo[1.1.1]pentanyl]methanone (5.75 g, 21.3 mmol, 81% yield) as a solid.

Step 2. To a solution of (2-amino-6-methyl-3-pyridyl)-[3-(trifluoromethyl)-1-bicyclo[1.1.1]pentanyl]methanone (1 eq, 5.75 g, 21.3 mmol) in Acetic acid (80 mL) was added bromine (1.1 eq, 1.2 mL, 23.4 mmol) dropwise, and the reaction mixture was stirred at rt for 10 min. The reaction mixture was diluted with water (2 mL) and basified to pH 10 with aq. NaOH (50% wt). Diluted with EtOAc and washed with water, brine, dried over MgSO₄ and concentrated under reduced pressure. The product was purified by silica gel column chromatography (5%-20% EtOAc in Hexanes) to afford (2-amino-5-bromo-6-methyl-3-pyridyl)-[3-(trifluoromethyl)-1-bicyclo[1.1.1]pentanyl]-methanone (A39, 6.1 g, 17.5 mmol, 82% yield). ¹H NMR (CHCl₃-d, 400 MHz): δ_{H} 8.15 (1H, s), 2.52 (3H, s), 2.46 (6H, s).

### Intermediate A40: 6-bromo-2-[(2R,4S)-2-(1-cyclopropylpyrazol-4-yl)tetrahydropyran-4-yl]-7-methyl-4-[3-(trifluoromethyl)-1-bicyclo[1.1.1]pentanyl]pyrido[2,3-d]pyrimidine

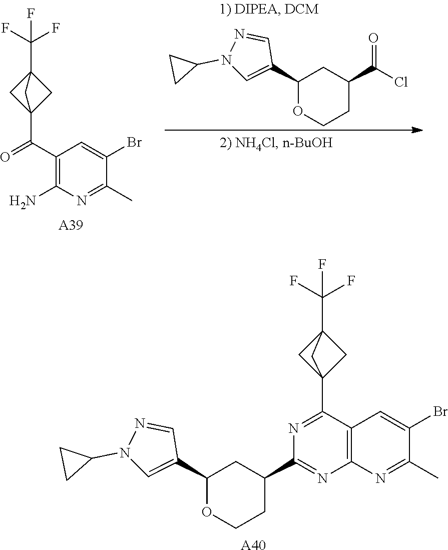

Step 1. To a flame-dried flask containing (2R,4S)-2-(1-cyclopropylpyrazol-4-yl)-tetrahydropyran-4-carbonyl chloride (1.35 eq, 268 mg, 1.05 mmol) was added anhydrous DCM (2 mL) under Ar and the resulting solution was stirred a 0° C. To a separate vial was weighed (2-amino-5-bromo-6-methyl-3-pyridyl)-[3-(trifluoromethyl)-1-bicyclo[1.1.1]pentanyl]methanone (A39, 1 eq, 272 mg, 0.78 mmol), which was dissolved in anhydrous DCM (4 mL) and supplemented with pyridine (3 eq, 0.19 mL, 2.34 mmol). This solution was added dropwise via syringe to the flask containing the acyl chloride. The resulting mixture was stirred at 0° C. under Argon and warmed to rt overnight. After 16 h, full conversion was observed by LC/MS. The reaction mixture was concentrated under reduced pressure and dissolved in EtOAc (100 mL). The solution was washed with 5% citric acid (aq.) and brine. The organic layer was collected, dried over Na₂SO₄, filtered and concentrated under reduced pressure. The crude mixture was purified by silica gel column chromatography (20-70% EtOAc in DCM) to afford (2R,4S)-N-[5-bromo-6-methyl-3-[3-(trifluoromethyl)bicyclo[1.1.1]pentane-1-carbonyl]-2-pyridyl]-2-(1-cyclopropyl-pyrazol-4-yl)tetrahydropyran-4-carboxamide (297 mg, 0.52 mmol, 67% yield) as a foam.

Step 2. A solution of (2R,4S)-N-[5-bromo-6-methyl-3-[3-(trifluoromethyl)-bicyclo[1.1.1]pentane-1-carbonyl]-2-pyridyl]-2-(1-cyclopropylpyrazol-4-yl)tetrahydropyran-4-carboxamide (1 eq, 232 mg, 0.41 mmol) and ammonium acetate (20 eq, 630 mg, 8.18 mmol) in 1-butanol (5 mL), was heated to 115° C. with stirring for 1 h. The reaction mixture was cooled to rt, diluted with EtOAc (50 mL) and washed with 5% citric acid (aq.), sat. NaHCO₃(aq), and brine. The organic layer was collected, dried over Na₂SO₄, filtered, and concentrated under reduced pressure. The crude mixture was purified by silica gel column chromatography (50-100% EtOAc in hexanes) to afford 6-bromo-2-[(2R,4S)-2-(1-cyclopropylpyrazol-4-yl)tetrahydropyran-4-yl]-7-methyl-4-[3-(trifluoromethyl)-1-bicyclo[1.1.1]pentanyl]pyrido[2,3-d]pyrimidine (A40, 209 mg, 0.38 mmol, 93% yield) as a foam.

### Intermediate A43: 2-[(2R,4S)-2-(1-cyclopropylpyrazol-4-yl)tetrahydropyran-4-yl]-7-methyl-6-methylsulfanyl-4-[3-(trifluoromethyl)-1-bicyclo[1.1.1]pentanyl]pyrido[2,3-d]pyrimidine

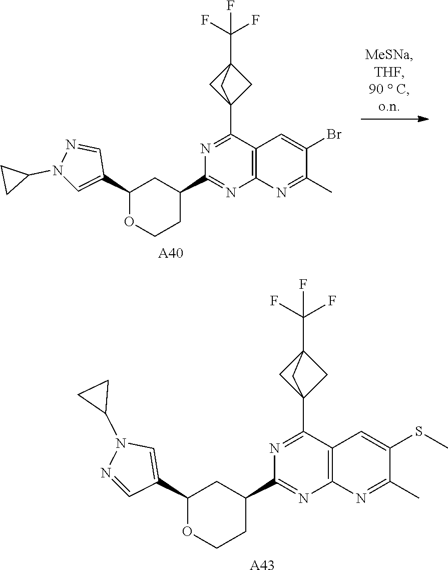

To a solution of 6-bromo-2-[(2R,4S)-2-(1-cyclopropylpyrazol-4-yl)tetrahydropyran-4-yl]-7-methyl-4-[3-(trifluoromethyl)-1-bicyclo[1.1.1]pentanyl]pyrido[2,3-d]pyrimidine (A40, 1 eq, 100 mg, 0.18 mmol) in THF (1 mL) was added sodium thiomethoxide (1.4 eq, 18 mg, 0.26 mmol), and the solution was stirred at 90° C. overnight. Cooled down, diluted with EtOAc, washed with water, brine, Na₂SO₄, and concentrated. The residue was purified by silica gel column chromatography (50-100% EtOAc in DCM) to afford 2-[(2R,4S)-2-(1-cyclopropylpyrazol-4-yl)tetrahydropyran-4-yl]-7-methyl-6-methylsulfanyl-4-[3-(trifluoromethyl)-1-bicyclo[1.1.1]pentanyl]pyrido[2,3-d]pyrimidine (A43, 76 mg, 0.15 mmol, 83% yield) as a foam.

### Method Int-15: Synthesis of A41-A42

### Intermediate A41: 4-amino-2-chloro-6-cyclobutyl-pyrimidine-5-carbaldehyde

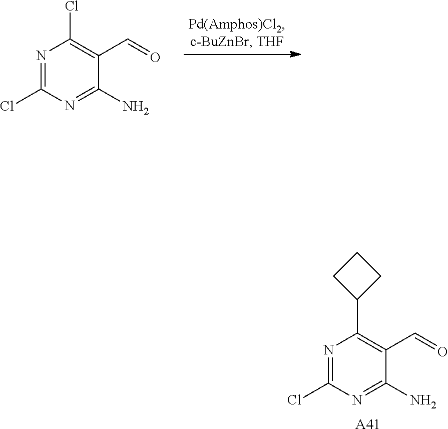

A solution of 4-amino-2,6-dichloro-pyrimidine-5-carbaldehyde (1 eq, 200 mg, 1.04 mmol) in anhydrous THF (10 mL) was degassed, cooled to 0° C., and stirred under Ar. Pd(amphos)Cl₂ (0.05 eq, 37 mg, 0.052 mmol) was added, followed by chloro(cyclobutyl)zinc (2 eq, 4.2 mL, 2.08 mmol) dropwise. The reaction mixture was stirred at 0° C., gradually warming to rt overnight. The reaction mixture was quenched with sat. ammonium chloride and diluted with EtOAc (150 mL). The phases were separated, and the organic phase was washed with water and brine. The organic layer was collected, dried over Na₂SO₄, filtered, and concentrated under reduced pressure. The crude mixture was purified by silica gel column chromatography (0-20% EtOAc in hexanes) to afford 4-amino-2-chloro-6-cyclobutyl-pyrimidine-5-carbaldehyde (A41, 181 mg, 0.86 mmol, 82% yield) as a solid.

### Intermediate A42: 4-amino-6-cyclobutyl-2-[(2S,6R)-2-(1-cyclopropylpyrazol-4-yl)-6-methyl-morpholin-4-yl]pyrimidine-5-carbaldehyde

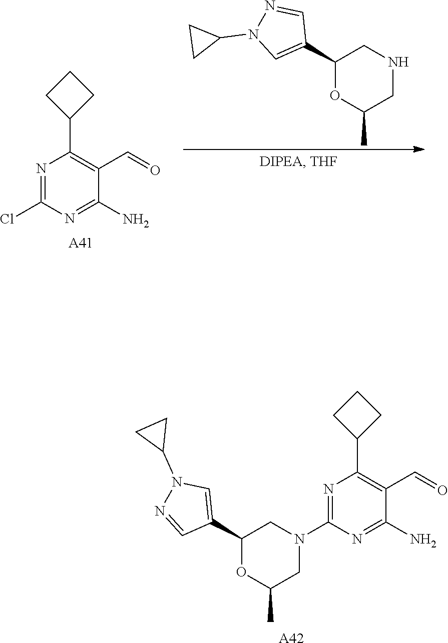

To a solution of (2S,6R)-2-(1-cyclopropylpyrazol-4-yl)-6-methyl-morpholine (2 eq, 355 mg, 1.71 mmol) in THF (10 mL) was added 4-amino-2-chloro-6-cyclobutyl-pyrimidine-5-carbaldehyde (A41, 1 eq, 181 mg, 0.86 mmol) and DIPEA (3 eq, 0.45 mL, 2.57 mmol), and the reaction was stirred at 70° C. overnight. The reaction mixture was quenched with water and diluted with EtOAc (150 mL). The phases were separated, and the organic phase was washed with water and brine. The organic layer was collected, dried over Na₂SO₄, filtered, and concentrated under reduced pressure. The crude mixture was purified by silica gel column chromatography (5-100% EtOAc in DCM) to afford 4-amino-6-cyclobutyl-2-[(2S,6R)-2-(1-cyclopropylpyrazol-4-yl)-6-methyl-morpholin-4-yl]pyrimidine-5-carbaldehyde (A42, 87 mg, 0.23 mmol, 27% yield) as a solid.

### Method Int-16: Synthesis of A67

### Synthesis of chloro-(4-cyano-2-fluoro-phenyl)zinc

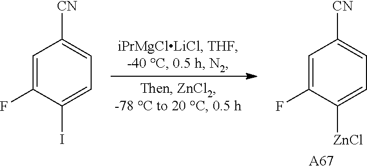

To a solution of 3-fluoro-4-iodo-benzonitrile (1.00 eq, 1.00 g, 4.05 mmol) in THF (20 mL) was added iPrMgCl LiCl (1.10 eq, 3.4 mL, 4.45 mmol) at −40° C. under N₂. The mixture was stirred at −40° C. for 0.5 h. Then ZnCl₂ (1.20 eq, 9.7 mL, 4.86 mmol) was added at −70° C. under N₂. The mixture was stirred at 15° C. for 0.5 h. The reaction mixture was used directly for the next step.

An analogous method was followed to obtain the following compounds.

| Int # | Intermediate | Starting Material | Characterization |
|---|---|---|---|
| A77 | 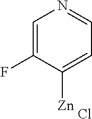 | 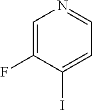 | The reaction mixture was used directly for the next step. (3-fluoropyridin-4-yl)zinc(II) chloride (A77) |
| | A77 | (3-fluoro-4-iodo-pyridine) | |

### Method Int-17: Synthesis of Intermediate A70

### Synthesis of A69

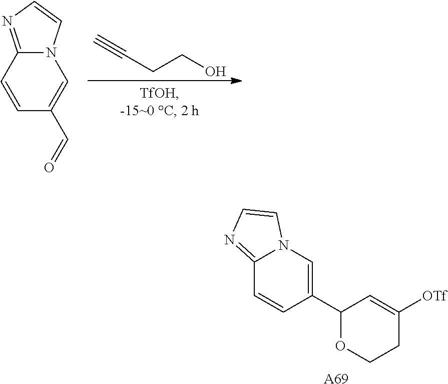

To a solution of imidazo[1,2-a]pyridine-6-carbaldehyde (1.00 eq, 2.00 g, 13.7 mmol) in DCM (50 mL) was added 3-butyn-1-ol (1.00 eq, 1.0 mL, 13.7 mmol) at −15° C. under N₂. The mixture was stirred at −15° C. for 30 min. Then, TfOH (3.00 eq, 3.6 mL, 41.1 mmol) was dropwise and the mixture was stirred at −10~0° C. for 2 h. The reaction mixture was adjusted to pH=7 with NaHCO₃ aqueous solution. The reaction mixture was poured into H₂O (100 mL) and extracted with DCM (3×100 mL). The combined organic layers were dried over Na₂SO₄, filtered and concentrated under reduced pressure and purified by reversed-phase column chromatography (water (FA)-MeCN, B %: 40%-50%), concentrated under reduced pressure and the residual aqueous solution was lyophilized to afford (6-imidazo[1,2-a]pyridin-6-yl-3,6-dihydro-2H-pyran-4-yl) trifluoromethanesulfonate (A69, 2.00 g, 5.74 mmol, 42% yield) as an oil. LC/MS (ESI, m/z⁺): [M+H]⁺=349.0; ¹H NMR (400 MHz, CDCl₃) δ ppm 2.49 (br d, J=3.30 Hz, 1H), 2.66-2.80 (m, 1H), 3.93 (td, J=7.92, 4.22 Hz, 1H), 4.11-4.20 (m, 1H), 5.39 (br d, J=2.45 Hz, 1H), 5.96 (s, 1H), 7.44 (d, J=9.29 Hz, 1H), 7.74 (s, 2H), 7.88 (d, J=9.41 Hz, 1H), 8.33 (s, 1H).

### Synthesis of A70

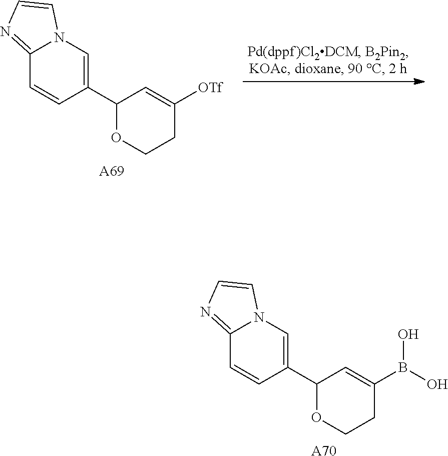

To a mixture of AcOK (4.0 eq, 2.25 g, 22.9 mmol), B₂pin₂ (1.5 eq, 2.18 g, 8.59 mmol) and [6-([1,2,4]triazolo[1,5-a]pyridin-6-yl)-3,6-dihydro-2H-pyran-4-yl]trifluoromethanesulfonate (1.0 eq, 2.00 g, 5.73 mmol) in 1,4-Dioxane (50 mL) was added Pd(dppf)Cl₂DCM (0.10 eq, 468 mg, 0.573 mmol) under N₂ atmosphere. The reaction mixture was heated at 90° C. for 2 h under N₂ atmosphere. The resulting mixture was filtered, concentrated under reduced pressure, and purified by reversed-phase HPLC (0.1% o FA condition) to afford [6-([1,2,4]triazolo[1,5-a]pyridin-6-yl)-3,6-dihydro-2H-pyran-4-yl]boronic acid (A70, 900 mg, 3.67 mmol, 64% o yield) as an oil. LC/MS (ESI, m/z): [M+H]=327.2. NH NMR (400 MHz, METHANOL-d4) δ ppm 2.07-2.25 (m, 1H) 2.30-2.45 (m, 1H) 3.68-3.84 (m, 1H) 3.90-4.07 (m, 1H) 5.23 (brd, J=2.25 Hz, 1H) 6.22 (brd, J=3.13 Hz, 1H) 7.40 (dd, 1=9.32, 1.31 Hz, 1H) 7.57-7.70 (m, 2H) 7.88 (s, 1H), 8.48 (s, 1H).

An analogous method was followed to obtain the following compounds.

| Int-# | Intermediate | Starting Material | Characterization |
|---|---|---|---|
| A73 | 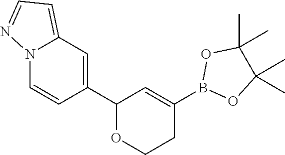 [SMILES: CC1(C)OB(C2=CC(c3ccn4nccc4c3)OCC2)OC1(C)C] | pyrazolo[1,5- a]pyridine-5- carbaldehyde | The residue was purified by reversed-phase chromatography (water (FA)-MeCN, B %: 35%- 40%) and lyophilized to give 5-[4-(4,4,5,5- tetramethyl-1,3,2-dioxaborolan-2-yl)-3,6- dihydro-2H-pyran-6-yl]pyrazolo[1,5-a]pyridine (A73, 1.4 g, 3.86 mmol, 79% yield) as a solid. LC/MS (ESI, m/z⁺): [M + H]⁺ = 327.1. ¹H NMR (400 MHz, CDCl₃) δ = 8.42 (br d, J = 6.0 Hz, 1H), 7.94 (br s, 1H), 7.53 (br s, 1H), 6.79 (br d, J = 6.4 Hz, 1H), 6.60-6.45 (m, 2H), 5.19 (br s, 1H), |
| | A73 | | 4.08-3.99 (m, 1H), 3.84-3.68 (m, 1H), 2.50-2.33 |
| | | | (m, 1H), 2.27-2.18 (m, 1H), 1.34-1.34 (m, 1H), |
| | | | 1.28 (s, 11H). |
| A76 | 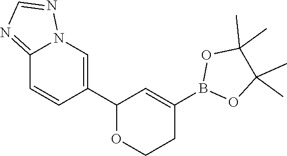 [SMILES: CC1(C)OB(C2=CC(c3ccc4ncnn4c3)OCC2)OC1(C)C] | [1,2,4]triazolo- [1,5- a]pyridine-6- carbaldehyde | The reaction mixture was poured into H₂O (100 mL) and extracted with EtOAc (3 × 100 mL). The combined organic layers were dried over Na₂SO₄, filtered, concentrated under reduced pressure, and purified by reversed-phase chromatography (water (FA)-MeCN, B %: 35%- 40%) and lyophilized to give 6-[4-(4,4,5,5- tetramethyl-1,3,2-dioxaborolan-2-yl)-3,6- dihydro-2H-pyran-6-yl]-[1,2,4]triazolo-[1,5- a]pyridine (A76, 350 mg, 1.02 mmol, 53% yield) |
| | A76 | | as a solid. LC/MS (ESI, m/z⁺): [M + H]⁺ = 328.1. |
| | | | ¹H NMR (400 MHz, CDCl₃) δ = 8.63 (br s, 1H), |
| | | | 8.50-8.32 (m, 1H), 7.77 (br d, J = 9.3 Hz, 1H), |
| | | | 7.65-7.48 (m, 1H), 6.57 (br d, J = 1.4 Hz, 1H), |
| | | | 6.37 (br s, 1H), 5.27 (br s, 1H), 4.08-3.92 (m, |
| | | | 1H), 3.85-3.67 (m, 1H), 2.51-2.15 (m, 2H), 1.34- |
| | | | 1.20 (m, 4H). |
| A84 | 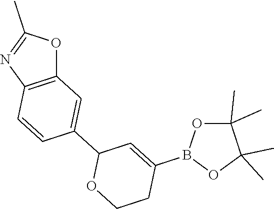 [SMILES: Cc1nc2ccc(C3C=C(B4OC(C)(C)C(C)(C)O4)CCO3)cc2o1] | 2-methyl-1,3- benzoxazole- 6- carbaldehyde | The residue was purified by reversed-phase chromatography (water (FA)-MeCN, B %: 35%- 50%) and lyophilized to afford 2-methyl-6-[4- (4,4,5,5-tetramethyl-1,3,2-dioxaborolan-2-yl)- 3,6-dihydro-2H-pyran-6-yl]-1,3-benzoxazole (A84, 1.30 g, 3.81 mmol, 61% yield) as an oil. LC/MS (ESI, m/z⁺): 342.1 [M + H]⁺. ¹H NMR (400 MHz, CDCl₃) δ = 7.60 (d, J = 8.1 Hz, 1H), 7.51 (s, 1H), 7.32 (dd, J = 1.4, 8.1 Hz, 1H), 6.59 (d, J = 1.6 Hz, 1H), 5.34-5.20 (m, 1H), 4.09-3.99 (m, 1H), 3.80 (ddd, J = 3.9, 9.1, 11.3 Hz, 1H), 2.67-2.59 (m, 3H), 2.44 (dtd, J = 2.6, 5.8, 17.5 Hz, 1H), 2.21 (br dd, J = 2.1, 17.4 Hz, 1H), 1.27 (s, 12H). |
| | A84 | | |
| A86 | 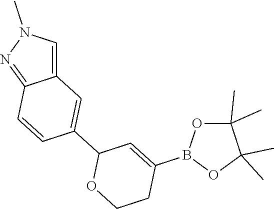 [SMILES: Cn1cc2cc(C3C=C(B4OC(C)(C)C(C)(C)O4)CCO3)ccc2n1] | 2-methyl- indazole-5- carbaldehyde | The residue was purified by Prep-HPLC (Condition 4, Gradient 1) to afford 2-methyl-5-[4- (4,4,5,5-tetramethyl-1,3,2-dioxaborolan-2-yl)- 3,6-dihydro-2H-pyran-6-yl]indazole (A86, 1.5 g, 4.41 mmol, 61% yield) as an oil. LC/MS (ESI, m/z⁺): [M + H]⁺ = 341.3. ¹H NMR (400 MHz, CDCl₃) δ = 7.85 (s, 1H), 7.66 (d, J = 8.9 Hz, 1H), 7.60 (s, 1H), 7.29 (br dd, J = 1.3, 8.9 Hz, 1H), 6.62 (br d, J = 1.4 Hz, 1H), 5.23 (br d, J = 2.3 Hz, 1H), 4.21 (s, 3H), 4.09-3.97 (m, 1H), 3.78 (ddd, J = 3.9, 8.9, 11.3 Hz, 1H), 2.43 (dtd, J = 2.8, 5.7, 14.4 Hz, 1H), 2.21 (br dd, J = 2.2, 17.4 Hz, 1H), 1.27 (s, 12H). |
| | A86 | | |
| A121 | 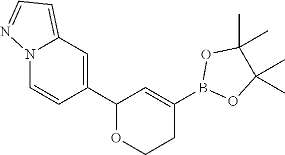 [SMILES: CC1(C)OB(C2=CC(c3ccn4nccc4c3)OCC2)OC1(C)C] | pyrazolo[1,5- a]pyridine-5- carbaldehyde | Modification: Step 1 was stirred from 0 °C. to rt overnight. The residue was purified by silica gel column chromatography (5-50% EtOAc in hexanes) to afford 5-[4-(4,4,5,5-tetramethyl-1,3,2- dioxaborolan-2-yl)-3,6-dihydro-2H-pyran-6- yl]pyrazolo[1,5-a]pyridine (A121, 2.4 g, 7.4 mmol, 91% yield). ESI-MS (m/z)+: 327.3 (obs). |
| | A121 | | |

### Method Int-18: Synthesis of Intermediate A81

### Synthesis of A78

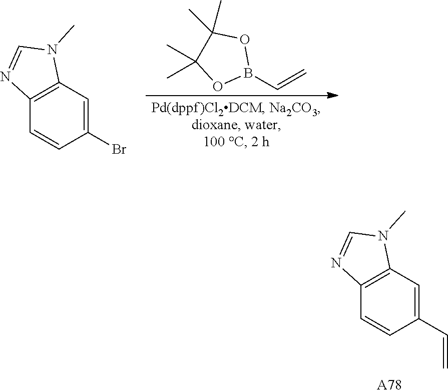

To a solution of 6-bromo-1-methyl-1H-benzo[d]imidazole (1.0 eq, 100 mg, 0.308 mmol in 1,4-Dioxane (50 mL) and Water (10 mL) were added 4,4,5,5-tetramethyl-2-vinyl-1,3,2-dioxaborolane (2.50 eq, 5.0 mL, 29.6 mmol), Na₂CO₃ (3.00 eq, 3.77 g, 35.5 mmol) and Pd(dppf)Cl₂ ·DCM (0.10 eq, 967 mg, 1.18 mmol) in one portion, then the mixture was stirred at 100° C. for 12 h under N₂. Ethyl acetate (200 mL) and water (50 mL) were added to the reaction mixture, the organic phase was separated and then was washed with brine (40 mL), dried over Na₂SO₄, filtered and concentrated under reduced pressure to give the residue. The residue was purified by silica gel column chromatography (50% EtOAc in PE, to 9% MeOH in EtOAc) to obtain 1-methyl-6-vinyl-benzimidazole (A78, 1.3 g, 8.22 mmol, 69% yield) as an oil. LC/MS (ESI, m/z⁺): [M+H]⁺=159.1; ¹H NMR (400 MHz, DMSO-d6) δ ppm 3.83 (s, 3H), 5.23 (dd, J=10.94, 0.81 Hz, 1H), 5.85 (dd, J=17.64, 0.88 Hz, 1H), 6.86 (dd, J=17.64, 10.88 Hz, 1H), 7.37 (dd, J=8.38, 1.50 Hz, 1H), 7.59 (d, J=8.38 Hz, 1H), 7.65 (d, J=1.13 Hz, 1H), 8.17 (s, 1H).

### Synthesis of A79

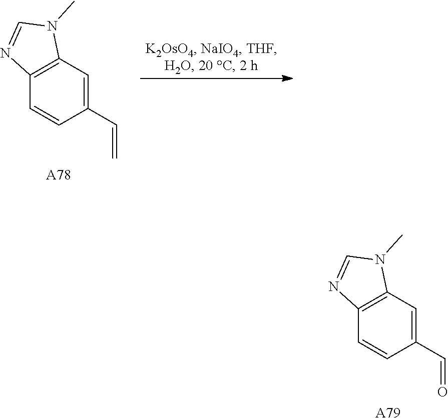

To a solution of 1-methyl-6-vinyl-benzimidazole (1.00 eq, 1.50 g, 9.48 mmol) in THF (15 mL) and water (15 mL) was added K₂OsO₄ (0.10 eq, 315 mg, 0.948 mmol), then the mixture was stirred at 25° C. for 15 min NaIO₄ (2.50 eq, 5.07 g, 23.7 mmol) was added, then the mixture was stirred at 25° C. for 2 h. Then Na₂SO₃ solution (10 mL) and water (30 mL) was added. The mixture was extracted with DCM (5×30 mL). The organic phase was separated, dried over Na₂SO₄, filtered, and concentrated to give 3-methylbenzimidazole-5-carbaldehyde (A79, 1.2 g, 7.49 mmol, 79% yield) as a solid. LC/MS (ESI, m/z⁺): [M+H]⁺=160.9; ¹H NMR (400 MHz, DMSO-d6) δ ppm 3.94 (s, 3H), 7.71-7.89 (m, 2H), 8.21 (br s, 1H), 8.45 (br s, 1H), 10.08 (s, 1H).

### Synthesis of A80

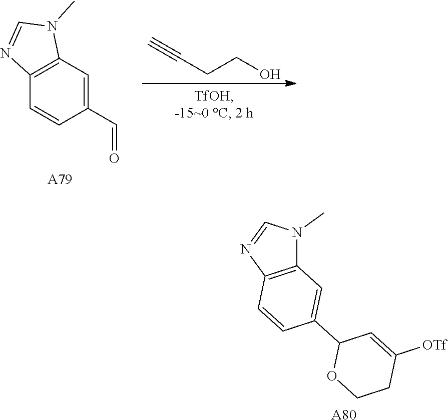

To a solution of 3-methylbenzimidazole-5-carbaldehyde (1.0 eq, 500 mg, 3.12 mmol) in DCM (1 mL) was added 3-butyn-1-ol (1.0 eq, 0.24 mL, 3.12 mmol) at −15° C. under N₂. The mixture was stirred at −15° C. for 30 min. Then TfOH (3.0 eq, 0.83 mL, 9.36 mmol) was added dropwise and the mixture was stirred at −10~0° C. for 2 h. The reaction mixture was diluted with water (10 mL) and extracted with EtOAc (3×15 mL). The combined organic layers were dried over Na₂SO₄, filtered and concentrated under reduced pressure to give the residue. The residue was purified by reversed-phase HPLC (0.1% FA condition) to give [6-(3-methylbenzimidazol-5-yl)-3,6-dihydro-2H-pyran-4-yl]trifluoromethanesulfonate (A80, 650 mg, 1.79 mmol, 57% yield) as an oil. LC/MS (ESI, m/z⁺): 363.1 [M+H]⁺; ¹H NMR (400 MHz, CDCl₃) δ ppm 2.46 (br dd, J=16.99, 3.06 Hz, 1H), 2.77 (dtd, J=14.09, 5.46, 5.46, 2.75 Hz, 1H), 3.93 (td, J=7.92, 4.34 Hz, 1H), 4.01 (s, 3H), 4.15-4.22 (m, 1H), 5.46 (br d, J=2.32 Hz, 1H), 5.99 (s, 1H), 7.43 (d, J=8.44 Hz, 1H), 7.55 (s, 1H), 7.87 (d, J=8.44 Hz, 1H), 8.65 (s, 1H), 8.61-8.74 (m, 1H).

### Synthesis of A81

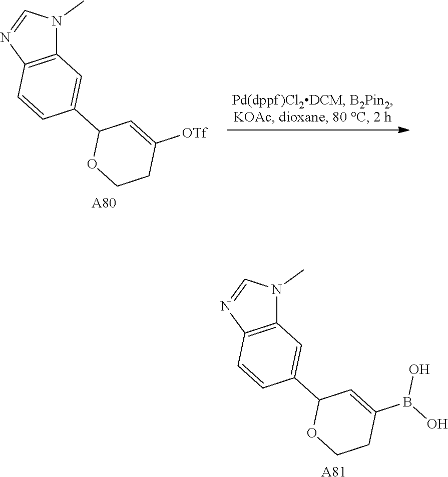

To a mixture of AcOK (4.0 eq, 649 mg, 6.62 mmol), B₂pin₂ (1.5 eq, 631 mg, 2.48 mmol) and [6-(3-methylbenzimidazol-5-yl)-3,6-dihydro-2H-pyran-4-yl]trifluoromethanesulfonate (1.0 eq, 600 mg, 1.66 mmol) in 1,4-Dioxane (12 mL) was added Pd(dppf)Cl₂ DCM (0.10 eq, 135 mg, 0.166 mmol) under N₂ atmosphere. The reaction mixture was heated at 90° C. for 2 h under N₂ atmosphere. The reaction mixture was filtered, concentrated under reduced pressure, and purified by reversed-phase HPLC (0.1% FA condition) to give [6-(3-methylbenzimidazol-5-yl)-3,6-dihydro-2H-pyran-4-yl]boronic acid (A81, 100 mg, 0.387 mmol, 23% yield). LC/MS (ESI, m/z⁺): [M+H]=341.1; ¹H NMR (400 MHz, DMSO-d6) δ ppm 3.58-3.71 (m, 1H), 3.83 (s, 3H), 3.86-3.98 (m, 1H), 5.20-5.30 (m, 1H), 6.52 (d, J=14.79 Hz, 1H), 7.18 (d, J=8.44 Hz, 1H), 7.45-7.52 (m, 1H), 7.58-7.66 (m, 3H), 8.16 (s, 1H).

An analogous method was followed to obtain the following intermediate.

| Int # | Intermediate | Starting Material | Characterization |
|---|---|---|---|
| A82 | 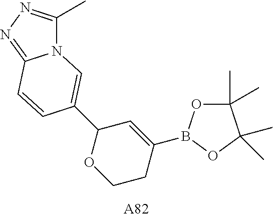 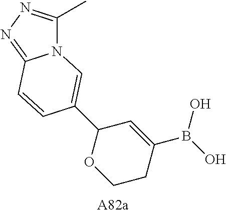 | 6-bromo-3- methyl[1,2,4]- triazolo[4,3- a]pyridine | The residue was purified by reversed-phase chromatography (water (FA)-MeOH, B %: 30%- 60%) and lyophilized to give [6-(3-methyl- [1,2,4]triazolo[4,3-a]pyridin-6-yl)-3,6-dihydro- 2H-pyran-4-yl]boronic acid (A82a, 280 mg, 1.08 mmol, 98% yield) and 3-methyl-6-[4- (4,4,5,5- tetramethyl-1,3,2-dioxaborolan-2- yl)-3,6-dihydro-2H-pyran-6-yl]-[1,2,4]triazolo [4,3-a]pyridine (A82, 0.10 g, 0.29 mmol, 27% yield) as a solid. A82: LC/MS (ESI, m/z⁺): [M + H]⁺ = 260.1. A82a: LC/MS (ESI, m/z⁺): [M + H]⁺ = 260.1. ¹H NMR (400 MHz, METHANOL-d4) δ = 8.61 (br s, 1H), 7.72-7.58 (m, 2H), 6.57-6.12 (m, 1H), 5.28 (br s, 1H), 4.02-3.91 (m, 1H), 3.74 (br d, J = 9.1 Hz, 1H), 2.51 (s, 3H), 2.36 (br dd, J = 5.6, 7.9 Hz, 1H), 2.24-2.11 (m, 1H). |
| A85 | 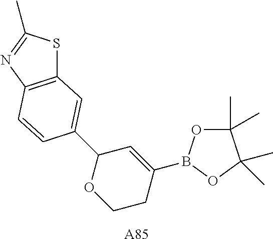 | 6-bromo-2- methylbenzo- [d]thiazole | Modification: Step 1 was stirred at 100 °C. for 12 h. Step 4 was stirred at 90 °C. for 2 h. The residue was purified by silica gel column chromatography (0-100% EtOAc in PE) to afford 2-methyl-6-[4-(4,4,5,5-tetramethyl-1,3,2- dioxaborolan-2-yl)-3,6-dihydro-2H-pyran-6-yl]- 1,3-benzothiazole (A85, 1.6 g, 3.58 mmol, 84% yield) as a solid. LC/MS (ESI, m/z⁺): [M + H]⁺ = 358.1; Tr = 0.593 min. ¹H NMR (400 MHz, CDCl₃) δ = 7.92 (d, J = 8.4 Hz, 1H), 7.87-7.84 (m, 1H), 7.47-7.43 (m, 1H), 6.61 (s, 1H), 5.30 (br d, J = 2.3 Hz, 1H), 4.06 (ddd, J = 3.4, 5.1, 11.2 Hz, 1H), 3.86-3.76 (m, 1H), 2.84 (s, 3H), 2.52-2.39 (m, 1H), 2.22 (br dd, J = 2.4, 17.6 Hz, 1H), 1.28 (s, 12H). |

### Method Int-19: Synthesis of A91

### Synthesis of A87

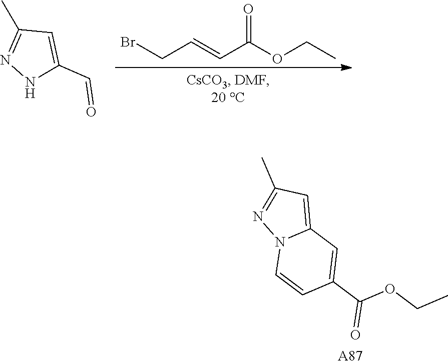

To a solution of 3-methyl-1H-pyrazole-5-carbaldehyde (1.0 eq, 5.00 g, 45.4 mmol) and ethyl (E)-4-bromobut-2-enoate (1.2 eq, 7.4 mL, 54.5 mmol) in DMF (150 mL) was added Cs₂CO₃ (2.1 eq, 31.07 g, 95.4 mmol) and then the mixture was stirred for 4 h at 25° C. The resulting mixture was filtered and concentrated under reduced pressure to give a residue. The residue was extracted with EtOAc (3×300 mL). The combined organic layers were washed with brine (300 mL), separated, and dried over Na₂SO₄, and filtered. The filtrate was concentrated under reduced pressure and purified by silica gel column chromatography (9% EtOAc in PE) to afford ethyl 2-methylpyrazolo[1,5-a]pyridine-5-carboxylate (A87, 600 mg, 2.94 mmol, 6% yield) as an oil. LC/MS (ESI, m/z⁺): 205.1 [M+H]⁺.

### Synthesis of A88

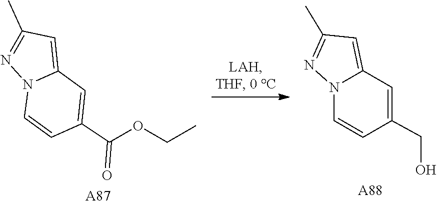

To a solution of ethyl 2-methylpyrazolo[1,5-a]pyridine-5-carboxylate (A87, 1.0 eq, 570 mg, 2.79 mmol) in THF (10 mL) was added LAH (3.0 eq, 3.3 mL, 8.37 mmol) dropwise at 0° C., and the mixture was stirred for 2 h at 0° C. sat. potassium sodium tartrate (aqu.) (10 mL) was added to the mixture slowly and then the mixture was stirred for 1 h at 25° C. The resulting mixture was extracted with EtOAc (3×10 mL) and washed with brine (10 mL). The organic layer was separated, dried over Na₂SO₄, filtered, and concentrated under reduced pressure to afford (2-methylpyrazolo[1,5-a]pyridin-5-yl)methanol (A88, 400 mg, 2.47 mmol, 88% yield) as a solid. LC/MS (ESI, m/z⁺): 163.0 [M+H]⁺. ¹H NMR (400 MHz, CDCl₃) δ ppm 2.48 (s, 3H) 4.69 (s, 2H), 6.24 (s, 1H), 6.63 (dd, J=7.09, 1.34 Hz, 1H), 7.36 (s, 1H), 8.29 (d, J=7.09 Hz, 1H).

### Synthesis of A89

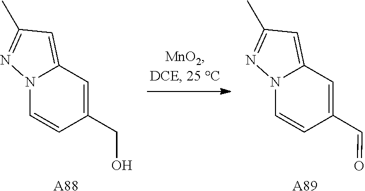

To a solution of (2-methylpyrazolo[1,5-a]pyridin-5-yl)methanol (A88, 1.0 eq, 380 mg, 2.34 mmol) in DCE (5 mL) was added MnO₂ (5.0 eq, 1.02 g, 11.7 mmol) and then the mixture was stirred for 14 h at 25° C. The reaction was filtered. MnO₂ (5.00 eq, 1.02 g, 11.7 mmol) was added, and the mixture was stirred for 12 h at 45° C. The reaction was filtered and concentrated under reduced pressure to afford 2-methylpyrazolo[1,5-a]pyridine-5-carbaldehyde (A89, 330 mg, 2.06 mmol, 88% yield) as a solid. LC/MS (ESI, m/z⁺): 161.0 [M+H]⁺.

### Synthesis of A90

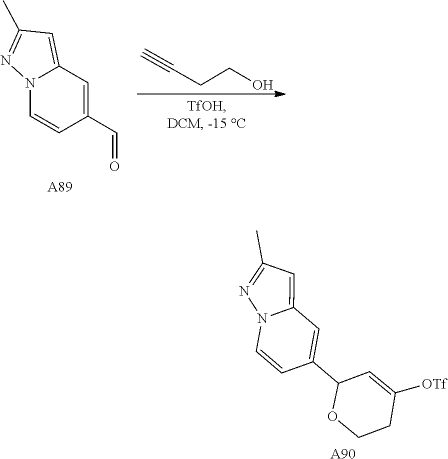

A mixture of 2-methylpyrazolo[1,5-a]pyridine-5-carbaldehyde (A89, 1.0 eq, 280 mg, 1.75 mmol) and but-3-yn-1-ol (1.0 eq, 0.13 mL, 1.75 mmol) in DCM (3 mL) was stirred for 0.5 h at −15° C. TfOH (3.0 eq, 0.46 mL, 5.24 mmol) was added, and the reaction was stirred for 1 h at −15° C. NaHCO₃(aq. 30 mL) was added to the mixture slowly and the solution was extracted with EtOAc (2×20 mL). The combined organic layers were separated, dried over Na₂SO₄, and filtered. The filtrate was concentrated under reduced pressure and purified by prep-HPLC (FA) to give [6-(2-methylpyrazolo[1,5-a]pyridin-5-yl)-3,6-dihydro-2H-pyran-4-yl]trifluoromethanesulfonate (A90, 60 mg, 0.129 mmol, 7% yield) as a gum. LC/MS (ESI, m/z⁺): 362.9 [M+H]⁺. ¹H NMR (400 MHz, CDCl₃) δ ppm 2.49 (s, 4H) 2.62-2.77 (m, 1H), 3.84-3.95 (m, 1H), 4.07-4.17 (m, 1H), 5.27 (br d, J=2.08 Hz, 1H), 5.94 (s, 1H), 6.31 (s, 1H), 6.66 (d, J=7.09 Hz, 1H), 7.37 (s, 1H), 8.38 (br d, J=7.34 Hz, 1H).

### Synthesis of A91

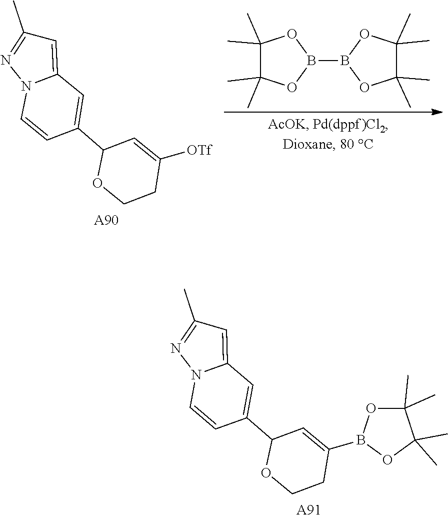

To a solution of [6-(2-methylpyrazolo[1,5-a]pyridin-5-yl)-3,6-dihydro-2H-pyran-4-yl]trifluoromethanesulfonate (1.0 eq, 60 mg, 0.13 mmol), AcOK (4.0 eq, 51 mg, 0.52 mmol) and B₂pin₂ (1.5 eq, 49 mg, 0.19 mmol) in 1,4-Dioxane (1.5 mL) was added Pd(dppf)Cl₂ DCM (0.10 eq, 11 mg, 0.013 mmol) under N₂ atmosphere, then the solution was stirred for 2 h at 80° C. The reaction solution was extracted with EtOAc (3×20 mL) and water (30 mL). The combined organic layer was washed with brine (30 mL), dried over Na₂SO₄, and filtered. The filtrate was concentrated under reduced pressure and purified by prep-HPLC (FA) to give 2-methyl-5-[4-(4,4,5,5-tetramethyl-1,3,2-dioxaborolan-2-yl)-3,6-dihydro-2H-pyran-6-yl]pyrazolo[1,5-a]pyridine (A91, 60 mg, contained EtOAc) as a solid. LC/MS (ESI, m/z⁺): 341.1 [M+H]⁺. ¹H NMR (400 MHz, CDCl₃) δ ppm 1.25-1.32 (m, 12H), 2.18-2.27 (m, 1H), 2.41 (dtd, J=11.66, 5.86, 5.86, 2.50 Hz, 1H), 2.48 (s, 3H), 3.73-3.84 (m, 1H), 4.03 (ddd, J=11.26, 5.19, 3.56 Hz, 1H), 5.15 (br d, J=2.50 Hz, 1H), 6.24 (s, 1H), 6.56 (d, J=1.63 Hz, 1H), 6.69 (dd, J=7.13, 1.88 Hz, 1H), 7.35-7.43 (m, 1H), 8.32 (d, J=7.25 Hz, 1H).

### Method Int-20: Synthesis of A93

### Synthesis of A92

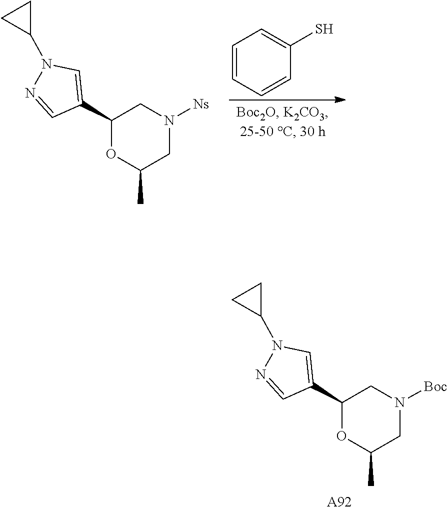

A mixture of (2S,6R)-2-(1-cyclopropyl-1H-pyrazol-4-yl)-6-methyl-4-((4-nitrophenyl)sulfonyl)-morpholine (1.0 eq, 500 mg, 1.27 mmol), benzenethiol (3.0 eq, 0.39 mL, 3.82 mmol), K₂CO₃ (4.0 eq, 704 mg, 5.10 mmol) in MeCN (35 mL) and DMSO (0.71 mL) was stirred at 50° C. for 16 h. Boc₂O (5.0 eq, 1.5 mL, 6.37 mmol) was added, and the mixture was stirred at 25° C. for 14 h. The mixture was concentrated under water pump and purified by reversed-phase column [water (NH₃ H₂O)-MeCN; B %: 30%-60%) and lyophilized to give tert-butyl (2S,6R)-2-(1-cyclopropyl-1H-pyrazol-4-yl)-6-methylmorpholine-4-carboxylate (A92, 370 mg, 1.29 mmol, 94% yield) as an oil. LC/MS (ESI, m/z⁺): [M+H]⁺=308.1. ¹H NMR (400 MHz, CDCl₃) δ=7.46 (s, 2H), 4.47-4.40 (m, 1H), 4.15-3.83 (m, 2H), 3.71-3.62 (m, 1H), 3.55 (tt, J=3.8, 7.3 Hz, 1H), 2.90-2.71 (m, 1H), 2.56 (br d, J=1.1 Hz, 1H), 1.48 (s, 9H), 1.22 (d, J=6.2 Hz, 3H), 1.13-1.08 (m, 2H), 1.03-0.96 (m, 2H).

### Synthesis of A93

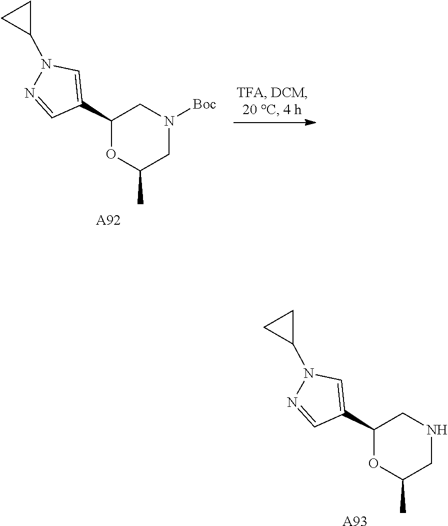

A solution of tert-butyl (2S,6R)-2-(1-cyclopropyl-1H-pyrazol-4-yl)-6-methylmorpholine-4-carboxylate (A92, 1.0 eq, 350 mg, 1.14 mmol) in TFA (1.0 eq, 0.50 mL, 1.14 mmol) and DCM (4 mL) was stirred at 20° C. for 4 h. TFA (1.0 eq, 0.50 mL) was added and the mixture was stirred at 20° C. for 0.5 h. The reaction mixture was poured into 50% NaOH aqueous solution (20 mL) until pH=14 and extracted with DCM (3×20 mL). The combined organic layers were dried over Na₂SO₄, filtered and concentrated under reduced pressure to give (2S,6R)-2-(1-cyclopropyl-1H-pyrazol-4-yl)-6-methylmorpholine (A93, 210 mg, 1.01 mmol, 89% yield) as an oil. LC/MS (ESI, m/z):. [M+H]⁺=208.2.

¹H NMR (400 MHz, CDCl₃) δ=7.45 (d, J=4.4 Hz, 2H), 4.49 (dd, J=2.1, 10.5 Hz, 1H), 3.78-3.66 (m, 1H), 3.54 (tt, J=3.8, 7.2 Hz, 1H), 3.01 (dd, J=0.9, 12.3 Hz, 1H), 2.90 (br d, J=11.9 Hz, 1H), 2.82-2.74 (m, 1H), 2.54 (dd, J=10.8, 11.8 Hz, 1H), 1.18 (d, J=6.3 Hz, 3H), 1.12-1.07 (m, 2H), 1.02-0.95 (m, 2H).

### Method Int-21: Synthesis of A10

### Synthesis of A96

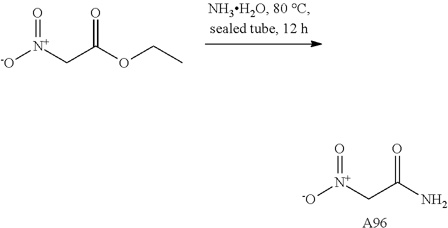

In a sealed tube, a mixture of ethyl 2-nitroacetate (1.0 eq, 15.0 g, 113 mmol) in NH₃·H₂O (5.32 eq, 50 mL, 600 mmol) was stirred at 80° C. for 12 h. The mixture was cooled to 20° C., poured into water (100 mL) and concentrated under reduced pressure to half-volume. HCl (1 N, 20 mL) was added to adjust pH=1-2, and the mixture was extracted with EtOAc (6×300 mL). The organic layers were dried over Na₂SO₄, filtered and concentrated under reduced pressure to afford 2-nitroacetamide (A96, 10.0 g, 96.1 mmol, 85% yield) as a solid. ¹H NMR (400 MHz, DMSO-d6) δ=7.85 (br s, 1H), 7.63 (br s, 1H), 5.28 (s, 2H).

### Synthesis of A97

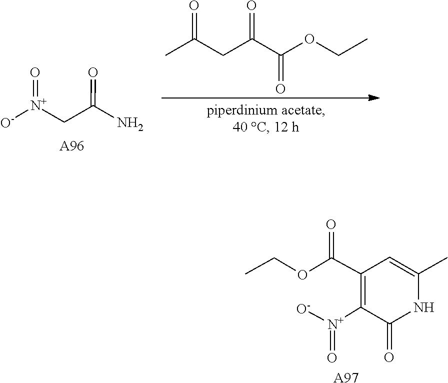

To a mixture of 2-nitroacetamide (A96, 1.0 eq, 20.0 g, 192 mmol) and ethyl 2,4-dioxopentanoate (1.1 eq, 33.44 g, 211 mmol) in water (300 mL) was added piperidinium acetate (1.0 eq, 27.9 g, 192 mmol), and the reaction was stirred at 40° C. for 12 h. The mixture was extracted with EtOAc (4×500 mL), and the organic phase was concentrated under reduced pressure to give a residue. The residue was triturated in EtOAc (20 mL) and filtered. The filtrate was concentrated under reduced pressure and purified by column chromatography (50-100% EtOAc in hexane) to afford ethyl 6-methyl-3-nitro-2-oxo-1H-pyridine-4-carboxylate (A97, 8.0 g, 35.4 mmol, 18% yield) as a solid. LC/MS (ESI, m/z⁺): [M+H]⁺=227.1. ¹H NMR (400 MHz, DMSO-d6) δ=13.45-12.81 (m, 1H), 6.41 (s, 1H), 4.29 (q, J=7.1 Hz, 2H), 2.32 (s, 3H), 1.24 (t, J=7.1 Hz, 3H).

### Synthesis of A98

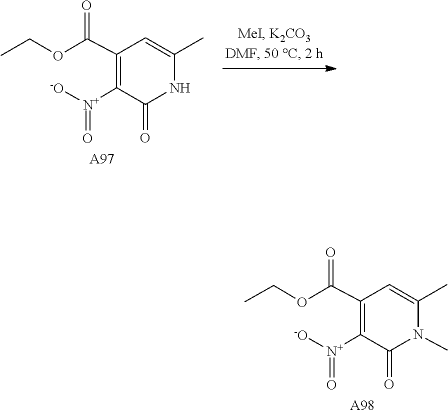

To a solution of ethyl 6-methyl-3-nitro-2-oxo-1H-pyridine-4-carboxylate (A97, 1.0 eq, 12.50 g, 55.3 mmol) and K₂CO₃ (2.0 eq, 15.28 g, 111 mmol) in DMF (100 mL) was added methyl iodide (3.0 eq, 10 mL, 166 mmol), and the reaction was stirred at 50° C. for 2 h. The resulting mixture was concentrated under reduced pressure and purified by column chromatography (18-100% EtOAc in hexane) to afford ethyl 1,6-dimethyl-3-nitro-2-oxo-pyridine-4-carboxylate (A98, 11.0 g, 45.8 mmol, 83% yield) as a solid. LC/MS (ESI, m/z⁺): [M+H]⁺=241.1. ¹H NMR (400 MHz, METHANOL-d4) δ=6.62 (s, 1H), 4.36 (q, J=7.2 Hz, 2H), 3.66 (s, 3H), 2.56 (s, 3H), 1.33 (t, J=7.2 Hz, 3H).

### Synthesis of A99

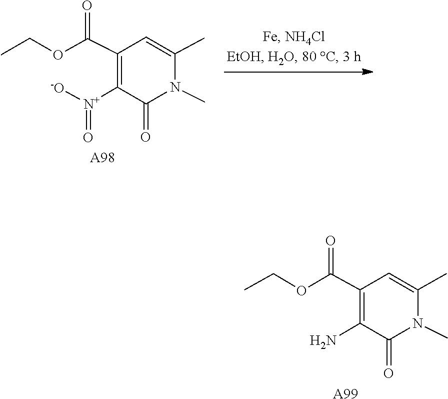

To a solution of ethyl 1,6-dimethyl-3-nitro-2-oxo-pyridine-4-carboxylate (A98, 1.0 eq, 11.0 g, 45.8 mmol) in EtOH (150 mL) and water (40 mL), were added NH₄Cl (5.0 eq, 12.25 g, 229 mmol) and Fe (5.0 eq, 12.79 g, 229 mmol), then the mixture was stirred at 80° C. for 3 h. The mixture was filtered, and the filter cake was washed with EtOAc/MeOH (9:1, 300 mL). The filtrate was extracted with EtOAc (4×400 mL) and the combined organic layers were concentrated under reduced pressure to give a solid. The solid was washed with water (30 mL) and concentrated to give ethyl 3-amino-1,6-dimethyl-2-oxo-pyridine-4-carboxylate (A99, 9.0 g, 42.8 mmol, 93% yield) as a solid. LC/MS (ESI, m/z⁺): [M+H]⁺=211.1. ¹H NMR (400 MHz, DMSO-d6) δ=6.66 (br s, 2H), 6.31 (d, J=0.9 Hz, 1H), 4.25 (q, J=7.1 Hz, 2H), 3.46 (s, 3H), 2.25 (d, J=0.7 Hz, 3H), 1.29 (t, J=7.1 Hz, 3H).

### Synthesis of A100

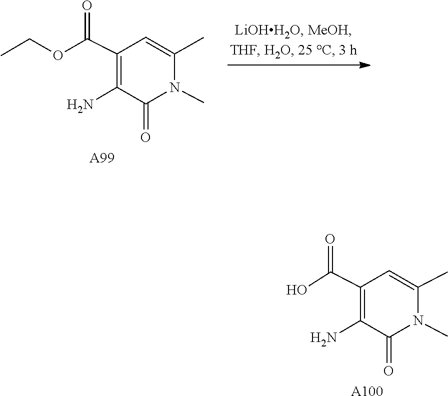

To a solution of ethyl 3-amino-1,6-dimethyl-2-oxo-pyridine-4-carboxylate (A99, 1.0 eq, 9.0 mg, 42.8 mmol) in THF (80 mL), methanol (80 mL) and water (80 mL) was added LiOH H₂O (2.0 eq, 3.59 g, 85.6 mmol), and the reaction was stirred at 25° C. for 12 h. The resulting mixture was adjusted to pH 2-3 with 1 N HCl (aq.), which resulted in solid formation. The mixture was filtered and the solid was determined to be desired product. The filtrate was extracted with EtOAc/MeOH (8×500 mL/50 mL). The combined organic phase was concentrated under reduced pressure to give a solid. The two batches of solid were combined to afford 3-amino-1,6-dimethyl-2-oxo-pyridine-4-carboxylic acid (A100, 7.50 g, 41.2 mmol, 96% yield) as a solid. LC/MS (ESI, m/z⁺): [M+H]⁺=183.0. ¹H NMR (400 MHz, DMSO-d6) δ=6.30 (br s, 1H), 3.45 (br s, 3H), 2.23 (br s, 3H).

### Synthesis of A101

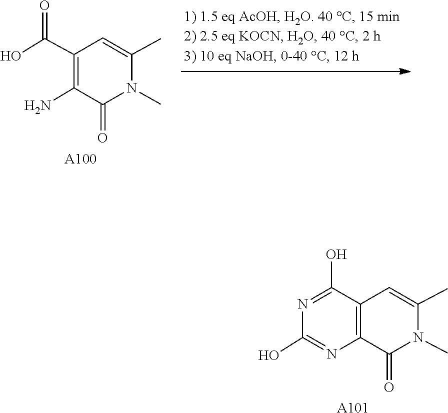

To a suspension of 3-amino-1,6-dimethyl-2-oxo-pyridine-4-carboxylic acid (A100, 1.0 eq, 5.00 g, 27.4 mmol) in water (100 mL) was added AcOH (1.5 eq, 2.47 g, 41.2 mmol), then the mixture was stirred at 40° C. for 15 min. Potassium cyanate (2.5 eq, 5.57 g, 68.6 mmol) in water (30 mL) was added dropwise, and the reaction was stirred at 40° C. for 1 h. NaOH (10.0 eq, 10.98 g, 274 mmol) was added at 0° C., the mixture was stirred at 40° C. for 2 h. Water (50 mL) was added into the mixture, and the pH was adjusted to 7 with HCl (aq.), which resulted in solid formation. The mixture was filtered and the filter cake was washed with water (3×10 mL) and concentrated to afford 2,4-dihydroxy-6,7-dimethyl-pyrido[3,4-d]pyrimidin-8-one (A101, 5.20 g, 25.1 mmol, 91% yield) as a solid. LC/MS (ESI, m/z⁺): [M+H]⁺=208.0. ¹H NMR (400 MHz, DMSO-d6) δ=11.92-10.20 (m, 2H), 6.47 (br s, 1H), 3.51 (br s, 3H), 2.37 (br s, 3H).

### Method Int-22: Synthesis of A126

### Synthesis of A125

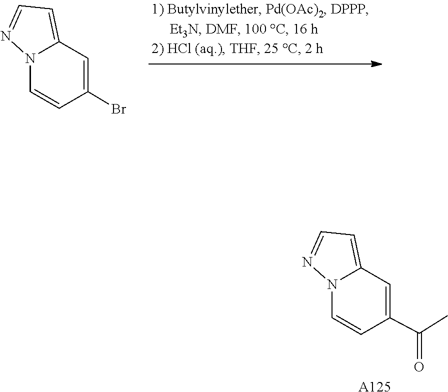

5-bromopyrazolo[1,5-a]pyridine (1.0 eq, 3.30 g, 16.7 mmol), butyl vinyl ether (10.0 eq, 22 mL, 167 mmol), TEA (1.2 eq, 2.1 mL, 20.1 mmol) and DPPP (0.3 eq, 2.07 g, 5.02 mmol) were diluted in DMF (1.7 mL). The resulting mixture was degassed under argon and Pd(OAc)₂ (10 mol %, 376 mg, 1.67 mmol) was added. The resulting mixture was stirred overnight at 100° C. The reaction was quenched with water and extracted with DCM (×2). The combined organic layers were washed with brine, dried over Na₂SO₄, filtered, and concentrated under reduced pressure. The resulting mixture was diluted with THF (5.0 mL) and 2.0 N HCl (aq.) (0.5 mL) was added. The resulting mixture was stirred at room temperature for 2 h. The reaction was quenched with NaHCO₃ and extracted with DCM (×3). The combined organic layers were washed with brine, dried over Na₂SO₄, and filtered. The filtrate was concentrated under reduced pressure and purified by normal phase column chromatography (5-40% EtOAc in hexanes) to afford 1-pyrazolo[1,5-a]pyridin-5-ylethanone (A125, 1.84 g, 11.5 mmol, 69% yield) as a solid. ESI-MS (m/z)+: 161.1 (obs).

### Synthesis of A126

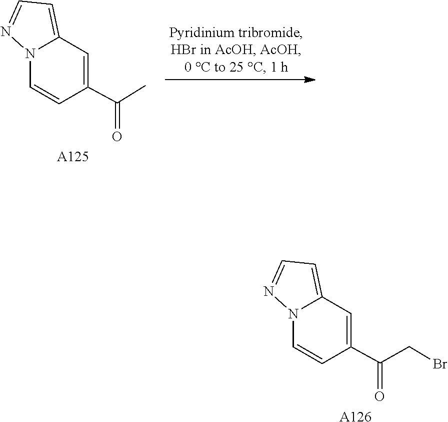

To a solution of 1-pyrazolo[1,5-a]pyridin-5-ylethanone (A125, 1.0 eq, 1.74 g, 10.9 mmol) in 33% HBr in AcOH (14.0 eq, 8.7 mL, 152 mmol) at room temperature was added bromine (0.95 eq, 0.53 mL, 10.3 mmol) and the reaction mixture was stirred at room temperature for 30 min. The reaction was quenched with NaHCO₃ and extracted with DCM (×2). The combined organic layers were washed with brine, dried over Na₂SO₄, and filtered. The filtrate was concentrated under reduced pressure and purified by normal phase column chromatography (0-30% EtOAc in hexanes) to afford 2-bromo-1-pyrazolo[1,5-a]pyridin-5-yl-ethanone (A126, 2.17 g, 9.1 mmol, 84% yield) as a solid. ESI-MS (m/z)+: 241.1 (obs). ¹H-NMR (CHCl₃-d, 400 MHz): δ_{H} 8.48 (dt, J=7.3, 0.9 Hz, 1H), 8.23 (t, J=0.9 Hz, 1H), 8.03 (d, J=2.3 Hz, 1H), 7.28 (dd, J=7.4, 1.9 Hz, 1H), 6.79 (dd, J=2.4, 0.8 Hz, 1H), 4.42 (s, 2H).

### Method Int-23: Synthesis of A141

### Synthesis of A139

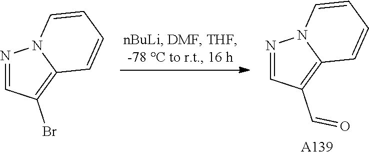

3-bromopyrazolo[1,5-a]pyridine (1.0 eq, 1.0 g, 5.08 mmol) was charged into a flame-dried round-bottomed flask equipped with a Teflon-coated magnetic stirring bar. The flask was placed under nitrogen and anhydrous THF (25 mL) was added. The resulting solution was cooled to −78° C. in a dry-ice/acetone bath and a solution of n-BuLi (1.6 M in hexanes, 1.1 eq, 3.1 mL, 5.6 mmol) was added dropwise. The reaction mixture was stirred for 0.5 h at −78° C. and NN-dimethylformamide (1.5 eq, 589 μL, 7.6 mmol) was added. The reaction mixture was continued to stir at −78° C., gradually warming to RT overnight. The reaction was quenched with sat. NaHCO₃(aq.) and extracted with DCM (×3). The combined organic layers were washed with brine, dried over Na₂SO₄, and filtered. The filtrate was concentrated under reduced pressure and purified by normal phase column chromatography (0-30% EtOAc in hexanes) to afford pyrazolo[1,5-a]pyridine-3-carbaldehyde (A139, 0.27 g, 1.86 mmol, 37% yield). ¹H NMR (400 MHz, CHCl₃-d): δ_{H} 10.86 (s, 1H), 8.06 (d, J=2.3 Hz, 1H), 7.80 (dd, J=8.8, 1.3 Hz, 1H), 7.46 (d, J=5.7 Hz, 1H), 7.20 (dd, J=8.8, 7.0 Hz, 1H), 6.70 (d, J=2.5 Hz, 1H). ESI-MS (m/z)+: 147.1 (obs).

### Synthesis of A140

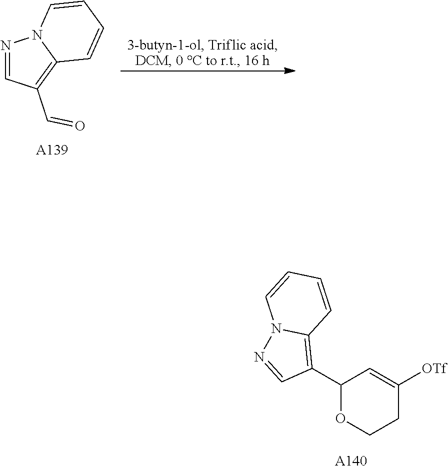

Triflic acid (3.0 eq, 0.50 mL, 5.58 mmol) was added dropwise to a solution of pyrazolo[1,5-a]pyridine-3-carbaldehyde (A139, 1.0 eq, 272 mg, 1.86 mmol) and 3-butyn-1-ol (1.1 eq, 0.15 mL, 1.95 mmol) in DCM (18 mL) at 0° C. The resulting mixture was allowed to warm up to room temperature under stirring overnight. The reaction was carefully quenched with NaHCO₃ and extracted with DCM (×3). The combined organic layers were washed with brine, dried over Na₂SO₄, and filtered. The filtrate was concentrated under reduced pressure and purified by normal phase column chromatography (5-30% EtOAc in hexanes) to afford (6-pyrazolo[1,5-a]pyridin-3-yl-3,6-dihydro-2H-pyran-4-yl) trifluoromethanesulfonate (A140, 151 mg, 0.43 mmol, 23% yield) as an oil. ¹H NMR (400 MHz, CHCl₃-d): δ_{H} 7.99 (d, J=2.3 Hz, 1H), 7.55 (d, J=8.0 Hz, 1H), 7.13 (dd, J=8.9, 7.1 Hz, 1H), 6.88 (d, J=6.9 Hz, 1H), 6.58 (d, J=2.3 Hz, 1H), 6.23 (s, 2H), 4.19-4.14 (m, 1H), 4.03-3.97 (m, 1H), 2.70-2.63 (m, 1H), 2.55-2.49 (m, 1H). ESI-MS (m/z)+: 349.1 (obs).

### Synthesis of A141

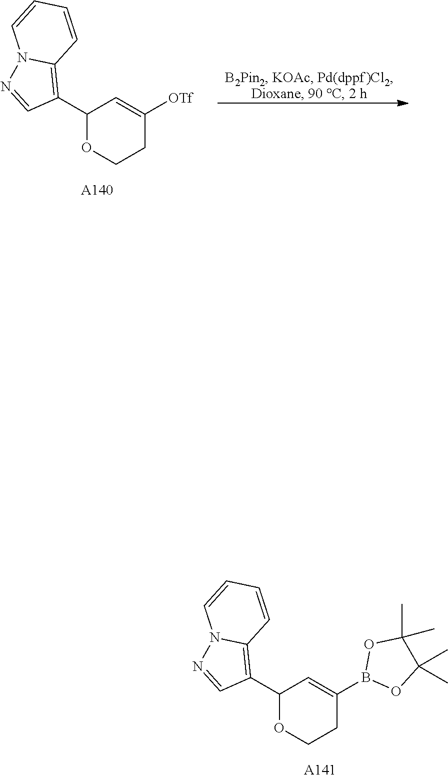

B₂pin₂ (1.9 eq, 0.21 g, 0.83 mmol), potassium acetate (6.4 eq, 0.27 g, 2.76 mmol), (6-pyrazolo-[1,5-a]pyridin-3-yl-3,6-dihydro-2H-pyran-4-yl) trifluoromethanesulfonate (A140, 1.0 eq, 151 mg, 0.43 mmol), Pd(dppf)Cl₂ (0.16 eq, 0.05 g, 0.07 mmol) and 1,4-dioxane (4 mL) were added to a glass vial equipped with a Teflon-coated magnetic stirring bar. The resulting mixture was degassed with argon for 5 min and stirred for 2 h at 90° C. The reaction was cooled to RT and diluted with DCM and H₂O. The organic layer was collected, and the aqueous layer was extracted with DCM (×3). The combined organic layers were dried over Na₂SO₄ and filtered. The filtrate was concentrated under reduced pressure and purified by normal phase column chromatography (5-40% EtOAc in hexanes) to afford 3-[4-(4,4,5,5-tetramethyl-1,3,2-dioxaborolan-2-yl)-3,6-dihydro-2H-pyran-6-yl]pyrazolo[1,5-a]pyridine (A141, 90 mg, 0.28 mmol, 64% yield) as an oil. ESI-MS (m/z)+: 327.3 (obs).

An analogous method was followed to obtain the following compound.

| Int # | Structure | Starting Material | Characterization |
|---|---|---|---|
| A118a and A118b | 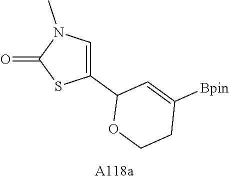 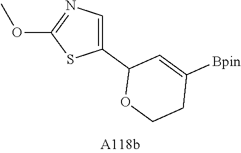 | 5-bromo-2- methoxy- thiazole | The residue was purified by silica gel flash column chromatography (10-100% EtOAc in Hexanes) to yield 3-methyl-5-[4-(4,4,5,5-tetramethyl-1,3,2- dioxaborolan-2-yl)-3,6-dihydro-2H-pyran-6- yl]thiazol-2-one (A118a, 33 mg, 0.1 mmol, 34% yield) and 2-methoxy-5-[4-(4,4,5,5-tetramethyl- 1,3,2-dioxaborolan-2-yl)-3,6-dihydro-2H-pyran-6- yl]thiazole (A118b, 40 mg, 0.12 mmol, 42% yield) as oils. A118a: ESI-MS (m/z)+: 324.3 (obs). ¹H NMR (CHCl₃-d, 400 MHz): δ_{H} 6.46 (d, J = 10.1 Hz, 2H), 5.00 (s, 1H), 3.85-3.81 (m, 1H), 3.74-3.68 (m, 1H), 3.27 (s, 3H), 2.22 (s, 3H), 1.26 (s, 12H). A118b: ESI-MS (m/z)+: 324.3 (obs). ¹H NMR (CHCl₃-d, 400 MHz): δ_{H} 6.97 (s, 1H), 6.54 (s, 1H), 5.24 (s, 1H), 4.02 (s, 3H), 3.89-3.84 (m, 1H), 3.74- 3.69 (m, 1H), 2.23 (dd, J = 24.6, 20.0 Hz, 2H), 1.36-1.26 (m, 12H). |

### Method Int-24: Synthesis of A148

### Synthesis of A144

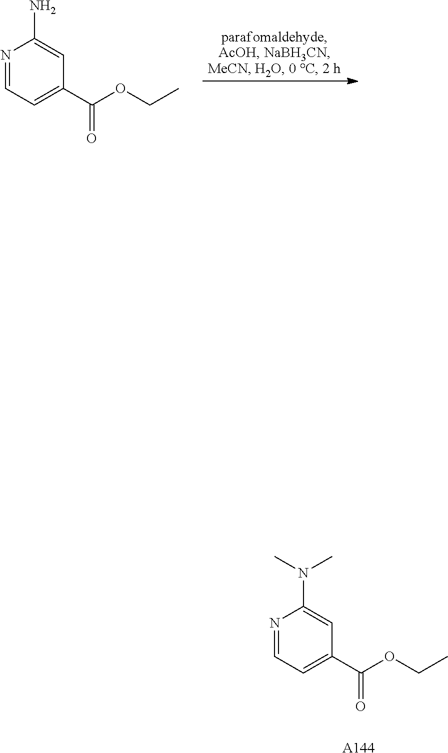

To a flame-dried round-bottom flask equipped with a Teflon-coated magnetic stirring bar was added ethyl 2-aminopyridine-4-carboxylate (1.0 eq, 1.28 g, 7.70 mmol), MeCN (42 mL) and water (8 mL). The flask was cooled to 0° C., acetic acid (5.0 eq, 2.2 mL, 38.5 mmol) and paraformaldehyde (10.0 eq, 5.7 mL, 77.0 mmol) were added, and the resulting mixture was stirred at 0° C. for 15 min. Sodium cyanoborohydride (3.0 eq, 1.45 g, 23.1 mmol) was added, and the reaction mixture was allowed to warm up gradually to room temperature over a period of 2 h. The reaction mixture was quenched with sat. NaHCO₃ and diluted with EtOAc (150 mL). The layers were separated, and the organic layer was washed with brine, dried over Na₂SO₄, and filtered. The filtrate was concentrated under reduced pressure and purified by silica gel column chromatography (1-5% MeOH in DCM) to afford ethyl 2-(dimethylamino)-pyridine-4-carboxylate (A144, 1.49 g, 7.67 mmol, 99% yield) as an oil. ESI-MS (m/z)+: 195.2 (obs). ¹H NMR (400 MHz, DMSO-d6) δ_{H} 8.19 (dd, J=5.0, 0.7 Hz, 1H), 6.97 (s, 1H), 6.93-6.91 (m, 1H), 6.04-5.95 (m, 1H), 4.34-4.21 (m, 5H), 3.04 (d, J=13.5 Hz, 6H), 1.42-1.13 (m, 3H).

### Synthesis of A145

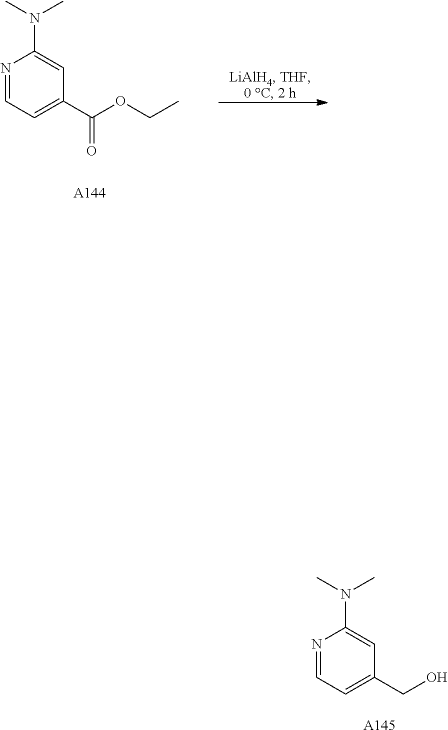

A solution of LiAlH₄ (2.0 M in THF, 3.5 mL, 1.0 eq, 7.0 mmol) was added dropwise to a solution of ethyl 2-(dimethylamino)pyridine-4-carboxylate (A144, 1.0 eq, 1.36 g, 7.0 mmol) in THF (70 mL) at 0° C., and the reaction was stirred for 2 h at room temperature. The resulting mixture was quenched with NaHCO₃ and the aqueous layer was extracted with EtOAc (×3). The combined organic layers were washed with brine, dried over Na₂SO₄, filtered, and concentrated under reduced pressure to afford [2-(dimethylamino)-4-pyridyl]methanol (A145, 526 mg, 3.46 mmol, 49% yield). The crude was used in the next step without further purification. ESI-MS (m/z)+: 153.1 (obs).

### Synthesis of A146

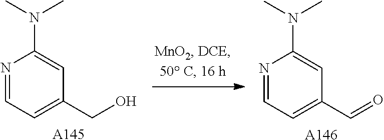

[2-(dimethylamino)-4-pyridyl]methanol (A145, 1.0 eq, 688 mg, 4.52 mmol) was stirred in DCE (45 mL) at 50° C. overnight in presence of MnO₂ (10.0 eq, 3.9 g, 45.2 mmol). The resulting mixture was filtered through a celite pad, concentrated under reduced pressure, and purified by normal phase column chromatography (20-60% EtOAc in hexanes) to afford 2-(dimethylamino)pyridine-4-carbaldehyde (A146, 350 mg, 2.33 mmol, 52% yield). ¹H NMR (400 MHz, CHCl₃-d) δ_{H} 9.90 (s, 1H), 8.30 (d, J=5.0 Hz, 1H), 6.86 (d, J=5.0 Hz, 1H), 6.83 (s, 1H), 3.10 (s, 6H). ESI-MS (m/z+H₂O)+: 169.1 (obs).

### Synthesis of A147

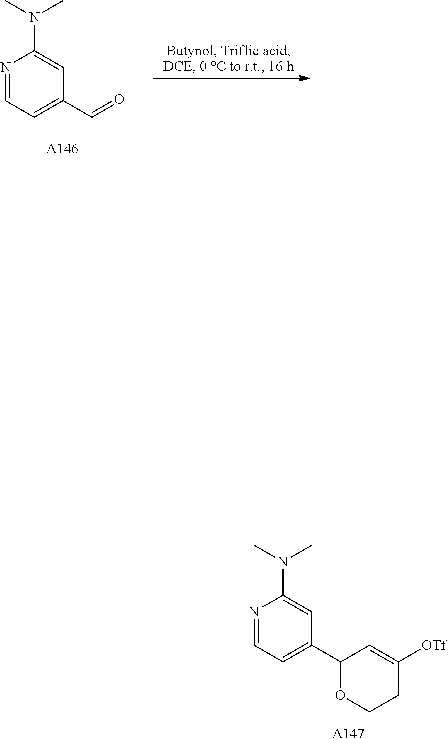

Triflic acid (3.0 eq, 0.62 mL, 6.99 mmol) was added dropwise to a solution of 2-(dimethylamino)pyridine-4-carbaldehyde (A146, 1.0 eq, 350 mg, 2.33 mmol) and 3-butyn-1-ol (1.0 eq, 0.18 mL, 2.33 mmol) in DCE (23 mL) at 0° C. The resulting mixture was allowed to warm up to room temperature under stirring overnight. The reaction was carefully quenched with NaHCO₃ and extracted with DCM (×3). The combined organic layers were washed with brine, dried over Na₂SO₄, and filtered. The filtrate was concentrated under reduced pressure and purified by normal phase column chromatography (5-40% EtOAc in hexanes) to afford [6-[2-(dimethylamino)-4-pyridyl]-3,6-dihydro-2H-pyran-4-yl]trifluoromethanesulfonate (A147, 260 mg, 0.74 mmol, 32% yield). ESI-MS (m/z)+: 353.1 (obs). ¹H NMR (400 MHz, CHCl₃-d) δ_{H} 8.14 (d, J=5.3 Hz, 1H), 6.48-6.46 (m, 2H), 5.89 (s, 1H), 5.14 (d, J=2.3 Hz, 1H), 4.12-4.07 (m, 1H), 3.88-3.82 (m, 1H), 3.07 (s, 6H), 2.65 (d, J=25.4 Hz, 1H), 2.39 (dd, J=16.9, 3.0 Hz, 1H).

### Synthesis of A148

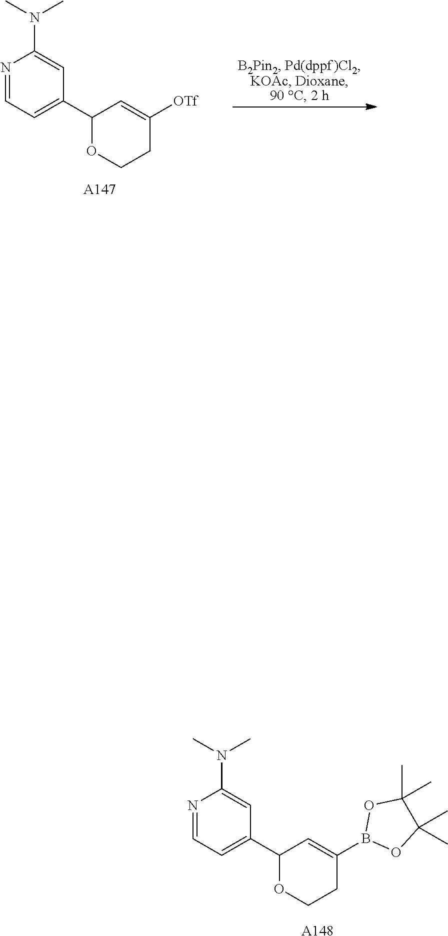

B₂pin₂ (1.2 eq, 227 mg, 0.90 mmol), KOAc (4.0 eq, 294 mg, 3.0 mmol), [6-[2-(dimethylamino)-4-pyridyl]-3,6-dihydro-2H-pyran-4-yl]trifluoromethanesulfonate (A147, 1.0 eq, 264 mg, 0.75 mmol), Pd(dppf)Cl₂ (0.1 eq, 55 mg, 0.075 mmol) and 1,4-dioxane (7.0 mL) were added to a glass vial equipped with a Teflon-coated magnetic stirring bar. The resulting mixture was degassed with argon for 5 min and the reaction was stirred for 2 h at 90° C. The reaction was quenched with water, the aqueous layer was extracted with EtOAc (×3). The combined organic layers were washed with brine, dried over Na₂SO₄, and filtered. The filtrate was concentrated under reduced pressure and purified by normal phase column chromatography (20-80% EtOAc in hexanes) to afford N,N-dimethyl-4-[4-(4,4,5,5-tetramethyl-1,3,2-dioxaborolan-2-yl)-3,6-dihydro-2H-pyran-6-yl]pyridin-2-amine (A148, 116 mg, 0.35 mmol, 47% yield). ESI-MS (m/z)+: 331.3 (obs).

### Example A2: Synthesis of Exemplary Compounds

### Method 1: Synthesis of Example 1 and 3

### Example Synthesis of Example 1: methyl 2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxylate

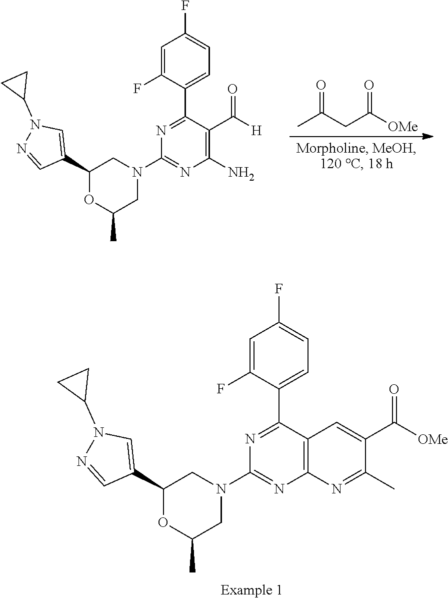

To a flame-dried microwave flask, was added 4-amino-2-[(2S,6R)-2-(1-cyclopropyl-pyrazol-4-yl)-6-methyl-morpholin-4-yl]-6-(2,4-difluorophenyl)pyrimidine-5-carbaldehyde (A2, 1 eq, 0.9 mmol, 300 mg), methyl 3-oxobutanoate (2 eq, 343 μL, 3.18 mmol), morpholine (1 eq, 134 μL, 1.59 mmol) and MeOH (1.6 mL). The flask was capped and stirred in an oil bath at 120° C. for 18 h. The mixture was cooled to 23° C. and poured in a separatory funnel, washed with brine (20 mL), and extracted with EtOAc (3×15 mL). The combined organic layers were dried over Na₂SO₄, filtered, and concentrated under reduced pressure. The crude was purified by reversed-phase flash chromatography (30-70% MeCN in water (0.1% formic acid)) to afford methyl 2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxylate (Example 1, 450 mg, 0.87 mmol, 54% yield) as a solid. LC/MS (ESI, m/z⁺): 521.2 [M+H]⁺. ¹H NMR (CHCl₃-d, 400 MHz): δ_{H} 8.38 (1H, d, J=3.6 Hz), 7.56 (2H, s), 7.52 (2H, s), 7.02-7.11 (2H, m), 4.90-5.27 (1H, m), 4.57 (1H, d, J=10.0 Hz), 3.87 (3H, s), 3.78 (1H, d, J=7.2 Hz), 3.56 (2H, s), 3.04-3.10 (1H, m), 2.96 (3H, s), 2.79-2.87 (1H, m), 1.31 (3H, d, J=5.6 Hz), 1.10 (2H, t, J=3.8 Hz), 0.99 (2H, d, J=7.0 Hz). ¹⁹F NMR (CHCl₃-d, 376 MHz): δ_{F}−105.6 (1F, m), −108.7 (1F, m).

### Example 3: 4-(2,4-difluorophenyl)-7-methyl-2-[-(2S,6R)-2-(1-cyclopropylpyrazol-4-yl)-6-methyl-morpholin-4-yl]pyrido[2,3-d]-pyrimidine-6-carboxylic acid

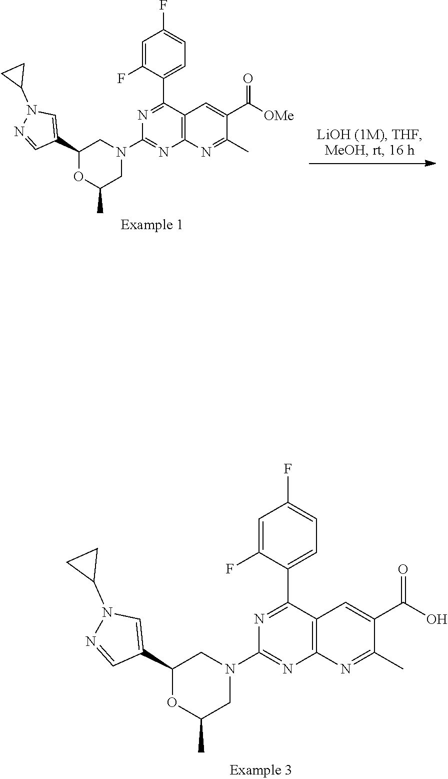 [SMILES: COC(=O)c1cc2c(-c3ccc(F)cc3F)nc(N3C[C@@H](C)O[C@@H](c4cnn(C5CC5)c4)C3)nc2nc1C ; "LiOH (1M), THF,MeOH, rt, 16 h" ; Cc1nc2nc(N3C[C@@H](C)O[C@@H](c4cnn(C5CC5)c4)C3)nc(-c3ccc(F)cc3F)c2cc1C(=O)O]

Methyl 2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxylate (Example 1, 1 eq, 165 mg, 0.32 mmol), LiOH (3 eq, 23 mg, 0.95 mmol) and H₂O (1 mL) were stirred at 23° C. in a mixture of MeOH (2.1 mL) and THF (4.2 mL) for 16 h. AcOH 10% (aq., 10 mL) was added to adjust the pH around 6. The mixture was poured in a separative funnel, washed with brine (20 mL) and extracted with DCM (3×15 mL). The combined organic layers were dried over Na₂SO₄, filtered, concentrated under reduced pressure, and purified by reversed-phase flash chromatography (10-35% MeCN in water (0.1% formic acid)) to afford 4-(2,4-difluorophenyl)-7-methyl-2-[-(2S,6R)-2-(1-cyclopropylpyrazol-4-yl)-6-methyl-morpholin-4-yl]pyrido[2,3-d]-pyrimidine-6-carboxylic acid (Example 3, 160 mg, 0.32 mmol, 99% yield) as a solid. LC/MS (ESI, m/z⁺): 507.4 [M+H]⁺. ¹H NMR (CH₃OH-d4, 400 MHz): δ_{H} 8.50 (1H, d, J=3.4 Hz), 7.76-7.79 (1H, m), 7.69-7.74 (1H, m), 7.52-7.58 (1H, m), 7.20-7.29 (2H, m), 4.95-5.06 (1H, m), 4.61-4.69 (1H, m), 3.80-3.88 (1H, m), 3.62-3.69 (1H, m), 3.08-3.22 (2H, m), 2.87-2.94 (4H, m), 1.27-1.35 (6H, m), 1.03-1.12 (5H, m). ¹⁹F NMR (CH₃OH-d4, 376 MHz): δ_{F}−107.6 (1F, m), −111.2 (1F, m).

### Method 2 Synthesis of Example 2

### Example 2: 2-((2S,6R)-2-(1-cyclopropyl-1H-pyrazol-4-yl)-6-methylmorpholino)-4-(2,4-difluorophenyl)-N,N-7-trimethylpyrido[2,3-d]pyrimidine-6-carboxamide

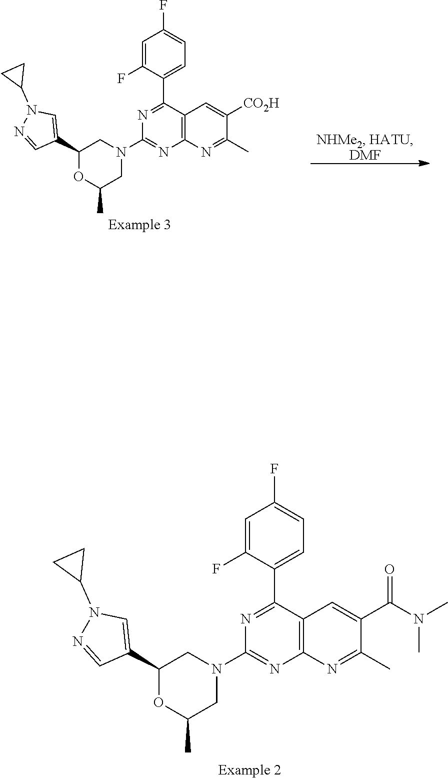

To a solution of 2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxylic acid (Example 3, 1 eq, 58 mg, 0.115 mmol) in DMF (2 mL) was added HATU (1.1 eq, 48 mg, 0.127 mmol) and dimethylamine (2 M in THF, 5 eq, 0.29 mL, 0.58 mmol), and the solution was stirred at 25° C. for 12 h. The reaction mixture was diluted with EtOAc, and the organic phase was washed with water, brine, dried over Na₂SO₄, and filtered. The filtrate was concentrated under reduced pressure and purified by reverse phase chromatography (5-100% MeCN in water (0.1% FA)) to afford after lyophilization 2-((2S,6R)-2-(1-cyclopropyl-1H-pyrazol-4-yl)-6-methylmorpholino)-4-(2,4-difluorophenyl)-N,N-7-trimethylpyrido[2,3-d]pyrimidine-6-carboxamide (Example 2, 28 mg, 0.05 mmol, 46% yield) as a solid. ¹H NMR (CHCl₃-d, 400 MHz): δ_{H} 7.63 (1H, d, J 3.8 Hz), 7.51-7.55 (3H, m), 7.06 (1H, t, J 8.2 Hz), 6.97 (1H, td, J 9.3, 2.4 Hz), 4.94-5.09 (2H, m), 4.57 (1H, d, J 10.7 Hz), 3.76-3.82 (1H, m), 3.55 (1H, tt, J 7.2, 3.8 Hz), 3.12 (3H, s), 3.02-3.09 (1H, m), 2.78-2.85 (4H, m), 2.65 (3H, s), 1.30 (3H, d, J 6.2 Hz), 1.08-1.12 (2H, m), 0.95-1.00 (2H, m). ¹⁹F NMR (CHCl₃-d, 376 MHz): δ_{F}−105.9, −108.8. LC/MS (ESI, m/z⁺): 534.3 [M+H]⁺.

### Method 3 Synthesis of Examples 4 and 5

### Example 4: 1-[2-[(2S,6R)-2-(1-cyclopropylpyrazol-4-yl)-6-methyl-morpholin-4-yl]-4-(2,4-difluorophenyl)-7-methyl-pyrido[2,3-d]pyrimidin-6-yl]ethenone

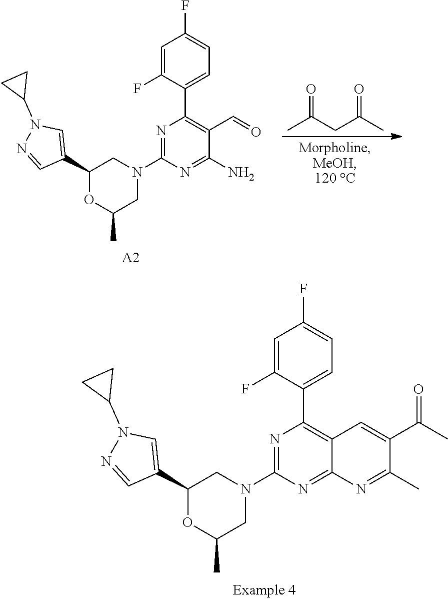

Pentane-2,4-dione (1 eq, 42 μL, 0.45 mmol) and morpholine (0.5 eq, 19 μL, 0.23 mmol) were added to a solution of 4-amino-2-[(2S,6R)-2-(1-cyclopropylpyrazol-4-yl)-6-methyl-morpholin-4-yl]-6-(2,4-difluorophenyl)pyrimidine-5-carbaldehyde (A2, 1 eq, 200 mg, 0.45 mmol) in MeOH (1.5 mL), and the reaction was stirred at 120° C. for 18 h. The mixture was cooled to room temperature and a saturated solution of NaHCO₃ (aq.) (20 mL) was added and the mixture was extracted with EtOAc (3×30 mL). The combined organic layers were washed with brine, dried over Na₂SO₄, and filtered. The filtrate was concentrated under reduced pressure and purified by reverse phase chromatography (30-55% MeCN in water (0.1% formic acid)) to afford 1-[2-[(2S,6R)-2-(1-cyclopropylpyrazol-4-yl)-6-methyl-morpholin-4-yl]-4-(2,4-difluorophenyl)-7-methyl-pyrido[2,3-d]pyrimidin-6-yl]ethenone (Example 4, 35 mg, 0.07 mmol, 15% yield) as a solid. LC/MS (ESI, m/z⁺): 505.3 [M+H]⁺. ¹H NMR (CHCl₃-d, 400 MHz): δ_{H} 8.16 (1H, d, J=4.1 Hz), 7.60 (1H, t, J=8.1 Hz), 7.51 (1H, s), 7.24 (1H, s), 7.11-7.09 (1H, m), 7.03 (1H, t, J=9.4 Hz), 5.24-4.88 (1H, m), 4.57 (1H, d, J=10.0 Hz), 3.80 (1H, d, J=8.7 Hz), 3.55 (1H, s), 3.07 (1H, dd, J=13.4, 10.9 Hz), 2.90 (3H, s), 2.84 (1H, dd, J=13.4, 10.7 Hz), 2.52 (3H, s), 1.98-1.81 (2H, m), 1.31 (3H, d, J=6.2 Hz), 1.11-1.08 (2H, m), 1.00 (2H, t, J=6.9 Hz). ¹⁹F NMR (CHCl₃-d, 376 MHz): δ_{F}−108.7 (1F, m), −105.2 (1F, m).

### Example 5: 1-[2-[(2S,6R)-2-(1-cyclopropylpyrazol-4-yl)-6-methyl-morpholin-4-yl]-4-(2,4-difluorophenyl)-7-methyl-pyrido[2,3-d]pyrimidin-6-yl]-N-methoxy

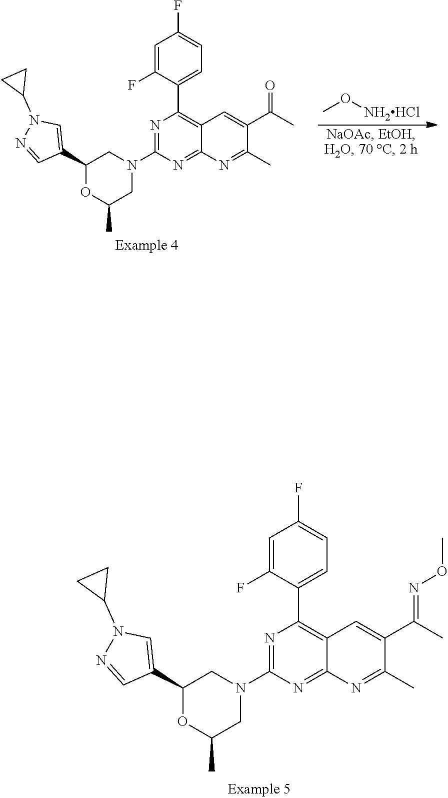

### Example 4 Example 5

A solution of 1-[2-[(2S,6R)-2-(1-cyclopropylpyrazol-4-yl)-6-methyl-morpholin-4-yl]-4-(2,4-difluorophenyl)-7-methyl-pyrido[2,3-d]pyrimidin-6-yl]ethanone (Example 4, 1 eq, 20 mg, 0.04 mmol), O-methylhydroxylamine hydrochloride (2.7 eq, 9 mg, 0.11 mmol) and sodium acetate (4.4 eq, 14 mg, 0.18 mmol) in a mixture of EtOH (0.5 mL) and water (1.5 mL) was heated at 70° C. for 2 h. Water (20 mL) was added, and the reaction was extracted with EtOAc (2×30 mL). The combined organic layers were washed with brine, dried over Na₂SO₄, and filtered. The filtrate was concentrated under reduced pressure and purified by silica gel column chromatography (0-10% MeOH in DCM), followed by reverse phase chromatography (30-65% MeCN in water (0.1% formic acid)) to afford 1-[2-[(2S,6R)-2-(1-cyclopropylpyrazol-4-yl)-6-methyl-morpholin-4-yl]-4-(2,4-difluorophenyl)-7-methyl-pyrido[2,3-d]pyrimidin-6-yl]-N-methoxy-ethanimine (Example 5, 5.5 mg, 0.01 mmol, 26% yield) as a solid. LC/MS (ESI, m/z⁺): 534.4 [M+H]⁺. ¹H NMR (CHCl₃-d, 400 MHz): δ_{H} 7.62 (1H, d, J=3.7 Hz), 7.51-7.57 (3H, m), 7.06 (1H, td, J=8.2, 2.3 Hz), 6.96-7.01 (1H, m), 4.93-5.11 (1H, m), 4.56-4.59 (1H, m), 3.95 (3H, s), 3.76-3.84 (1H, m), 3.55 (1H, tt, J=7.3, 3.8 Hz), 3.05 (1H, dd, J=13.4, 10.9 Hz), 2.81 (1H, dd, J=13.4, 10.6 Hz), 2.72 (3H, s), 2.15 (3H, s), 1.30 (3H, d, J=6.2 Hz), 1.08-1.12 (2H, m), 0.96-1.03 (2H, m). ¹⁹F NMR (CHCl₃-d, 376 MHz): δ_{F}−106.2 (1F, s), −108.7 (1F, s).

### Method 4 Synthesis of Examples 6, 22, 33, and 44

### Example 6: (2S,6R)-2-(1-cyclopropylpyrazol-4-yl)-4-[4-(2,4-difluorophenyl)-7-methyl-6-(3-methyl-1,2,4-oxadiazol-5-yl)-pyrido[2,3-d]pyrimidin-2-yl]-6-methyl-morpholine

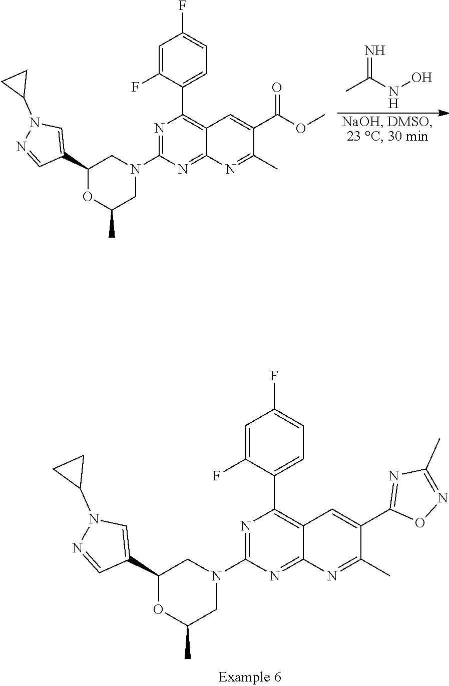

To a room temperature solution of N-hydroxyacetamidine (1 eq, 14 mg, 0.19 mmol) and sodium hydroxide (1.5 eq, 12 mg, 0.29 mmol) in DMSO (1 mL) was added methyl 2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxylate (Example 1, 1 eq, 100 mg, 0.19 mmol), and the mixture was stirred at room temperature for 30 min. The mixture was washed with brine (20 mL), extracted with EtOAc (30 mL), and the organic layer was dried over Na₂SO₄, and filtered. The filtrate was concentrated under reduced pressure and purified by reverse phase chromatography (30-70% o MeCN in water (0.1% o formic acid)), followed by prep-HPLC (Condition 2, Gradient 1) to afford (2S,6R)-2-(1-cyclopropylpyrazol-4-yl)-4-[4-(2,4-difluorophenyl)-7-methyl-6-(3-methyl-1,2,4-oxadiazol-5-yl)-pyrido[2,3-d]pyrimidin-2-yl]-6-methyl-morpholine (Example 6, 22 mg, 0.04 mmol, 21% yield), as a solid. LC/MS (ESI, m/z⁺): 545.3 [M+H].

¹H NMR (CHCl₃-d, 400 MHz): δ_{H} 8.48 (11H, d, J=3.6 Hz), 7.51-7.61 (3H, m), 6.99-7.15 (1H, m), 4.90-5.27 (1H, m), 4.53-4.62 (1H, m), 3.76-3.85 (1H, m), 3.50-3.59 (1H, m), 3.06-3.12 (4H, m), 2.83-2.89 (1H, m), 2.46 (3H, s), 1.31 (3H, s), 1.07-1.10 (2H, m), 0.96-1.01 (2H, m). ¹⁹F NMR (CHCl₃-d, 376 MHz): δ_{F}−108.6 (1F, s), −105.2 (1F, s).

An analogous method was followed to obtain the following compounds.

| Ex # | Structure | Starting Material | Characterization |
|---|---|---|---|
| 22 | 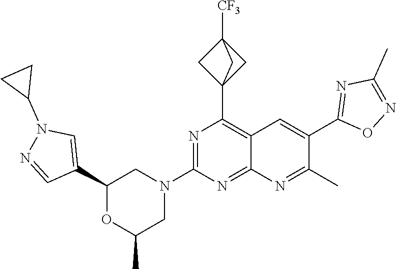 [SMILES: Cc1noc(-c2cc3c(C45CC(C(F)(F)F)(C4)C5)nc(N4C[C@@H](C)O[C@@H](c5cnn(C6CC6)c5)C4)nc3nc2C)n1] | methyl 2-[(2S,6R)-2-(1- cyclopropyl-pyrazol-4- yl)-6-methyl- morpholin-4-yl]-7- methyl-4-[3- (trifluoromethyl)-1- bicyclo[1.1.1]pentanyl]- pyrido[2,3- d]pyrimidine-6- carboxylate, and N-hydroxy-acetamidine | (2S,6R)-2-(1-cyclopropyl- pyrazol-4-yl)-6-methyl-4-[7- methyl-6-(3-methyl-1,2,4- oxadiazol-5-yl)-4-[3- (trifluoromethyl)-1- bicyclo[1.1.1]pentanyl]- pyrido[2,3-d]pyrimidin-2- yl]morpholine, Example 22. ¹H NMR (CHCl₃-d, 400 MHz): δ_{H} 8.96 (1H, s), 7.52-7.54 (2H, m), 4.92-5.23 (2H, m), 4.57 (1H, d, J = 10.6 Hz), 3.80 (1H, ddd, J = 10.6, 6.1,2.6 Hz), 3.56-3.60 (1H, m), 3.06 (4H, s), 2.83 (1H, t, J = 12.0 Hz), 2.59-2.62 (6H, m), 2.52 (3H, s), 1.32-1.34 (3H, m), 1.09-1.16 (2H, m), 1.02 |
| | | | (2H, d, J = 6.8 Hz). ¹⁹F NMR |
| | | | (CHCl₃-d, 376 MHz): δ_{F} −73.0 |
| | | | (3F, s). LC/MS (m/z): 567.3 |
| | | | [M + H]⁺. |
| 33 | 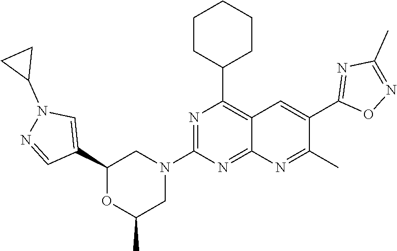 [SMILES: Cc1noc(-c2cc3c(C4CCCCC4)nc(N4C[C@@H](C)O[C@@H](c5cnn(C6CC6)c5)C4)nc3nc2C)n1] | methyl 4-cyclohexyl-2- [(2S,6R)-2-(1- cyclopropyl-pyrazol-4- yl)-6-methyl- morpholin-4-yl]-7- methyl-pyrido [2,3-d]- pyrimidine-6- carboxylate, and N-hydroxy-acetamidine | (2S,6R)-4-[4-cyclohexyl-7- methyl-6-(3-methyl-1,2,4- oxadiazol-5-yl)-pyrido[2,3- d]pyrimidin-2-yl]-2-(1- cyclopropylpyrazol-4-yl)-6- methyl-morpholine, Example 33. ¹H NMR (CHCl₃-d, 400 MHz): δ_{H} 8.84 (1H, s), 7.50- 7.55 (2H, m), 4.97-5.23 (2H, m), 4.57 (1H, d, J = 10.7 Hz), 3.76-3.81 (1H, m), 3.58 (1H, t, J = 6.1 Hz), 3.34-3.41 (1H, m), 3.03-3.09 (4H, m), 2.79-2.85 (1H, m), 2.51 (3H, s), 1.90 (5H, d, J = 12.2 Hz), 1.62-1.77 (2H, m), 1.52 (1H, s), 1.32-1.34 (4H, |
| | | | m), 1.12 (2H, d, J = 8.7 Hz), |
| | | | 1.01 (2H, d, J = 7.0 Hz). |
| | | | LC/MS (m/z): 515.4 [M + H]⁺. |
| 44 | 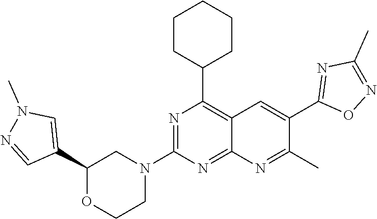 [SMILES: Cc1noc(-c2cc3c(C4CCCCC4)nc(N4CCO[C@@H](c5cnn(C)c5)C4)nc3nc2C)n1] | methyl 4-cyclohexyl-7- methyl-2-[(2S)-2-(1- methyl-pyrazol-4-yl)- morpholin-4-yl]- pyrido[2,3-d]- pyrimidine-6- carboxylate, and N-hydroxy-acetamidine | (2S)-4-[4-cyclohexyl-7- methyl-6-(3-methyl-1,2,4- oxadiazol-5-yl)-pyrido [2,3-d]- pyrimidin-2-yl]-2-(1-methyl- pyrazol-4-yl) morpholine, Example 44. ¹H NMR (CHCl₃-d, 400 MHz): δ_{H} 8.85 (1H, s), 7.55 (1H, s), 7.44 (1H, s), 4.97-5.19 (1H, m), 4.92 (1H, br s), 4.58 (1H, dd, J = 10.1, 2.7 Hz), 4.09 (1H, br s), 3.91 (3H, s), 3.78 (1H, dd, J = 12.5, 10.2 Hz), 3.40 2H, t, J = 13.0 Hz), 3.04 (3H, s), 2.52 |
| | | | (3H, s), 1.91 (4H, d, J = |
| | | | 12.1 Hz), 1.82 (1H, d, J = |
| | | | 10.9 Hz), 1.71 (1H, d, J = |
| | | | 10.8 Hz), 1.54 (2H, t, J = |
| | | | 13.8 Hz), 1.28-1.37 (2H, m). |
| | | | LC/MS (m/z): 475.4 [M + H]⁺. |

### Method 5 Synthesis of Example 7

### Synthesis of A5

### Intermediate A5: 4-(2,4-difluorophenyl)-N-(2-hydroxyethyl)-7-methyl-2-[(2S,6R)-2-(1-cyclopropylpyrazol-4-yl)-6-methyl-morpholin-4-yl]pyrido [2,3-d]pyrimidine-6-carbozamide

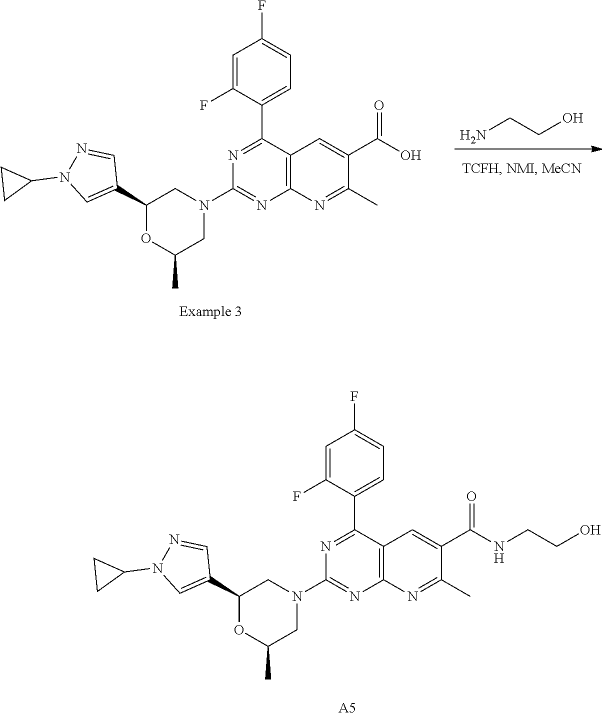

To a flame-dried microwave flask equipped with a Teflon-coated magnetic stirring bar, was added 2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carb xylic acid (Example 3, 1 eq, 50 mg, 0.1 mmol), ethanolamine (2 eq, 12 μL, 0.2 mmol), 1-methylimidazole (3.5 eq, 27 μL, 0.35 mmol) and MeCN (1.3 mL). The mixture was cooled down to 0° C., [chloro(dimethylamino)-methylene]dimethylammonium hexafluorophosphate (1.15 eq, 32 mg, 0.11 mmol) was added and the obtained solution was stirred at 23° C. for 16 h. The reaction was washed with water (20 mL) and extracted with EtOAc (2×20 mL). The combined organic

layers were washed with brine (20 mL), dried over Na₂SO₄, filtered, and concentrated under reduced pressure. The crude was purified by reverse phase flash chromatography (30-70% ACN in water (0.1% formic acid)) to afford 4-(2,4-difluorophenyl)-N-(2-hydroxyethyl)-7-methyl-2-[(2S,6R)-2-(1-cyclopropyl-pyrazol-4-yl)-6-methyl-morpholin-4-yl]pyrido[2,3-d]pyrimidine-6-carboxamide (A5, 52 mg, 96% yield) as a solid. LC/MS (ESI, m/z⁺): 550.3 [M+H]⁺.

### Example 7: (2S,6R)-2-(1-cyclopropylpyrazol-4-yl)-4-[4-(2,4-difluorophenyl)-6-(4,5-dihydrooxazol-2-yl)-7-methyl-pyrido[2,3-d]pyrimidin-2-yl]-6-methyl-morpholine

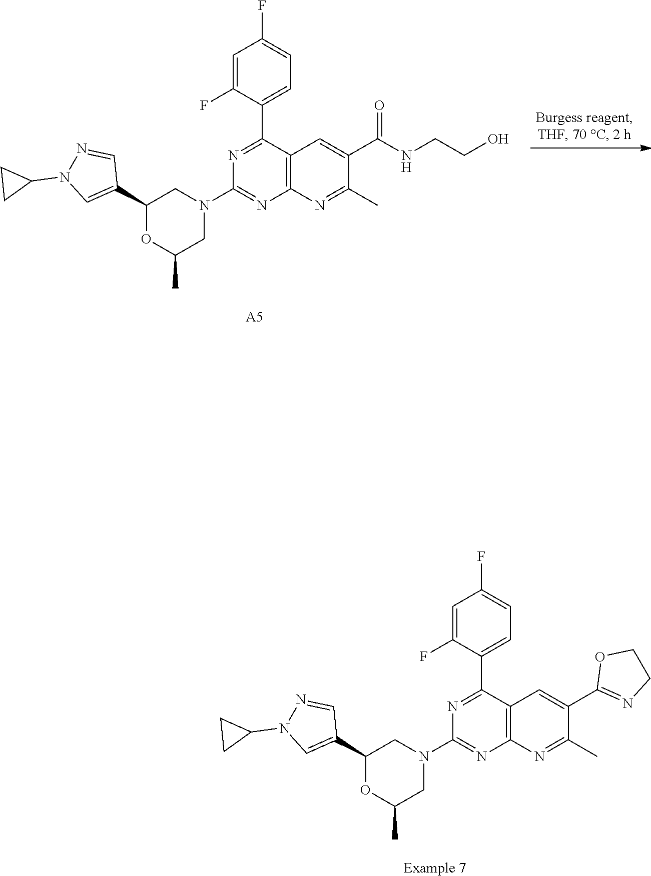

Burgess reagent (1.2 eq, 10 mg, 0.044 mmol) was added at room temperature to a solution of 2-[(2S,6R)-2-(1-cyclopropylpyrazol-4-yl)-6-methyl-morpholin-4-yl]-4-(2,4-difluorophenyl)-N-(2-hydroxyethyl)-7-methyl-pyrido[2,3-d]pyrimidine-6-carboxamide (A5, 1 eq, 20 mg, 0.036 mmol) and anhydrous THF (0.5 mL), and the mixture was stirred at 50° C. for 16h. Saturated solution of NaHCO₃ (20 mL) was added, the organic phase was extracted with EtOAc (3×20 mL), washed with brine (20 mL), dried over Na₂SO₄, and filtered. The filtrate was concentrated under reduced pressure and purified by normal phase column chromatography (50-100% EtOAc in Hexanes, followed by 0-5% MeOH in DCM), followed by reverse phase chromatography (20-50% MeCN in water (0.1% formic acid)) to afford (2S,6R)-2-(1-cyclopropylpyrazol-4-yl)-4-[4-(2,4-difluorophenyl)-6-(4,5-dihydrooxazol-2-yl)-7-methyl-pyrido[2,3-d]pyrimidin-2-yl]-6-methyl-morpholine (Example 7, 7.5 mg, 39% yield) as a solid. LC/MS (ESI, m/z⁺): 525.5 [M+H]⁺. ¹H NMR (CHCl₃-d, 400 MHz): δ_{H} 8.42 (1H, d, J=3.7 Hz), 7.71 (1H, s), 7.53-7.60 (3H, m), 7.39 (1H, t, J=5.6 Hz), 7.01-7.12 (2H, m), 6.55 (1H, s), 5.92 (1H, s), 4.94-5.26 (2H, m), 4.33-4.57 (9H, m), 3.77-3.85 (6H, m), 3.59-3.64 (13H, m), 3.06-3.14 (2H, m), 2.97 (3H, s), 2.77-2.95 (3H, m), 2.15-2.29 (2H, m), 1.73-1.79 (1H, m), 1.42-1.46 (2H, m), 1.32 (3H, d, J=5.9 Hz), 1.11 (2H, s), 1.01 (2H, d, J=7.0 Hz). ¹⁹F NMR (CHCl₃-d, 376 MHz): δ_{F}−105.6 (1F, s), −108.6 (1F, s).

### Method 6 Synthesis of Example 8

### Example 8:methyl 2-((2R,4S)-2-(1-cyclopropyl-1H-pyrazol-4-yl)tetrahydro-2H-pyran-4-yl)-4-(2,4-difluorophenyl)-7-methylpyrido[2,3-d]-pyrimidine-6-carboxylate

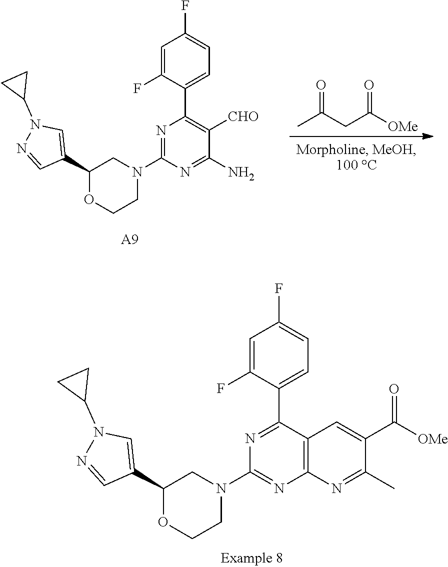

To a flame-dried microwave vial equipped with a Teflon-coated magnetic stirring bar was added methyl 3-oxobutanoate (2 eq, 0.11 mL, 1.01 mmol) and morpholine (1 eq, 0.044 mL, 0.51 mmol), and the vial was sealed and stirred at RT under Ar (balloon). After 24 h, a solution of 4-amino-2-[(2R,4S)-2-(1-cyclopropylpyrazol-4-yl)tetrahydropyran-4-yl]-6-(2,4-difluorophenyl)-pyrimidine-5-carbaldehyde (A9, 1 eq, 215 mg, 0.51 mmol) in anhydrous MeOH (0.51 mL) was added to the vial. The vial was sealed and heated to 100° C. with stirring. After 20 h, the reaction mixture was cooled to RT, and concentrated under reduced pressure. The crude mixture was purified by silica gel column chromatography (10%-40% Acetone in Hexanes) to afford a crude product. The crude product was further purified by prep-HPLC (Condition 2, Gradient 2) to afford methyl 2-((2R,4S)-2-(1-cyclopropyl-1H-pyrazol-4-yl)tetrahydro-2H-pyran-4-yl)-4-(2,4-difluorophenyl)-7-methylpyrido[2,3-d]-pyrimidine-6-carboxylate (Example 8, 57 mg, 0.14 mmol, 28% yield) as a solid. ¹H-NMR (CHCl₃-d, 400 MHz): δ_{H} 8.63 (d, J=4.06 Hz, 1H), 7.64-7.71 (m, 1H), 7.48 (d, J=3.54 Hz, 2H), 7.12-7.17 (m, 1H), 7.03-7.08 (m, 1H), 4.52-4.56 (m, 1H), 4.23-4.27 (m, 1H), 3.94 (s, 3H), 3.75-3.82 (m, 1H), 3.49-3.56 (m, 2H), 3.08 (s, 3H), 2.39-2.44 (m, 1H), 2.15-2.25 (m, 3H), 1.06-1.10 (m, 2H), 0.94-0.99 (m, 2H). LC/MS: Tr=1.50 min; [M+H]⁺ 506.4.

### Method 7 Synthesis of Example 9

### Example 9:2-((2R,4S)-2-(1-cyclopropyl-1H-pyrazol-4-yl)tetrahydro-2H-pyran-4-yl)-4-(2,4-difluorophenyl)-7-methylpyrido-[2,3-d]pyrimidine-6-carboxylic acid

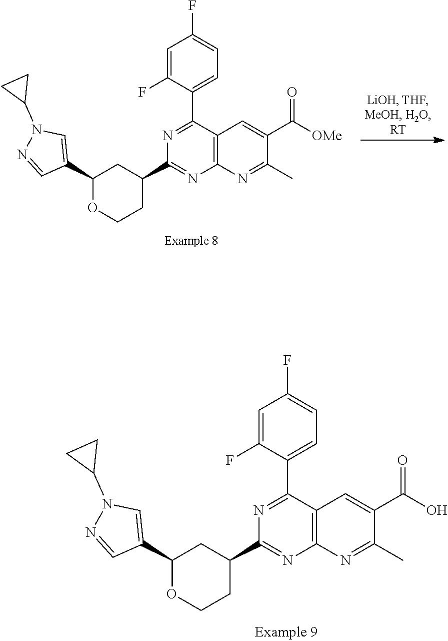 [SMILES: COC(=O)c1cc2c(-c3ccc(F)cc3F)nc([C@H]3CCO[C@@H](c4cnn(C5CC5)c4)C3)nc2nc1C ; Cc1nc2nc([C@H]3CCO[C@@H](c4cnn(C5CC5)c4)C3)nc(-c3ccc(F)cc3F)c2cc1C(=O)O]

To a round-bottomed flask equipped with a Teflon-coated magnetic stirring bar was added 2-((2R,4S)-2-(1-cyclopropyl-1H-pyrazol-4-yl)tetrahydro-2H-pyran-4-yl)-4-(2,4-difluorophenyl)-7-methylpyrido[2,3-d]pyrimidine-6-carboxylate-1 (Example 8, 1 eq, 429 mg, 849 μmol), MeOH (7.2 mL) and THF (14.4 mL). The resulting solution was stirred at RT. A solution of Lithium hydroxide (3 eq, 61 mg, 2.55 mmol) in water (3.6 mL) was added. The reaction mixture was stirred at RT for 15 h. The reaction mixture was concentrated under reduced pressure and the crude residue was diluted with DCM. The pH was adjusted to 6 by adding a solution of 10% of AcOH in water. The reaction was extracted with DCM (×3). The combined organic extracts were dried over Na₂SO₄, filtered, and concentrated under reduced pressure to afford 2-((2R,4S)-2-(1-cyclopropyl-1H-pyrazol-4-yl)tetrahydro-2H-pyran-4-yl)-4-(2,4-difluorophenyl)-7-methylpyrido[2,3-d]pyrimidine-6-carboxylic acid (Example 9, 386 mg, 790 μmol, 93% yield) as a solid. ¹H-NMR (CHCl₃-d, 400 MHz): δ_{H} 8.63 (d, J=4.06 Hz, 1H), 7.64-7.71 (m, 1H), 7.48 (d, J=3.54 Hz, 2H), 7.12-7.17 (m, 1H), 7.03-7.08 (m, 1H), 4.52-4.56 (m, 1H), 4.23-4.27 (m, 1H), 3.94 (s, 3H), 3.75-3.82 (m, 1H), 3.49-3.56 (m, 2H), 3.08 (s, 3H), 2.39-2.44 (m, 1H), 2.15-2.25 (m, 3H), 1.06-1.10 (m, 2H), 0.94-0.99 (m, 2H). LC/MS: Tr=1.36 min; [M+H]⁺ 492.3.

### Method 8 Synthesis of Example 19

### Example 19

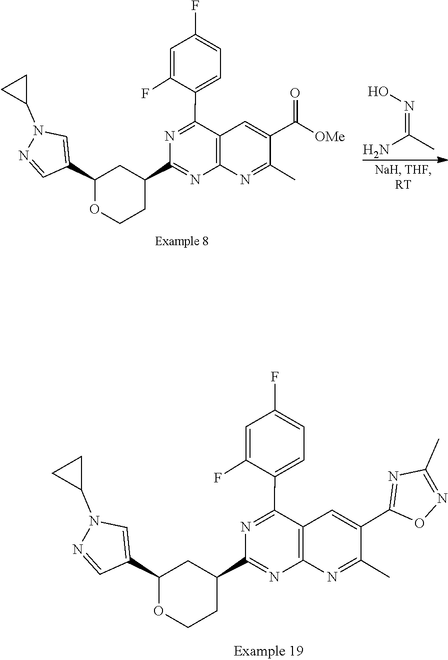

To a flame-dried microwave vial equipped with a Teflon-coated magnetic stirring bar was added methyl 4-(2,4-difluorophenyl)-7-methyl-2-[rac-(2R,4S)-2-(1-cyclopropylpyrazol-4-yl)-tetrahydropyran-4-yl]pyrido[2,3-d]pyrimidine-6-carboxylate (Example 8, 1 eq, 35 mg, 0.07 mmol), anhydrous THF (1 mL) and N-hydroxyacetamidine (1.2 eq, 6.2 mg, 0.084 mmol), and the resulting solution was stirred for 1 h at RT under Ar. Sodium hydride (1.2 eq, 3.4 mg, 0.084 mmol) was added and the reaction mixture was stirred at RT. After 30 min, the reaction mixture was cooled to 0° C. in an ice bath and quenched dropwise with sat. NH₄Cl (aq) (1 mL). The reaction mixture was diluted with EtOAc (25 mL) and washed with sat. NH₄Cl (aq) and brine. The organic layer was collected, dried over Na₂SO₄, and filtered. The filtrate was concentrated under reduced pressure and purified by silica gel column chromatography (30%-100% EtOAc in DCM) to afford 5-[4-(2,4-difluorophenyl)-7-methyl-2-[rac-(2R,4S)-2-(1-cyclopropylpyrazol-4-yl)-tetrahydropyran-4-yl]pyrido[2,3-d]pyrimidin-6-yl]-3-methyl-1,2,4-oxadiazole (Example 19, 20 mg, 0.036 mmol, 52% yield) as a solid. The product was isolated as a 13:1 mixture of cis:trans diastereomers by ¹H NMR. (cis diastereomer reported). ¹H NMR (CHCl₃-d, 400 MHz): δ_{H} 8.80 (1H, d, J=4.1 Hz), 7.71 (1H, td, J=8.3, 6.2 Hz), 7.50 (1H, s), 7.49 (1H, s), 7.17 (1H, td, J=8.2, 2.3 Hz), 7.06-7.11 (1H, m), 4.56 (1H, d, J=11.3 Hz), 4.25-4.29 (1H, m), 3.77-3.84 (1H, m), 3.53-3.60 (2H, m), 3.21 (3H, s), 2.52 (3H, s), 2.44 (1H, d, J=13.3 Hz), 2.17-2.27 (3H, m), 1.07-1.10 (2H, m), 0.95-1.00 (2H, m). ¹⁹F NMR (CHCl₃-d, 376 MHz): δ_{F}−104.4-104.5 (1F, m), −108.7-108.8 (1F, m). LC/MS: Tr=1.59 min; [M+H]⁺ 530.3.

### Method 9 Synthesis of Example 10

### Example 10: (2S,6R)-2-(1-cyclopropylpyrazol-4-yl)-4-[4-(2,4-difluorophenyl)-7-methyl-6-(5-methyl-1,3,4-thiadiazol-2-yl)pyrido[2,3-d]pyrimidin-2-yl]-6-methyl-morpholine

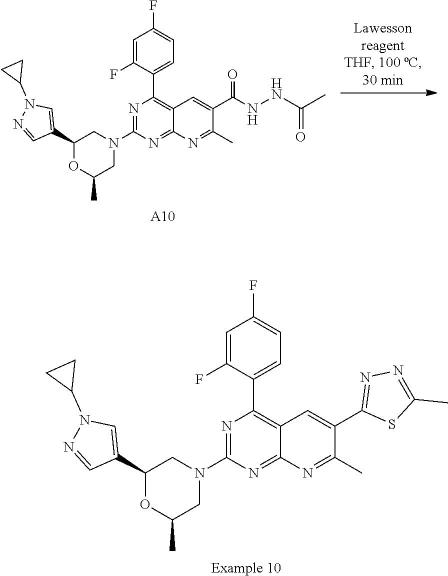 [SMILES: CC(=O)NNC(=O)c1cc2c(-c3ccc(F)cc3F)nc(N3C[C@@H](C)O[C@@H](c4cnn(C5CC5)c4)C3)nc2nc1C ; "A10" ; Cc1nnc(-c2cc3c(-c4ccc(F)cc4F)nc(N4C[C@@H](C)O[C@@H](c5cnn(C6CC6)c5)C4)nc3nc2C)s1 ; "Example 10"]

Lawesson reagent (1.5 eq, 16 mg, 0.067 mmol) was added to a solution of N-acetyl-2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carbohydrazide (A10, 1 eq, 25 mg, 0.044 mmol) in THF (1 mL), and the mixture was stirred at 100° C. for 30 min. The reaction was cooled to rt, brine (30 mL) was added, and the organic phase was extracted with EtOAc (2×20 mL). The combined organic layers were dried over Na₂SO₄, filtered, concentrated under reduced pressure, and purified by reverse phase chromatography (40-75% MeCN in water (0.1% formic acid)) to afford (2S,6R)-2-(1-cyclopropylpyrazol-4-yl)-4-[4-(2,4-difluorophenyl)-7-methyl-6-(5-methyl-1,3,4-thiadiazol-2-yl)pyrido[2,3-d]pyrimidin-2-yl]-6-methyl-morpholine (Example 10, 12 mg, 0.021 mmol, 46% yield) as a solid. LC/MS (ESI, m/z⁺): 561.2 [M+H]⁺.

¹H NMR (CHCl₃-d, 400 MHz): δ_{H} 8.12 (1H, s), 7.53-7.58 (3H, m), 7.09 (1H, s), 7.01 (1H, s), 4.92-5.25 (2H, m), 4.61 (1H, s), 3.83 (1H, br s), 3.57 (1H, s), 3.10 (1H, t, J=12.2 Hz), 2.93 (3H, s), 2.84-2.89 (4H, m), 1.33 (3H, s), 1.11 (2H, s), 1.01 (2H, s). ¹⁹F NMR (CHCl₃-d, 376 MHz): δ_{F}−105.6 (1F, s), −108.6 (1F, s).

### Method 10 Synthesis of Example 12

### Example 12: (2S,6R)-2-(1-cyclopropylpyrazol-4-yl)-4-[4-(2,4-difluorophenyl)-7-methyl-6-(5-methyl-1,3,4-oxadiazol-2-yl)pyrido[2,3-d]pyrimidin-2-yl]-6-methyl-morpholine

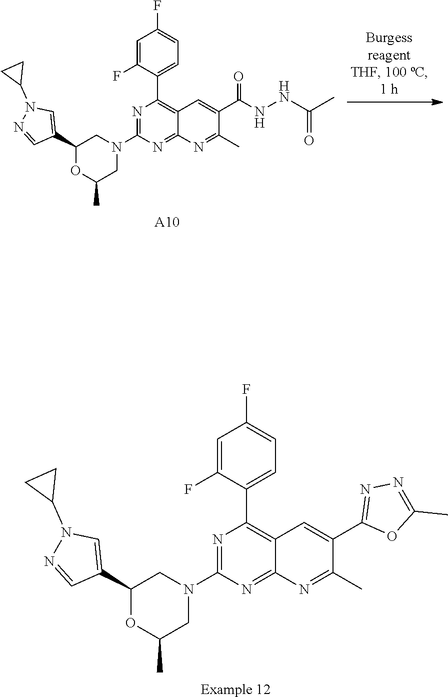 [SMILES: CC(=O)NNC(=O)c1cc2c(-c3ccc(F)cc3F)nc(N3C[C@@H](C)O[C@@H](c4cnn(C5CC5)c4)C3)nc2nc1C ; "A10" ; Cc1nnc(-c2cc3c(-c4ccc(F)cc4F)nc(N4C[C@@H](C)O[C@@H](c5cnn(C6CC6)c5)C4)nc3nc2C)o1 ; "Example 12"]

To a solution of N′-acetyl-2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carbohydrazide (1 eq, 25 mg, 0.044 mmol) in THF (1 mL) was added Burgess reagent (1.1 eq, 12 mg, 0.049 mmol). The mixture was stirred at 100° C. for 1 h. Brine (30 mL) was added and the organic phase was extracted with EtOAc (2×25 mL). The combined organic layers were dried over Na₂SO₄, filtered, and concentrated under reduced pressure. The crude was purified by reverse phase column chromatography (40-75% MeCN in water (0.1% formic acid)) to afford (2S,6R)-2-(1-cyclopropylpyrazol-4-yl)-4-[4-(2,4-difluorophenyl)-7-methyl-6-(5-methyl-1,3,4-oxadiazol-2-yl)pyrido[2,3-d]pyrimidin-2-yl]-6-methyl-morpholine (Example 12, 11 mg, 0.021 mmol, 46% yield) as a solid. LC/MS (ESI, m/z⁺): 545.3 [M+H]⁺. ¹H NMR (CHCl₃-d, 400 MHz): δ_{H} 8.30-8.31 (1H, m), 7.50-7.60 (3H, m), 7.00-7.13 (2H, m), 4.92-5.27 (1H, m), 4.54-4.62 (1H, m), 3.77-3.84 (1H, m), 3.52-3.60 (1H, m), 3.06-3.12 (4H, m), 2.82-2.89 (1H, m), 1.31-1.33 (4H, m), 1.08-1.12 (1H, m), 0.97-1.03 (2H, m). ¹⁹F NMR (CHCl₃-d, 376 MHz): δ_{F}−105.4 (1F, s), −108.6 (1F, s).

### Method 11 Synthesis of Example 11

### Example 11: (2S,6R)-2-(1-cyclopropylpyrazol-4-yl)-4-[4-(2,4-difluorophenyl)-7-methyl-6-(3-methylisoxazol-5-yl)pyrido[2,3-d]pyrimidin-2-yl]-6-methyl-morpholine

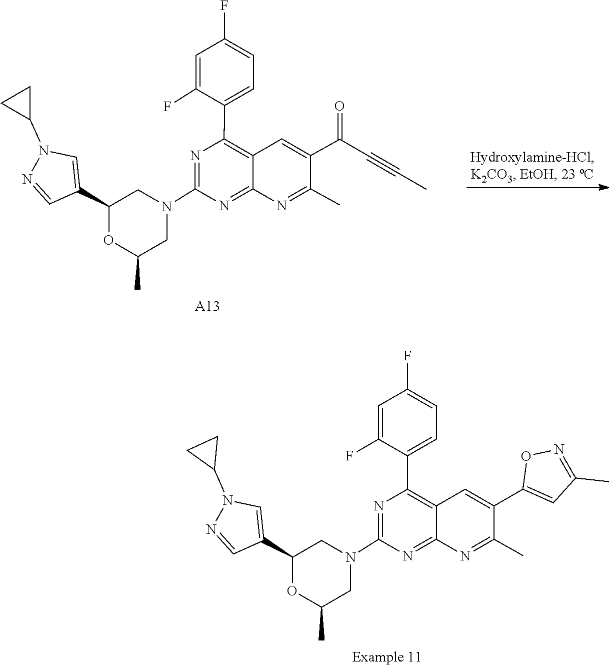

In a flame dried vial was added 1-[2-[(2S,6R)-2-(1-cyclopropylpyrazol-4-yl)-6-methyl-morpholin-4-yl]-4-(2,4-difluorophenyl)-7-methyl-pyrido[2,3-d]pyrimidin-6-yl]but-2-yn-1-one (A13, 1 eq, 15 mg, 0.028 mmol), sodium azide (5 eq, 9.2 mg, 0.14 mmol), acetic acid (5 eq, 8.1 uL, 0.14 mmol), and triethylamine (2 eq, 7.9 uL, 0.057 mmol) in THF (0.6 mL) under nitrogen atmosphere. The resulting mixture was stirred at room temperature for 36 h and water (20 mL) was added to the reaction. The organic phase was extracted with EtOAc (2×20 mL) and the combined organic layers were washed with brine, dried over Na₂SO₄, filtered and concentrated under reduced pressure. The crude was purified by reverse phase chromatography (30-75% MeCN in water (0.1% ammonium formate)) to afford (2S,6R)-2-(1-cyclopropylpyrazol-4-yl)-4-[4-(2,4-difluorophenyl)-7-methyl-6-(3-methylisoxazol-5-yl)pyrido[2,3-d]pyrimidin-2-yl]-6-methyl-morpholine (Example 11, 11 mg, 0.019 mmol, 68% yield) as a solid. LC/MS (ESI, m/z⁺): 544.2 [M+H]⁺. ¹H NMR (CHCl₃-d, 400 MHz): δ_{H} 8.14 (1H, d, J=3.6 Hz), 7.52-7.60 (3H, m), 7.07-7.13 (1H, m), 6.99-7.05 (1H, m), 6.32 (1H, s), 4.89-5.21 (1H, m), 4.56-4.62 (1H, m), 3.79-3.87 (1H, m), 3.53-3.60 (1H, m), 3.09 (1H, dd, J=13.4, 11.0 Hz), 2.87 (3H, t, J=0.7 Hz), 2.38 (3H, s), 1.31-1.36 (3H, m), 1.10-1.13 (2H, m), 0.96-1.03 (2H, m). ¹⁹F NMR (CHCl₃-d, 376 MHz): δ_{F}−105.7 (1F, m), −108.7 (1F, m).

### Method 12 Synthesis of Example 13

### Example 13: (2S,6R)-2-(1-cyclopropylpyrazol-4-yl)-4-[4-(2,4-difluorophenyl)-7-methyl-6-(5-methyloxazol-2-yl)pyrido[2,3-d]pyrimidin-2-yl]-6-methyl-morpholine

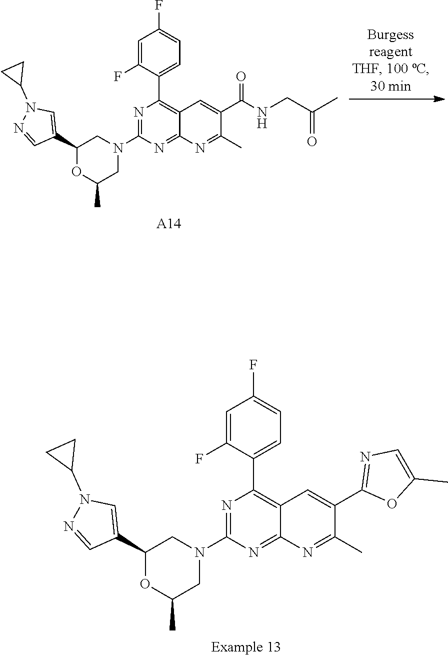 [SMILES: CC(=O)CNC(=O)c1cc2c(-c3ccc(F)cc3F)nc(N3C[C@@H](C)O[C@@H](c4cnn(C5CC5)c4)C3)nc2nc1C ; "A14" ; Cc1cnc(-c2cc3c(-c4ccc(F)cc4F)nc(N4C[C@@H](C)O[C@@H](c5cnn(C6CC6)c5)C4)nc3nc2C)o1 ; "Example 13"]

To a solution of N-acetonyl-2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxamide (A14, 1 eq, 10 mg, 0.018 mmol) in THF (1 mL) was added Burgess reagent (2 eq, 8.5 mg, 0.036 mmol), and the reaction was stirred at 100° C. for 1 h. The resulting mixture was cooled to room temperature, brine (30 mL) was added, and the organic phase was extracted with EtOAc (2×25 mL). The combined organic layers were dried over Na₂SO₄, and filtered. The filtrate was concentrated under reduced pressure and purified by reverse phase column chromatography (40-75% MeCN in water (0.1% formic acid)) to afford (2S,6R)-2-(1-cyclopropylpyrazol-4-yl)-4-[4-(2,4-difluorophenyl)-7-methyl-6-(5-methyloxazol-2-yl)pyrido[2,3-d]pyrimidin-2-yl]-6-methyl-morpholine (Example 13, 12 mg, 0.021 mmol, 46% yield) as a solid. LC/MS (ESI, m/z⁺): 544.3 [M+H]⁺. ¹H NMR (CHCl₃-d, 400 MHz): δ_{H} 8.31 (1H, d, J=3.6 Hz), 7.54-7.61 (1H, m), 7.50-7.53 (2H, m), 7.06-7.11 (1H, m), 7.00-7.05 (1H, m), 6.85-6.86 (1H, m), 4.99-5.22 (1H, m), 4.55-4.61 (1H, m), 3.78-3.84 (2H, m), 3.52-3.60 (1H, m), 3.04-3.10 (4H, m), 2.81-2.87 (1H, m), 2.38 (3H, d, J=1.2 Hz), 1.30-1.32 (3H, m), 1.07-1.13 (2H, m), 0.96-1.03 (2H, m). ¹⁹F NMR (CHCl₃-d, 376 MHz): δ_{F}−106.0 (1F, s), −108.7 (1F, s).

### Method 13 Synthesis of Example 15

### Example 15: 4-[1-[4-(2,4-difluorophenyl)-7-methyl-6-(3-methyl-1,2,4-oxadiazol-5-yl)pyrido[2,3-d]pyrimidin-2-yl]-3-piperidyl]morpholine

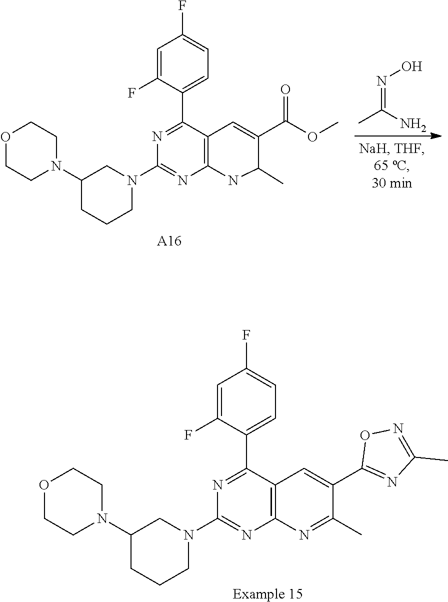

To a solution of methyl 4-(2,4-difluorophenyl)-7-methyl-2-(3-morpholino-1-piperidyl)pyrido[2,3-d]pyrimidine-6-carboxylate (1 eq, 90 mg, 0.19 mmol) and N′-hydroxyacetamidine (1.2 eq, 17 mg, 0.22 mmol) in THF was added sodium hydride (1.2 eq, 8.9 mg, 0.22 mmol). The reaction was stirred at 65° C. for 30 min. The reaction mixture was cooled down, quenched with water and diluted with EtOAc. The organic phase was washed with brine, dried over anhydrous Na₂SO₄, and filtered. The filtrate was concentrated under reduced pressure and purified by silica gel column chromatography (0-10% MeOH in DCM), followed by prep-HPLC (Condition 2, Gradient 4) to afford 4-[1-[4-(2,4-difluorophenyl)-7-methyl-6-(3-methyl-1,2,4-oxadiazol-5-yl)pyrido[2,3-d]pyrimidin-2-yl]-3-piperidyl]morpholine (Example 15, 17 mg, 0.03 mmol, 18% yield) as a solid. ¹H NMR (CHCl₃-d, 400 MHz): δ_{H} 8.45 (1H, d, J 3.5 Hz), 7.52-7.59 (1H, m), 7.00-7.14 (2H, m), 4.86-4.92 (1H, m), 3.67-3.74 (5H, m), 3.17-3.25 (1H, m), 3.05 (4H, s), 2.54-2.74 (4H, m), 2.46 (4H, s), 2.29-2.38 (1H, m), 2.02-2.07 (1H, m), 1.88-1.93 (1H, m), 1.58-1.64 (1H, m). ¹⁹F NMR (CHCl₃-d, 376 MHz): δ_{F}−105.5, −108.6, −108.9. LC/MS (ESI, m/z⁺): 508.3 [M+1]⁺.

### Method 14 Synthesis of Example 29

### Example 29

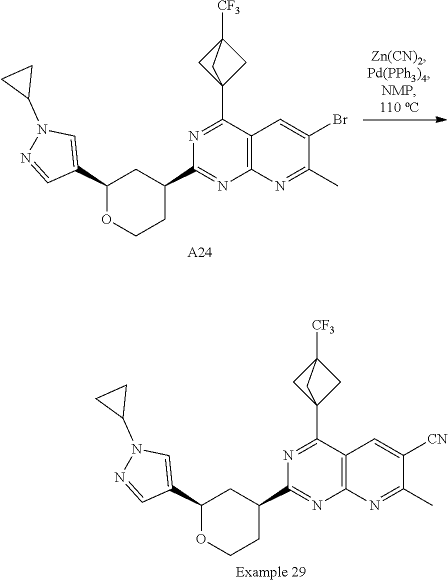

To a flame-dried microwave vial equipped with a Teflon-coated magnetic stirring bar was added Zn(CN)₂ (2 eq, 47 mg, 0.40 mmol), Pd(PPh₃)₄(0.1 eq, 23 mg, 0.02 mmol), 6-bromo-2-[(2R,4S)-2-(1-cyclopropylpyrazol-4-yl)tetrahydropyran-4-yl]-7-methyl-4-[3-(trifluoromethyl)-1-bicyclo[1.1.1]pentanyl]pyrido[2,3-d]pyrimidine (A24, 1 eq, 110 mg, 0.20 mmol) and anhydrous NMP (2 mL). The vial was sealed, degassed under Ar, and the reaction mixture was stirred at 110° C. After 6 h, the reaction mixture was cooled to RT. Full conversion was observed by LC/MS. The reaction mixture was diluted with EtOAc (50 mL) and washed with sat. NH₄Cl (aq) and brine. The organic layer was dried over Na₂SO₄, filtered, and concentrated under reduced pressure. The crude reaction mixture was purified by silica gel column chromatography (10-40% acetone in hexanes) to afford 100 mg of crude product. The product was further purified by prep-HPLC (Condition 2, Gradient 3) and lyophilization. The product was further purified by silica gel column chromatography (0-10% MeOH in DCM) to afford 2-[(2R,4S)-2-(1-cyclopropylpyrazol-4-yl)tetrahydropyran-4-yl]-7-methyl-4-[3-(trifluoromethyl)-1-bicyclo[1.1.1]pentanyl]pyrido[2,3-d]pyrimidine-6-carbonitrile (Example 29, 47 mg, 0.094 mmol, 47% yield) as a solid. ¹H NMR (CHCl₃-d, 400 MHz): δ_{H} 8.82 (1H, s), 7.49 (1H, s), 7.48 (1H, s), 4.54 (1H, dd, J=11.4, 2.1 Hz), 4.25 (1H, dt, J=11.2, 3.2 Hz), 3.76-3.83 (1H, m), 3.55 (1H, tt, J=7.2, 3.8 Hz), 3.42-3.50 (1H, m), 3.02 (3H, s), 2.66 (6H, s), 2.37 (1H, d, J=13.8 Hz), 2.08-2.16 (3H, m), 1.07-1.11 (2H, m), 0.97-1.01 (2H, m). ¹⁹F NMR (CHCl₃-d, 376 MHz): δ_{F}−73.1 (3F, s). LC/MS: Tr=4.47 min; [M+H]⁺ 495.4.

### Method 15 Synthesis of Example 16

### Example 16:2-[2-[(2R,4S)-2-(1-cyclopropylpyrazol-4-yl)tetrahydropyran-4-yl]-7-methyl-4-[3-(trifluoromethyl)-1-bicyclo[1.1.1]pentanyl]pyrido[2,3-d]pyrimidin-6-yl]-4-methyl-oxazole

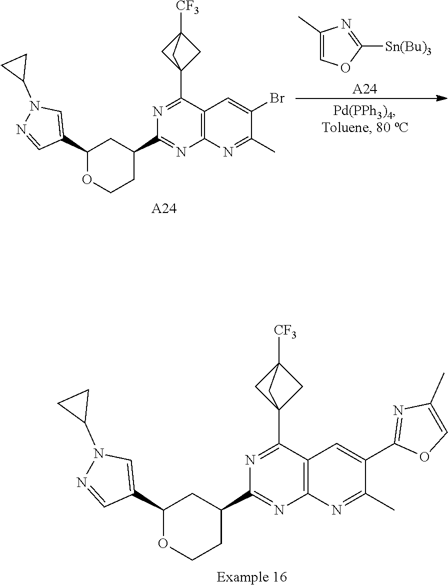

To a flame-dried round-bottom flask equipped with a Teflon-coated magnetic stirring bar was added 6-bromo-2-[(2R,4S)-2-(1-cyclopropylpyrazol-4-yl)tetrahydropyran-4-yl]-7-methyl-4-[3-(trifluoromethyl)-1-bicyclo[1.1.1]pentanyl]pyrido[2,3-d]pyrimidine (A24, 1 eq, 41 mg, 0.075 mmol), tributyl-(4-methyloxazol-2-yl)stannane (A25, 2 eq, 56 mg, 0.15 mmol) and Pd(Ph₃)₄(0.1 eq, 8.7 mg, 0.008 mmol). The vial was sealed, purged under Ar, and anhydrous Toluene (1 mL) was added. The resulting solution was degassed for 5 min under Ar and heated to 80° C. with stirring. After 6 h, additional Pd(PPh₃)₄(0.1 eq, 8.7 mg, 0.008 mmol) and tributyl-(4-methyloxazol-2-yl)stannane (A25, 2 eq, 56 mg, 0.15 mmol) were added. The reaction mixture was heated to 95° C. with stirring overnight. After 18 h, the reaction mixture was cooled to RT and diluted with EtOAc (50 mL). The resulting solution was washed with sat. NH₄Cl (aq) and brine. The organic layer was collected, dried over Na₂SO₄, filtered, and concentrated under reduced pressure. The crude mixture was purified by silica gel column chromatography (30-100% EtOAc in DCM) to afford 2-[2-[(2R,4S)-2-(1-cyclopropylpyrazol-4-yl)-tetrahydropyran-4-yl]-7-methyl-4-[3-(trifluoromethyl)-1-bicyclo[1.1.1]pentanyl]pyrido[2,3-d]pyrimidin-6-yl]-4-methyl-oxazole (Example 16, 34 mg, 0.061 mmol, 81% yield) as a solid. ¹H NMR (CHCl₃-d, 400 MHz): δ_{H} 9.06 (1H, s), 7.59 (1H, d, J=1.4 Hz), 7.50 (1H, s), 7.49 (1H, s), 4.55 (1H, dd, J=11.4, 2.1 Hz), 4.23-4.27 (1H, m), 3.80 (1H, td, J=11.3, 3.5 Hz), 3.53-3.59 (1H, m), 3.40-3.48 (1H, m), 3.18 (3H, s), 2.68 (6H, s), 2.35-2.40 (1H, m), 2.32 (3H, d, J=1.2 Hz), 2.12-2.20 (3H, m), 1.08-1.12 (2H, m), 0.95-1.00 (2H, m). ¹⁹F NMR (CHCl₃-d, 376 MHz): δ_{F}−73.0 (3F, s). LC/MS: Tr=1.77 min; [M+H]⁺ 551.3.

### Method 16 Synthesis of Examples 26-28

### Example 26: 2-((2R,4S)-2-(1-cyclopropyl-1H-pyrazol-4-yl)tetrahydro-2H-pyran-4-yl)-7-methyl-6-(1-methyl-1H-pyrazol-4-yl)-4-(3-(trifluoromethyl)bicyclo[1.1.1]pentan-1-yl)pyrido[2,3-d]pyrimidine

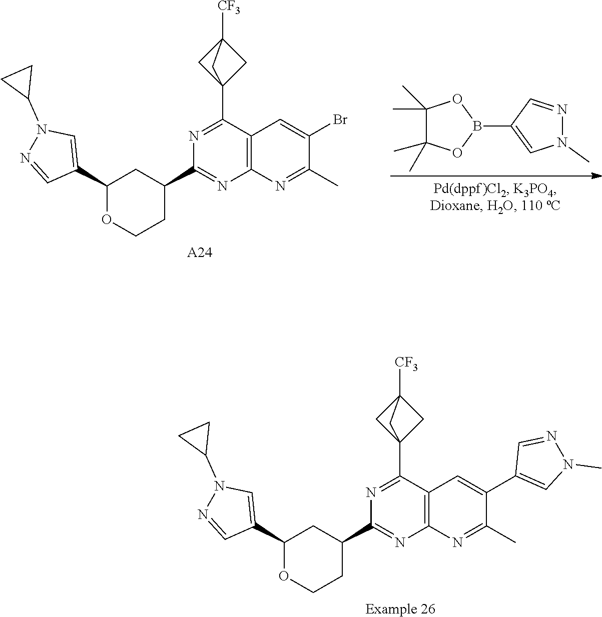

To a microwave vial equipped with a Teflon-coated magnetic stirring bar was added 6-bromo-2-[(2R,4S)-2-(1-cyclopropylpyrazol-4-yl)tetrahydropyran-4-yl]-7-methyl-4-[3-(trifluoromethyl)-1-bicyclo[1.1.1]pentanyl]pyrido[2,3-d]pyrimidine (A24, 1 eq, 50 mg, 0.091 mmol), 1-methyl-4-(4,4,5,5-tetramethyl-1,3,2-dioxaborolan-2-yl)-1H-pyrazole (2 eq, 38 mg, 0.18 mmol), K₃Ph₄ (3 eq, 58 mg, 0.27 mmol), Pd(dppf)Cl₂DCM (0.05 eq, 3.7 mg, 0.005 mmol), 1,4-dioxane (2 mL) and H₂ (1 mL). The vial was sealed, and the resulting solution was degassed under Ar. The reaction mixture was heated to 110° C. with stirring. After 30 min, the reaction mixture was cooled to RT and diluted with EtOAc and brine. The layers were separated, and the organic layer was washed with brine, collected, dried over Na₂SO₄, filtered, and concentrated under reduced pressure. The crude mixture was purified by silica gel column chromatography (50-100% o EtOAc in DCM, followed by 0-10% MeOH in DCM) to afford 2-((2R,4S)-2-(1-cyclopropyl-NH-pyrazol-4-yl)tetrahydro-2H-pyran-4-yl)-7-methyl-6-(1-methyl-1H-pyrazol-4-yl)-4-(3-(trifluoromethyl)bicyclo[1.1.1]pentan-1-yl)pyrido[2,3-d]pyrimidine (Example 26, 46 mg, 0.08 mmol, 91% P yield) as a solid. ¹H NMR (CHCl₃-d, 400 MHz): δ_{H} 8.32 (H, s), 7.68 ((H, s), 7.58 (0H, s), 7.48 (2H, d, J=2.7 Hz), 4.53 (1H, dd, J=11.4, 2.0 Hz), 4.22-4.26 (1H, m), 4.03 (3H, s), 3.75-3.82 (H, m), 3.52-3.56 (1H, m), 3.38-3.44 (1H, n), 2.85 (3H, s), 2.61 (6H, s), 2.35-2.39 (H, s), 2.09-2.19 (3H, m), 1.06-1.09 (2H, m), 0.94-0.99 (2H, m). ¹⁹F NMR (CHCl₃-d, 376 MHz): δ_{F}−73.1 (3F, s). LC/MS: [M+H]⁺ 550.4.

An analogous method was followed to obtain the following compounds.

| Ex # | Structure | Starting Material | Characterization |
|---|---|---|---|
| 27 | 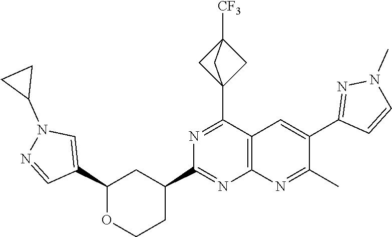 [SMILES: Cc1nc2nc([C@H]3CCO[C@@H](c4cnn(C5CC5)c4)C3)nc(C34CC(C(F)(F)F)(C3)C4)c2cc1-c1ccn(C)n1] | 1-methyl-3-(4,4,5,5- tetramethyl-1,3,2- dioxaborolan-2-yl)- 1H-pyrazole | 2-((2R,4S)-2-(1-cyclopropyl-1H- pyrazol-4-yl)tetrahydro-2H-pyran- 4-yl)-7-methyl-6-(1-methyl-1H- pyrazol-3-yl)-4-(3- (trifluoromethyl)- bicyclo[1.1.1]pentan-1- yl)pyrido[2,3-d]pyrimidine, Example 27. ¹H NMR (CHCl₃-d, 400 MHz): δ_{H} 8.63 (1H, s), 7.50 (1H, d, J = 2.3 Hz), 7.49 (2H, s), 6.52 (1H, d, J = 2.3 Hz), 4.53 (1H, dd, J = 11.4, 2.0 Hz), 4.22-4.26 (1H, m), 4.03 (3H, s), 3.75-3.82 (1H, m), 3.54 (1H, tt, J = 7.2, 3.8 Hz), 3.38-3.44 (1H, m), 2.93 (3H, |
| | | | s), 2.63 (6H, s), 2.35-2.40 (1H, m), |
| | | | 2.10-2.19 (3H, m), 1.06-1.10 (2H, |
| | | | m), 0.94-0.99 (2H, m). ¹⁹F NMR |
| | | | (CHCl₃-d, 376 MHz): δ_{F} −73.1 (3F, |
| | | | s). LC/MS (m/z): 550.3 [M + H]⁺. |
| 28 | 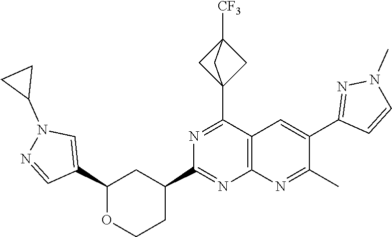 [SMILES: Cc1nc2nc([C@H]3CCO[C@@H](c4cnn(C5CC5)c4)C3)nc(C34CC(C(F)(F)F)(C3)C4)c2cc1-c1ccn(C)n1] | 1-methyl-4-(4,4,5,5- tetramethyl-1,3,2- dioxaborolan-2-yl)- 1H-1,2,3-triazole | 2-((2R,4S)-2-(1-cyclopropyl-1H- pyrazol-4-yl)tetrahydro-2H-pyran- 4-yl)-7-methyl-6-(1-methyl-1H- 1,2,3-triazol-4-yl)-4-(3- (trifluoromethyl)- bicyclo[1.1.1]pentan-1- yl)pyrido[2,3-d]pyrimidine, Example 28. ¹H NMR (CHCl₃-d, 400 MHz): δ_{H} 8.36 (1H, s), 7.80 (1H, s), 7.48 (2H, d, J = 4.5 Hz), 4.55 (1H, dd, J = 11.4, 2.1 Hz), 4.23-4.27 (1H, m), 3.93 (3H, s), 3.76-3.83 (1H, m), 3.55 (1H, tt, J = 7.2, 3.8 Hz), 3.43-3.49 (1H, m), 2.62 (3H, s), 2.61 (6H, s), 2.35-2.40 |
| | | | (1H, m), 2.10-2.16 (3H, m), 1.07- |
| | | | 1.11 (2H, m), 0.95-1.00 (2H, m). |
| | | | ¹⁹F NMR (CHCl₃-d, 376 MHz): δ_{F} |
| | | | −73.1 (3F, s). LC/MS (m/z): 551.4 |
| | | | [M + H]⁺. |

### Method 17 Synthesis of Example 23

### Example 23: 2-(2-((2R,4S)-2-(1-cyclopropyl-1H-pyrazol-4-yl)tetrahydro-2H-pyran-4-yl)-7-methyl-4-(3-(trifluoromethyl)bicyclo[1.1.1]pentan-1-yl)pyrido[2,3-d]pyrimidin-6-yl)-5-methyl-1,3,4-oxadiazole

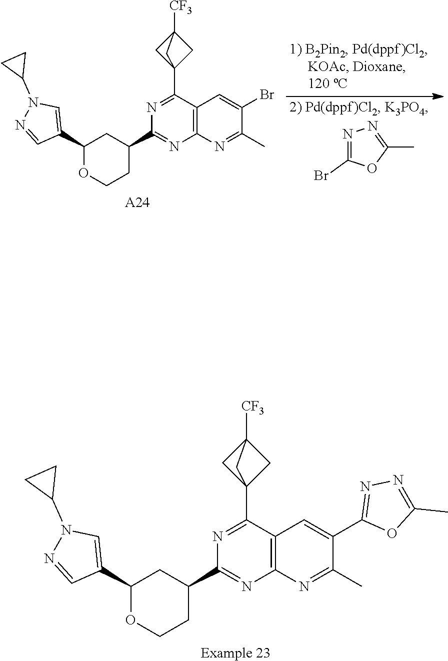

To a microwave vial equipped with a Teflon-coated magnetic stirring bar was charged 6-bromo-2-[(2R,4S)-2-(1-cyclopropylpyrazol-4-yl)tetrahydropyran-4-yl]-7-methyl-4-[3-(trifluoromethyl)-1-bicyclo[1.1.1]pentanyl]pyrido[2,3-d]pyrimidine (A24, 1 eq, 32 mg, 0.058 mmol), B₂pin₂ (1.2 eq, 18 mg, 0.07 mmol), KOAc (2 eq, 17 mg, 0.18 mmol), Pd(dppf)Cl₂ (0.05 eq, 2.1 mg, 0.003 mmol) and anhydrous 1,4-dioxane (1 mL). The vial was sealed and degassed under Ar, and the mixture was irradiated at 120° C. in a microwave reactor for 1 h with stirring. The reaction mixture was cooled to RT, and to it, was added 2-bromo-5-methyl-1,3,4-oxadiazole (1 eq, 9.5 mg, 0.058 mmol), K₃PO₄ (2 eq, 25 mg, 0.12 mmol) and Pd(dppf)Cl₂ (0.05 eq, 2.1 mg, 0.003 mmol). The vial was again sealed, degassed under Ar, and heated to 120° C. for 16 h. The reaction mixture was cooled to RT and diluted with EtOAc and H₂O. The layers were separated, and the organic layer was washed with brine, collected, dried over Na₂SO₄, filtered, and concentrated under reduced pressure. The crude mixture was purified by silica gel column chromatography (0-10% MeOH in DCM), and further purified by prep-HPLC (Condition 2, Gradient 1) to afford 2-(2-((2R,4S)-2-(1-cyclopropyl-1H-pyrazol-4-yl)tetrahydro-2H-pyran-4-yl)-7-methyl-4-(3-(trifluoromethyl)bicyclo[1.1.1]pentan-1-yl)pyrido[2,3-d]pyrimidin-6-yl)-5-methyl-1,3,4-oxadiazole (Example 23, 2.6 mg, 0.005 mmol, 8% yield) as a solid. ¹H NMR (CHCl₃-d, 400 MHz): δ_{H} 9.06 (1H, s), 7.50 (2H, s), 4.55 (1H, d, J=11.4 Hz), 4.26 (1H, d, J=11.3 Hz), 3.81 (1H, t, J=11.0 Hz), 3.56 (1H, s), 3.43-3.48 (1H, m), 3.18 (3H, s), 2.74 (3H, s), 2.68 (6H, s), 2.38 (1H, d, J=13.4 Hz), 2.12-2.20 (3H, m), 1.09 (2H, s), 0.99 (2H, d, J=7.6 Hz). ¹⁹F NMR (CHCl₃-d, 376 MHz): δ_{F}−73.1 (3F, s). LC/MS: [M+H]⁺ 552.3.

### Method 18: Synthesis of Example 18

### Example 18: 2-[(2R,4S)-2-(1-cyclopropylpyrazol-4-yl)tetrahydropyran-4-yl]-4-(2,4-difluorophenyl)-7-methyl-pyrido[2,3-d]pyrimidine-6-carbonitrile

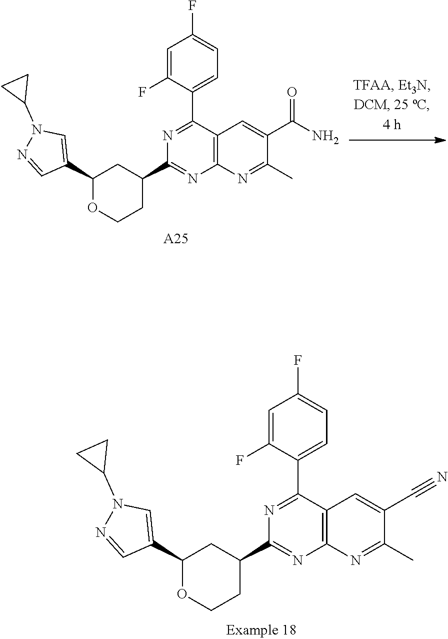 [SMILES: Cc1nc2nc([C@H]3CCO[C@@H](c4cnn(C5CC5)c4)C3)nc(-c3ccc(F)cc3F)c2cc1C(N)=O ; "A25" ; Cc1nc2nc([C@H]3CCO[C@@H](c4cnn(C5CC5)c4)C3)nc(-c3ccc(F)cc3F)c2cc1C#N]

To a solution of 2-[(2R,4S)-2-(1-cyclopropylpyrazol-4-yl)tetrahydropyran-4-yl]-4-(2,4-difluorophenyl)-7-methylpyrido[2,3-d]pyrimidine-6-carboxamide (1 eq, 44 mg, 0.09 mmol) in DCM (1 mL) was added TEA (3 eq, 0.037 mL, 0.27 mmol) and trifluoroacetic anhydride (1.2 eq, 0.015 mL, 0.11 mmol), and the solution was stirred at 25° C. for 4 h. The mixture was diluted with EtOAc, the organic phase was washed with water, brine, dried over anhydrous Na₂SO₄, and filtered. The filtrate was concentrated under reduced pressure and purified by silica gel column chromatography (30-100% EtOAc in DCM) to afford 2-[(2R,4S)-2-(1-cyclopropylpyrazol-4-yl)tetrahydropyran-4-yl]-4-(2,4-difluorophenyl)-7-methyl-pyrido[2,3-d]pyrimidine-6-carbonitrile (Example 18, 33 mg, 0.07 mmol, 78% yield) as a solid.

¹H NMR (CHCl₃-d, 400 MHz): δ_{H} 8.43 (1H, d, J 4.2 Hz), 7.68 (1H, td, J 8.3, 6.2 Hz), 7.48 (2H, d, J 3.9 Hz), 7.14-7.19 (1H, m), 7.05-7.10 (1H, m), 4.54 (1H, dd, J 11.4, 2.1 Hz), 4.23-4.27 (1H, m), 3.79 (1H, dd, J 14.9, 10.7 Hz), 3.51-3.57 (2H, m), 3.04 (3H, s), 2.40-2.44 (1H, m), 2.13-2.23 (3H, m), 1.06-1.10 (2H, m), 0.94-1.01 (2H, m). ¹⁹F NMR (CHCl₃-d, 376 MHz): δ_{F}−108.7, −103.6. LC/MS (ESI, m/z⁺): 573.3 [M+1]⁺.

### Method 19: Synthesis of Example 21

### Example 21: 2-[(2S,6R)-2-(1-cyclopropylpyrazol-4-yl)-6-methyl-morpholin-4-yl]difluorophenyl)-7-methyl-pyrido[2,3-d]pyrimidine-6-carbonitrile

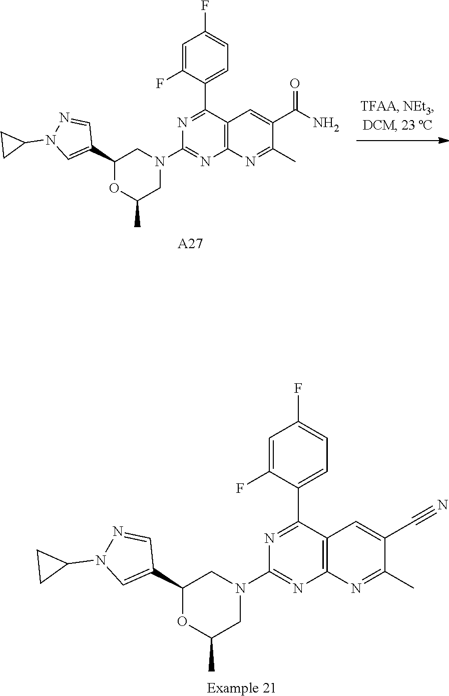

To a stirred solution of 2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carboxamide (A27, 1 eq, 42 mg, 0.08 mmol) and triethylamine (3 eq, 35 μL, 0.25 mmol) in DCM (1.1 mL), was added slowly trifluoroacetic anhydride (1.2 eq, 14 μL, 0.1 mmol). The resulting mixture was stirred at 23° C. for 4 h and saturated NaHCO₃(aq., 20 mL) was added. The organic phase was extracted with EtOAc (3×20 mL), washed with brine (20 mL), dried over Na₂SO₄ and concentrated under reduced pressure. The crude was purified by reverse phase chromatography (30-70% ACN in water (0.1% formic acid)) to afford 2-[(2S,6R)-2-(1-cyclopropylpyrazol-4-yl)-6-methyl-morpholin-4-yl]-4-(2,4-difluorophenyl)-7-methyl-pyrido[2,3-d]pyrimidine-6-carbonitrile (Example 21, 38 mg, 94% yield) as a solid. LC/MS (ESI, m/z⁺): 488.3 [M+H]⁺. ¹H NMR (CHCl₃-d, 400 MHz): δ_{H} 8.06 (1H, d, J=3.6 Hz), 7.50-7.56 (3H, m), 7.06-7.13 (1H, m), 6.98-7.05 (1H, m), 4.86-5.21 (2H, m), 4.53-4.61 (1H, m), 3.75-3.84 (1H, m), 3.51-3.58 (1H, m), 3.04-3.12 (1H, m), 2.83-2.89 (4H, m), 1.31-1.32 (3H, m), 1.09 (2H, t, J=3.8 Hz), 0.99 (2H, d, J=6.6 Hz). ¹⁹F NMR (CHCl₃-d, 376 MHz): δ_{F}−104.6 (1F, s), −108.5 (1F, s).

### Method 20: Synthesis of Example 24

### Example 24: 2-[(2R,4S)-2-(1-cyclopropylpyrazol-4-yl)tetrahydropyran-4-yl]-6-fluoro-7-methyl-4-[3-(trifluoromethyl)-1-bicyclo[1.1.1]pentanyl]-pyrido[2,3-d]pyrimidine PGP-181 C₃

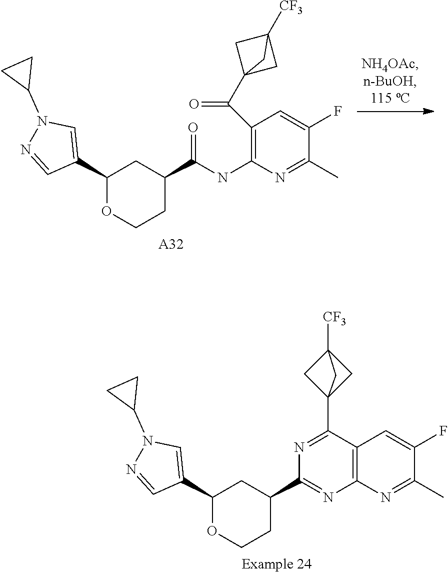

A vial was charged with (2R,4S)-2-(1-cyclopropylpyrazol-4-yl)-N-[5-fluoro-6-methyl-3-[3-(trifluoromethyl)bicyclo[1.1.1]pentane-1-carbonyl]-2-pyridyl]tetrahydropyran-4-carboxamide (A32, 1 eq, 88 mg, 0.17 mmol), ammonium acetate (26 eq, 348 mg, 4.5 mmol) and n-Butanol (2 mL). The vial was capped and heated to 115° C. with stirring for 2 h. Brine (20 mL) was added, and the organic phase was extracted with EtOAc (3×20 mL), washed with brine (30 mL), dried over Na₂SO₄, filtered, and concentrated under reduced pressure. The crude was purified by reverse phase column chromatography (30-75% MeCN in water (0.1% formic acid)) to afford 2-[(2R,4S)-2-(1-cyclopropylpyrazol-4-yl)tetrahydropyran-4-yl]-6-fluoro-7-methyl-4-[3-(trifluoromethyl)-1-bicyclo[1.1.1]pentanyl]-pyrido[2,3-d]pyrimidine (Example 24, 80 mg, 94% yield) as a solid. LC/MS (ESI, m/z⁺): 488.4 [M+H]⁺. ¹H NMR (CHCl₃-d, 400 MHz): δ_{H} 8.03 (1H, d, J=8.5 Hz), 7.48 (2H, d, J=2.6 Hz), 4.53 (1H, dd, J=11.4, 2.1 Hz), 4.21-4.25 (1H, m), 3.74-3.81 (1H, m), 3.54 (1H, tt, J=7.2, 3.8 Hz), 3.38-3.43 (1H, m), 2.79 (3H, d, J=2.9 Hz), 2.61 (7H, s), 2.36 (1H, dd, J=13.4, 3.0 Hz), 2.06-2.15 (3H, m), 1.55 (3H, s), 1.52 (1H, s), 1.06-1.10 (2H, m), 0.94-0.99 (2H, m). ¹⁹F NMR (CHCl₃-d, 376 MHz): δ_{F}−73.2 (3F, s), −120.6 (1F, s).

### Method 21: Synthesis of Example 25

### Example 25

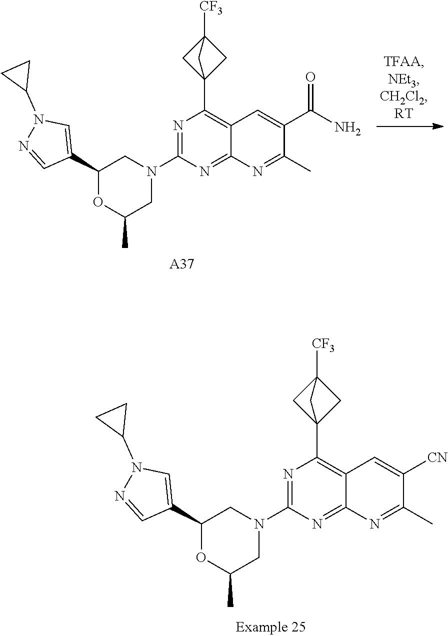 [SMILES: Cc1nc2nc(N3C[C@@H](C)O[C@@H](c4cnn(C5CC5)c4)C3)nc(C34CC(C(F)(F)F)(C3)C4)c2cc1C(N)=O ; "A37" ; Cc1nc2nc(N3C[C@@H](C)O[C@@H](c4cnn(C5CC5)c4)C3)nc(C34CC(C(F)(F)F)(C3)C4)c2cc1C#N]

To a flame-dried, round-bottomed flask equipped with a Teflon-coated magnetic stirring bar was charged 2-[(2S,6R)-2-(1-cyclopropylpyrazol-4-yl)-6-methyl-morpholin-4-yl]-7-methyl-4-[3-(trifluoromethyl)-1-bicyclo[1.1.1]pentanyl]pyrido[2,3-d]pyrimidine-6-carboxamide (A37, 1 eq, 70 mg, 0.13 mmol) in anhydrous DCM (2 mL). The vessel was sealed, purged under Ar, and cooled to 0° C. with stirring. NEt₃ (3 eq, 55 μL, 0.4 mmol) was added, followed by the drop-wise addition oftrifluoroacetic anhydride (1.2 eq, 22 μL, 0.16 mmol). The reaction mixture was stirred at 0° C. for 1 h and then at RT for 3 hrs. The reaction mixture was diluted with EtOAc (50 mL) and washed with sat. NH₄Cl (aq) and brine. The organic layer was collected, dried over Na₂SO₄, filtered, and concentrated under reduced pressure onto Celite. The crude mixture was purified by silica gel column chromatography (15-50% EtOAc in DCM) to afford 2-[(2S,6R)-2-(1-cyclopropylpyrazol-4-yl)-6-methyl-morpholin-4-yl]-7-methyl-4-[3-(trifluoromethyl)-1-bicyclo[1.1.1]pentanyl]pyrido[2,3-d]pyrimidine-6-carbonitrile (Example 25, 54 mg, 0.11 mmol, 79% yield) as a solid. ¹H NMR (CHCl₃-d, 400 MHz): δ_{H} 8.47 (1H, s), 7.51-7.54 (2H, m), 4.89-5.19 (2H, m), 4.55 (1H, d, J=10.9 Hz), 3.74-3.82 (1H, m), 3.56-3.60 (1H, m), 3.02-3.11 (1H, m), 2.79-2.86 (4H, m), 2.58 (6H, d, J=12.0 Hz), 1.33 (3H, dd, J=10.2, 6.2 Hz), 1.10-1.14 (2H, m), 1.00-1.03 (2H, m). ¹⁹F NMR (CHCl₃-d, 376 MHz): δ_{F}−73.1 (3F, s). LC/MS (m/z⁺): 510.4 [M+H]⁺.

### Method 22: Synthesis of Examples 31-32

### Example 31: 2-[(2R,4S)-2-(1-cyclopropylpyrazol-4-yl)tetrahydropyran-4-yl]-7-methyl-6-methylsulfonyl-4-[3-(trifluoromethyl)-1-bicyclo[1.1.1]pentanyl]pyrido[2,3-d]pyrimidine

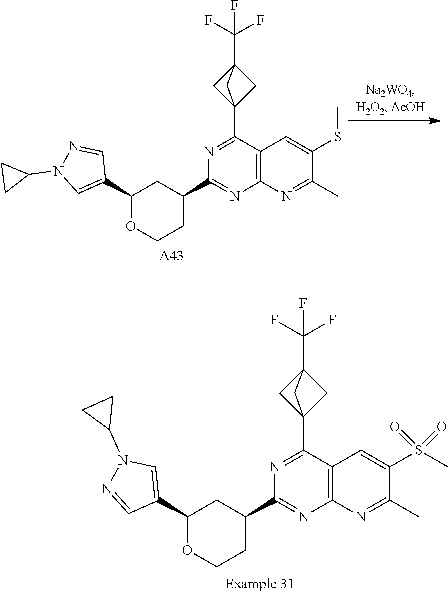

To a solution of 2-[(2R,4S)-2-(1-cyclopropylpyrazol-4-yl)tetrahydropyran-4-yl]-7-methyl-6-methylsulfanyl-4-[3-(trifluoromethyl)-1-bicyclo[1.1.1]pentanyl]pyrido[2,3-d]pyrimidine (A43, 1 eq, 37 mg, 0.072 mmol) in AcOH (1 mL) was added sodium tungstate dihydrate (2 eq, 47 mg, 0.14 mmol) and hydrogen peroxide (30%) (20 eq, 0.15 mL, 1.44 mmol). The reaction was stirred for 1 h then poured into saturated aqueous NaHCO₃ and extracted with EtOAc. The EtOAc extract was washed with brine, dried over anhydrous Na₂SO₄ and concentrated under reduced pressure. The residue obtained was purified by silica gel column chromatography (50-100% EtOAc in DCM) to afford 2-[(2R,4S)-2-(1-cyclopropyl-pyrazol-4-yl)tetrahydropyran-4-yl]-7-methyl-6-methylsulfonyl-4-[3-(trifluoromethyl)-1-bicyclo[1.1.1]pentanyl]pyrido[2,3-d]pyrimidine (Example 31, 18 mg, 0.033 mmol, 46% yield) as a solid.

¹H NMR (CHCl₃-d, 400 MHz): δ_{H} 9.25 (1H, s), 7.48 (2H, d, J=3.9 Hz), 4.54 (1H, dd, J=11.4, 2.1 Hz), 4.25 (1H, d, J=11.6 Hz), 3.79 (1H, d, J=3.6 Hz), 3.48-3.57 (1H, m), 3.25 (3H, s), 3.15 (3H, s), 2.66 (8H, s), 2.36 (1H, d, J=13.4 Hz), 2.07-2.16 (4H, m), 0.94-1.09 (5H, m). ¹⁹F NMR (CHCl₃-d, 376 MHz): δ_{F}−73.1. LC/MS (ESI, m/z⁺): 548.3 [M+H]⁺.

### Example 32: 2-[(2R,4S)-2-(1-cyclopropylpyrazol-4-yl)tetrahydropyran-4-yl]-7-methyl-6-methylsulfinyl-4-[3-(trifluoromethyl)-1-bicyclo[1.1.1]pentanyl]pyrido[2,3-d]pyrimidine

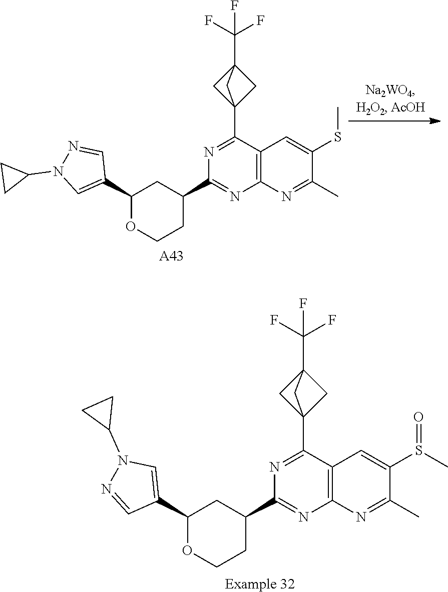

To a solution of 2-[(2R,4S)-2-(1-cyclopropylpyrazol-4-yl)tetrahydropyran-4-yl]-7-methyl-6-methylsulfanyl-4-[3-(trifluoromethyl)-1-bicyclo[1.1.1]pentanyl]pyrido[2,3-d]pyrimidine (A43, 1 eq, 32 mg, 0.062 mmol) in AcOH (1 mL) was added sodium tungstate dihydrate (2 eq, 41 mg, 0.124 mmol) and hydrogen peroxide (30%) (2 eq, 0.013 mL, 0.124 mmol). The reaction mixture was stirred at room temperature for 5 h. The mixture was poured into aqueous saturated NaHCO₃ and extracted with EtOAc. The EtOAc extract was washed with brine, dried over anhydrous Na₂SO₄ and concentrated under reduced pressure. The residue obtained was purified by silica gel column chromatography (50-100% EtOAc in DCM) then lyophilized to afford 2-[(2R,4S)-2-(1-cyclopropylpyrazol-4-yl)tetrahydropyran-4-yl]-7-methyl-6-methylsulfinyl-4-[3-(trifluoromethyl)-1-bicyclo[1.1.1]pentanyl]pyrido[2,3-d]pyrimidine, Example 32, as a solid. ¹H NMR (CHCl₃-d, 400 MHz): δ_{H} 9.09 (1H, s), 7.48 (2H, d, J=3.0 Hz), 4.53 (1H, dd, J=11.4, 2.1 Hz), 4.22-4.26 (1H, m), 3.75-3.82 (1H, m), 3.54 (1H, tt, J=7.2, 3.8 Hz), 2.83 (3H, s), 2.77 (3H, s), 2.33-2.38 (1H, m), 2.08-2.18 (3H, m), 1.05-1.09 (2H, m), 0.93-1.01 (2H, m). LC/MS (ESI, m/z⁺): 432.3 [M+H]⁺.

### Method 23: Synthesis of Examples 35, 45, and 46

### Example 35: 4-cyclohexyl-2-[(2S,6R)-2-(1-cyclopropylpyrazol-4-yl)-6-methyl-morpholin-4-yl]-7-methyl-pyrido[2,3-d]pyrimidine-6-carbonitrile

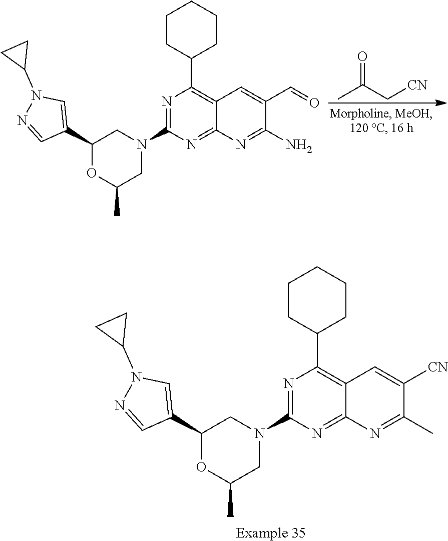

To a solution of 4-amino-6-cyclohexyl-2-[(2S,6R)-2-(1-cyclopropylpyrazol-4-yl)-6-methyl-morpholin-4-yl]pyrimidine-5-carbaldehyde (1 eq, 200 mg, 0.49 mmol) in MeOH (1.5 mL) was added 3-oxobutanenitrile (2 eq, 75 μL, 0.97 mmol) and morpholine (1 eq, 41 μL, 0.49 mmol). The resulting mixture was stirred at 120° C. for 16 h and aqueous NaHCO₃ (100 mL) was added. The mixture was extracted three times with EtOAc (3×50 mL) and the combined organic layers were washed with brine, dried over Na₂SO₄, filtered and concentrated under reduced pressure. The crude product was purified by silica gel column chromatography (10-80% o EtOAc in Hexanes, followed by 0-10% MeOH in DCM) to afford 4-cyclohexyl-2-[(2S,6R)-2-(1-cyclopropylpyrazol-4-yl)-6-methyl-morpholin-4-yl]-7-methyl-pyrido[233-d]pyrimidine-6-carbonitrile (Example 35, 110 mg, 0.24 mmol, 49J yield) as a solid. LC/MS (ESI, m/z⁺): 458.4 [M+H]⁺. ¹H NMR (CHCl₃-d, 400 MHz): δ_{H} 8.41 (11H, s), 7.49-7.52 (2H, m), 4.93-5.19 (1H, m), 4.53-4.57 (1H, m), 3.74-3.81 (1H, m), 3.54-3.60 (1H, m), 3.20-3.28 (1H, s), 2.99-3.09 (1H, m), 2.76-2.85 (4H, i), 1.78-1.94 (5H, i), 1.29-1.36 (4H, i), 1.08-115 (2H, m), 0.97-1.02, (2H, m).

An analogous method was followed to obtain the following compounds.

| Ex # | Structure | Starting Material | Characterization |
|---|---|---|---|
| 45 | 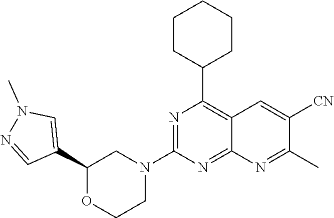 [SMILES: Cc1nc2nc(N3CCO[C@@H](c4cnn(C)c4)C3)nc(C3CCCCC3)c2cc1C#N] | 4-amino-6- cyclohexyl-2- [(2S)-2-(1-methyl- pyrazol-4-yl)- morpholin-4-yl]- pyrimidine-5- carbaldehyde | 4-cyclohexyl-7-methyl-2-[(2S)-2-(1- methyl-pyrazol-4-yl)morpholin-4- yl]pyrido[2,3-d]pyrimidine-6- carbonitrile, Example 45. ¹H NMR (CHCl₃-d, 400 MHz): δ_{H} 8.43 (1H, s), 7.54 (1H, s), 7.43 (1H, d, J = 11.4 Hz), 4.95-5.15 (1H, m), 4.90 (1H, t, J = 13.0 Hz), 4.57 (1H, dd, J = 10.2, 2.7 Hz), 4.09 (1H, t, J = 12.6 Hz), 3.90 (3H, s), 3.77 (1H, t, J = 11.5 Hz), 3.20-3.42(3H, m), 2.85 (3H, s), 1.82-1.93 (5H, m), 1.64-1.73 (2H, m), 1.44-1.51 (2H, m), 1.34 (1H, t, J = 13.6 Hz). LC/MS (m/z): 418.4 [M + H]⁺. |
| 46 | 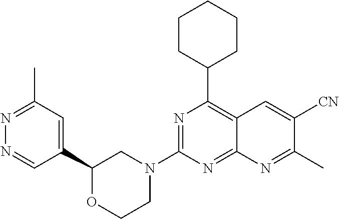 [SMILES: Cc1cc([C@H]2CN(c3nc(C4CCCCC4)c4cc(C#N)c(C)nc4n3)CCO2)cnn1] | 4-amino-6- cyclohexyl-2-[2- (6-methyl- pyridazin-4-yl)- morpholin-4-yl]- pyrimidine-5- carbaldehyde | 4-cyclohexyl-7-methyl-2-[2-(6- methyl-pyridazin-4-yl)morpholin-4- yl]-pyrido[2,3-d]pyrimidine-6- carbonitrile, Example 46. ¹H NMR (CHCl₃-d, 400 MHz): δ_{H} 9.08-9.19 (1H, m), 8.47 (1H, s), 7.41-7.49 (1H, m), 5.14-5.28 (1H, m), 5.02 (1H,d, J = 13.7 Hz), 4.60 (1H, d, J = 10.3 Hz), 4.16-4.25 (1H, m), 3.79-3.85 (1H, m), 3.26-3.39 (2H, m), 3.01-3.12 (1H,), 2.87 (3H, s), 2.77 (3H, s), 1.93 (5H, br s), 1.86 (3H, d, J = 15.4 Hz), 1.66-1.74 (2H, m), 1.50-1.56 (3H, m), 1.29-1.38 (1H, m). LC/MS (m/z): 430.4 [M + H]⁺. |

### Method 24: Synthesis of Example 42

### Example 42: 4-cyclobutyl-2-((2S,6R)-2-(1-cyclopropyl-1H-pyrazol-4-yl)-6-methylmorpholino)-7-methylpyrido[2,3-d]pyrimidine-6-carbonitrile

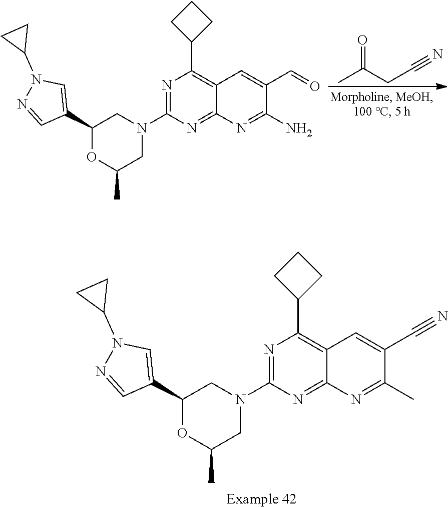

To a solution of Morpholine (2 eq, 42 μL, 0.47 mmol) in MeOH (1 mL) was added 3-oxobutanenitrile (2 eq, 36 μL, 0.47 mmol), and the solution was stirred for 30 min and then added to a solution of 4-amino-6-cyclobutyl-2-[(2S,6R)-2-(1-cyclopropylpyrazol-4-yl)-6-methyl-morpholin-4-yl]pyrimidine-5-carbaldehyde (A42, 1 eq, 90 mg, 0.24 mmol) in MeOH (1 mL). The mixture was stirred at 100° C. for 5 h. The reaction mixture was quenched with water and diluted with EtOAc (50 mL). The phases were separated, and the organic phase was washed with water and brine. The organic layer was collected, dried over anhydrous Na₂SO₄, filtered, and concentrated under reduced pressure. The crude mixture was purified by silica gel column chromatography (40-100% EtOAc in DCM) to afford 4-cyclobutyl-2-((2S,6R)-2-(1-cyclopropyl-1H-pyrazol-4-yl)-6-methylmorpholino)-7-methylpyrido[2,3-d]pyrimidine-6-carbonitrile (Example 42, 18 mg, 0.042 mmol, 18% yield) as a solid. ¹H NMR (CHCl₃-d, 400 MHz): δ_{H} 8.25 (1H, s), 7.51-7.56 (2H, m), 5.14-5.23 (1H, m), 5.04-5.11 (1H, m), 4.58 (1H, dd, J=10.9, 2.5 Hz), 4.07-4.14 (1H, m), 3.76-3.83 (1H, m), 3.55-3.61 (1H, m), 3.03-3.14 (1H, m), 2.81-2.89 (4H, m), 2.41-2.60 (3H, m), 2.15-2.24 (1H, m), 1.92-2.03 (1H, m), 1.29-1.36 (4H, m), 1.09-1.15 (2H, m), 1.00-1.06 (2H, m). LC/MS (ESI, m/z⁺): 430.4 [M+H]⁺.

### Method 25: Synthesis of Example 67

### Synthesis of A44

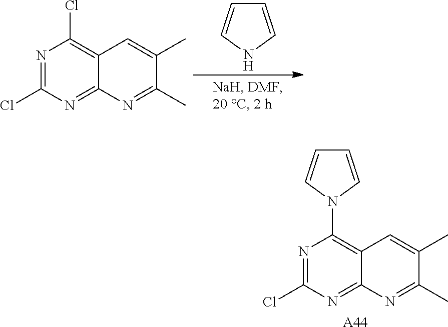

To a solution of pyrrole (1.20 eq, 0.074 mL, 1.05 mmol) in DMF (1 mL) was added NaH (1.30 eq, 46 mg, 1.14 mmol) in one portion under N₂, then the mixture was stirred at 20° C. for 5 min under N₂. Then this mixture was added into a solution of 2,4-dichloro-6,7-dimethyl-pyrido[2,3-d]pyrimidine (1.0 eq, 200 mg, 0.877 mmol) in DMF (1 mL), and the mixture was stirred at 20° C. for 2 h under N₂. The reaction mixture was added into water (50 mL), extracted with EtOAc (3×40 mL), and washed with brine (3×40 mL). The combined organic layers were dried over Na₂SO₄, filtered and concentrated under reduced pressure to give a residue. The residue was purified by silica gel column chromatography (0-70% EtOAc in Petroleum ether; 35 mL/min) to afford 2-chloro-6,7-dimethyl-4-pyrrol-1-yl-pyrido[2,3-d]pyrimidine (A44, 70 mg, 0.27 mmol, 31% yield) as a solid. LC/MS (ESI, m/z⁺): 259.0 [M+H]⁺. ¹H NMR (400 MHz, CDCl₃) δ=8.33 (s, 1H), 7.50-7.45 (m, 2H), 6.56-6.50 (m, 2H), 2.80 (s, 3H), 2.53 (s, 3H).

### Synthesis of Example 67

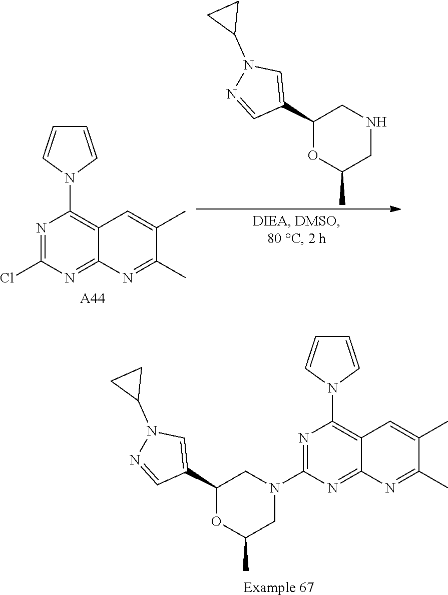

To a solution of (2S,6R)-2-(1-cyclopropylpyrazol-4-yl)-6-methyl-morpholine (1.1 eq, 71 mg, 0.34 mmol) and DIEA (5.0 eq, 0.26 mL, 1.55 mmol) in DMSO (2 mL) was added 2-chloro-6,7-dimethyl-4-pyrrol-1-yl-pyrido[2,3-d]pyrimidine (A44, 1.0 eq, 80 mg, 0.31 mmol) in one portion under N₂, and the reaction was stirred at 80° C. for 2 h under N₂. The resulting mixture was filtered, concentrated under reduced pressure, and purified by prep-HPLC (Condition 3) and lyophilized to give (2S,6R)-2-(1-cyclopropyl-pyrazol-4-yl)-4-(6,7-dimethyl-4-pyrrol-1-yl-pyrido[2,3-d]pyrimidin-2-yl)-6-methyl-morpholine (Example 67, 101 mg, 0.23 mmol, 73% yield) as a solid. LC/MS (ESI, m/z⁺): 430.2 [M+H]⁺.

¹H NMR (400 MHz, CDCl₃) δ=8.04 (s, 1H), 7.54 (d, J=4.9 Hz, 2H), 7.44-7.35 (m, 2H), 6.47 (t, J=2.1 Hz, 2H), 5.27-4.74 (m, 3H), 4.58 (dd, J=2.5, 10.9 Hz, 1H), 3.89-3.74 (m, 1H), 3.64-3.53 (m, 1H), 3.05 (dd, J=11.0, 13.3 Hz, 1H), 2.81 (dd, J=10.6, 13.3 Hz, 1H), 2.68 (s, 4H), 2.39 (s, 4H), 1.32 (d, J=6.3 Hz, 4H), 1.17-1.09 (m, 2H), 1.08-0.96 (m, 2H).

### Method 26: Synthesis of Example 68

### Synthesis of A45

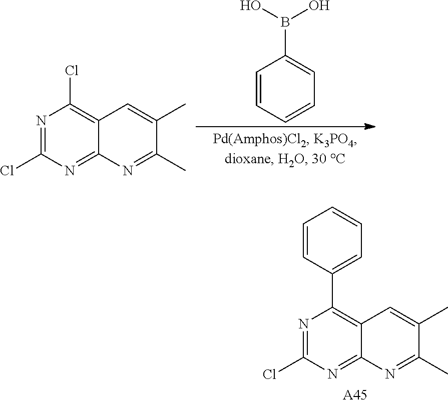

A mixture of 2,4-dichloro-6,7-dimethyl-pyrido[2,3-d]pyrimidine (1.0 eq, 50 mg, 0.22 mmol), K₃PO₄ (3.0 eq, 140 mg, 0.66 mmol), Pd(AmPhos)Cl₂ (0.10 eq, 16 mg, 0.022 mmol) and phenylboronic acid (0.90 eq, 24 mg, 0.197 mmol) in 1,4-Dioxane (1 mL) and water (0.1 mL) was stirred under N₂ at 30° C. for 1 h. The solution (combined with other 3 batches) was extracted with EtOAc (3×20 mL) and water (20 mL). The combined organic layers were washed with brine (3×60 mL), dried over Na₂SO₄, and filtered. The filtrate was concentrated under reduced pressure and purified by prep-HPLC (FA) and lyophilized to give 2-chloro-6,7-dimethyl-4-phenyl-pyrido[2,3-d]pyrimidine (A45, 100 mg, 0.37 mmol, 169% yield) as a solid. 2D NMR confirmed the regio-selectivity. LC/MS (ESI, m/z⁺): 270.0 [M+H]⁺. ¹H NMR (400 MHz, CDCl₃) δ=8.13 (br s, 1H), 7.82-7.74 (m, 2H), 7.62 (br s, 3H), 2.80 (br s, 3H), 2.48 (br s, 3H).

### Synthesis of Example 68

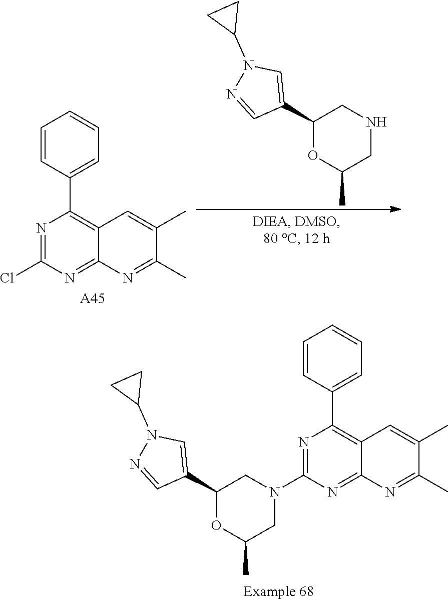

To a solution of (2S,6R)-2-(1-cyclopropylpyrazol-4-yl)-6-methyl-morpholine (1.1 eq, 76 mg, 0.37 mmol) in DMSO (2 mL) were added DIEA (5.0 eq, 0.28 mL, 1.67 mmol) and 2-chloro-6,7-dimethyl-4-phenyl-pyrido[2,3-d]pyrimidine (A45, 1.0 eq, 90 mg, 0.33 mmol), and the mixture was stirred at 80° C. for 12 h under N₂. The solution was purified by prep-HPLC (FA) and lyophilized to afford (2S,6R)-2-(1-cyclopropylpyrazol-4-yl)-4-(6,7-dimethyl-4-phenylpyrido[2,3-d]pyrimidin-2-yl)-6-methyl-morpholine (Example 68, 99 mg, 0.22 mmol, 64% yield) as a solid. LC/MS (ESI, m/z⁺): 441.2 [M+H]⁺. ¹H NMR (400 MHz, CDCl₃) δ=7.84 (s, 1H), 7.75-7.68 (m, 2H), 7.61-7.52 (m, 5H), 5.19-5.11 (m, 1H), 5.04 (br dd, J=2.0, 13.6 Hz, 1H), 4.61 (dd, J=1.8, 10.6 Hz, 1H), 3.89-3.78 (m, 1H), 3.58 (tt, J=3.5, 7.2 Hz, 1H), 3.12-3.00 (m, 1H), 2.86-2.77 (m, 1H), 2.68 (s, 3H), 2.34 (s, 3H), 1.33 (d, J=6.2 Hz, 3H), 1.17-1.09 (m, 2H), 1.05-0.96 (m, 2H).

An analogous method was followed to obtain the following compounds.

| Compound | Starting Material | Characterization |
|---|---|---|
| 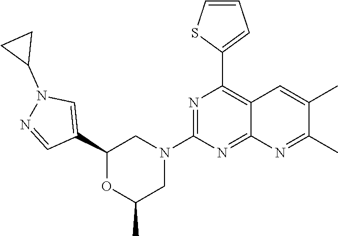 [SMILES: Cc1cc2c(-c3cccs3)nc(N3C[C@@H](C)O[C@@H](c4cnn(C5CC5)c4)C3)nc2nc1C] | 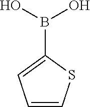 | Modification: Step 1 was performed using Pd(dppf)Cl₂ and K₂CO₃. Step 2 was ran for 2 h. The residue was purified by prep-HPLC (FA) and lyophilized to afford (2S,6R)-2-(1-cyclopropyl-pyrazol-4-yl)-4-[6,7- dimethyl-4-(2-thienyl)pyrido-[2,3-d]pyrimidin-2-yl]-6- methyl-morpholine (Example 69, 81 mg, 0.17 mmol, 67% yield) as a solid. LC/MS (ESI, m/z⁺): 447.2 [M + H]⁺. ¹H NMR (400 MHz, CDCl₃) δ = 8.24 (s, 1H), 7.76 (d, J = 3.7 Hz, 1H), 7.61 (d, J = 5.0 Hz, 1H), 7.56 (d, J = 5.4 Hz, 2H), 7.25 (dd, J = 3.8, 4.9 Hz, 1H), 5.18-4.94 (m, 2H), 4.61 (dd, J = 2.6, 10.9 Hz, 1H), 3.88-3.78 (m, 1H), 3.63-3.53 (m, 1H), 3.06 (dd, J = 11.1, 13.1 Hz, 1H), 2.82 (dd, J = 10.8, 13.3 Hz, 1H), 2.68 (s, 3H), 2.41 (s, 3H), 1.33 (d, J = 6.2 Hz, 3H), 1.16-1.11 (m, 2H), 1.05-0.99 (m, 2H). |
| Example 69 | | |

### Method 27: Synthesis of Example 81

### Synthesis of A51

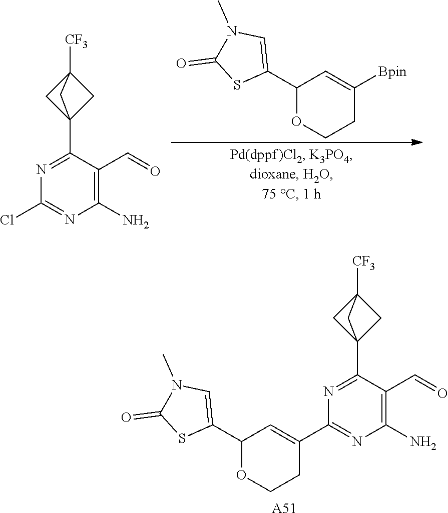

A vial equipped with a Teflon-coated magnetic stirring bar was charged with 4-amino-2-chloro-6-[3-(trifluoromethyl)-1-bicyclo[1.1.1]pentanyl]pyrimidine-5-carbaldehyde (1.05 eq, 247 mg, 0.85 mmol), 3-methyl-5-[4-(4,4,5,5-tetramethyl-1,3,2-dioxaborolan-2-yl)-3,6-dihydro-2H-pyran-6-yl]thiazol-2-one (A118a, 1.0 eq, 286 mg, 0.81 mmol), K₃PO₄ (2.5 eq, 427 mg, 2.0 mmol), Pd(dppf)Cl₂ DCM (0.1 eq, 66 mg, 0.081 mmol), 1,4-dioxane (5 mL) and water (0.5 mL). The vial was sealed, degassed under argon, and stirred at 75° C. for 2 h. The reaction mixture was cooled down to RT and filtered over celite, rinsing with EtOAc. The filtrate was concentrated under reduced pressure and purified by silica gel flash column chromatography (20-100% EtOAc in Hexanes) to yield 4-amino-2-[6-(3-methyl-2-oxo-thiazol-5-yl)-3,6-dihydro-2H-pyran-4-yl]-6-[3-(trifluoromethyl)-1-bicyclo[1.1.1]pentanyl]pyrimidine-5-carbaldehyde (A51, 138 mg, 0.27 mmol, 33% yield) as a solid. ESI-MS (m/z)+: 453.2 (obs).

### Synthesis of A52

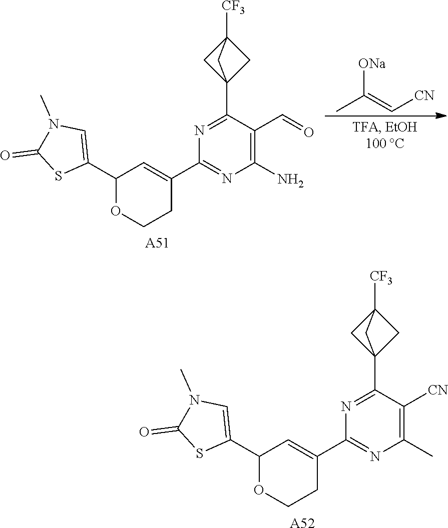

To a glass vial equipped with a Teflon-coated magnetic stirring bar was added 4-amino-2-[6-(3-methyl-2-oxo-thiazol-5-yl)-3,6-dihydro-2H-pyran-4-yl]-6-[3-(trifluoromethyl)-1-bicyclo[1.1.1]pentanyl]-pyrimidine-5-carbaldehyde (A51, 1.0 eq, 138 mg, 0.24 mmol), [(Z)-2-cyano-1-methyl-vinyloxy]-sodium (3.0 eq, 77 mg, 0.73 mmol) and ethanol (2 mL), and the reaction was stirred at RT for 10 min. TFA (5.0 eq, 94 μL, 1.2 mmol) was then added and the reaction mixture was stirred at 100° C. for 24 h. The resulting mixture was then cooled to RT and diluted with EtOAc. The resulting solution was washed with sat. NaHCO₃(aq.) and brine. The organic layer was collected, dried over anhydrous Na₂SO₄, and filtered. The filtrate was concentrated under reduced pressure onto celite (10 g) and purified by silica gel flash column chromatography (30-100% EtOAc in Hexanes) to yield 7-methyl-2-[6-(3-methyl-2-oxo-thiazol-5-yl)-3,6-dihydro-2H-pyran-4-yl]-4-[3-(trifluoromethyl)-1-bicyclo[1.1.1]pentanyl]pyrido[2,3-d]pyrimidine-6-carbonitrile (A52, 26 mg, 0.052 mmol, 21% yield) as a solid. ESI-MS (m/z)+: 500.3 (obs). ¹H NMR (CHCl₃-d, 400 MHz): δ_{H} 8.80 (s, 1H), 7.63 (t, J=1.8 Hz, 1H), 6.56 (s, 1H), 5.34-5.33 (m, 1H), 4.12-4.07 (m, 1H), 3.97-3.91 (m, 1H), 3.32 (s, 3H), 3.01 (s, 3H), 2.87-2.94 (m, 2H), 2.66 (s, 6H). ¹⁹F NMR (CHCl₃-d, 376 MHz): δ_{F}−73.0 (s, 3F).

### Synthesis of Example 81

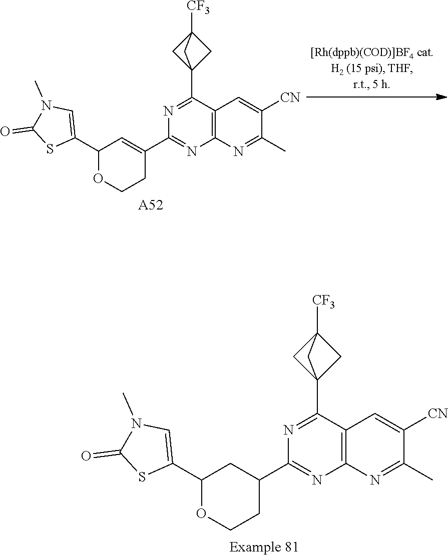

A round-bottomed flask equipped with a Teflon-coated magnetic stirring bar was charged with [Rh(dppb)(COD)] BF₄ (0.27 eq, 10 mg, 0.014 mmol). The flask was purged with argon before adding a solution of 7-methyl-2-[6-(3-methyl-2-oxo-thiazol-5-yl)-3,6-dihydro-2H-pyran-4-yl]-4-[3-(trifluoromethyl)-1-bicyclo[1.1.1]pentanyl]pyrido[2,3-d]pyrimidine-6-carbonitrile (A52, 1.0 eq, 26 mg, 0.052 mmol) in THF (1 mL). The heterogeneous reaction mixture was then purged with H₂ and stirred at 55° C. under H₂ (15 psi) for 5 h. The reaction mixture was cooled to RT and filtered over celite and a small layer of SiO₂, rinsing with EtOAc. The filtrate was concentrated under reduced pressure and purified by silica gel flash column chromatography (0-50% EtOAc:EtOH (3:1) in Hexanes) to yield 7-methyl-2-[rac-(2R,4S)-2-(3-methyl-2-oxo-thiazol-5-yl)tetrahydropyran-4-yl]-4-[3-(trifluoromethyl)-1-bicyclo[1.1.1]pentanyl]pyrido[2,3-d]pyrimidine-6-carbonitrile (Example 81, 2.5 mg, 0.005 mmol, 10% yield) as a solid. ¹H NMR (CHCl₃-d, 400 MHz): δ_{H} 8.82 (s, 1H), 6.54 (s, 1H), 4.44 (dd, J=10.3, 0.7 Hz, 1H), 4.28-4.24 (m, 1H), 3.76 (td, J=11.4, 3.2 Hz, 1H), 3.48-3.40 (m, 1H), 3.32 (s, 1H), 3.30 (s, 3H), 3.03 (s, 1H), 3.03 (s, 3H), 2.66 (s, 6H), 2.38-2.35 (m, 1H), 2.14-2.04 (m, 3H). ¹⁹F NMR (CHCl₃-d, 376 MHz): δ_{F}−73.0 (s, 3F). ESI-MS (m/z)+: 502.3 (obs).

### Method 28: Synthesis of Example 65

### Synthesis of A53

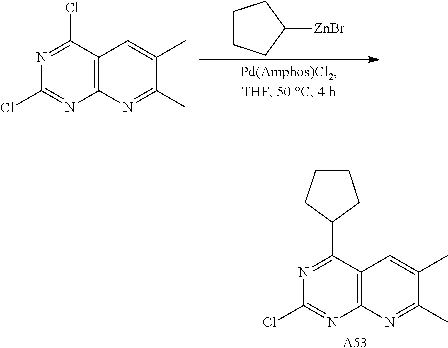

Bromo(cyclopentyl)zinc (1.0 eq, 141 mg, 0.66 mmol) was added dropwise to a 50° C. solution of 2,4-dichloro-6,7-dimethyl-pyrido[2,3-d]pyrimidine (1.0 eq, 150 mg, 0.66 mmol) and Pd(amphos)Cl₂ (0.05 eq, 23 mg, 0.03 mmol) in THF (13 mL), and the reaction was stirred for 4 h at 50° C. The reaction was quenched with NaHCO₃ and was extracted with EtOAc (×3). The combined organic layers were washed with brine, dried over Na₂SO₄, and filtered. The filtrate was concentrated under reduced pressure and purified by normal phase column chromatography (0-50% EtOAc in hexanes) to afford 2-chloro-4-cyclopentyl-6,7-dimethyl-pyrido[2,3-d]pyrimidine (A53, 59.0 mg, 0.22 mmol, 34% yield). ESI-MS (m/z)+: 262.2 (obs). ¹H NMR (CHCl₃-d, 400 MHz): δ_{H} 8.11 (1H, s), 3.81-3.88 (1H, m), 2.70 (3H, s), 2.47 (3H, s), 2.02-2.05 (4H, m), 1.85-1.89 (2H, m), 1.72-1.74 (2H, m).

### Synthesis of Example 65

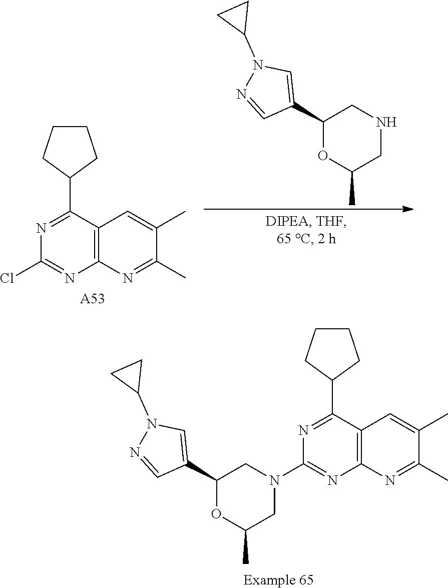

(2S,6R)-2-(1-cyclopropylpyrazol-4-yl)-6-methyl-morpholine (1.0 eq, 47 mg, 0.23 mmol), 2-chloro-4-cyclopentyl-6,7-dimethyl-pyrido[2,3-d]pyrimidine (A53, 1.0 eq, 59 mg, 0.23 mmol) and DIPEA (3.0 eq, 0.12 mL, 0.68 mmol) were stirred at 70° C. in THF (2.3 mL) for 2 h. The reaction was concentrated to dryness and purified by reversed-phase column chromatography (35-75% MeCN in water with 0.1% formic acid) to afford (2S,6R)-4-(4-cyclopentyl-6,7-dimethylpyrido[2,3-d]pyrimidin-2-yl)-2-(1-cyclopropylpyrazol-4-yl)-6-methylmorpholine (Example 65, 19 mg, 0.044 mmol, 19% yield) as a solid. ESI-MS (m/z)+: 433.4 (obs). ¹H NMR (CHCl₃-d, 400 MHz): δ_{H} 7.94 (1H, s), 7.53 (2H, s), 4.98 (2H, br s), 4.56 (1H, dd, J=10.9, 2.6 Hz), 3.78 (2H, t, J=7.8 Hz), 3.58 (1H, tt, J=7.2, 3.8 Hz), 3.01 (1H, t, J=12.1 Hz), 2.77 (1H, t, J=11.7 Hz), 2.69 (3H, s), 2.39 (3H, s), 1.98-2.04 (4H, m), 1.85 (2H, s), 1.74-1.76 (2H, m), 1.31 (3H, d, J=6.2 Hz), 1.11-1.15 (2H, m), 0.97-1.02 (2H, m).

### Method 29: Synthesis of Example 66

### Synthesis of A54

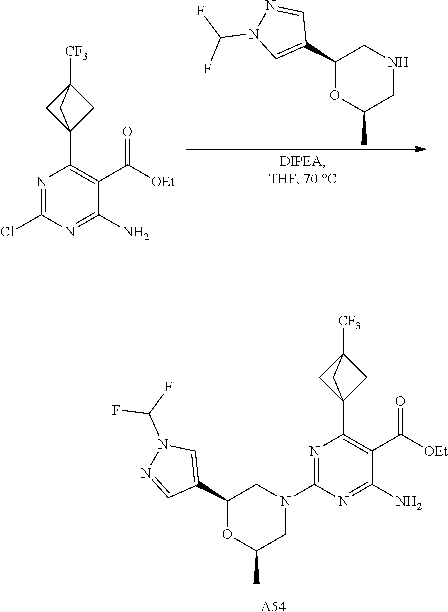

A vial was charged with ethyl 4-amino-2-chloro-6-[3-(trifluoromethyl)-1-bicyclo[1.1.1]pentanyl]-pyrimidine-5-carboxylate (A33, 1.0 eq, 200 mg, 0.6 mmol) and (2S,6R)-2-[1-(difluoromethyl)pyrazol-4-yl]-6-methylmorpholine (2.3 eq, 300 mg, 1.4 mmol) in anhydrous THF (1 mL) and DIPEA (3.0 eq, 311 μL, 1.8 mmol) was added. The vial was capped and stirred at 85° C. for 3 h. The reaction mixture was cooled down to RT, quenched with water and the resulting solid was filtered and rinsed with EtOAc and water. The filtrate was extracted with EtOAc (3×20 mL). The combined organic layers were washed with brine, dried with Na₂SO₄, and filtered. The filtrate was concentrated under reduced pressure and purified by silica gel flash chromatography (0-35% EtOAc in Hexanes) to yield ethyl 4-amino-2-[(2S,6R)-2-[1-(difluoromethyl)pyrazol-4-yl]-6-methylmorpholin-4-yl]-6-[3-(trifluoromethyl)-1-bicyclo[1.1.1]pentanyl]pyrimidine-5-carboxylate (A54, 163 mg, 0.3 mmol, 53% yield) as a solid. ESI-MS (m/z)+: 517.1 (obs). ¹H NMR (CHCl₃-d, 400 MHz): δ_{H} 7.87-7.91 (1H, m), 7.69-7.74 (1H, m), 7.26 (1H, t, J=60 Hz), 4.71-4.91 (1H, m), 4.54-4.60 (1H, m), 4.35-4.40 (2H, m), 3.71-3.78 (1H, m), 2.84-2.95 (1H, m), 2.66-2.75 (1H, m), 2.34 (6H, s), 1.40-1.44 (3H, m), 1.31 (3H, d, J=6.1 Hz). ¹⁹F NMR (CHCl₃-d, 376 MHz): δ_{F}−73.1 (s, 3F), −93.4 (d, J=62.1 Hz, 2F).

### Synthesis of A55

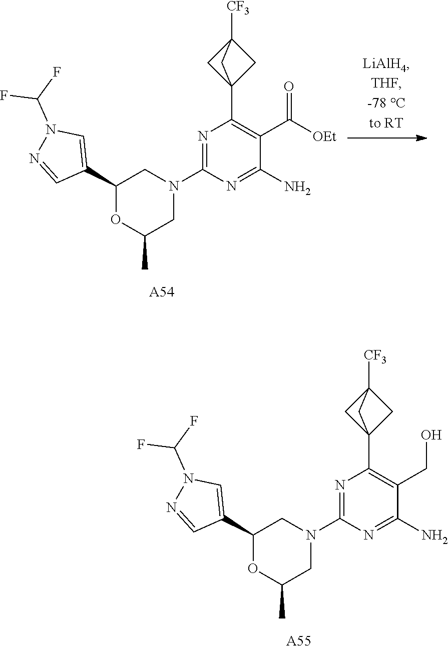

To a flame-dried round-bottom flask equipped with a Teflon-coated magnetic stirring bar was charged ethyl 4-amino-2-[(2S,6R)-2-[1-(difluoromethyl)pyrazol-4-yl]-6-methyl-morpholin-4-yl]-6-[3-(trifluoromethyl)-1-bicyclo[1.1.1]pentanyl]pyrimidine-5-carboxylate (A54, 1.0 eq, 162 mg, 0.31 mmol) and anhydrous THF (4 mL). The flask was sealed, purged under Ar and cooled to −78° C. with stirring. LAH (2.0 M in THF) (3.0 eq, 0.47 mL, 0.94 mmol) was added dropwise to the round-bottom flask. The reaction mixture continued to stir at this temperature, gradually warming to RT overnight. After 18 h, the reaction mixture was diluted with THF (4 mL) and cooled to −78° C. Sat. Na₂SO₄ (aq.) (1 mL) was added carefully and the reaction mixture was stirred at RT for 1 h. The resulting mixture was filtered over a short bed of Celite, washed with EtOAc (35 mL) and the filtrate was concentrated under reduced pressure to yield [4-amino-2-[(2S,6R)-2-[1-(difluoromethyl)pyrazol-4-yl]-6-methyl-morpholin-4-yl]-6-[3-(trifluoromethyl)-1-bicyclo[1.1.1]pentanyl]pyrimidin-5-yl]methanol (A55, 80 mg, 0.17 mmol, 54% yield) as a solid. ESI-MS (m/z)+: 475.3 (obs).

### Synthesis of A56

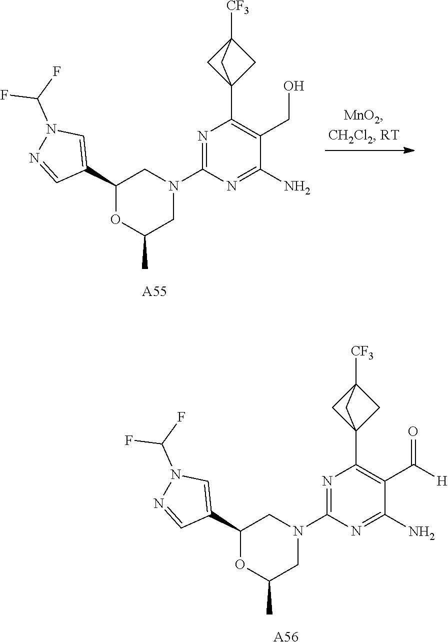

To a flask equipped with a Teflon-coated magnetic stirring bar was added [4-amino-2-[(2S,6R)-2-[1-(difluoromethyl)pyrazol-4-yl]-6-methyl-morpholin-4-yl]-6-[3-(trifluoromethyl)-1-bicyclo[1.1.1]pentanyl]pyrimidin-5-yl]methanol (A55, 1.0 eq, 80 mg, 0.17 mmol) and MnO₂ (15.0 eq, 259 mg, 2.5 mmol). The flask was sealed, purged under Ar and anhydrous DCM (3 mL) was added, and the reaction was stirred overnight for 16 h at RT. The reaction mixture was filtered over a short pad of Celite, washing with DCM:MeOH (9:1). The filtrate was concentrated under reduced pressure to yield 4-amino-2-[(2S,6R)-2-[1-(difluoromethyl)pyrazol-4-yl]-6-methyl-morpholin-4-yl]-6-[3-(trifluoromethyl)-1-bicyclo[1.1.1]pentanyl]pyrimidine-5-carbaldehyde (A56, 76 mg, 0.16 mmol, 95% yield) as a solid. ESI-MS (m/z)+: 473.3 (obs). ¹H NMR (CHCl₃-d, 400 MHz): δ_{H} 10.21 (1H, s), 8.65-8.74 (1H, m), 7.88 (1H, s), 7.71 (1H, s), 7.19 (1H, t, J=62 Hz), 5.47-5.54 (1H, m), 4.71-5.03 (2H, m), 4.56 (1H, dd, J=10.9, 2.6 Hz), 3.71-3.76 (1H, m), 2.86-2.93 (1H, m), 2.70 (1H, dd, J=13.3, 10.7 Hz), 2.42 (6H, s), 1.31 (3H, d, J=6.2 Hz). ¹⁹F NMR (CHCl₃-d, 376 MHz): δ_{F}−73.1 (s, 3F), −93.4 (d, J=62.1 Hz, 2F).

### Synthesis of Example 66

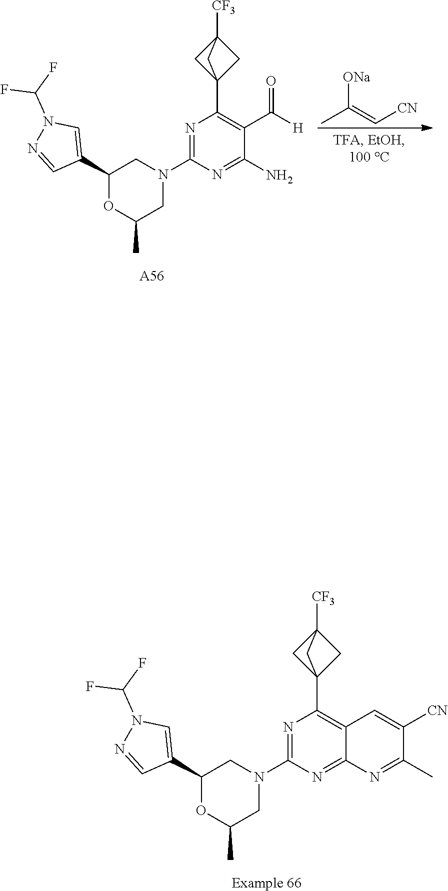

To a microwave vial equipped with a Teflon-coated magnetic stirring bar was added [(Z)-2-cyano-1-methyl-vinyloxy]sodium (3.0 eq, 41 mg, 0.38 mmol) and ethanol (1 mL), and the resulting mixture was stirred at RT for 5 min. TFA (4.0 eq, 40 μL, 0.515 mmol) was then added, followed by 4-amino-2-[(2S,6R)-2-[1-(difluoromethyl)pyrazol-4-yl]-6-methyl-morpholin-4-yl]-6-[3-(trifluoromethyl)-1-bicyclo[1.1.1]pentanyl]pyrimidine-5-carbaldehyde (A56, 1.0 eq, 76 mg, 0.13 mmol). The reaction mixture was sealed and stirred at 100° C. for 16 h. After cooling down to RT, the reaction mixture was poured into a mixture of sat. NaHCO₃(aq.) (10 mL) and EtOAc (15 mL). The aqueous and organic layers were partitioned, and the aqueous layer was extracted with EtOAc (3×15 mL). The combined organic extracts were washed with brine, dried over Na₂SO₄, and filtered. The filtrate was concentrated under reduced pressure and purified by silica gel flash chromatography (1-40% EtOAc in Hexanes) to yield 2-[(2S,6R)-2-[1-(difluoromethyl)pyrazol-4-yl]-6-methyl-morpholin-4-yl]-7-methyl-4-[3-(trifluoromethyl)-1-bicyclo[1.1.1]pentanyl]pyrido[2,3-d]pyrimidine-6-carbonitrile (Example 66, 24 mg, 0.045 mmol, 35% yield) as a solid. ESI-MS (m/z)+: 520.3 (obs). ¹H NMR (CHCl₃-d, 400 MHz): δ_{H} 8.51 (1H, s), 7.90 (1H, s), 7.72-7.75 (1H, m), 7.24 (1H, td, J=62.1, 8.2 Hz), 4.91-5.27 (2H, m), 4.60-4.66 (1H, m), 3.77-3.85 (1H, m), 3.01-3.12 (1H, m), 2.83-2.96 (3H, m), 2.59-2.62 (6H, m), 1.35 (4H, dd, J=7.4, 5.9 Hz). ¹⁹F NMR (CHCl₃-d, 376 MHz): δ_{F}−73.1 (s, 3F), −93.4 (d, J=62.1 Hz, 2F).

### Method 30: Synthesis of Example 71 and 70

### Synthesis of A57

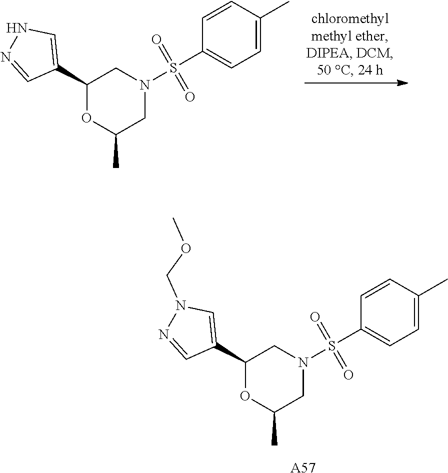

Chloromethyl methyl ether (1.10 eq, 0.13 mL, 1.71 mmol) was added dropwise to a solution of (2R,6S)-2-methyl-4-(p-tolylsulfonyl)-6-(1H-pyrazol-4-yl)morpholine (1.0 eq, 500 mg, 1.56 mmol) and DIPEA (1.2 eq, 0.33 mL, 1.87 mmol) in DCM (2 mL) at 0° C. and the reaction mixture was stirred for 2 h at 50° C. The reaction was quenched by addition of 1 N HCl (10 mL) and the reaction mixture was extracted with DCM (3×10 mL). The combined organic phases were washed with brine, dried over MgSO₄ and filtered. The filtrate was concentrated under reduced pressure and purified by silica gel column chromatography (0-100% EtOAc in hexanes) to afford (2S,6R)-2-(1-(2,2-difluoroethyl)-1H-pyrazol-4-yl)-6-methyl-4-tosylmorpholine (A57, 497 mg, 1.36 mmol, 87% yield) as a foam. ¹H NMR (CHCl₃-d, 400 MHz): δ_{H} 7.62 (2H, d, J=8.1 Hz), 7.49 (2H, d, J=9.4 Hz), 7.34 (2H, d, J=8.0 Hz), 5.31 (2H, s), 4.62-4.68 (1H, m), 3.80-3.87 (1H, m), 3.74 (1H, d, J=11.5 Hz), 3.62 (1H, d, J=11.4 Hz), 3.30 (3H, s), 2.43 (3H, s), 2.22 (1H, t, J=11.0 Hz), 2.01-2.06 (1H, m), 1.18 (3H, d, J=6.3 Hz). ESI-MS (m/z⁺): 366.3 (obs).

### Synthesis of A58

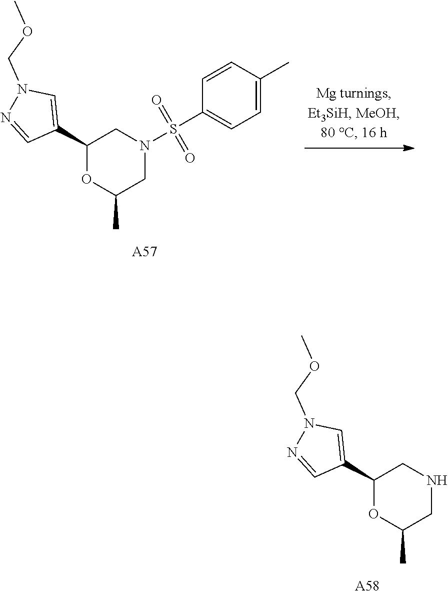

A flask was charged with (2S,6R)-2-(1-(2,2-difluoroethyl)-1H-pyrazol-4-yl)-6-methyl-4-tosylmorpholine (A57, 1.0 eq, 0.50 g, 1.37 mmol), triethylsilane (30.0 eq, 6.6 mL, 41.0 mmol) and Mg turnings (30.0 eq, 998 mg, 41.0 mmol). The reaction flask was equipped with a reflux condenser and then heated at 80° C. for 16 h. The resulting mixture was filtered over a short pad of Celite and rinsed with DCM (50 mL) and MeOH (50 mL). The filtrate was concentrated under reduced pressure to yield a solid containing magnesium residue. The solid was dissolved in 1M HCl (50 mL) and DCM (50 mL) was added. The pH of the aqueous phase was adjusted to pH=14 using 10% NaOH and the organic phase was collected. The aqueous phase was extracted with EtOAc (3×50 mL) and the combined organic phases were washed with brine, dried over MgSO₄, and filtered. The filtrate was concentrated under reduced pressure to afford (2S,6R)-2-(1-(methoxymethyl)-1H-pyrazol-4-yl)-6-methylmorpholine (A58, 170 mg, 0.80 mmol, 59% yield) as an oil. ¹H NMR (CHCl₃-d, 400 MHz): δ_{H} 7.53 (1H, d, J=8.6 Hz), 5.29-5.33 (2H, m), 4.52-4.55 (1H, m), 3.73 (1H, d, J=9.2 Hz), 3.31 (2H, d, J=4.0 Hz), 3.04 (1H, d, J=12.4 Hz), 2.91 (1H, d, J=12.5 Hz), 2.77 (1H, t, J=11.6 Hz), 2.54 (1H, t, J=11.4 Hz), 2.22 (1H, s), 1.17-1.21 (2H, m).

### Synthesis of A59

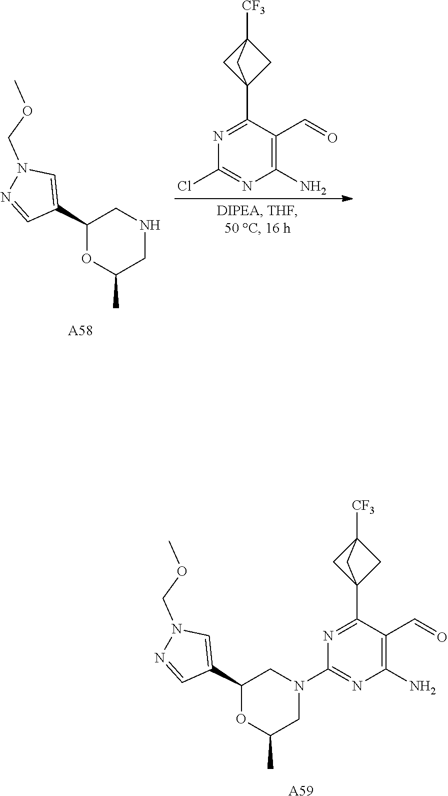

A vial was charged with (2S,6R)-2-(1-(methoxymethyl)-1H-pyrazol-4-yl)-6-methyl-morpholine (A58, 1.1 eq, 80 mg, 0.38 mmol), 4-amino-2-chloro-6-[3-(trifluoromethyl)-1-bicyclo[1.1.1]pentanyl]-pyrimidine-5-carbaldehyde (1.0 eq, 100 mg, 0.34 mmol), DIPEA (3.0 eq, 0.18 mL, 1.0 mmol) and THF (1.65 mL). The vial sealed with a screw cap with pressure relief septum and the reaction was stirred at 50° C. for 16 h. The reaction was cooled down to room temperature and diluted with DCM (20 mL) and 1N HCl (20 mL). The organic phase was collected, and the aqueous phase was extracted with DCM (3×20 mL). The combined organic phases were washed with brine, dried over MgSO₄ and concentrated under reduced pressure. The residue was purified by silica gel column chromatography (0-40% EtOAc in Hexanes) to afford 4-amino-2-((2S,6R)-2-(1-(methoxymethyl)-1H-pyrazol-4-yl)-6-methylmorpholino)-6-(3-(trifluoromethyl)bicyclo[1.1.1]pentan-1-yl)pyrimidine-5-carbaldehyde (A59, 165 mg, 0.29 mmol, 85% yield). ESI-MS (m/z⁺): 467.4 (obs).

### Synthesis of Example 71

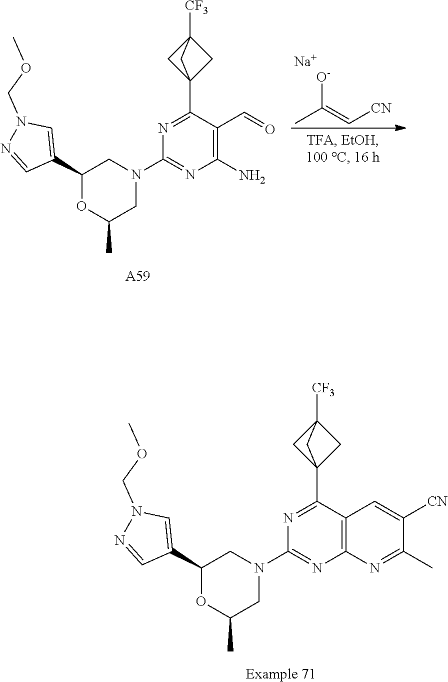

To a microwave vial equipped with a Teflon-coated magnetic stirring bar was sodium (Z)-1-cyanoprop-1-en-2-olate (3.0 eq, 41 mg, 0.39 mmol) and ethanol (1.0 mL), and the reaction was stirred at room temperature. Trifluoroacetic acid (4.0 eq, 40 μL, 0.52 mmol) was added and the reaction mixture was stirred at room temperature for 5 min. 4-amino-2-[(2S,6R)-2-[1-(methoxymethyl)-pyrazol-4-yl]-6-methyl-morpholin-4-yl]-6-[3-(trifluoromethyl)-1-bicyclo[1.1.1]pentanyl]pyrimidine-5-carbaldehyde (A59, 1.0 eq, 75 mg, 0.13 mmol) was added and the reaction mixture was stirred at 100° C. overnight. The reaction was cooled down to room temperature, diluted with EtOAc (20 mL) and quenched with NaHCO₃ (20 mL). The organic phase was collected, and the aqueous phase was extracted with EtOAc (3×20 mL). The combined organic phases were washed with brine, dried over MgSO₄, filtered, and concentrated under reduced pressure. The residue was purified by silica gel column chromatography (0-100% EtOAc in hexanes), followed by reversed-phase column chromatography (5-9% ACN in water (0.1% formic acid)), to afford (2-((2S,6R)-2-(1-(methoxymethyl)-1H-pyrazol-4-yl)-6-methylmorpholino)-7-methyl-4-(3-(trifluoromethyl)bicyclo[1.1.1]pentan-1-yl)pyrido[2,3-d]pyrimidine-6-carbonitrile (Example 71, 28 mg, 0.05 mmol, 42% yield). ESI-MS (m/z⁺): 514.3 (obs). ¹H NMR (DMSO-d₆, 400 MHz): δ_{H} 9.01 (1H, s), 7.97 (1H, d, J=4.8 Hz), 7.60 (1H, s), 5.35 (2H, s), 4.75-4.92 (2H, m), 4.55 (1H, d, J=10.7 Hz), 3.70-3.74 (1H, m), 3.23 (3H, t, J=6.2 Hz), 3.02-3.14 (1H, m), 2.76-2.87 (1H, m), 2.69 (3H, d, J=4.7 Hz), 2.62 (6H, d, J=9.7 Hz), 1.22 (3H, d, J=6.1 Hz). ¹⁹F NMR (DMSO-d₆, 376 MHz): δ_{F}−71.3 (3F, s).

An analogous method was followed to obtain the following compound.

| Ex # | Structure | Starting Material | Characterization |
|---|---|---|---|
| 70 | 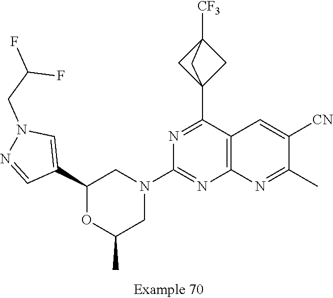 [SMILES: Cc1nc2nc(N3C[C@@H](C)O[C@@H](c4cnn(CC(F)F)c4)C3)nc(C34CC(C(F)(F)F)(C3)C4)c2cc1C#N] | (2R,6S)-2-methyl-6- (1H-pyrazol-4-yl)-4- tosyl-morpholine; 2,2-difluoroethyl trifluoromethane- sulfonate | 2-((2S,6R)-2-(1-(2,2-difluoroethyl)- 1H-pyrazol-4-yl)-6-methyl- morpholino)-7-methyl-4-(3- (trifluoromethyl)bicyclo[1.1.1]pentan- 1-yl)pyrido[2,3-d]pyrimidine-6- carbonitrile (Example 70). ¹H NMR (DMSO-d₆, 400 MHz): δ_{H} 9.01 (1H, s), 7.85 (1H, s), 7.58 (1H, s), 6.18- 6.46 (1H, m), 4.75-4.91 (2H, m), 4.53-4.64 (3H, m), 3.72 (1H, dd, J = 10.0, 6.2 Hz), 3.00-3.12 (1H, m), 2.74-2.85 (1H, m), 2.69 (3H, s), 2.62 (6H, d, J = 10.1 Hz), 1.22 (3H, d, J = 6.0 Hz). ESI-MS (m/z⁺): 534.3. |

### Method 31: Synthesis of Example 72

### Synthesis of A60

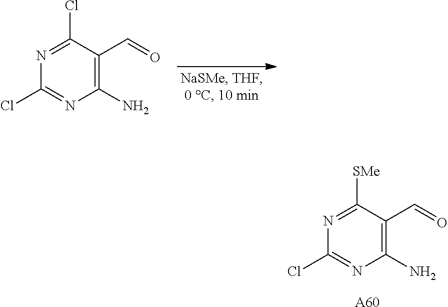

To a solution of 4-amino-2,6-dichloro-pyrimidine-5-carbaldehyde (1.0 eq, 3.67 g, 19.1 mmol) in THF (20 mL) was added sodium thiomethoxide (1.0 eq, 1.34 g, 19.1 mmol), and the reaction mixture was stirred at 0° C. for 10 min. Water was added and the mixture was filtered through a Buchner funnel. The funnel was rinsed with water and heptanes, and the recovered solid was dried under high vacuum overnight to afford 4-amino-2-chloro-6-methylsulfanyl-pyrimidine-5-carbaldehyde (A60, 3.18 g, 15.6 mmol, 82% yield) as a solid (mixture of the two regioisomers). ESI-MS (m/z)+: 204.0 (obs).

### Synthesis of A61

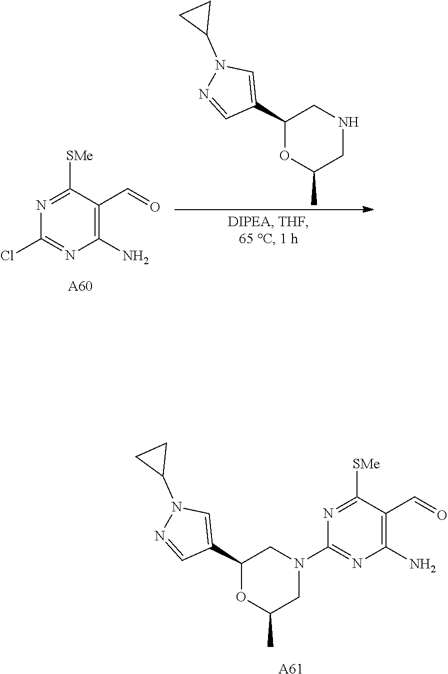

A stirred mixture of 4-amino-2-chloro-6-methylsulfanyl-pyrimidine-5-carbaldehyde (A60, 1.0 eq, 2.26 g, 11.1 mmol), (2S,6R)-2-(1-cyclopropylpyrazol-4-yl)-6-methyl-morpholine (1.0 eq, 2.30 g, 11.1 mmol) in anhydrous THF (50 mL) and DIPEA (1.0 eq, 1.9 mL, 11.1 mmol) was stirred at 70° C. for 1 h. The reaction was cooled down to room temperature and concentrated under reduced pressure. The residue was dissolved in EtOAc (150 mL), washed with 0.5 M HCl (aq), and brine. The aqueous layer was extracted with EtOAc (50 mL) and the combined organic layers were dried over Na₂SO₄, and filtered. The filtrate was concentrated under reduced pressure and purified by silica gel flash chromatography (30-70% EtOAc in hexanes) to afford 4-amino-6-methylsulfanyl-2-[(2S,6R)-2-(1-cyclopropylpyrazol-4-yl)-6-methylmorpholin-4-yl]pyrimidine-5-carbaldehyde (A61, 1.96 g, 5.23 mmol, 47% yield) as a solid. ESI-MS (m/z)+: 375.2 (obs).

### Synthesis of A62

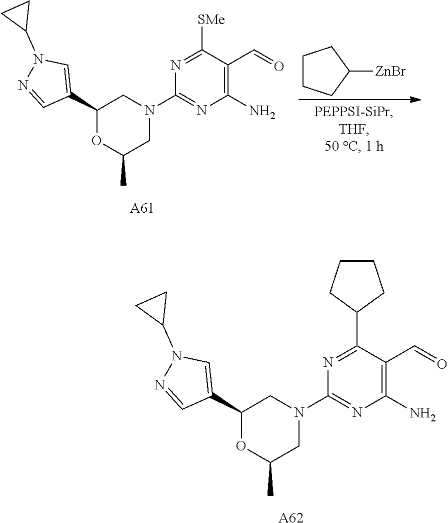

4-amino-6-methylsulfanyl-2-[(2S,6R)-2-(1-cyclopropylpyrazol-4-yl)-6-methyl-morpholin-4-yl]pyrimidine-5-carbaldehyde (A61, 1.0 eq, 623 mg, 1.66 mmol) was dissolved in THF (14 mL) and the solution was bubbled with argon for 5 min. PEPPSI-SIPr (0.1 eq, 113 mg, 0.17 mmol) was added followed by dropwise addition of bromo(cyclopentyl)zinc (2.0 eq, 6.7 mL, 3.33 mmol). The resulting mixture was stirred at 50° C. for 1 h. The reaction was quenched with sat. NaHCO₃(aq.) and extracted with EtOAc (×3). The combined organic layers were washed with brine, dried over Na₂SO₄, filtered, and concentrated under reduced pressure. The residue was purified by normal phase column chromatography (20-80% EtOAc in hexanes) and reversed-phase column chromatography (25-80% MeCN in water (0.1% formic acid)) to afford 4-amino-6-cyclopentyl-2-[(2S,6R)-2-(1-cyclopropylpyrazol-4-yl)-6-methyl-morpholin-4-yl]pyrimidine-5-carbaldehyde (A62, 62 mg, 0.16 mmol, 9%) as a solid. ESI-MS (m/z)+: 397.4 (obs).

### Synthesis of Example 72

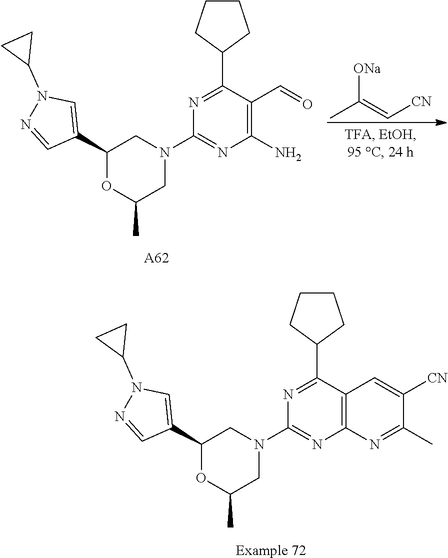

To a microwave vial were added 4-amino-6-cyclopentyl-2-[(2S,6R)-2-(1-cyclopropyl-pyrazol-4-yl)-6-methylmorpholin-4-yl]pyrimidine-5-carbaldehyde (A62, 1.0 eq, 62 mg, 0.16 mmol) and [(Z)-2-cyano-1-methyl-vinyloxy]sodium (3.0 eq, 49 mg, 0.47 mmol) in EtOH (1 mL). The vial was purged with argon, sealed and TFA (1.0 eq, 0.012 mL, 0.16 mmol) was added. The reaction was stirred at 95° C. for 24 h. The reaction was quenched with water and extracted with DCM (×3). The combined organic layers were washed with brine, dried over Na₂SO₄, filtered and concentrated under reduced pressure. The residue was purified by normal phase column chromatography (50-100% EtOAc in hexanes), followed by reversed-phase column chromatography (25-75% MeCN in water (0.1% formic acid)) to afford 4-cyclopentyl-2-[(2S,6R)-2-(1-cyclopropyl-pyrazol-4-yl)-6-methyl-morpholin-4-yl]-7-methyl-pyrido[2,3-d]pyrimidine-6-carbonitrile (Example 72, 25 mg, 0.056 mmol, 36% yield) as a solid.

¹H NMR (CHCl₃-d, 400 MHz): δ_{H} 8.42 (1H, s), 7.51-7.53 (2H, m), 4.93-5.19 (2H, m), 4.54 (1H, d, J=10.3 Hz), 3.69-3.79 (2H, m), 3.57 (1H, s), 3.04 (1H, q, J=11.7 Hz), 2.83 (4H, s), 1.93-2.04 (4H, br m), 1.79 (4H, br s), 1.30 (3H, t, J=7.6 Hz), 1.11 (2H, br s), 1.00 (2H, s). ESI-MS (m/z)+: 444.4 (obs).

### Method 32: Synthesis of Example 73

### Synthesis of A63

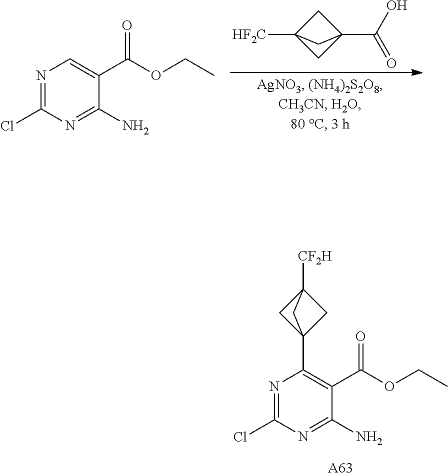

To a flask equipped with a Teflon-coated magnetic stir bar was added ethyl 4-amino-2-chloro-pyrimidine-5-carboxylate (1.0 eq, 460 mg, 2.28 mmol), 3-(difluoromethyl)bicyclo[1.1.1]pentane-1-carboxylic acid (1.3 eq, 481 mg, 2.97 mmol) and AgNO₃ (1.3 eq, 501 mg, 2.97 mmol). MeCN (8 mL) and water (4 mL) were added, and the reaction mixture was vigorously stirred at 80° C. After 5 min, ammonium persulfate (3.3 eq, 1.72 g, 7.53 mmol) was added. The reaction mixture was fitted with a reflux condenser and stirred at 80° C. for 3 h. The reaction mixture was cooled to room temperature and concentrated under reduced pressure. The residue was diluted with EtOAc (150 mL), sat. NaHCO₃ (50 mL) and brine (50 mL), filtered over a short pad of celite, and the layers were separated. The organic layer was collected, washed with brine, dried over Na₂SO₄, and filtered. The filtrate was concentrated under reduced pressure and purified by silica gel column chromatography (5-25% EtOAc in hexanes) to afford ethyl 4-amino-2-chloro-6-(3-(difluoromethyl)bicyclo[1.1.1]pentan-1-yl)pyrimidine-5-carboxylate (A63, 0.31 g, 0.97 mmol, 42% yield) as a solid. ESI-MS (m/z⁺): 318.1 (obs).

### Synthesis of A64

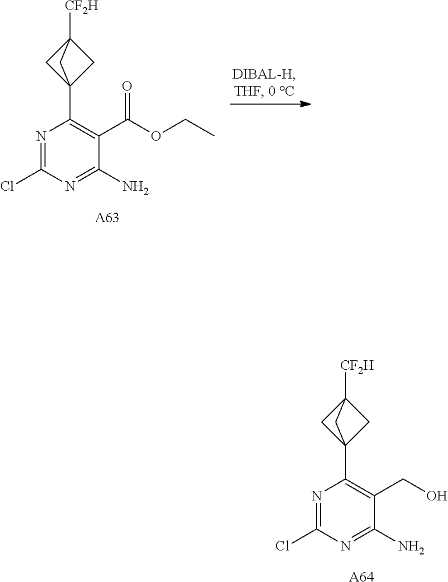

To a stirring solution of ethyl 4-amino-2-chloro-6-(3-(difluoromethyl)bicyclo[1.1.1]pentan-1-yl)pyrimidine-5-carboxylate (A63, 1.0 eq, 287 mg, 0.90 mmol) in THF (4.5 mL) at 0° C. was slowly added DIBAL-H (25% in toluene, 3.0 eq, 1.6 mL, 2.71 mmol) and the reaction mixture was stirred in an ice-bath for 1 h. The reaction was quenched by reverse addition in sodium potassium tartrate (10% in water, 50 mL), EtOAc (50 mL) was added, and the reaction was stirred at room temperature for 1 h. The organic phase was collected, and the aqueous phase was extracted with EtOAc (3×50 mL). The combined organic layers were washed with NH₄Cl (100 mL), brine, dried over MgSO₄, and filtered. The filtrate was concentrated under reduced pressure to afford (4-amino-2-chloro-6-(3-(difluoromethyl)-bicyclo[1.1.1]pentan-1-yl)pyrimidin-5-yl)methanol (A64, 186 mg, 0.68 mmol, 75% yield). ESI-MS (m/z⁺): 276.2 (obs).

### Synthesis of A65

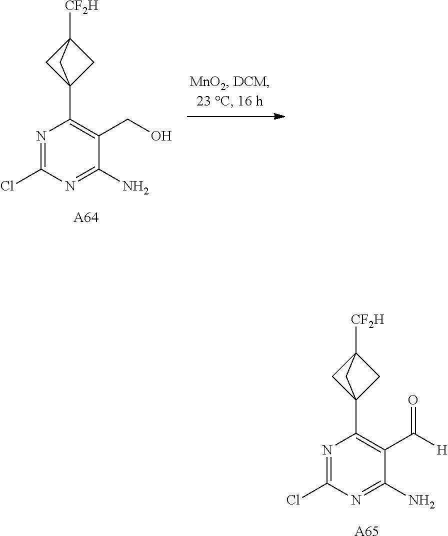

In a flask equipped with a Teflon-coated magnetic stirring bar, was added 4-amino-2-chloro-6-(3-(difluoromethyl)bicyclo[1.1.1]pentan-1-yl)pyrimidin-5-yl)methanol (A64, 1.0 eq, 186 mg, 0.68 mmol) and MnO₂ (15.0 eq, 880 mg, 10.1 mmol). The flask was purged under argon, anhydrous DCM (16 mL) was added and the reaction mixture was stirred overnight for 16 h at room temperature. The reaction mixture was filtered through a Celite pad, rinsed with DCM (30 mL), and concentrated under reduced pressure to afford 4-amino-2-chloro-6-(3-(difluoromethyl)bicyclo[1.1.1]pentan-1-yl)pyrimidine-5-carbaldehyde (A65, 184 mg, 0.67 mmol, 99% yield) as an oil. ESI-MS (m/z⁺): 274.2 (obs).

### Synthesis of A66

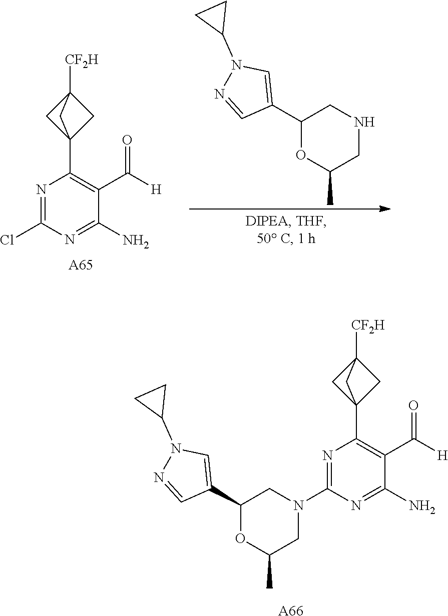

A vial was filled with (6R)-2-(1-cyclopropyl-1H-pyrazol-4-yl)-6-methylmorpholine (2.5 eq, 95 mg, 0.46 mmol), 4-amino-2-chloro-6-(3-(difluoromethyl)bicyclo[1.1.1]pentan-1-yl)pyrimidine-5-carbaldehyde (A65, 1.0 eq, 50 mg, 0.18 mmol), DIPEA (3.0 eq, 0.1 mL, 0.55 mmol) and THF (1.8 mL). The vial was sealed with a screw cap with pressure relief septum and the reaction was stirred at 50° C. for 1 h. The reaction was cooled to room temperature and diluted with DCM (20 mL) and 1N HCl (20 mL). The organic phase was collected, and the aqueous phase was extracted with DCM (3×20 mL). The combined organic phases were washed with brine, dried over MgSO₄ and filtered. The filtrate was concentrated under reduced pressure and purified by silica gel column chromatography (0-40% EtOAc in hexanes) to afford 4-amino-2-((2S,6R)-2-(1-cyclopropyl-1H-pyrazol-4-yl)-6-methylmorpholino)-6-(3-(difluoromethyl)bicyclo[1.1.1]pentan-1-yl)pyrimidine-5-carbaldehyde (A66, 80 mg, 0.16 mmol, 85% yield). ESI-MS (m/z⁺): 445.4 (obs).

### Synthesis of Example 73

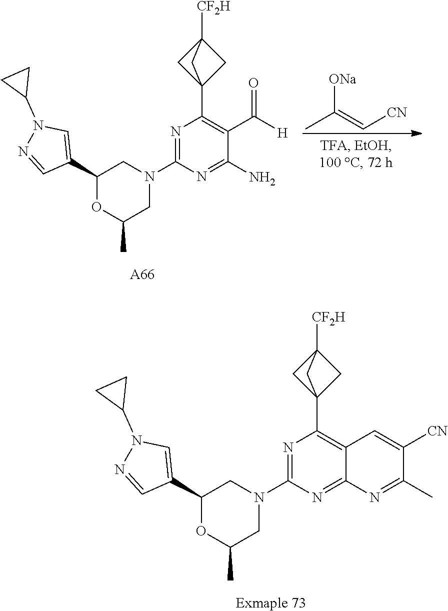

[(Z)-2-cyano-1-methyl-vinyloxy]sodium (3.0 eq, 46 mg, 0.44 mmol) and EtOH (1.0 mL) were added to a microwave vial equipped with a magnetic stirring bar. The mixture was stirred at room temperature for 5 min then TFA (4.0 eq, 45 μL, 0.59 mmol) was added. After a few minutes, 4-amino-2-((2S,6R)-2-(1-cyclopropyl-1H-pyrazol-4-yl)-6-methylmorpholino)-6-(3-(difluoromethyl)-bicyclo[1.1.1]pentan-1-yl)pyrimidine-5-carbaldehyde (A66, 1.0 eq, 68 mg, 0.15 mmol) was added and the reaction mixture was stirred at 100° C. over the weekend. The reaction mixture was cooled to room temperature and diluted with EtOAc (50 mL), washed with sat. NH₄Cl (50 mL), sat. NaHCO₃ (50 mL), dried over MgSO₄, and filtered. The filtrate was concentrated under reduced pressure and purified by silica gel column chromatography (5-100% EtOAc in hexanes) to afford 2-((2S,6R)-2-(1-cyclopropyl-1H-pyrazol-4-yl)-6-methylmorpholino)-4-(3-(difluoromethyl)bicyclo[1.1.1]pentan-1-yl)-7-methylpyrido[2,3-d]pyrimidine-6-carbonitrile (Example 73, 30 mg, 0.06 mmol, 41% yield). ESI-MS (m/z⁺): 492.3 (obs).

¹H NMR (DMSO-d₆, 400 MHz): δ_{H} 8.96 (1H, s), 7.83 (1H, s), 7.45 (1H, s), 6.00-6.28 (1H, m), 4.75-4.89 (2H, m), 4.47-4.50 (1H, m), 3.66-3.72 (2H, m), 2.97-3.14 (1H, m), 2.72-2.85 (1H, m), 2.69 (3H, s), 2.44 (6H, d, J=11.7 Hz), 1.20 (3H, d, J=6.2 Hz), 0.98-1.03 (2H, m), 0.90-0.97 (2H, m). ¹⁹F NMR (DMSO-d₆, 376 MHz): δ_{F}−122.3 (2F, d, J 48.0 Hz).

### Method 33: Synthesis of Example 74

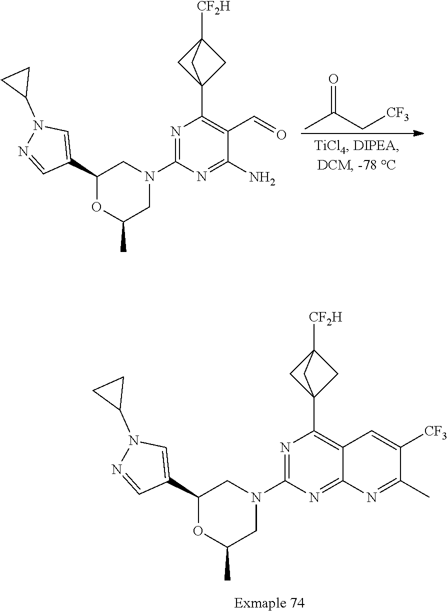

TiCl₄ (3.0 eq, 0.16 mL, 1.5 mmol) was added to a solution of 4,4,4-trifluorobutan-2-one (3.0 eq, 0.23 mL, 1.5 mmol) in anhydrous DCM (3 mL) at −78° C., and the reaction mixture was warmed to 0° C. and stirred for 30 min. The mixture was then cooled to −78° C. and DIPEA (4.0 eq, 0.35 mL, 2.0 mmol) was added dropwise. The mixture was warmed to 0° C. and stirred for 30 min. The mixture was cooled to −45° C. and a solution of 4-amino-2-[rac-(2S,6R)-2-(1-cyclopropylpyrazol-4-yl)-6-methylmorpholin-4-yl]-6-[3-(trifluoromethyl)-1-bicyclo[1.1.1]pentanyl]pyrimidine-5-carbaldehyde (1.0 eq, 231 mg, 0.50 mmol) in anhydrous DCM (3 mL) was added to the reaction mixture. The reaction mixture was gradually warmed to 0° C. over the course of 1 h and stirred at this temp for an additional h. The reaction mixture was quenched with sat. NH₄Cl (aq) and diluted with DCM and H₂O. The layers were separated, and the aqueous layer was extracted with DCM (×2). The combined organic extracts were dried over anhydrous MgSO₄, filtered, and concentrated under reduced pressure. The residue was purified by silica gel flash-column chromatography (10-50% Acetone in Hexanes), followed by HPLC (55-75% MeCN in 10 mM ammonium formate (aq) (pH=3.8)) to afford (2S,6R)-2-(1-cyclopropyl-1H-pyrazol-4-yl)-6-methyl-4-(7-methyl-6-(trifluoromethyl)-4-(3-(trifluoromethyl)-bicyclo[1.1.1]pentan-1-yl)pyrido[2,3-d]pyrimidin-2-yl)morpholine (Example 74, 50 mg, 0.09 mmol, 69% yield) as a solid. ¹H NMR (CHCl₃-d, 400 MHz): δ_{H} 8.48 (1H, s), 7.54 (2H, s), 4.88-5.19 (2H, m), 4.56 (1H, d, J=10.8 Hz), 3.74-3.82 (1H, m), 3.56-3.61 (1H, m), 3.02-3.09 (1H, m), 2.79-2.84 (4H, m), 2.58 (6H, d, J=8.0 Hz), 1.33 (3H, s), 1.13 (2H, s), 1.03 (2H, d, J=7.0 Hz). ¹⁹F NMR (CHCl₃-d, 376 MHz): δ_{F}−61.6 (3F, s), −73.1 (3F, s). ESI-MS (m/z)+: 553.4 (obs).

An analogous method was followed to obtain the following compounds.

| Ex # | Structure | Starting Materials | Characterization |
|---|---|---|---|
| 90 | 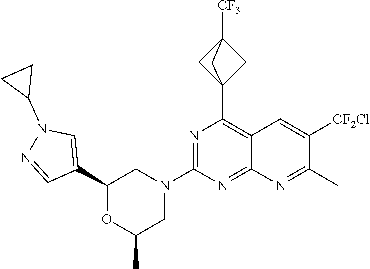 | 4-amino-2-[rac- (2S,6R)-2-(1- cyclopropylpyrazol-4- yl)-6-methylmorpholin- 4-yl]-6-[3- (trifluoromethyl)-1- bicyclo[1.1.1]pentanyl]- pyrimidine-5- carbaldehyde; 4,4,4-trifluorobutan-2- one | (2S,6R)-4-(6-(chloro- difluoromethyl)-7-methyl-4-(3- (trifluoromethyl)- bicyclo[1.1.1]pentan-1- yl)pyrido[2,3-d]pyrimidin-2-yl)- 2-(1-cyclopropyl-1H-pyrazol-4- yl)-6-methylmorpholine (Example 90). ESI-MS (m/z)+: 569.3. ¹H NMR (CHCl₃-d, 400 MHz): δ_{H} 8.47 (1H, s), 7.54 (2H, s), 5.12 (1H, dd, J = 50.1, 13.6 Hz), 4.95 (1H, dd, J = 38.1, 13.4 Hz), 4.56 (1H, d, J = 10.7 Hz), 3.73-3.81 (1H, m), 3.55-3.60 (1H, m), 3.02-3.10 (1H, m), 2.92 (3H, s), 2.83 (1H, t, J = 12.1 Hz), 2.58 (6H, d, J = 9.4 Hz), 1.32 |
| | Example 90 | | (3H, s), 1.12-1.14 (2H, m), 1.02- |
| | | | 1.04 (2H, m). ¹⁹F NMR (CHCl₃- |
| | | | d, 376 MHz): δ_{F} −48.3 (2F, s), |
| | | | −73.1 (3F, s). |

### Method 34: Synthesis of Example 91

### Synthesis of A68

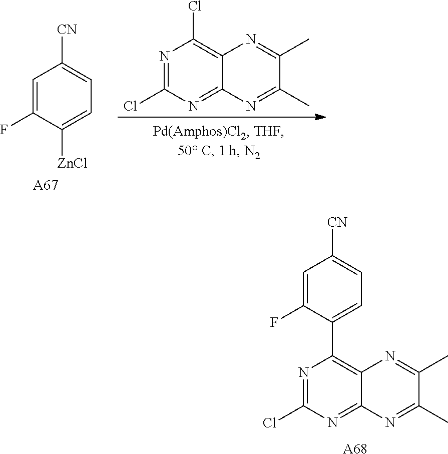

To a mixture of 2,4-dichloro-6,7-dimethyl-pteridine (1.00 eq, 200 mg, 0.873 mmol) and Pd(amphos)Cl₂ (0.10 eq, 62 mg, 0.0873 mmol) in THF (1 mL) was added chloro-(4-cyano-2-fluoro-phenyl)zinc (2.20 eq, 14 mL, 1.92 mmol) at 15° C. under N₂. The reaction mixture was heated to 50° C. for 1 h. The reaction was quenched by aq. NH₄Cl (20 mL) and H₂O (20 mL), and extracted with EtOAc (3×30 mL). The combined organic layers were dried over anhydrous Na₂SO₄, filtered, concentrated under reduced pressure, and purified by silica gel column chromatography (0-50% EtOAc in Petroleum ether) to afford 4-(2-chloro-6,7-dimethyl-pteridin-4-yl)-3-fluoro-benzonitrile (A68, 130 mg, 0.414 mmol, 47% yield) as a solid. LC/MS (ESI, m/z⁺): [M+H]⁺=314.2. ¹H NMR (400 MHz, CDCl₃) δ=7.93-7.83 (m, 1H), 7.67 (dd, J=1.3, 7.9 Hz, 1H), 7.56 (dd, J=1.3, 9.1 Hz, 1H), 2.87 (s, 3H), 2.74 (s, 3H).

### Synthesis of Example 91

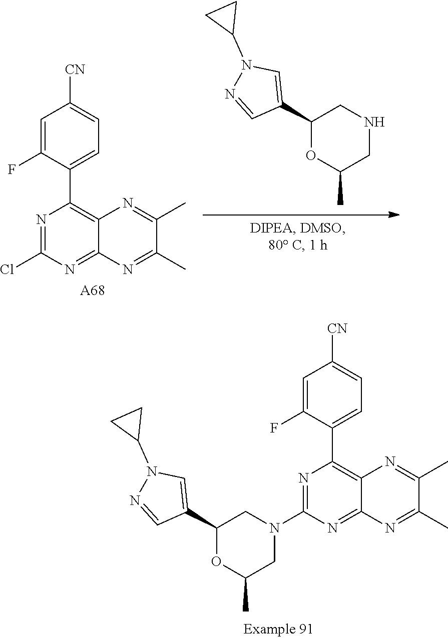

To a solution of (2S,6R)-2-(1-cyclopropylpyrazol-4-yl)-6-methyl-morpholine (1.10 eq, 29 mg, 0.140 mmol) and DIEA (5.00 eq, 0.11 mL, 0.638 mmol) in DMSO (1 mL) was added 4-(2-chloro-6,7-dimethyl-pteridin-4-yl)-3-fluoro-benzonitrile (1.00 eq, 40 mg, 0.128 mmol) in one portion under N₂. Then the mixture was stirred at 80° C. for 2 h under N₂. The reaction was purified by Prep-HPLC (Condition 3, Gradient 1) to afford 4-[2-[(2S,6R)-2-(1-cyclopropylpyrazol-4-yl)-6-methyl-morpholin-4-yl]-6,7-dimethylpteridin-4-yl]-3-fluoro-benzonitrile (Example 91, 35 mg, 0.0704 mmol, 55% yield) as a solid. LC/MS (ESI, m/z⁺): [M+H]⁺=485.3. ¹H NMR (400 MHz, CDCl₃) δ=7.78 (br d, J=7.1 Hz, 1H), 7.61 (d, J=8.0 Hz, 1H), 7.55-7.49 (m, 3H), 5.19-4.80 (m, 2H), 4.64-4.56 (m, 1H), 3.82 (ddd, J=2.2, 6.3, 10.1 Hz, 1H), 3.61-3.52 (m, 1H), 3.08 (dd, J=11.0, 13.4 Hz, 1H), 2.84 (dd, J=10.8, 13.3 Hz, 1H), 2.72 (s, 3H), 2.57 (s, 3H), 1.32 (d, J=6.1 Hz, 3H), 1.17-1.06 (m, 2H), 1.05-0.94 (m, 2H).

An analogous method was followed to obtain the following compounds.

| Ex # | Compound | Starting Material | Characterization |
|---|---|---|---|
| 96 | 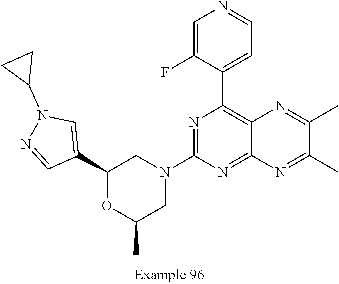 [SMILES: Cc1nc2nc(N3C[C@@H](C)O[C@@H](c4cnn(C5CC5)c4)C3)nc(-c3ccncc3F)c2nc1C] | 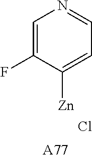 | The residue was purified by prep-HPLC (Condition 3) and lyophilized to give (2S,6R)-2-(1-cyclopropylpyrazol-4- yl)-4-[4-(3-fluoro-4-pyridyl)-6,7-dimethylpteridin-2- yl]-6-methyl-morpholine (Example 96, 87 mg, 0.185 mmol, 77% yield) as a solid. LC/MS (ESI, m/z⁺): 461.2 [M + H]⁺. ¹H NMR (400 MHz, CDCl₃) δ = 8.65 (s, 1H), 8.60 (d, J = 4.8 Hz, 1H), 7.61 (t, J = 5.3 Hz, 1H), 7.57-7.52 (m, 2H), 5.21-4.81 (m, 2H), 4.68-4.55 (m, 1H), 3.92-3.75 (m, 1H), 3.58 (tt, J = 3.7, 7.2 Hz, 1H), 3.09 (dd, J = 10.9, 13.3 Hz, 1H), 2.86 (dd, J = 10.7, 13.3 Hz, 1H), 2.73 (s, 3H), 2.59 (s, 3H), 1.34 (d, J = 6.1 Hz, 3H), 1.15-1.09 (m, 2H), 1.05-0.98 (m, 2H). |
| | | ((3- | |
| | | fluoropyridin- | |
| | | 4-yl)zinc(II) | |
| | | chloride) | |

### Method 35: Synthesis of Example 94

### Synthesis of A71

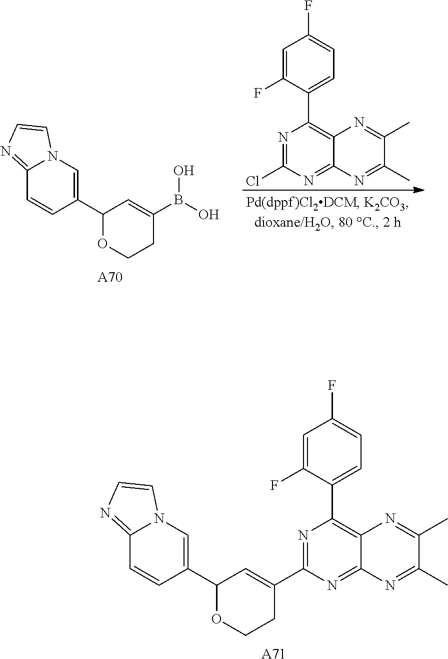

To a solution of 2-chloro-4-(2,4-difluorophenyl)-6,7-dimethyl-pteridine (1.00 eq, 500 mg, 1.50 mmol), (6-imidazo[1,2-a]pyridin-6-yl-3,6-dihydro-2H-pyran-4-yl)boronic acid (1.05 eq, 418 mg, 1.57 mmol) and K₂CO₃ (3.00 eq, 378 mg, 4.50 mmol) in 1,4-Dioxane (10 mL) and Water (1 mL) was added Pd(dppf)Cl₂ DCM (0.10 eq, 110 mg, 0.15 mmol) under N₂. The reaction mixture was stirred at 80° C. for 2 h under N₂ atmosphere. The mixture was poured into H₂O (50 mL) and extracted with EtOAc (3×50 mL). The combined organic layers were dried over anhydrous Na₂SO₄, filtered, concentrated under reduced pressure, and purified by silica gel column chromatography (0-100% EtOAc in PE) to give 4-(2,4-difluorophenyl)-2-(6-imidazo[1,2-a]pyridin-6-yl-3,6-dihydro-2H-pyran-4-yl)-6,7-dimethyl-pteridine (A71, 400 mg, 0.85 mmol, 57% yield) as a solid. LC/MS (ESI, m/z⁺): [M+H]⁺=471.2; ¹H NMR (400 MHz, CDCl₃) δ ppm 2.74 (s, 3H) 2.85 (s, 3H) 3.06 (dt, J=2.85, 1.64 Hz, 2H), 3.94-4.06 (m, 1H), 4.27 (dt, J=11.41, 4.49 Hz, 1H), 5.48 (d, J=2.50 Hz, 1H), 6.95-7.03 (m, 1H), 7.08 (ddd, J=8.82, 6.82, 2.38 Hz, 1H), 7.30 (d, J=1.63 Hz, 1H), 7.57 (s, 1H), 7.61-7.68 (m, 3H) 7.73-7.82 (m, 1H), 8.24 (s, 1H).

### Synthesis of A 72

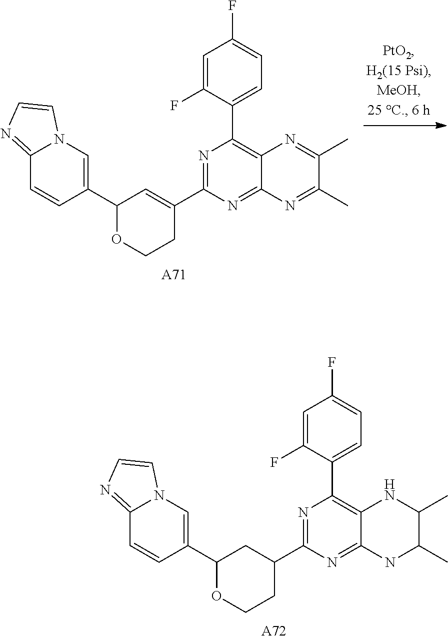

A mixture 4-(2,4-difluorophenyl)-2-(6-imidazo[1,2-a]pyridin-6-yl-3,6-dihydro-2H-pyran-4-yl)-6,7-dimethyl-pteridine (1.00 eq, 200 mg, 0.425 mmol) in Methanol (5 mL) was degassed with N₂ (×3). PtO₂ (0.50 eq, 48 mg, 0.213 mmol) was added. The mixture was degassed with H₂ (×3) and stirred at 25° C. under H₂ (15 psi, balloon) for 6 h. The mixture was filtered through celite and the filtrate was concentrated under reduced pressure to afford crude 4-(2,4-difluorophenyl)-2-(2-imidazo[1,2-a]pyridin-6-yltetrahydropyran-4-yl)-6,7-dimethyl-5,6,7,8-tetrahydropteridine (A72, 190 mg, 0.399 mmol, 94% yield) as an oil. The crude was used for next step directly. LC/MS (ESI, m/z⁺): 477.2 [M+H]⁺.

### Synthesis of Example 94

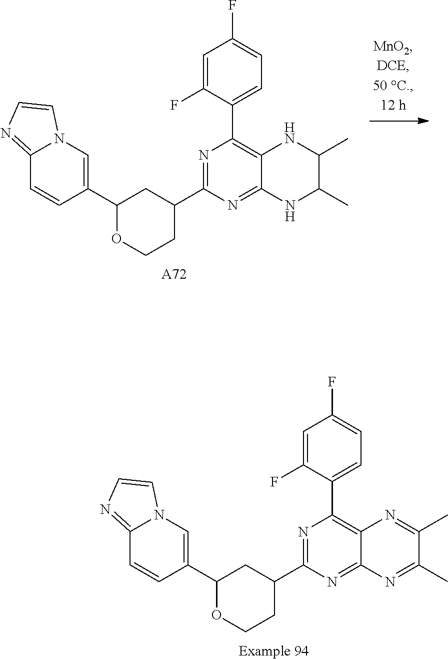

The mixture of 4-(2,4-difluorophenyl)-2-(2-imidazo[1,2-a]pyridin-6-yltetrahydropyran-4-yl)-6,7-dimethyl-5,6,7,8-tetrahydropteridine (1.00 eq, 200 mg, 0.420 mmol), MnO₂ (10.0 eq, 365 mg, 4.20 mmol) in DCE (10 mL) was stirred at 50° C. for 16 h. The reaction mixture was filtered and concentrated under reduced pressure to give a residue. The residue was purified by silica gel column chromatography (50% EtOAc in PE to 6% MeOH in EtOAc) to give a crude product. The crude product was purified by prep-TLC (9% MeOH in DCM), followed by prep-HPLC (Condition 3) and lyophilized to give 4-(2,4-difluorophenyl)-2-(2-imidazo[1,2-a]pyridin-6-yltetrahydropyran-4-yl)-6,7-dimethyl-pteridine (Example 94, 3.2 mg, 0.0067 mmol, 2% yield) as a solid and 4-(2,4-difluorophenyl)-2-(2-imidazo[1,2-a]pyridin-6-yltetrahydropyran-4-yl)-6,7-dimethyl-pteridine (as a mixture of diastereromers, 4.9 mg, 0.0099 mmol, 2.4% yield) as a solid. Example 94 (cis): LC/MS (ESI, m/z⁺): [M+H]⁺=473.2. ¹H NMR (400 MHz, CDCl₃) δ ppm 2.09-2.18 (m, 1H), 2.22-2.32 (m, 2H), 2.57 (br d, J=13.26 Hz, 1H), 2.74 (s, 3H), 2.85 (s, 3H), 3.59-3.73 (m, 1H), 3.89 (td, J=11.66, 2.94 Hz, 1H), 4.34-4.43 (m, 1H), 4.67 (br d, J=10.76 Hz, 1H), 6.98-7.05 (m, 1H), 7.10 (td, J=8.10, 2.44 Hz, 1H), 7.48 (br d, J=8.50 Hz, 1H), 7.63 (br s, 1H), 7.70-7.82 (m, 2H), 7.88-8.00 (m, 1H), 8.38 (br s, 1H). (mixture of diastereomers): LC/MS (ESI, m/z⁺): [M+H]⁺=473.2. ¹H NMR (400 MHz, CDCl₃) δ ppm 2.10-2.16 (m, 1H), 2.23-2.33 (m, 2H), 2.52-2.62 (m, 1H), 2.74 (s, 3H) 2.85 (s, 3H) 3.54-3.74 (m, 1H), 3.89 (td, J=11.60, 3.06 Hz, 1H), 4.28-4.45 (m, 1H), 4.66 (br d, J=10.76 Hz, 1H), 7.01 (td, J=9.41, 2.31 Hz, 1H), 7.10 (td, J=8.16, 1.94 Hz, 1H), 7.42-7.49 (m, 1H), 7.58-7.66 (m, 1H), 7.69-7.79 (m, 2H), 7.92 (br d, J=8.13 Hz, 1H), 8.37 (br s, 1H).

An analogous method was followed to obtain the following compounds.

| Ex # | Compound | Starting Material | Characterization |
|---|---|---|---|
| 113 | 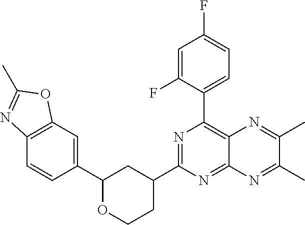 | 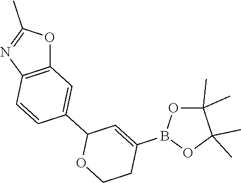 | Modification: Step 2 was stirred at 0 °C for 3 h. Step 3 was stirred at 50 ºC for 24 h. The residue was purified by prep- HPLC (Condition 5), followed by Prep-TLC (91% EtOAc in PE) and lyophilized to afford 6-[4-[4-(2,4- difluorophenyl)-6,7-dimethyl- pteridin-2-yl]tetrahydropyran-2-yl]- 2-methyl-1,3-benzoxazole (Example 113, cis, 74 mg, 0.15 mmol, 37% yield) as a solid. LC/MS (ESI, m/z⁺): 488.2 [M + H]⁺. |
| | Example 113 | A84 | ¹H NMR (400 MHz, CDCl₃) δ = |
| | | | 7.75 (td, J = 7.1, 8.5 Hz, 1H), 7.63- |
| | | | 7.56 (m, 2H), 7.38-7.33 (m, 1H), |
| | | | 7.12-7.05 (m, 1H), 7.00 (dt, J = 2.4, |
| | | | 9.4 Hz, 1H), 4.68 (dd, J = 1.8, |
| | | | 11.3 Hz, 1H), 4.44-4.34 (m, 1H), 3.88 |
| | | | (dt, J = 2.9, 11.7 Hz, 1H), 3.64 (tt, J = |
| | | | 3.9, 11.8 Hz, 1H), 2.84 (s, 3H), 2.73 |
| | | | (s, 3H), 2.64 (s, 3H), 2.47 (td, J = 1.8, |
| | | | 13.3 Hz, 1H), 2.36-2.10 (m, 3H). |
| | | | Note: The product is unstable in |
| | | | acidic condition. |
| 115 | 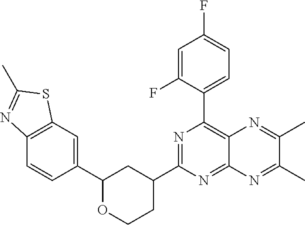 | 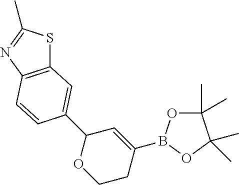 | Modification: Step 2 was stirred at 20 °C. for 24 h. The residue was purified by Prep- HPLC (Condition 3) and lyophilized to afford 6-[4-[4-(2,4- difluorophenyl)-6,7-dimethyl- pteridin-2-yl]tetrahydropyran-2-yl]- 2-methyl-1,3-benzothiazole (Example 115, 102 mg, 0.20 mmol, 54% yield) as a solid. LC/MS (ESI, m/z⁺): [M + H]⁺ = 504.1. ¹H NMR (400 MHz, DMSO-d6) δ = 8.06 (d, J = 1.4 Hz, 1H), 7.86 (d, J = 8.4 Hz, 1H), 7.80 (dt, J = 6.8, 8.4 Hz, 1H), |
| | Example 115 | A85 | 7.53-7.49 (m, 1H), 7.48-7.43 (m, |
| | | | 1H), 7.31 (dt, J = 2.0, 8.4 Hz, 1H), |
| | | | 4.77-4.69 (m, 1H), 4.30-4.20 (m, |
| | | | 1H), 3.90-3.77 (m, 1H), 3.65-3.54 |
| | | | (m, 1H), 2.78 (d, J = 5.9 Hz, 6H), |
| | | | 2.65 (s, 3H), 2.40 (br dd, J = 1.4, |
| | | | 12.9 Hz, 1H), 2.15 (br d, J = 13.3 Hz, |
| | | | 1H), 2.09-1.98 (m, 1H), 1.95-1.82 |
| | | | (m, 1H). |
| 114 | 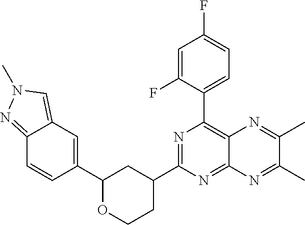 | 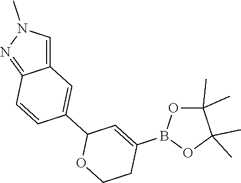 | Modification: Step 1 was stirred at 80 °C. for 12 h. Step 2 was stirred at 25 °C. for 4 h. Step 3 was stirred at 50 °C. for 24 h. The residue was purified by Prep- HPLC (Condition 3, Gradient 2) to afford 4-(2,4-difluorophenyl)-6,7- dimethyl-2-[2-(2-methylindazol-5- yl)tetrahydropyran-4-yl]pteridine (Example 114, cis, 246 mg, 0.475 mmol, 47% yield) as a solid. LC/MS (ESI, m/z⁺): 487.2 [M + H]⁺. ¹H NMR (400 MHz, CDCl₃) δ = 7.85 (s, 1H), 7.74 (dt, J = 6.6, 8.2 Hz, |
| | Example 114 | A86 | 1H), 7.70-7.62 (m, 2H), 7.35 (dd, J = |
| | | | 1.5, 8.9 Hz, 1H), 7.11-7.04 (m, 1H), |
| | | | 6.99 (dt, J = 2.4, 9.4 Hz, 1H), 4.62 |
| | | | (dd, J = 1.7, 11.2 Hz, 1H), 4.42-4.34 |
| | | | (m, 1H), 4.20 (s, 3H), 3.93-3.82 (m, |
| | | | 1H), 3.63 (s, 1H), 2.83 (s, 3H), 2.72 |
| | | | (s, 3H), 2.45 (br d, J = 1.6 Hz, 1H), |
| | | | 2.28 (br d, J = 4.4 Hz, 1H), 2.26-2.17 |
| | | | (m, 2H). |
| 116 | 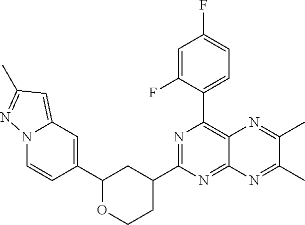 | 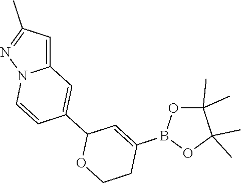 | Modification: Step 1 was stirred at 50 °C. for 2 h. Step 2 was stirred at 25 °C. for 6 h. Step 3 was stirred at 50 °C. for 6 h. The residue was purified by prep- TLC (50% EtOAc in PE) to afford 4-(2,4-difluorophenyl)-6,7- dimethyl-2-[2-(2-methylpyrazolo- [1,5-a]pyridin-5-yl)tetrahydropyran- 4-yl]pteridine (Example 116, cis, 9.3 mg, 0.0185 mmol, 23% yield) as a solid. LC/MS (ESI, m/z+): 487.1 [M + H]⁺. ¹H NMR (400 MHz, CDCl₃) δ ppm 2.12 (q, J = 12.09 Hz, |
| | Example 116 | A91 | 1H), 2.20-2.32 (m, 2H), 2.42-2.53 |
| | | | (m, 4H), 2.73 (s, 3H) 2.84 (s, 3H) |
| | | | 3.56-3.68 (m, 1H), 3.86 (td, J = 11.19, |
| | | | 4.00 Hz, 1H), 4.38 (br dd, J = 10.19, |
| | | | 2.81 Hz, 1H), 4.56 (br d, J = |
| | | | 10.26 Hz, 1H), 6.24 (s, 1H), 6.73 (dd, |
| | | | J = 7.25, 1.63 Hz, 1H), 6.96-7.12 (m, |
| | | | 2H), 7.45 (s, 1H), 7.71-7.80 (m, 1H), |
| | | | 8.31 (d, J = 7.25 Hz, 1H). |

### Method 36: Synthesis of Example 93

### Synthesis of A74

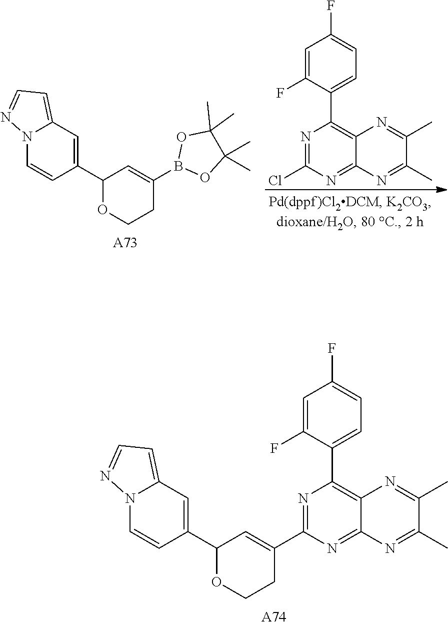

To a solution of 2-chloro-4-(2,4-difluorophenyl)-6,7-dimethyl-pteridine (1.1 eq, 621 mg, 2.02 mmol), 5-[4-(4,4,5,5-tetramethyl-1,3,2-dioxaborolan-2-yl)-3,6-dihydro-2H-pyran-6-yl]pyrazolo[1,5-a]pyridine (1.0 eq, 600 mg, 1.84 mmol) in 1,4-Dioxane (15 mL) and water (1.5 mL) were added Pd(dppf)Cl₂ DCM (0.1 eq, 135 mg, 0.184 mmol) and K₂CO₃ (3.0 eq, 464 mg, 5.52 mmol), and the reaction was stirred at 80° C. for 2 h under N₂ atmosphere. The reaction mixture was poured into H₂O (100 mL) and extracted with DCM (3×100 mL). The combined organic layers were dried over Na₂SO₄, filtered and concentrated under reduced pressure to give a residue. The residue was purified by silica gel column chromatography (0-100% EtOAc in PE) to afford 4-(2,4-difluorophenyl)-6,7-dimethyl-2-(6-pyrazolo[1,5-a]pyridin-5-yl-3,6-dihydro-2H-pyran-4-yl)pteridine (A74, 800 mg, 1.60 mmol, 87% yield) as a solid. LC/MS (ESI, m/z⁺): [M+H]⁺=471.2. ¹H NMR (400 MHz, CDCl₃) δ=8.46 (d, J=7.3 Hz, 1H), 7.95 (d, J=2.1 Hz, 1H), 7.81-7.72 (m, 1H), 7.69-7.59 (m, 2H), 7.08 (dt, J=2.5, 8.1 Hz, 1H), 7.03-6.95 (m, 1H), 6.87 (dd, J=1.8, 7.2 Hz, 1H), 6.51 (d, J=1.6 Hz, 1H), 5.47 (br d, J=2.1 Hz, 1H), 4.38-4.23 (m, 1H), 4.06-3.95 (m, 1H), 3.06 (br d, J=1.6 Hz, 2H), 2.85 (s, 3H), 2.74 (s, 3H).

### Synthesis of A75

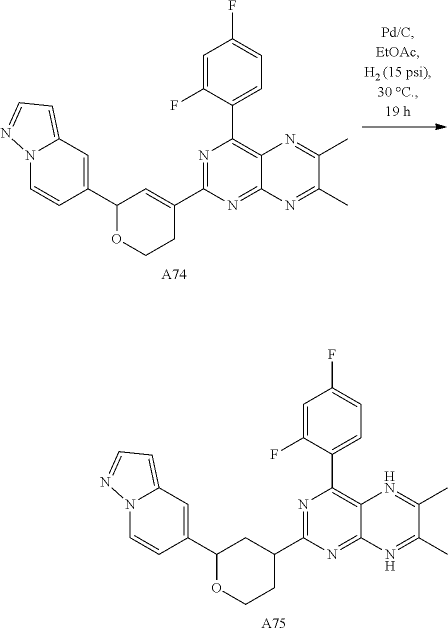

A mixture 4-(2,4-difluorophenyl)-6,7-dimethyl-2-(6-pyrazolo[1,5-a]pyridin-5-yl-3,6-dihydro-2H-pyran-4-yl)pteridine (1.0 eq, 20 mg, 0.043 mmol) in EtOAc (0.5 mL) was degassed with N₂ (×3). Pd/C (4.42 eq, 20 mg, 0.19 mmol) was added. The mixture was degassed with H₂ (×3) and stirred at 0° C. under H₂ (15 psi, balloon) for 1 h. The solution was stirred at 25° C. for 16 h, followed by stirring at 35° C. for another 2 h. The reaction mixture was filtered through a pad of celite and the filter cake was washed with DCM (100 mL) and EtOAc (100 mL). The filtrate was concentrated under reduced pressure to afford 4-(2,4-difluorophenyl)-6,7-dimethyl-2-(2-pyrazolo[1,5-a]pyridin-5-yltetrahydropyran-4-yl)-5,6,7,8-tetrahydropteridine (A75, 60 mg) as an oil. LC/MS (ESI, m/z⁺): [M+H]⁺=477.2.

### Synthesis of Example 93

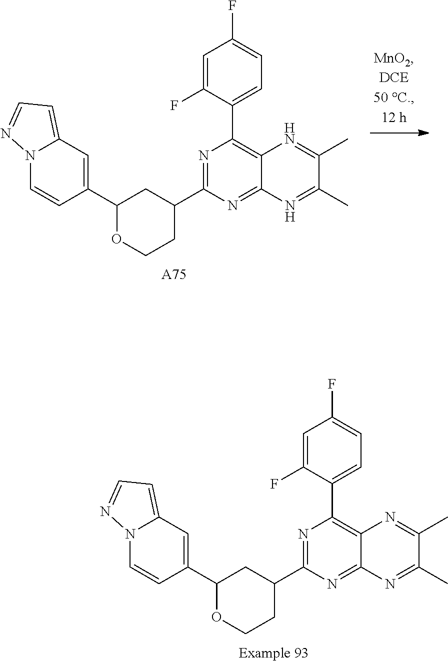

To a mixture 4-(2,4-difluorophenyl)-6,7-dimethyl-2-(2-pyrazolo[1,5-a]pyridin-5-yltetrahydropyran-4-yl)-5,6,7,8-tetrahydropteridine (1.0 eq, 60 mg, 0.126 mmol) in DCE (1 mL) was added MnO₂ (10.0 eq, 109 mg, 1.26 mmol), and the reaction was stirred at 50° C. for 12 h. The reaction mixture was filtered through a pad of celite and the filter cake was washed with DCM (50 mL). The filtrate was concentrated under reduced pressure and purified by Prep-TLC (100% EtOAc), followed by prep-HPLC (Condition 5, Gradient 1) to afford 4-(2,4-difluorophenyl)-6,7-dimethyl-2-(2-pyrazolo[1,5-a]pyridin-5-yltetrahydropyran-4-yl)pteridine (Example 93, cis, 13 mg, 0.027 mmol, 21 yield) as a solid. LC/MS (ESI, m/z⁺): [M+H]=473.2. ¹H NMR (400 MHz, CDCl₃) δ=8.44 (d, J=7.1 Hz, 1H), 7.93 (d, J=2.3 Hz, 1H), 7.75 (dt, J=6.6, 8.2 Hz, 1H), 7.59 (s, 1H), 7.14-7.06 (, 1H), 7.05-6.97 (m, H), 6.84 (dd, J=1.8, 7.1 Hz, 1H), 6.48 (d, J=1.9 Hz, 1H), 4.59 (d, J=9.8 Hz, 1H), 4.45-4.34 (m, 1H), 3.94-3.81 (m, 1H), 3.72-3.54 (m, 1H), 2.85 (s, 3H), 2.73 (s, 3H), 2.50 (br dd, J=2.1, 13.4 Hz, 1H), 2.32-2.22 (m, 2H), 2.19-2.08 (m, 1H).

An analogous method was followed to obtain the following compounds.

| Ex # | Compound | Starting Material | Characterization |
|---|---|---|---|
| 95 | 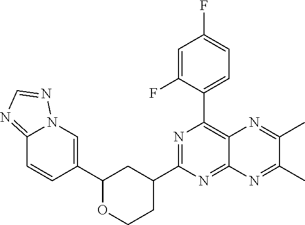 [SMILES: Cc1nc2nc(C3CCOC(c4ccc5ncnn5c4)C3)nc(-c3ccc(F)cc3F)c2nc1C] | 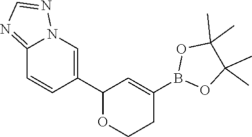 | The residue was purified by Prep- TLC (100% EtOAc), followed by prep-HPLC (Condition 5, Gradient 1) to afford 4-(2,4-difluorophenyl)-6,7- dimethyl-2-[2-([1,2,4]triazolo[1,5- alpyridin-6-yl)tetrahydropyran-4- yl]pteridine (Example 95, 24 mg, 0.049 mmol, 47% yield) as a solid. LC/MS (ESI, m/z⁺): [M + H]⁺ = 474.2. ¹H NMR (400 MHz, CDCl₃) δ = 8.70 (s, 1H), 8.34 (s, 1H), 7.80-7.70 (m, 2H), 7.61 (dd, J = 1.6, 9.1 Hz, 1H), 7.13-7.06 (m, 1H), 7.01 (ddd, J = 2.4, 9.0, 9.8 Hz, 1H), 4.68 (dd, J = 1.7, |
| | Example 95 | A76 | 11.4 Hz, 1H), 4.45-4.34 (m, 1H), |
| | | | 3.98-3.83 (m, 1H), 3.72-3.57 (m, 1H), |
| | | | 2.84 (s, 3H), 2.73 (s, 3H), 2.54 (br dd, |
| | | | J = 1.8, 13.2 Hz, 1H), 2.31-2.22 (m, |
| | | | 2H), 2.21-2.13 (m, 1H). |
| 98 | 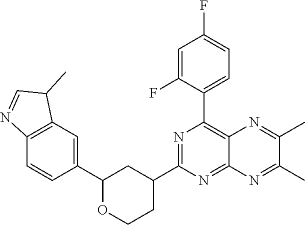 [SMILES: Cc1nc2nc(C3CCOC(c4ccc5c(c4)C(C)C=N5)C3)nc(-c3ccc(F)cc3F)c2nc1C] | 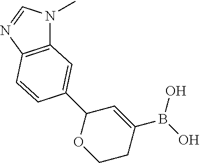 | Modification: Step 2 was ran in MeOH at 25 °C. for 12 h, then at 35 ºC for 2 h. The residue was purified by Prep- TLC (5% MeOH in DCM), followed by prep-HPLC (Condition 6) and lyophilized to give 4-(2,4- difluorophenyl)-6, 7-dimethyl-2-[2-(3- methylbenzimidazol-5- yl)tetrahydropyran-4-yl]pteridine (Example 98, 63.5 mg, 0.124 mmol, 34% yield) as a solid. LC/MS (ESI, m/z⁺): [M + H]⁺ = 487.2. ¹H NMR (400 MHz, CDCl₃) δ = 9.13 (s, 1H), |
| | Example 98 | A81 | 7.90 (d, J = 8.6 Hz, 1H), 7.80-7.71 |
| | | | (m, 2H), 7.56 (d, J = 8.7 Hz, 1H), |
| | | | 7.09 (dt, J = 2.3, 8.3 Hz, 1H), 7.01 |
| | | | (dt, J = 2.3, 9.4 Hz, 1H), 4.78 (br d, |
| | | | J = 10.0 Hz, 1H), 4.47-4.39 (m, 1H), |
| | | | 4.10 (s, 3H), 3.96-3.88 (m, 1H), 3.72- |
| | | | 3.62 (m, 1H), 2.84 (s, 3H), 2.73 (s, |
| | | | 3H), 2.53 (br d, J = 13.2 Hz, 1H), |
| | | | 2.33-2.25 (m, 2H), 2.21-2.11 (m, 1H). |
| 99 | 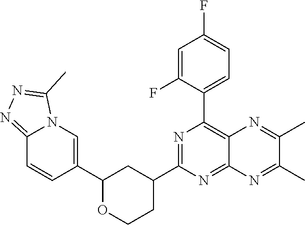 [SMILES: Cc1nc2nc(C3CCOC(c4ccc5nnc(C)n5c4)C3)nc(-c3ccc(F)cc3F)c2nc1C] | 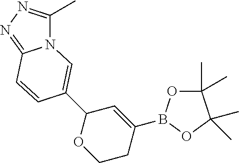 | Modification: Step 2 was stirred at 25 ºC for 12 h, then at 45 ° C. for 4 h. The residue was purified by Prep- TLC (5% MeOH in DCM) twice to afford 4-(2,4-difluorophenyl)-6,7- dimethyl-2-[2-(3-methyl[1,2,4]- triazolo[4,3-a]pyridin-6- yl)tetrahydropyran-4-yl]pteridine (Example 99, 66.3 mg, 0.124 mmol, 24% yield) as a solid. LC/MS (ESI, m/z⁺): [M + H]⁺ = 488.2. ¹H NMR (400 MHz, CDCl₃) δ = 8.57 (s, 1H), 7.80-7.71 (m, 1H), 7.64-7.52 (m, 2H), 7.10 (dt, J = 2.0, 8.2 Hz, 1H), 7.01 |
| | Example 99 | A82 | (dt, J = 2.3, 9.4 Hz, 1H), 4.65 (br d, J = |
| | | | 10.1 Hz, 1H), 4.43-4.35 (m, 1H), |
| | | | 3.93-3.83 (m, 1H), 3.69-3.58 (m, 1H), |
| | | | 2.85 (s, 3H), 2.74 (s, 3H), 2.51 (br d, |
| | | | J = 13.2 Hz, 1H), 2.32-2.23 (m, 2H), |
| | | | 2.22-2.12 (m, 1H). |

### Method 37: Synthesis of Example 104

### Synthesis of A83

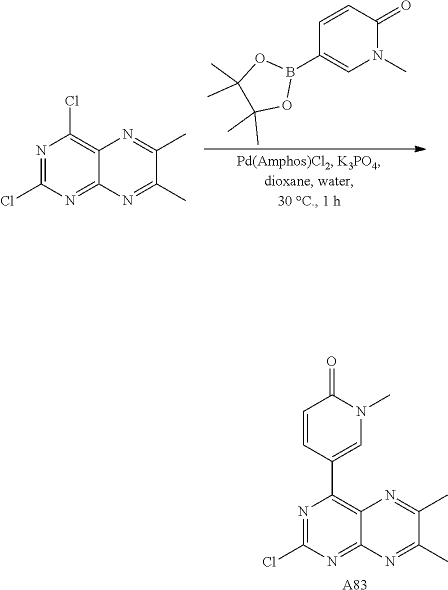

To a solution of 2,4-dichloro-6,7-dimethyl-pteridine (1.0 eq, 100 mg, 0.44 mmol) and 1-methyl-5-(4,4,5,5-tetramethyl-1,3,2-dioxaborolan-2-yl)pyridin-2-one (1.0 eq, 103 mg, 0.44 mmol) in 1,4-dioxane (5 mL) and water (1 mL) was added K₂CO₃ (3.0 eq, 181 mg, 1.31 mmol) and Pd(dppf)Cl₂ CH₂Cl₂ (0.10 eq, 35 mg, 0.044 mmol) under N₂, then the mixture was stirred for 1 h at 30° C. The reaction solution was extracted with DCM (2×10 mL) and washed with brine (10 mL). The organic layer was separated, dried over Na₂SO₄, and filtered. The filtrate was concentrated under reduced pressure and purified by prep-HPLC (FA) and lyophilized to give 5-(2-chloro-6,7-dimethyl-pteridin-4-yl)-1-methyl-pyridin-2-one (A83, 60 mg, 0.131 mmol, 30% yield) as a solid. LC/MS (ESI, m/z⁺): 302.0 [M+H]⁺. ¹H NMR (400 MHz, CDCl₃) δ ppm 2.84 (d, J=6.72 Hz, 6H), 2.87 (d, J=5.50 Hz, 3H), 3.75 (s, 3H), 6.73 (d, J=9.66 Hz, 1H), 8.78 (dd, J=9.72, 2.51 Hz, 1H), 9.38 (d, J=2.44 Hz, 1H).

### Synthesis of Example 104

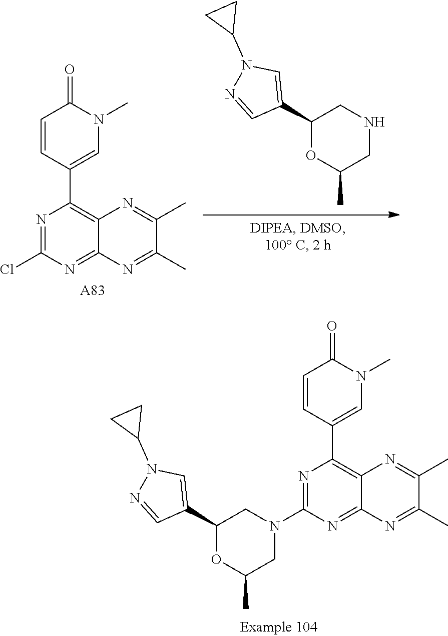

To a solution of 5-(2-chloro-6,7-dimethyl-pteridin-4-yl)-1-methyl-pyridin-2-one (1.0 eq, 55 mg, 0.182 mmol) and (2S,6R)-2-(1-cyclopropylpyrazol-4-yl)-6-methyl-morpholine (2.0 eq, 76 mg, 0.37 mmol) in DMSO (1 mL) was added DIEA (5.0 eq, 0.15 mL, 0.91 mmol), and the mixture was stirred at 100° C. for 2 h. The resulting mixture was filtered, concentrated under reduced pressure, and purified by prep-HPLC (Condition 7) and lyophilized to afford 5-[2-[(2S,6R)-2-(1-cyclopropylpyrazol-4-yl)-6-methyl-morpholin-4-yl]-6,7-dimethyl-pteridin-4-yl]-1-methyl-pyridin-2-one (Example 104, 19 mg, 0.04 mmol, 22% yield) as a solid. LC/MS (ESI, m/z⁺): 473.3 [M+H]⁺. ¹H NMR (400 MHz, DMSO-d6) δ ppm 0.93-0.99 (m, 2H), 1.01-1.08 (m, 2H), 1.24 (d, J=6.11 Hz, 3H), 2.63 (s, 6H), 2.73-2.83 (m, 1H), 2.99-3.13 (m, 1H), 3.60 (s, 3H), 3.68-3.77 (m, 2H), 4.53 (dd, J=11.00, 2.32 Hz, 1H), 4.71-4.94 (m, 2H), 6.54 (d, J=9.66 Hz, 1H), 7.50 (s, 1H), 7.87 (s, 1H), 8.34-8.62 (m, 1H), 9.35 (s, 1H).

### Method 38: Synthesis of Example 121

### Synthesis of A94

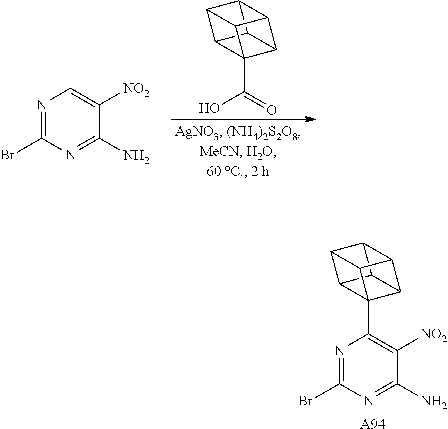

To a solution of 2-bromo-5-nitropyrimidin-4-amine (1.0 eq, 550 mg, 2.51 mmol) in H₂O (10 mL) and MeCN (20 mL) was added (NH₄)₂S₂O₈(3.0 eq, 1.72 g, 7.53 mmol), AgNO₃ (2.50 eq, 1.06 g, 6.28 mmol) and cubane-1-carboxylic acid (1.0 eq, 372 mg, 2.51 mmol), and the reaction was stirred at 60° C. for 2 h. The reaction mixture was quenched by addition Na₂SO₃ (40 mL) at 0° C. and then extracted with EtOAc (2×60 mL). The combined organic layers were washed with brine (50 mL), dried over Na₂SO₄, and filtered. The filtrate was concentrated under reduced pressure and purified by silica gel column chromatography (30-60% EtOAc in PE) to afford 2-bromo-6-cuban-1-yl-5-nitro-pyrimidin-4-amine (A94, 390 mg, 1.21 mmol, 48% yield) as a solid. LC/MS (ESI, m/z⁺): [M+H+2]=323.0. ¹H NMR (400 MHz, DMSO-d6) δ=4.28-4.17 (m, 3H), 4.03-3.94 (m, 4H).

### Synthesis of A95

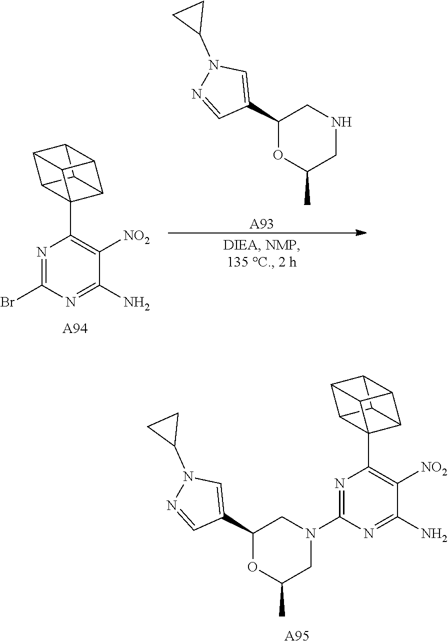

To a solution of 2-bromo-6-cuban-1-yl-5-nitro-pyrimidin-4-amine (A94, 1.0 eq, 190 mg, 0.59 mmol) in NMP (2 mL), were added (2S,6R)-2-(1-cyclopropyl-1H-pyrazol-4-yl)-6-methylmorpholine (A93, 1.0 eq, 123 mg, 0.59 mmol) and DIEA (3.0 eq, 0.29 mL, 1.77 mmol), and the reaction was heated to 135° C. for 2 h. The mixture was diluted with water (15 mL) and extracted with EtOAc (2×20 mL). The combined organic layers were washed with brine (20 mL), dried over Na₂SO₄, and filtered. The filtrate was concentrated under reduced pressure and purified by silica gel column chromatography (30-60% EtOAc in PE) to afford 6-cuban-1-yl-2-[(2S,6R)-2-(1-cyclopropylpyrazol-4-yl)-6-methyl-morpholin-4-yl]-5-nitro-pyrimidin-4-amine (A95, 120 mg, 0.268 mmol, 45% yield) as a solid. LC/MS (ESI, m/z⁺): [M+H]⁺=448.1. ¹H NMR (400 MHz, CDCl₃) δ=7.53 (d, J=2.8 Hz, 2H), 5.11-4.60 (m, 2H), 4.59-4.46 (m, 1H), 4.27 (br s, 3H), 4.08-3.86 (m, 4H), 3.74 (br d, J=3.4 Hz, 1H), 3.64-3.51 (m, 1H), 3.00-2.87 (m, 1H), 2.73-2.66 (m, 1H), 1.31 (br d, J=6.1 Hz, 3H), 1.19-1.09 (m, 2H), 1.06-0.98 (m, 2H).

### Synthesis of A96

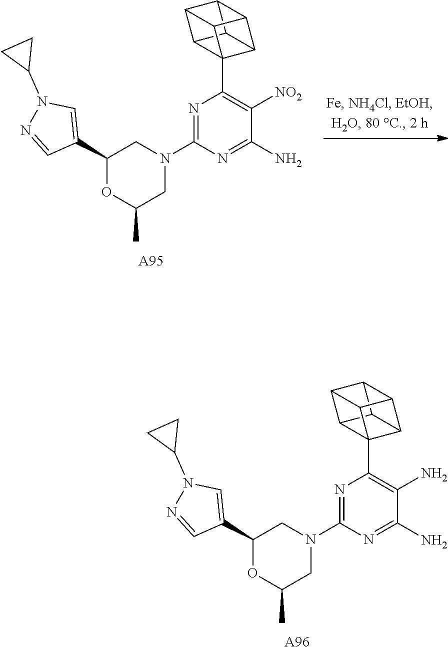

The mixture of 6-cuban-1-yl-2-[(2S,6R)-2-(1-cyclopropylpyrazol-4-yl)-6-methyl-morpholin-4-yl]-5-nitro-pyrimidin-4-amine (A95, 1.0 eq, 100 mg, 0.22 mmol), Fe (5.0 eq, 62 mg, 1.12 mmol) and NH₄Cl (6.0 eq, 72 mg, 1.34 mmol) in EtOH (4 mL) and H₂O (2 mL) was stirred at 80° C. for 2 h. The reaction was filtered and the filter cake was washed with DMF/EtOAc=1/1 (2×10 mL). The filtrate was added into brine (30 mL) and extracted with EtOAc (2×20 mL). The combined organic layers were washed with brine (3×10 mL), dried over Na₂SO₄, filtered and concentrated under reduced pressure to afford 6-cuban-1-yl-2-[(2S,6R)-2-(1-cyclopropylpyrazol-4-yl)-6-methyl-morpholin-4-yl]pyrimidine-4,5-diamine (A96, 90 mg, 0.216 mmol, 96% yield) as a solid. LC/MS (ESI, m/z⁺): [M+H]⁺=418.1. ¹H NMR (400 MHz, CDCl₃) δ=7.52 (s, 2H), 4.79-4.60 (m, 2H), 4.58-4.47 (m, 2H), 4.41-4.37 (m, 2H), 4.13-4.04 (m, 3H), 3.84-3.70 (m, 1H), 3.57 (td, J=3.6, 7.4 Hz, 1H), 2.80 (dd, J=10.9, 12.9 Hz, 1H), 2.57 (br dd, J=10.6, 12.8 Hz, 2H), 1.28 (d, J=6.3 Hz, 3H), 1.15-1.08 (m, 2H), 1.04-0.96 (m, 2H).

### Synthesis of Example 121

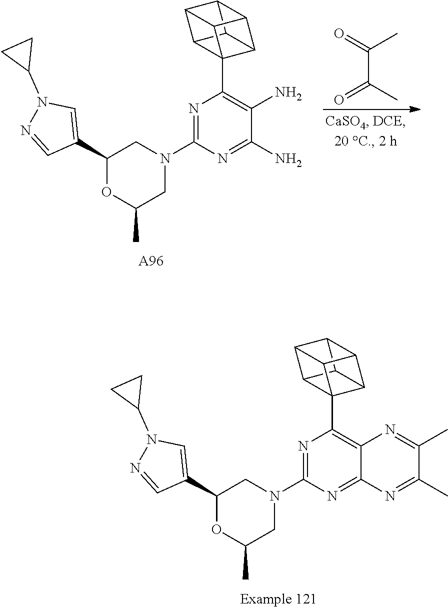

To a solution of 6-cuban-1-yl-2-[(2S,6R)-2-(1-cyclopropylpyrazol-4-yl)-6-methyl-morpholin-4-yl]pyrimidine-4,5-diamine (A96, 1.0 eq, 80 mg, 0.19 mmol) in DCE (4 mL) was added CaSO4 (5.0 eq, 130 mg, 0.96 mmol) and butane-2,3-dione (2.5 eq, 41 mg, 0.48 mmol), and the reaction was stirred at 20° C. for 2 h. The reaction mixture was filtered and concentrated under reduced pressure to give a residue. The residue was added into DMSO (2 mL) and DMF (2 mL). The precipitated solid was filtered. The filter cake was dissolved in DCM (1 mL) and purified by prep-TLC (9% MeOH in DCM) to afford (2S,6R)-4-(4-cuban-1-yl-6,7-dimethyl-pteridin-2-yl)-2-(1-cyclopropylpyrazol-4-yl)-6-methyl-morpholine (Example 121, 55 mg, 0.10 mmol, 53% yield) as a solid. LC/MS (ESI, m/z⁺): [M+H]⁺=468.2. ¹H NMR (400 MHz, CDCl₃) δ=7.59-7.51 (m, 2H), 5.11 (br d, J=13.5 Hz, 1H), 5.06-4.95 (m, 1H), 4.59 (dd, J=2.6, 10.9 Hz, 1H), 4.54-4.41 (m, 3H), 4.22-4.03 (m, 4H), 3.81 (ddd, J=2.6, 6.4, 10.5 Hz, 1H), 3.59 (tt, J=3.8, 7.3 Hz, 1H), 3.04 (dd, J=11.1, 13.0 Hz, 1H), 2.80 (dd, J=10.7, 13.2 Hz, 1H), 2.67 (s, 3H), 2.60 (s, 3H), 1.33 (d, J=6.1 Hz, 3H), 1.16-1.10 (m, 2H), 1.02 (dd, J=2.2, 7.2 Hz, 2H).

### Method 39: Synthesis of Example 136 and Example 138

### Synthesis of A102

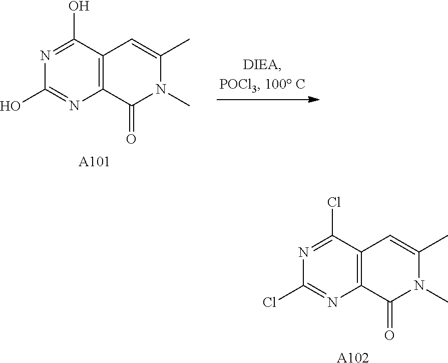

To a solution of 2,4-dihydroxy-6,7-dimethyl-pyrido[3,4-d]pyrimidin-8-one (A101, 1.0 eq, 2.50 g, 12.1 mmol) in POCl₃ (24.7 eq, 25 mL, 298 mmol) was added DIEA (1.0 eq, 1.56 g, 12.1 mmol) at 0° C. under N₂. The mixture was stirred at 100° C. for 1 h under N₂. The mixture was concentrated under reduced pressure and dissolved in EtOAc (20 mL). The mixture was added into NaHCO₃ solution (150 mL) slowly, below 40° C. The mixture was filtered to give crude starting material (1.7 g) in the filter cake. The filtrate was extracted with EtOAc (2×100 mL) and washed with brine (50 mL). The organic layer was dried over Na₂SO₄, filtered, and concentrated under reduced pressure to afford 2,4-dichloro-6,7-dimethyl-pyrido[3,4-d]pyrimidin-8-one (A102, 720 mg, 2.95 mmol, 25% yield) as a solid. LC/MS (ESI, m/z⁺): [M+H]⁺=243.9. ¹H NMR (400 MHz, CDCl₃) δ=6.58 (s, 1H), 3.67 (s, 3H), 2.53 (s, 3H).

### Synthesis of A103

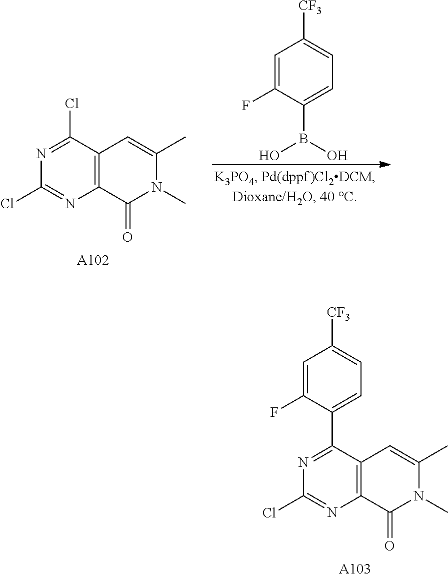 [SMILES: Cc1cc2c(Cl)nc(Cl)nc2c(=O)n1C ; OB(O)c1ccc(C(F)(F)F)cc1F ; "A102" ; Cc1cc2c(-c3ccc(C(F)(F)F)cc3F)nc(Cl)nc2c(=O)n1C ; "A103"]

To a solution of 2,4-dichloro-6,7-dimethyl-pyrido[3,4-d]pyrimidin-8-one (A102, 1.0 eq, 650 mg, 2.66 mmol) and [2-fluoro-4-(trifluoromethyl)phenyl]boronic acid (0.9 eq, 498 mg, 2.40 mmol) in 1,4-dioxane (39 mL) and water (3.9 mL) were added K₃PO₄ (2.0 eq, 1.13 g, 5.33 mmol) and Pd(dppf)Cl₂ DCM (0.1 eq, 220 mg, 0.266 mmol). The mixture was degassed with N₂ (×3) and the mixture was stirred at 40° C. for 1 h under N₂. The mixture was concentrated under reduced pressure and purified by silica gel flash column chomatography (0-100% EtOAc in PE) to afford 2-chloro-4-[2-fluoro-4-(trifluoromethyl)phenyl]-6,7-dimethyl-pyrido[3,4-d]pyrimidin-8-one (A103, 650 mg, 1.75 mmol, 66% yield) as a solid. LC/MS (ESI, m/z⁺): [M+H]⁺=372.0. ¹H NMR (400 MHz, DMSO-d6) δ=7.99 (d, J=9.9 Hz, 1H), 7.91-7.82 (m, 2H), 6.30 (d, J=2.1 Hz, 1H), 3.56 (s, 3H), 2.43 (s, 3H).

### Synthesis of A104

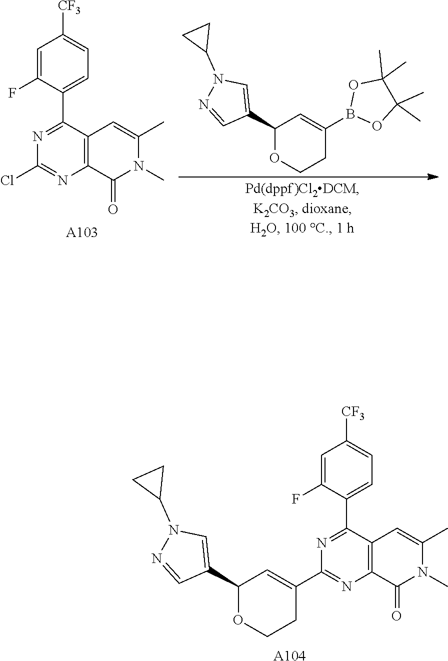

To a solution of 2-chloro-4-[2-fluoro-4-(trifluoromethyl)phenyl]-6,7-dimethyl-pyrido[3,4-d]pyrimidin-8-one (A103, 1.0 eq, 200 mg, 0.54 mmol) and 1-cyclopropyl-4-[(6R)-4-(4,4,5,5-tetramethyl-1,3,2-dioxaborolan-2-yl)-3,6-dihydro-2H-pyran-6-yl]pyrazole (1.30 eq, 221 mg, 0.70 mmol) in 1,4-dioxane (15 mL) and water (2 mL) were added K₂CO₃ (2.0 eq, 148 mg, 1.08 mmol) and Pd(dppf)Cl₂ DCM (0.15 eq, 67 mg, 0.081 mmol). The mixture was degassed with N₂ (×3) and the mixture was stirred at 100° C. for 1 h under N₂. The mixture was poured into water (30 mL) and extracted with EtOAc (2×100 mL). The organic layer was washed with brine (50 mL), dried over Na₂SO₄, and filtered. The filtrate was concentrated under reduced pressure and purified by column chromatography (50-100% EtOAc in hexane, then 4% MeOH in EtOAc) to afford 2-[(6R)-6-(1-cyclopropylpyrazol-4-yl)-3,6-dihydro-2H-pyran-4-yl]-4-[2-fluoro-4-(trifluoromethyl)phenyl]-6,7-dimethyl-pyrido[3,4-d]pyrimidin-8-one (A104, 220 mg, 0.42 mmol, 78% yield) as a solid. LC/MS (ESI, m/z⁺): [M+H]⁺=526.2. ¹H NMR (400 MHz, CDCl₃) δ=7.77-7.70 (m, 1H), 7.64 (br d, J=8.4 Hz, 1H), 7.54 (br d, J=9.6 Hz, 1H), 7.50 (br d, J=11.1 Hz, 3H), 6.11 (br d, J=3.0 Hz, 1H), 5.43 (br d, J=2.1 Hz, 1H), 3.91 (ddd, J=4.6, 6.9, 11.4 Hz, 1H), 3.68 (s, 3H), 3.56 (qd, J=3.7, 7.1 Hz, 1H), 3.05-2.82 (m, 2H), 2.43 (s, 3H), 1.32-1.22 (m, 1H), 1.13-1.08 (m, 2H), 1.03-0.96 (m, 2H).

### Synthesis of Example 136 and Example 138

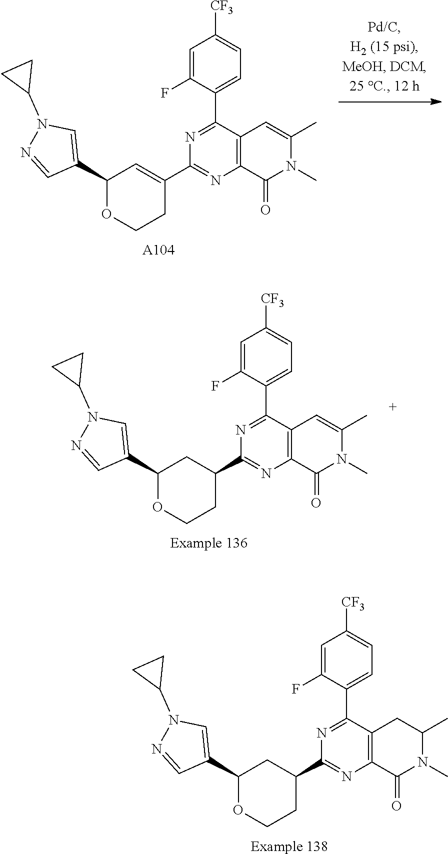

To a solution of 2-[(6R)-6-(1-cyclopropylpyrazol-4-yl)-3,6-dihydro-2H-pyran-4-yl]-4-[2-fluoro-4-(trifluoromethyl)phenyl]-6,7-dimethyl-pyrido[3,4-d]pyrimidin-8-one (A104, 1.0 eq, 60 mg, 0.114 mmol) in MeOH (4 mL) and DCM (2 mL) was added Pd/C (1.0 eq, 22 mg, 0.114 mmol) under N₂. The mixture was purged by H₂ (×3), and the reaction was stirred at 25° C. for 12 h under H₂ (15 psi). The reaction mixture was filtered, concentrated under reduced pressure, and purified by prep-TLC (3% MeOH in EtOAc) twice to afford 2-[(2R,4S)-2-(1-cyclopropylpyrazol-4-yl)tetrahydropyran-4-yl]-4-[2-fluoro-4-(trifluoromethyl)phenyl]-6,7-dimethyl-pyrido[3,4-d]pyrimidin-8-one (Example 136, 5.53 mg, 0.0088 mmol, 8% yield) as a gum and a crude residue (15 mg, solid). The crude residue was purified by prep-HPLC (Condition 8) and lyophilized to afford 2-[(2R,4S)-2-(1-cyclopropylpyrazol-4-yl)tetrahydropyran-4-yl]-4-[2-fluoro-4-(trifluoromethyl)phenyl]-6,7-dimethyl-5,6-dihydropyrido[3,4-d]pyrimidin-8-one formic acid (Example 138, 9.54 mg, 0.0164 mmol, 14% yield) as a solid. Example 136: LC/MS (ESI, m/z⁺): 528.2 [M+H]⁺. ¹H NMR (400 MHz, CDCl₃) δ=7.75-7.68 (m, 1H), 7.68-7.61 (m, 1H), 7.55 (br d, J=9.3 Hz, 1H), 7.51-7.40 (m, 2H), 6.10 (d, J=3.3 Hz, 1H), 4.53 (br d, J=9.6 Hz, 1H), 4.23 (br dd, J=4.2, 11.4 Hz, 1H), 3.83-3.73 (m, 1H), 3.71-3.66 (m, 3H), 3.60-3.50 (m, 2H), 2.43 (s, 3H), 2.37-2.29 (m, 1H), 2.20-2.04 (m, 2H), 1.12-1.06 (m, 2H), 1.01-0.95 (m, 2H); ¹⁹F NMR (400 MHz, DMSO-d6) δ =−62.95, −110.79. Example 138: (mixture of diastereomers): LC/MS (ESI, m/z⁺): 530.2 [M+H]⁺. ¹H NMR (400 MHz, CDCl₃) δ=7.71-7.63 (m, 2H), 7.55 (d, J=9.7 Hz, 1H), 7.23 (s, 2H), 6.11 (d, J=2.8 Hz, 1H), 3.68 (s, 3H), 3.65-3.57 (m, 2H), 3.54-3.45 (m, 2H), 2.52-2.45 (m, 1H), 2.43 (s, 3H), 2.40-2.28 (m, 2H), 2.21-1.98 (m, 4H), 1.07-1.01 (m, 2H), 0.97-0.90 (m, 2H); ¹⁹F NMR (400 MHz, CDCl₃) δ=−62.95, −110.93.

### Method 40: Synthesis of Example 134

### Synthesis of A105

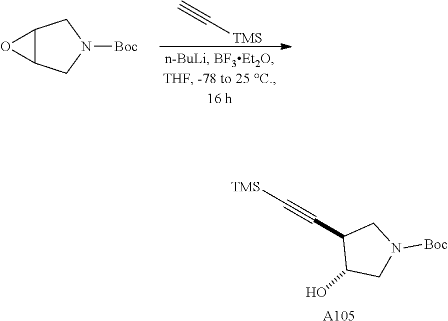

To a solution of ethynyltrimethylsilane (2.6 eq, 2.76 g, 28.1 mmol) in THF (30 mL) was added n-BuLi (2.0 eq, 8.6 mL, 21.6 mmol) over 5 mins at −78° C. under N₂. After 30 mins, BF₃ Et₂O (3.80 eq, 5.2 mL, 41.0 mmol) was added over 5 mins at −78° C. under N₂. After stirring for a further 0.5 h at −78° C., a solution of tert-butyl 6-oxa-3-azabicyclo[3.1.0]hexane-3-carboxylate (1.0 eq, 2.00 g, 10.8 mmol) in THF (30 mL) was added, and the reaction mixture was stirred at −78° C. for 1.5 h. The solution was warmed to 25° C. and stirred for 12 h. The mixture was poured into NH₄Cl solution (200 mL) and extracted with EtOAc (2×300 mL). The organic layers were washed with brine (3×200 mL), concentrated under reduced pressure, and purified by column chromatography (9-50% EtOAc in hexane) to afford tert-butyl (3R,4S)-3-hydroxy-4-(2-trimethylsilylethynyl)pyrrolidine-1-carboxylate (A105, 600 mg, 2.12 mmol, 20% yield) as a solid. LC/MS (ESI, m/z⁺): [M+H]⁺=228.2. ¹H NMR (400 MHz, CDCl₃) δ=4.35 (br d, J=4.6 Hz, 1H), 3.81-3.64 (m, 2H), 3.46-3.35 (m, 1H), 3.34-3.21 (m, 1H), 2.91 (br s, 1H), 1.71-1.64 (m, 1H, OH), 1.47 (s, 9H), 0.15 (s, 9H).

### Synthesis of A106

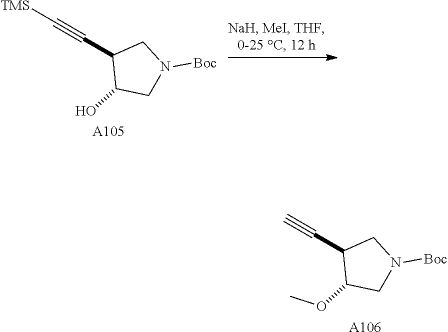

To a solution of tert-butyl (3R,4S)-3-hydroxy-4-(2-trimethylsilylethynyl)pyrrolidine-1-carboxylate (A105, 1.0 eq, 500 mg, 1.76 mmol) in THF (25 mL) was added NaH (4.0 eq, 282 mg, 7.06 mmol) at 0° C. under N₂, and the mixture was stirred at 0° C. for 0.5 h. Mel (4.0 eq, 1.00 g, 7.06 mmol) was added at 0° C. under N₂, the mixture was stirred at 25° C. for 12 h under N₂. The mixture was poured into NH₄Cl solution (50 mL) and extracted with EtOAc (100 mL). The organic layer was washed with brine (50 mL) and concentrated under reduced pressure to afford tert-butyl (3S,4R)-3-ethynyl-4-methoxy-pyrrolidine-1-carboxylate (A106, 450 mg, 2.00 mmol, 113% yield) as an oil. LC/MS (ESI, m/z⁺): [M+H-Bu]⁺=170.0. ¹H NMR (400 MHz, CDCl₃) δ=3.94-3.86 (m, 1H), 3.73-3.57 (m, 2H), 3.54-3.42 (m, 2H), 3.41 (s, 3H), 3.03-2.95 (m, 1H), 2.17 (d, J=2.1 Hz, 1H), 1.47 (s, 9H).

### Synthesis of A107

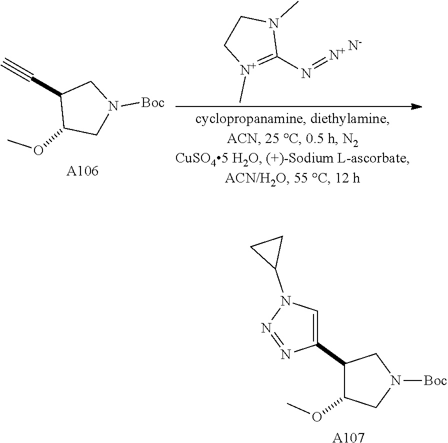

To a solution of cyclopropanamine (3.0 eq, 0.047 mL, 0.67 mmol) in MeCN (3 mL) were added 2-azido-1,3-dimethyl-4,5-dihydroimidazol-1-ium (3.0 eq, 190 mg, 0.67 mmol) and diethylamine (9.0 eq, 0.21 mL, 2.0 mmol), and the reaction was stirred at 25° C. for 0.5 h. Then, a mixture of (+)-Sodium L-ascorbate (0.5 eq, 22 mg, 0.11 mmol), CuSO₄·5H₂O (0.20 eq, 11 mg, 0.044 mmol) and tert-butyl (3S,4R)-3-ethynyl-4-methoxy-pyrrolidine-1-carboxylate (A106, 1.0 eq, 50 mg, 0.22 mmol) in water (0.2 mL) and MeCN (2 mL) was added to the reaction under N₂, and the mixture was stirred at 55° C. for 12 h. The reaction was quenched by H₂O (40 mL) and extracted with EtOAc (2×50 mL). The combined organic layers were dried over anhydrous Na₂SO₄, filtered, and concentrated under reduced pressure to give a residue. The residue was purified by reversed-phase-HPLC (FA) and lyophilized to afford tert-butyl (3R,4R)-3-(1-cyclopropyltriazol-4-yl)-4-methoxy-pyrrolidine-1-carboxylate (A107, 42 mg, 0.136 mmol, 61% yield) as an oil. LC/MS (ESI, m/z⁺): [M+H]⁺=253.1. ¹H NMR (400 MHz, CDCl₃) δ=7.38 (br s, 1H), 3.80 (dd, J=7.0, 10.9 Hz, 1H), 3.72 (dt, J=3.7, 7.3 Hz, 1H), 3.68-3.51 (m, 3H), 3.40 (br d, J=6.5 Hz, 4H), 1.63 (s, 1H), 1.47 (s, 9H), 1.26 (br s, 2H), 1.16 (br d, J=6.4 Hz, 2H).

### Synthesis of A108

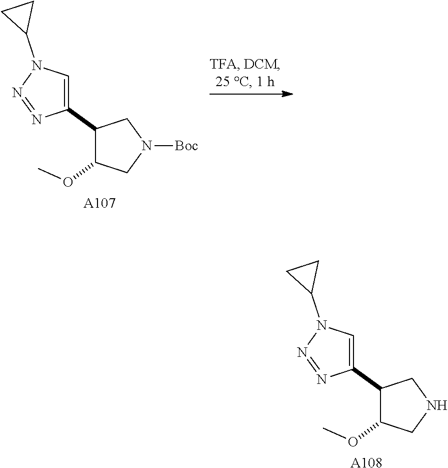

To a solution of tert-butyl (3R,4R)-3-(1-cyclopropyltriazol-4-yl)-4-methoxy-pyrrolidine-1-carboxylate (A107, 1.0 eq, 40 mg, 0.13 mmol) in DCM (3 mL) was added 2,2,2-trifluoroacetic acid (1.0 eq, 1.0 mL), the mixture was stirred at 25° C. for 1 h. The reaction mixture was concentrated under reduced pressure to afford crude 1-cyclopropyl-4-[(3R,4R)-4-methoxypyrrolidin-3-yl]triazole (A108, 30 mg) as an oil. LC/MS (ESI, m/z⁺): [M+H]⁺=209.1.

### Synthesis of Example 134

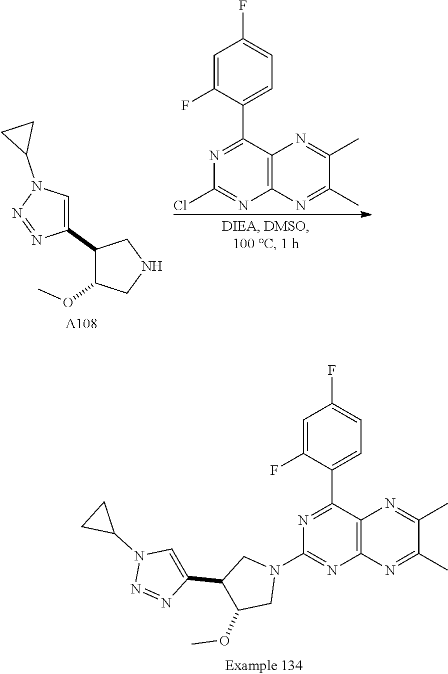

To a solution of 2-chloro-4-(2,4-difluorophenyl)-6,7-dimethyl-pteridine (1.5 eq, 55 mg, 0.18 mmol) and 1-cyclopropyl-4-[(3R,4R)-4-methoxypyrrolidin-3-yl]triazole (A108, 1.0 eq, 25 mg, 0.12 mmol) in DMSO (3 mL) was added DIEA (10.0 eq, 146 mg, 1.20 mmol), and the reaction was stirred at 100° C. for 1 h. The reaction mixture was filtered, concentrated under reduced pressure and purified by prep-HPLC (Condition 8) and lyophilized to afford 2-[3-(1-cyclopropyl-triazol-4-yl)-4-methoxy-pyrrolidin-1-yl]-4-(2,4-difluorophenyl)-6,7-dimethyl-pteridine formic acid (Example 134, trans, 44.5 mg, 0.084 mmol, 70% yield) as a solid. LC/MS (ESI, m/z⁺): [M+H]⁺=479.2. ¹H NMR (400 MHz, DMSO-d6) δ=8.10 (d, J=3.5 Hz, 1H), 7.76 (q, J=7.9 Hz, 1H), 7.44 (dt, J=2.2, 9.8 Hz, 1H), 7.33-7.23 (m, 1H), 4.27-4.16 (m, 1H), 4.04-3.84 (m, 4H), 3.81-3.65 (m, 2H), 3.36 (br d, J=7.7 Hz, 3H), 2.64 (s, 3H), 2.51 (br s, 3H), 1.13 (br s, 2H), 1.10-1.03 (m, 2H); ¹⁹F NMR (400 MHz, DMSO-d6) δ=−107.47.

### Method 41: Synthesis of Example 84

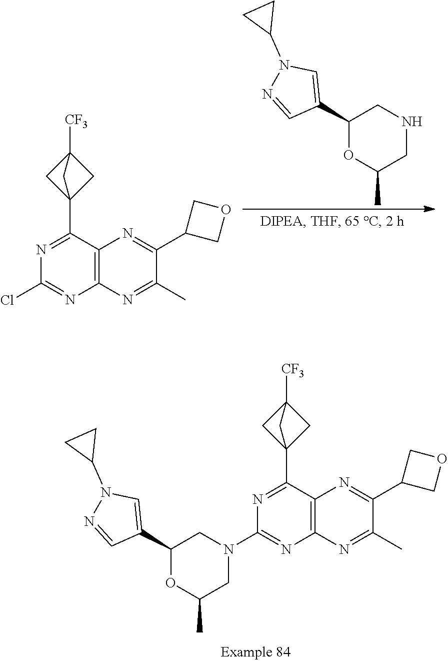 [SMILES: Cc1nc2nc(Cl)nc(C34CC(C(F)(F)F)(C3)C4)c2nc1C1COC1 ; C[C@@H]1CNC[C@H](c2cnn(C3CC3)c2)O1 ; Cc1nc2nc(N3C[C@@H](C)O[C@@H](c4cnn(C5CC5)c4)C3)nc(C34CC(C(F)(F)F)(C3)C4)c2nc1C1COC1]

A vial equipped with a Teflon-coated stirring bar was charged with 2-chloro-7-methyl-6-(oxetan-3-yl)-4-[3-(trifluoromethyl)-1-bicyclo[1.1.1]pentanyl]pteridine (1.0 eq, 62 mg, 0.17 mmol), (2S,6R)-2-(1-cyclopropylpyrazol-4-yl)-6-methyl-morpholine (1.1 eq, 38 mg, 0.18 mmol) and anhydrous THF (1.5 mL). DIPEA (3.0 eq, 87 μL, 0.5 mmol) was then added and the vial was stirred at 65° C. for 1 h. The reaction mixture was cooled down to RT, quenched with water and dissolved in EtOAc (10 mL). The aqueous phase was extracted with EtOAc (3×20 mL). The combined organic layers were washed with brine, dried over Na₂SO₄, and filtered. The filtrate was concentrated under reduced pressure and purified by silica gel column chromatography (50-100% EtOAc in Hexanes) to afford (2S,6R)-2-(1-cyclopropyl-pyrazol-4-yl)-6-methyl-4-[7-methyl-6-(oxetan-3-yl)-4-[3-(trifluoromethyl)-1-bicyclo[1.1.1]pentanyl]-pteridin-2-yl]morpholine (Example 84, 54 mg, 0.1 mmol, 60% yield) as a solid. ESI-MS (m/z)+: 542.4 (obs). ¹H NMR (400 MHz, CHCl₃-d) δ_{H} 7.58 (d, J=9.0 Hz, 2H), 5.10-4.90 (m, 6H), 4.66-4.57 (m, 2H), 3.79 (m, 1H), 3.62 (m, 1H), 3.04 (m, 1H), 2.85-2.79 (m, 1H), 2.61 (s, 6H), 2.55 (s, 3H), 1.33 (d, J=6.2 Hz, 3H), 1.15 (s, 2H), 1.06 (d, J=6.2 Hz, 2H). ¹⁹F NMR (CHCl₃-d, 376 MHz): δ_{F}−73.0 (s, 3F).

### Method 42: Synthesis of Example 86

### Synthesis of A109

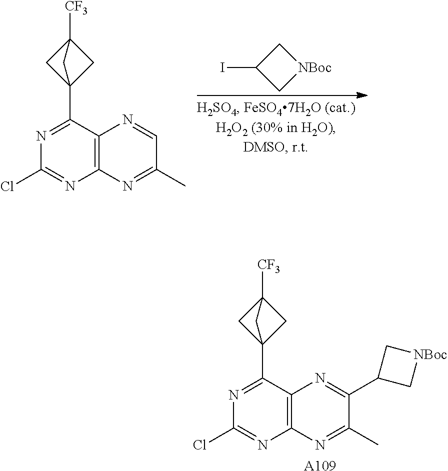

H₂O₂(30% in water, 3.0 eq, 495 μL, 4.8 mmol) was added dropwise over 1-2 min to a stirred solution of 2-chloro-7-methyl-4-[3-(trifluoromethyl)-1-bicyclo[1.1.1]pentanyl]pteridine (1.0 eq, 508 mg, 1.6 mmol), conc. sulfuric acid (2.0 eq, 172 μL, 3.2 mmol), tert-butyl 3-iodoazetidine-1-carboxylate (1.08 eq, 495 mg, 1.8 mmol), and Iron(II) sulfateheptahydrate (0.3 eq, 136 mg, 0.48 mmol) in DMSO (15 mL) at room temperature. After 1-2 min another portion of Iron(II) sulfateheptahydrate (0.3 eq, 136 mg, 0.48 mmol) was added and the reaction mixture was stirred at room temperature for 30 min. The reaction mixture was poured into a mixture of sat. NaHCO₃(aq.) (20 mL) and EtOAc (25 mL). The aqueous and organic layers were partitioned, and the aqueous layer was extracted with EtOAc (2×15 mL). The combined organic layers were washed with brine, dried with anhydrous Na₂SO₄, and filtered. The filtrate was concentrated under reduced pressure and purified by silica gel column chromatography (10-80% EtOAc in hexanes) to afford tert-butyl 3-[2-chloro-7-methyl-4-[3-(trifluoromethyl)-1-bicyclo[1.1.1]pentanyl]pteridin-6-yl]azetidine-1-carboxylate (A109, 255 mg, 0.43 mmol, 27% yield) as a solid. ESI-MS (m/z)-: 468.2 (obs).

### Synthesis of Example 86

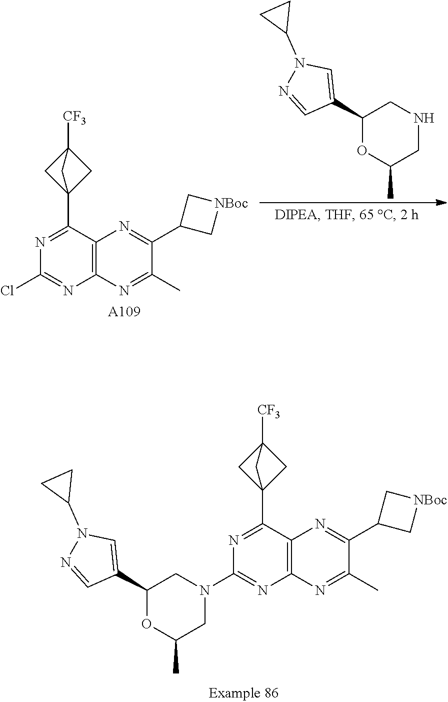

A vial was charged with tert-butyl 3-[2-chloro-7-methyl-4-[3-(trifluoromethyl)-1-bicyclo[1.1.1]pentanyl]pteridin-6-yl]azetidine-1-carboxylate (A109, 1.0 eq, 255 mg, 0.43 mmol) and (2S,6R)-2-(1-cyclopropylpyrazol-4-yl)-6-methyl-morpholine (1.1 eq, 99 mg, 0.48 mmol) and anhydrous THF (3 mL). DIPEA (3.0 eq, 227 μL, 1.3 mmol) was added, the vial was sealed and the reaction was stirred at 65° C. for 2 h. The reaction mixture was cooled down to RT, quenched with water and dissolved in EtOAc (10 mL). The aqueous phase was extracted with EtOAc (3×10 mL). The combined organic layers were washed with brine, dried over anhydrous Na₂SO₄, and filtered. The filtrate was concentrated under reduced pressure and purified by silica gel column chromatography (50-100% EtOAc in Hexanes) to afford tert-butyl 3-[2-[(2S,6R)-2-(1-cyclopropylpyrazol-4-yl)-6-methyl-morpholin-4-yl]-7-methyl-4-[3-(trifluoromethyl)-1-bicyclo[1.1.1]pentanyl]pteridin-6-yl]azetidine-1-carboxylate (Example 86, 180 mg, 0.28 mmol, 65% yield) as a solid. ESI-MS (m/z)+: 641.4 (obs). ¹H NMR (CHCl₃-d, 400 MHz): δ_{H} 7.58 (2H, d, J=9.8 Hz), 5.00-5.08 (2H, m), 4.59 (1H, dd, J=10.8, 2.7 Hz), 4.35 (2H, t, J=8.3 Hz), 4.25-4.31 (2H, m), 4.06-4.12 (1H, m), 3.77-3.85 (1H, m), 3.60-3.66 (1H, m), 3.01-3.08 (1H, m), 2.82 (1H, dd, J=13.4, 10.6 Hz), 2.62 (3H, s), 2.60 (6H, s), 1.47 (9H, s), 1.33 (3H, d, J=6.0 Hz), 1.14-1.18 (2H, m), 1.05-1.08 (2H, m). ¹⁹F NMR (CHCl₃-d, 376 MHz): δ_{F}−73.0 (3F, s).

### Method 43: Synthesis of Example 88

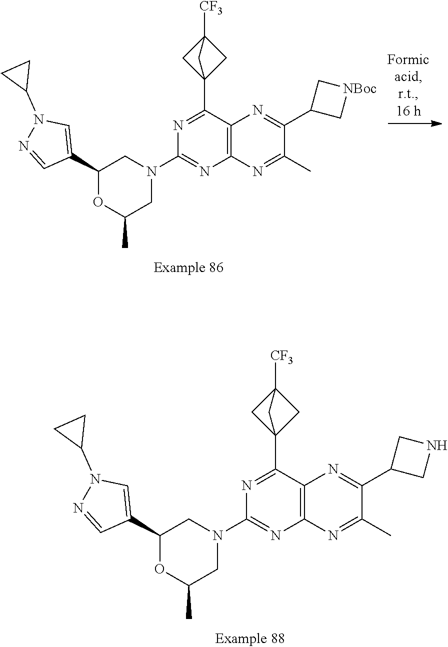 [SMILES: Cc1nc2nc(N3C[C@@H](C)O[C@@H](c4cnn(C5CC5)c4)C3)nc(C34CC(C(F)(F)F)(C3)C4)c2nc1C1CN(C(=O)OC(C)(C)C)C1 ; Cc1nc2nc(N3C[C@@H](C)O[C@@H](c4cnn(C5CC5)c4)C3)nc(C34CC(C(F)(F)F)(C3)C4)c2nc1C1CNC1]

TFA (20.0 eq, 289 μL, 3.8 mmol) was added to a solution of tert-butyl 3-[2-[(2S,6R)-2-(1-cyclopropylpyrazol-4-yl)-6-methyl-morpholin-4-yl]-7-methyl-4-[3-(trifluoromethyl)-1-bicyclo[1.1.1]pentanyl]pteridin-6-yl]azetidine-1-carboxylate (Example 86, 1.0 eq, 120 mg, 0.19 mmol) in DCM (1.9 mL) and the reaction was stirred at 20° C. for 2 h. The reaction mixture was concentrated under reduced pressure and purified by silica gel flash column chromatography (0-10% MeOH (with 1% NH₄OH) in DCM) to give a solid. The solid was dissolved in DCM and washed with NaOH (aq) 1M. The aqueous phase was further extracted with DCM, dried over anhydrous Na₂SO₄, filtered and concentrated under reduced pressure to afford (2S,6R)-4-[6-(azetidin-3-yl)-7-methyl-4-[3-(trifluoromethyl)-1-bicyclo[1.1.1]pentanyl]pteridin-2-yl]-2-(1-cyclopropylpyrazol-4-yl)-6-methyl-morpholine (Example 88, 74 mg, 0.14 mmol, 73% yield) as a solid. ESI-MS (m/z)+: 541.3 (obs). ¹⁹F NMR (CHCl₃-d, 376 MHz): δ_{F}−73.0 (3F, s). ¹H NMR (CHCl₃-d, 400 MHz): δ_{H} 7.52-7.53 (2H, m), 4.90-5.07 (2H, m), 4.55-4.58 (1H, m), 4.30-4.46 (3H, m), 4.14-4.25 (2H, m), 3.76-3.84 (1H, m), 3.55-3.61 (1H, m), 3.01-3.08 (1H, m), 2.77-2.83 (1H, m), 2.61 (6H, s), 2.58-2.58 (3H, s), 1.32 (3H, d, J=6.2 Hz), 1.11-1.13 (2H, m), 0.99-1.04 (2H, m).

### Method 44: Synthesis of Example 87

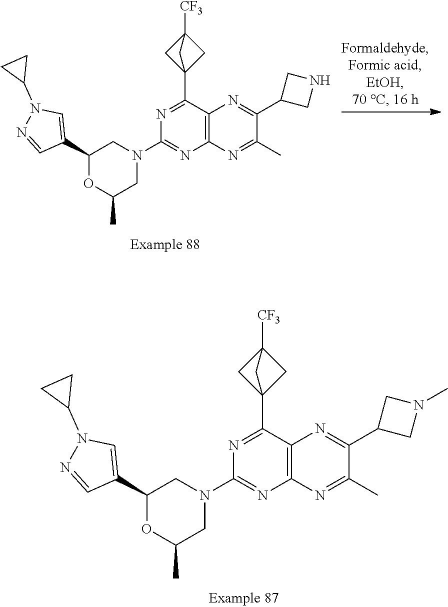 [SMILES: Cc1nc2nc(N3C[C@@H](C)O[C@@H](c4cnn(C5CC5)c4)C3)nc(C34CC(C(F)(F)F)(C3)C4)c2nc1C1CNC1 ; Cc1nc2nc(N3C[C@@H](C)O[C@@H](c4cnn(C5CC5)c4)C3)nc(C34CC(C(F)(F)F)(C3)C4)c2nc1C1CN(C)C1]

A vial was charged with (2S,6R)-4-[6-(azetidin-3-yl)-7-methyl-4-[3-(trifluoromethyl)-1-bicyclo[1.1.1]pentanyl]pteridin-2-yl]-2-(1-cyclopropylpyrazol-4-yl)-6-methyl-morpholine (Example 88, 1.0 eq, 64 mg, 0.12 mmol), formic acid (2.82 eq, 13 μL, 0.33 mmol), formaldehyde (37% solution in water, 1.45 eq, 13 μL, 0.17 mmol) and EtOH (6.5 mL). The reaction vial was sealed and stirred at 70° C. for 24 h. Additional formaldehyde was added (13 μL) and the reaction mixture stirred at 70° C. for another 24 h. The reaction mixture was taken up in heptanes and concentrated under reduced pressure to give a residue. The residue was purified by normal phase flash chromatography (10% MeOH (1% NH₄OH) in DCM), followed by reversed-phase column chromatography (10-90% ACN in (10 mM) NH₄CO₃ in water) to afford (2S,6R)-2-(1-cyclopropylpyrazol-4-yl)-6-methyl-4-[7-methyl-6-(1-methylazetidin-3-yl)-4-[3-(trifluoromethyl)-1-bicyclo[1.1.1]pentanyl]pteridin-2-yl]morpholine (Example 87, 11 mg, 0.02 mmol, 17% yield) as a solid. ESI-MS (m/z)+: 555.3 (obs). ¹⁹F NMR (CHCl₃-d, 376 MHz): δ^{F}−73.1 (3F, s). ¹H NMR (CHCl₃-d, 400 MHz): δ_{H} 7.54 (2H, t, J=0.9 Hz), 4.90-5.07 (2H, m), 4.55-4.59 (1H, m), 4.07-4.23 (3H, m), 3.56-3.83 (4H, m), 2.98-3.09 (1H, m), 2.76-2.85 (1H, m), 2.57-2.66 (12H, m), 1.33 (3H, d, J=6.0 Hz), 1.09-1.16 (2H, m), 1.00-1.06 (2H, m).

### Method 45: Synthesis of Example 100

### Synthesis of A110a and A110b

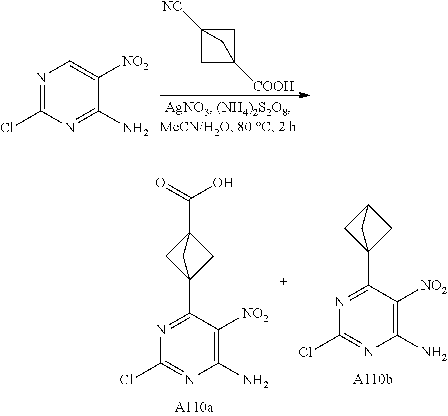 [SMILES: Nc1nc(Cl)ncc1[N+](=O)[O-] ; N#CC12CC(C(=O)O)(C1)C2 ; Nc1nc(Cl)nc(C23CC(C(=O)O)(C2)C3)c1[N+](=O)[O-] ; Nc1nc(Cl)nc(C23CC(C2)C3)c1[N+](=O)[O-]]

To a round-bottom flask equipped with a Teflon-coated magnetic stirring bar and a condenser was added 2-chloro-5-nitro-pyrimidin-4-amine (1.0 eq, 1.0 g, 5.73 mmol), 3-cyanobicyclo[1.1.1]pentane-1-carboxylic acid (1.3 eq, 1.02 g, 7.45 mmol) and AgNO₃ (1.3 eq, 1.26 g, 7.45 mmol). MeCN (20 mL) and Water (9.5 mL) were added, and the reaction mixture was vigorously stirred at 80° C. After 5 min, ammonium persulfate (3.3 eq, 4.31 g, 18.9 mmol) was added, and the reaction mixture was stirred at 80° C. for 2 h. The resulting mixture was cooled down to room temperature and filtered through celite rinsing with EtOAc. The filtrate was concentrated under reduced pressure to afford a 63/27 mixture of 3-(6-amino-2-chloro-5-nitro-pyrimidin-4-yl)bicyclo[1.1.1]pentane-1-carboxylic acid (A110a) and 6-(1-bicyclo[1.1.1]pentanyl)-2-chloro-5-nitro-pyrimidin-4-amine (A 110b) (1.0 g, 3.51 mmol, 61% yield) as a solid which was used directly in the next step. A110a: ESI-MS (m/z)+: 285.1 (obs). A110b: ESI-MS (m/z)+: 241.2 (obs).

### Synthesis of A111a and A111b

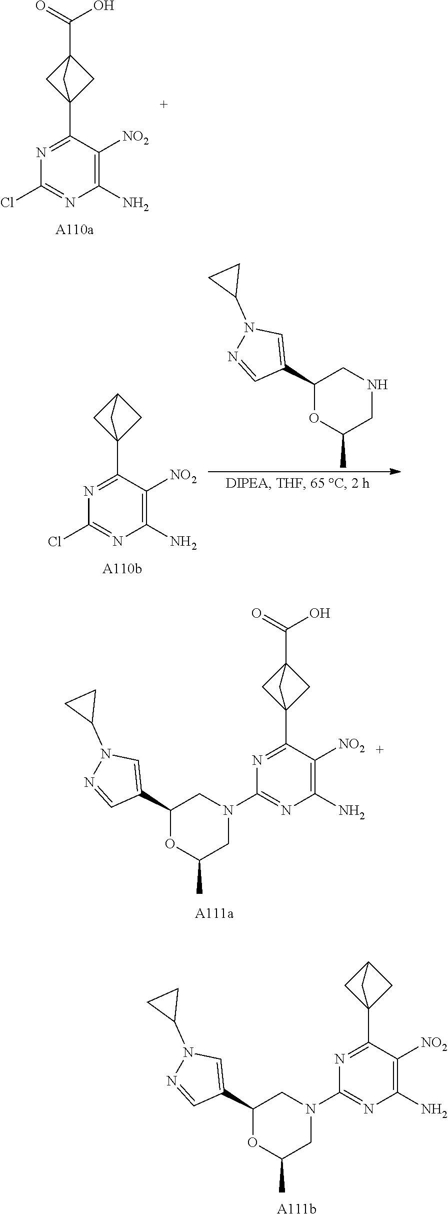

A round-bottomed flask equipped with a Teflon-coated magnetic stirring bar was charged with a 63/27 mixture of 3-(6-amino-2-chloro-5-nitro-pyrimidin-4-yl)bicyclo[1.1.1]pentane-1-carboxylic acid (A110a) and 6-(1-bicyclo[1.1.1]pentanyl)-2-chloro-5-nitro-pyrimidin-4-amine (A110b) (1.0 eq, 1.0 g, 2.99 mmol), (2S,6R)-2-(1-cyclopropylpyrazol-4-yl)-6-methyl-morpholine (1.1 eq, 688 mg, 3.28 mmol) and anhydrous THF (10 mL). DIPEA (3.0 eq, 1.56 mL, 8.96 mmol) was added and the reaction mixture was stirred at 65° C. for 1.5 h. The reaction mixture was cooled down, quenched with water (15 mL) and diluted with EtOAc (15 mL). The aqueous phase was extracted with EtOAc (3×50 mL) and the combined organic phases were set aside. The aqueous phase was then acidified to pH=3 with 1N HCl (aq.) and extracted with EtOAc (3×50 mL). The combined organic layers were washed with brine, dried over anhydrous Na₂SO₄, filtered and concentrated under reduced pressure to afford 3-[6-amino-2-[(2S,6R)-2-(1-cyclopropylpyrazol-4-yl)-6-methyl-morpholin-4-yl]-5-nitro-pyrimidin-4-yl]bicyclo[1.1.1]pentane-1-carboxylic acid (A111a) (588 mg, 1.06 mmol, 35% yield) as a solid. A111a: ESI-MS (m/z)+: 456.4 (obs). ¹H NMR (CHCl₃-d, 400 MHz): δ_{H} 7.53 (2H, s), 4.69-4.97 (2H, br m), 4.50 (1H, d, J=10.8 Hz), 3.72 (1H, s), 3.59 (1H, s), 2.94 (1H, t, J=12.2 Hz), 2.73 (1H, t, J=12.0 Hz), 2.54 (6H, s), 1.30 (3H, d, J=6.2 Hz), 1.01-1.11 (4H, m), 0.86 (1H, t, J=10.4 Hz).

The combined organic layers that were set aside were washed with brine, dried over anhydrous Na₂SO₄, and filtered. The filtrate was concentrated under reduced pressure and purified by silica gel flash column chromatography (5-100% EtOAc in Hexanes) to afford 6-(1-bicyclo[1.1.1]pentanyl)-2-[(2S,6R)-2-(1-cyclopropylpyrazol-4-yl)-6-methyl-morpholin-4-yl]-5-nitro-pyrimidin-4-amine (A111b) (112 mg, 0.25 mmol, 8% yield) as a solid. A111b: ESI-MS (m/z)+: 412.3 (obs). ¹H NMR (CHCl₃-d, 400 MHz): δ_{H} 7.51-7.55 (2H, m), 4.58-4.91 (2H, br m), 4.50 (1H, d, J=10.8 Hz), 3.71 (1H, s), 3.57 (1H, d, J=6.9 Hz), 2.93 (1H, t, J=12.0 Hz), 2.71 (1H, t, J=12.0 Hz), 2.22 (6H, s), 1.30 (4H, d, J=6.2 Hz), 1.06-1.11 (3H, m), 1.02 (2H, d, J=7.2 Hz).

### Synthesis of A112

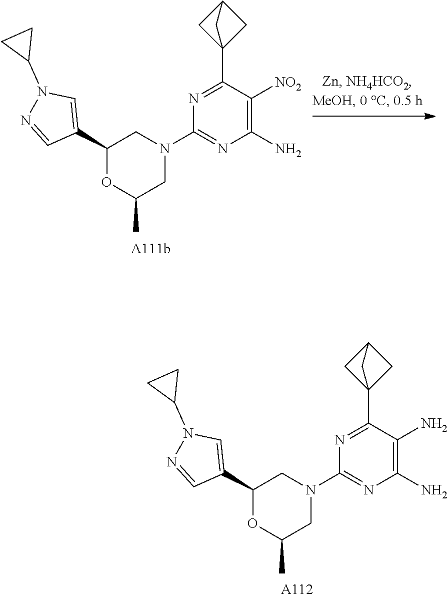

To a solution of 6-(1-bicyclo[1.1.1]pentanyl)-2-[(2S,6R)-2-(1-cyclopropylpyrazol-4-yl)-6-methyl-morpholin-4-yl]-5-nitro-pyrimidin-4-amine (A111b) (1.0 eq, 115 mg, 0.25 mmol) in MeOH (4 mL) at 0° C. was added ammonium formate (8.0 eq, 127 mg, 2.0 mmol), Zn powder (8.0 eq, 132 mg, 2.0 mmol) and the reaction mixture was stirred at 0° C. for 0.5 h. The reaction mixture was filtered through a pad of celite rinsing with DCM. The filtrate was concentrated under reduced pressure to afford 6-(1-bicyclo[1.1.1]pentanyl)-2-[(2S,6R)-2-(1-cyclopropylpyrazol-4-yl)-6-methyl-morpholin-4-yl]pyrimidine-4,5-diamine (A112, 95 mg, 0.25 mmol, 99% yield) as a solid which was used in the next step without further purification. ESI-MS (m/z)+: 382.3 (obs).

### Synthesis of Example 100

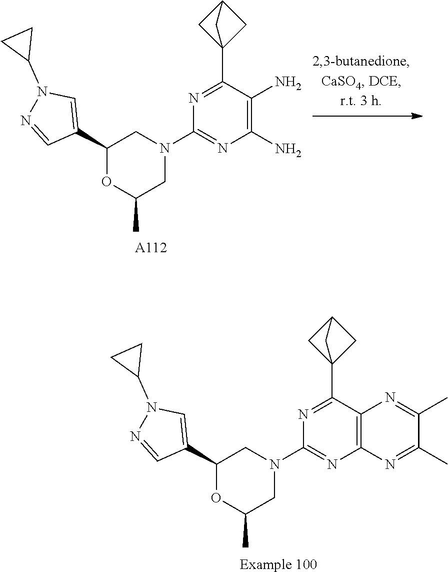

To a solution of 6-(1-bicyclo[1.1.1]pentanyl)-2-[(2S,6R)-2-(1-cyclopropylpyrazol-4-yl)-6-methyl-morpholin-4-yl]pyrimidine-4,5-diamine (A112, 1.0 eq, 95 mg, 0.25 mmol) in DCE (6 mL) was added CaSO₄ (10 eq, 339 mg, 2.5 mmol) and the suspension was stirred at room temperature for 3 h. The reaction mixture was filtered through a celite pad rinsing with DCM. The filtrate was concentrated under reduced pressure and purified by silica gel flash column chromatography (0-10% MeOH in DCM) to afford (2S,6R)-4-[4-(1-bicyclo[1.1.1]pentanyl)-6,7-dimethyl-pteridin-2-yl]-2-(1-cyclopropylpyrazol-4-yl)-6-methyl-morpholine (Example 100, 33 mg, 0.08 mmol, 31% yield) as a solid. ESI-MS (m/z)+: 432.4 (obs). ¹H NMR (DMSO-d₆, 400 MHz): δ_{H} 7.84 (1H, s), 7.46 (1H, s), 4.68-4.80 (2H, m), 4.49 (1H, dd, J=10.8, 2.5 Hz), 3.67-3.73 (2H, m), 2.96-3.06 (1H, m), 2.68-2.75 (1H, m), 2.61 (1H, s), 2.58 (6H, s), 2.35 (6H, s), 1.16-1.21 (3H, m), 0.91-1.05 (4H, m).

### Method 46: Synthesis of Example 102

### Synthesis of A113

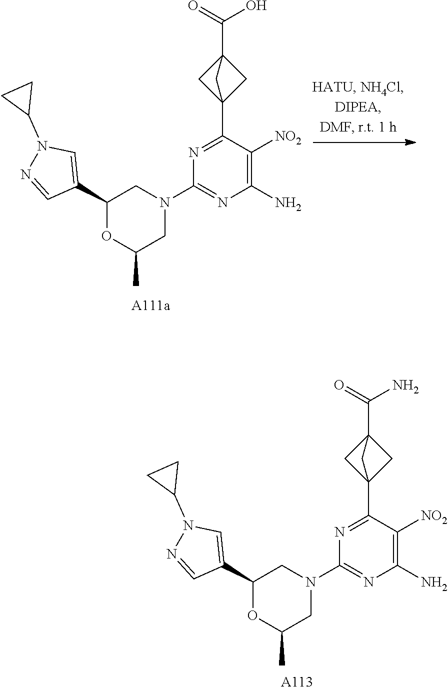

A reaction vial was charged with 3-[6-amino-2-[(2S,6R)-2-(1-cyclopropylpyrazol-4-yl)-6-methyl-morpholin-4-yl]-5-nitro-pyrimidin-4-yl]bicyclo[1.1.1]pentane-1-carboxylic acid (Alila, 1.0 eq, 540 mg, 0.97 mmol), NH₄Cl (5.0 eq, 260 mg, 4.9 mmol), HATU (1.1 eq, 407 mg, 1.1 mmol) and DMF (10 mL). DIPEA (3.0 eq, 510 μL, 2.9 mmol) was added dropwise and the reaction mixture was stirred at RT for 2 h. Water was added to the reaction mixture and the aqueous phase was extracted with DCM (3×45 mL). The combined organic layers were washed with water, brine, dried over anhydrous Na₂SO₄, and filtered. The filtrate was concentrated under reduced pressure and purified by silica gel flash column chromatography (0-10% MeOH in DCM) to afford 3-[6-amino-2-[(2S,6R)-2-(1-cyclopropylpyrazol-4-yl)-6-methyl-morpholin-4-yl]-5-nitro-pyrimidin-4-yl]bicyclo[1.1.1]pentane-1-carboxamide (A113, 500 mg, 0.88 mmol, 90% yield) as a solid. ESI-MS (m/z)+: 455.4 (obs).

### Synthesis of A114

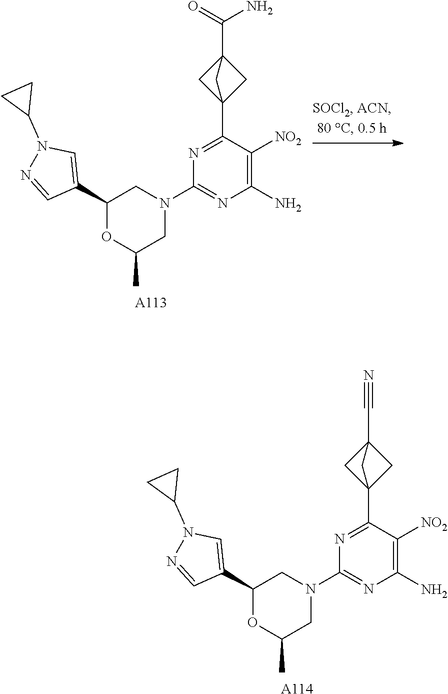

A reaction vial was charged with 3-[6-amino-2-[(2S,6R)-2-(1-cyclopropylpyrazol-4-yl)-6-methyl-morpholin-4-yl]-5-nitro-pyrimidin-4-yl]bicyclo[1.1.1]pentane-1-carboxamide (A113, 1.0 eq, 500 mg, 0.9 mmol) and MeCN (9.0 mL). To the suspension was added dropwise SOCl₂ (20 eq, 1.3 mL, 18.0 mmol), and the reaction was stirred at 80° C. for 0.5 h. The resulting mixture was concentrated under reduced pressure several times, using heptanes as an azeotrope. The residue was purified by silica gel flash column chromatography (0-10% MeOH in DCM) to yield a paste. The paste was dissolved in DCM (10 mL) and washed with sat. NaHCO₃(aq.) (2×15 mL), dried with anhydrous Na₂SO₄, filtered and concentrated under reduced pressure to yield 3-[6-amino-2-[(2S,6R)-2-(1-cyclopropylpyrazol-4-yl)-6-methyl-morpholin-4-yl]-5-nitro-pyrimidin-4-yl]bicyclo[1.1.1]pentane-1-carbonitrile (A114, 250 mg, 0.57 mmol, 63% yield) as a solid. ESI-MS (m/z)+: 437.3 (obs). ¹H NMR (DMSO-d₆, 400 MHz): δ_{H} 7.90 (2H, br s), 7.81 (1H, s), 7.43 (1H, s), 4.64 (2H, br s), 4.42 (1H, d, J=10.4 Hz), 3.65 (2H, d, J=20.1 Hz), 2.97 (1H, t, J=12.1 Hz), 2.66 (1H, s), 2.56 (6H, d, J=9.6 Hz), 1.15 (3H, br s), 0.91-0.99 (4H, m).

### Synthesis of A115

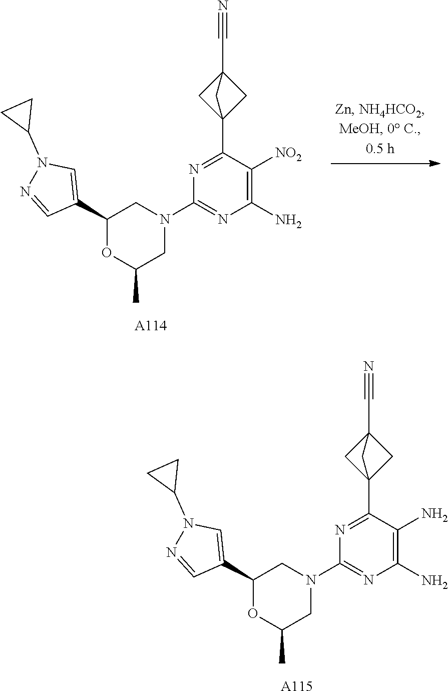

To a solution of 3-[6-amino-2-[(2S,6R)-2-(1-cyclopropylpyrazol-4-yl)-6-methyl-morpholin-4-yl]-5-nitro-pyrimidin-4-yl]bicyclo[1.1.1]pentane-1-carbonitrile (A114, 1.0 eq, 115 mg, 0.25 mmol) in MeOH (4 mL) at 0° C., was added ammonium formate (8.0 eq, 127 mg, 2.0 mmol) and Zn (8.0 eq, 132 mg, 2.0 mmol) and the reaction mixture was stirred at 0° C. for 0.5 h. The reaction mixture was filtered over a pad of celite rinsing with DCM. The filtrate was concentrated under reduced pressure to afford 3-[5,6-diamino-2-[(2S,6R)-2-(1-cyclopropylpyrazol-4-yl)-6-methylmorpholin-4-yl]pyrimidin-4-yl]-bicyclo[1.1.1]pentane-1-carbonitrile (A115, 240 mg, 0.59 mmol, 99% yield) as a solid. ESI-MS (m/z)+: 407.3 (obs).

### Synthesis of Example 102

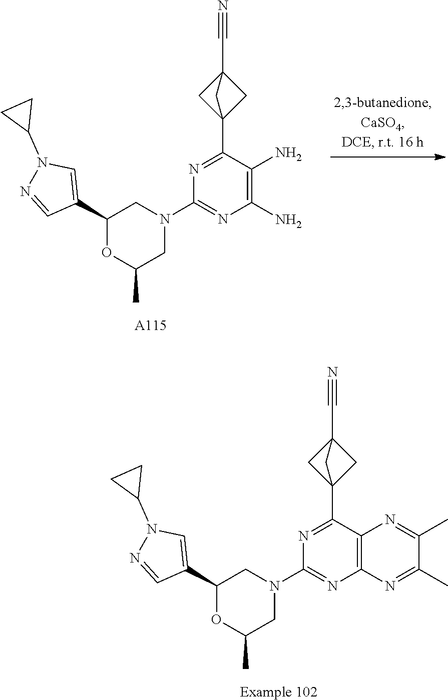

To a solution of 3-[5,6-diamino-2-[(2S,6R)-2-(1-cyclopropylpyrazol-4-yl)-6-methyl-morpholin-4-yl]pyrimidin-4-yl]bicyclo[1.1.1]pentane-1-carbonitrile (A115, 1.0 eq, 253 mg, 0.59 mmol) in DCE (8 mL) was added CaSO₄ (10 eq, 804 mg, 5.9 mmol), and the reaction was stirred at RT for 16 h under nitrogen. The reaction mixture was filtered through a pad of celite pad rinsing with DCM. The filtrate was concentrated under reduced pressure and purified by silica gel flash column chromatography (10-100% EtOAc in Hexanes) to afford 3-[2-[(2S,6R)-2-(1-cyclopropylpyrazol-4-yl)-6-methyl-morpholin-4-yl]-6,7-dimethyl-pteridin-4-yl]bicyclo[1.1.1]pentane-1-carbonitrile (Example 102, 147 mg, 0.32 mmol, 54% yield) as a solid. ESI-MS (m/z)+: 457.4 (obs). ¹H NMR (DMSO-d₆, 400 MHz): δ_{H} 7.83 (1H, s), 7.45 (1H, s), 4.67-4.76 (2H, m), 4.46-4.49 (1H, m), 3.66-3.72 (2H, m), 2.95-3.07 (1H, m), 2.79 (6H, s), 2.67-2.75 (1H, m), 2.58 (6H, s), 1.19 (3H, d, J=6.2 Hz), 0.93-1.05 (4H, m).

### Method 47: Synthesis of Example 106, Example 107, and Example 108

### Synthesis of A119

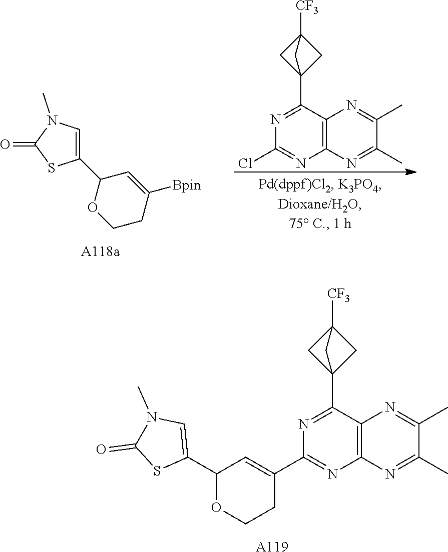

A reaction vial equipped with a Teflon-coated magnetic stirring bar was charged with 3-methyl-5-[4-(4,4,5,5-tetramethyl-1,3,2-dioxaborolan-2-yl)-3,6-dihydro-2H-pyran-6-yl]thiazol-2-one (A118a, 1.0 eq, 271 mg, 0.76 mmol), 2-chloro-6,7-dimethyl-4-[3-(trifluoromethyl)-1-bicyclo[1.1.1]pentanyl]pteridine (1.05 eq, 260 mg, 0.79 mmol), K₃PO₄ (2.5 eq, 400 mg, 1.89 mmol), Pd(dppf)Cl₂ DCM (0.1 eq, 62 mg, 0.076 mmol), 1,4-dioxane (5 mL), and water (0.5 mL). The vial was sealed, degassed under argon and stirred at 75° C. for 1 h. The reaction mixture was cooled to RT and filtered over celite, rinsing with EtOAc. The filtrate was concentrated under reduced pressure and purified by silica gel flash column chromatography (50-100% EtOAc in Hexanes) to afford 5-[4-[6,7-dimethyl-4-[3-(trifluoromethyl)-1-bicyclo[1.1.1]pentanyl]pteridin-2-yl]-3,6-dihydro-2H-pyran-6-yl]-3-methyl-thiazol-2-one (A119, 285 mg, 0.58 mmol, 77% yield) as a solid. ¹H NMR (CHCl₃-d, 400 MHz): δ_{H} 7.53-7.52 (m, 1H), 6.56 (s, 1H), 5.32 (d, J=2.3 Hz, 1H), 4.06 (s, 1H), 3.95 (d, J=5.9 Hz, 1H), 3.32 (s, 3H), 2.91 (t, J=2.4 Hz, 2H), 2.82 (s, 3H), 2.78 (s, 3H), 2.65 (s, 6H). ¹⁹F NMR (CHCl₃-d, 376 MHz): δ_{F}−73.0 (s, 3F). ESI-MS (m/z)+: 490.3 (obs).

### Synthesis of Example 106

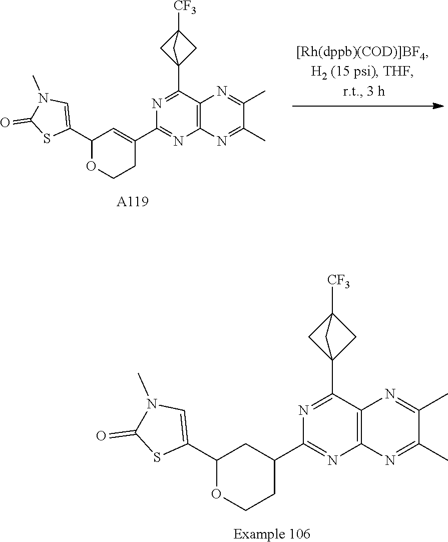

A round-bottomed flask equipped with a Teflon-coated magnetic stirring bar was charged with [Rh(dppb)(COD)] BF₄ (0.25 eq, 105 mg, 0.15 mmol). The flask was purged with argon before adding a solution of 5-[4-[6,7-dimethyl-4-[3-(trifluoromethyl)-1-bicyclo[1.1.1]pentanyl]pteridin-2-yl]-3,6-dihydro-2H-pyran-6-yl]-3-methyl-thiazol-2-one (A119, 1.0 eq, 285 mg, 0.58 mmol) in THF (5 mL). The heterogeneous reaction mixture was then purged with H₂ (15 psi) and stirred at RT under H₂ atmosphere for 5 h. The reaction mixture was filtered over celite, rinsing with EtOAc. The filtrate was concentrated under reduced pressure and purified by silica gel flash column chromatography (0-10% MeOH in DCM) to yield 3-methyl-5-[rac-(2R,4S)-4-[6,7-dimethyl-4-[3-(trifluoromethyl)-1-bicyclo[1.1.1]pentanyl]-pteridin-2-yl]tetrahydropyran-2-yl]thiazol-2-one ((rac)-Example 106, 258 mg, 0.53 mmol, 90% yield) as a solid. ESI-MS (m/z)+: 492.3 (obs). ¹H NMR (CHCl₃-d, 400 MHz): δ_{H} 6.55 (d, J=0.9 Hz, 1H), 4.43 (d, J=10.3 Hz, 1H), 4.27-4.23 (m, 1H), 3.76 (td, J=11.7, 3.1 Hz, 1H), 3.44-3.38 (m, 1H), 3.30 (s, 3H), 2.81 (s, 3H), 2.78 (s, 3H), 2.64 (s, 6H), 2.37 (d, J=14.6 Hz, 1H), 2.16-2.06 (m, 3H). ¹⁹F NMR (CHCl₃-d, 376 MHz): δ_{F}−72.9 (s, 3F).

### Synthesis of Example 107 and Example 108

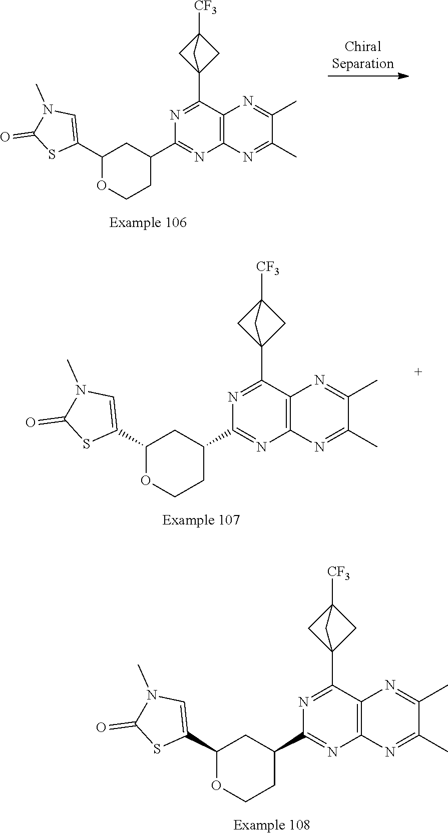

3-methyl-5-[rac-(2R,4S)-4-[6,7-dimethyl-4-[3-(trifluoromethyl)-1-bicyclo[1.1.1]pentanyl]-pteridin-2-yl]tetrahydropyran-2-yl]thiazol-2-one (Example 106, 258 mg, 0.53 mmol) was purified by Chiral HPLC (Condition 1, Gradient 1) to afford 5-[(2S,4R)-4-[6,7-dimethyl-4-[3-(trifluoromethyl)-1-bicyclo[1.1.1]pentanyl]pteridin-2-yl]tetrahydropyran-2-yl]-3-methyl-thiazol-2-one (Example 107, 83 mg, 0.17 mmol, 29% yield, 99% ee) as a solid and 5-[(2R,4S)-4-[6,7-dimethyl-4-[3-(trifluoromethyl)-1-bicyclo[1.1.1]pentanyl]pteridin-2-yl]tetrahydropyran-2-yl]-3-methyl-thiazol-2-one (Example 108, 79 mg, 0.16 mmol, 27% yield, 99% ee) as a solid. Example 107: ESI-MS (m/z)+: 492.3 (obs). ¹H NMR (CHCl₃-d, 400 MHz): δ_{H} 6.55 (d, J=0.9 Hz, 1H), 4.43 (d, J=10.3 Hz, 1H), 4.27-4.23 (m, 1H), 3.76 (td, J=11.7, 3.1 Hz, 1H), 3.44-3.38 (m, 1H), 3.30 (s, 3H), 2.81 (s, 3H), 2.78 (s, 3H), 2.64 (s, 6H), 2.37 (d, J=14.6 Hz, 1H), 2.16-2.06 (m, 3H). ¹⁹F NMR (CHCl₃-d, 376 MHz): δ_{F}−72.9 (s, 3F). Example 108: ESI-MS (m/z)+: 492.3 (obs). ¹H NMR (CHCl₃-d, 400 MHz): δ_{H} 6.55 (d, J=0.9 Hz, 1H), 4.43 (d, J=10.3 Hz, 1H), 4.27-4.23 (m, 1H), 3.76 (td, J=11.7, 3.1 Hz, 1H), 3.44-3.38 (m, 1H), 3.30 (s, 3H), 2.81 (s, 3H), 2.78 (s, 3H), 2.64 (s, 6H), 2.37 (d, J=14.6 Hz, 1H), 2.16-2.06 (m, 3H). ¹⁹F NMR (CHCl₃-d, 376 MHz): δ_{F}−72.9 (s, 3F).

### Method 48: Synthesis of Example 109 and Example 110

### Synthesis of A122

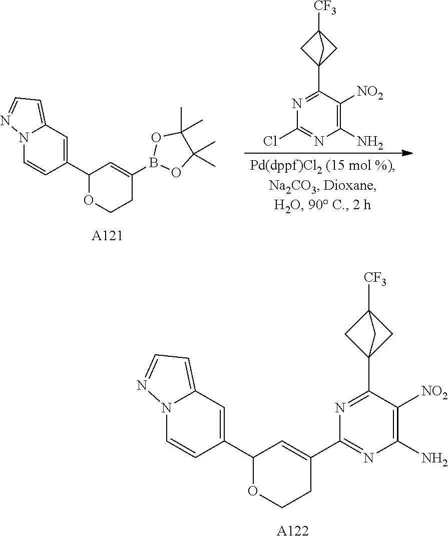

To a glass vial equipped with a Teflon-coated magnetic stirring bar was charged with 5-[4-(4,4,5,5-tetramethyl-1,3,2-dioxaborolan-2-yl)-3,6-dihydro-2H-pyran-6-yl]pyrazolo[1,5-a]pyridine (A121, 1.0 eq, 0.2 g, 0.61 mmol), 2-chloro-5-nitro-6-[3-(trifluoromethyl)-1-bicyclo[1.1.1]pentanyl]pyrimidin-4-amine (0.9 eq, 172 mg, 0.56 mmol), Pd(dppf)Cl₂ (0.15 eq, 75 mg, 0.092 mmol), 1,4-dioxane (9 mL) and 2.0 M Na₂CO₃ (aq.) (3.0 eq, 0.93 mL, 1.86 mmol). The vial was sealed, purged under argon and stirred at 90° C. for 3 h. The reaction was quenched with NaHCO₃ and extracted with DCM (×3). The combined organic layers were washed with brine, dried over Na₂SO₄, and filtered. The filtrate was concentrated under reduced pressure and purified by normal phase column chromatography (5-50% EtOAc in hexanes) to afford 5-nitro-2-(6-pyrazolo[1,5-a]pyridin-5-yl-3,6-dihydro-2H-pyran-4-yl)-6-[3-(trifluoromethyl)-1-bicyclo[1.1.1]pentanyl]pyrimidin-4-amine (A122, 60.0 mg, 0.13 mmol, 21% yield). ESI-MS (m/z)+: 473.3 (obs).

### Synthesis of A123 and A124

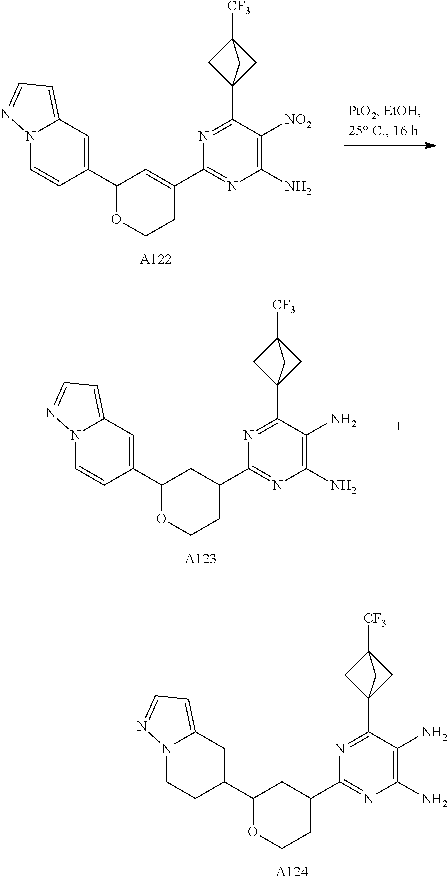

5-nitro-2-[rac-(6R)-6-pyrazolo[1,5-a]pyridin-5-yl-3,6-dihydro-2H-pyran-4-yl]-6-[3-(trifluoromethyl)-1-bicyclo[1.1.1]pentanyl]pyrimidin-4-amine (A122, 1.0 eq, 220 mg, 0.47 mmol) was dissolved in EtOH (4.7 mL), the mixture was purged with argon and PtO₂ (5.0 eq, 529 mg, 2.3 mmol) was added. The reaction was purged with hydrogen and was stirred under 1 atm of H₂ overnight. The reaction was purged with argon, filtered through a pad of celite and concentrated under reduced pressure. The crude material was purified by normal phase column chromatography (0-5% MeOH in DCM) to afford 2-[rac-2-pyrazolo[1,5-a]pyridin-5-yltetrahydropyran-4-yl]-6-[3-(trifluoromethyl)-1-bicyclo[1.1.1]pentanyl]pyrimidine-4,5-diamine (A123) and 2-(2-(4,5,6,7-tetrahydropyrazolo[1,5-a]pyridin-5-yl)tetrahydro-2H-pyran-4-yl)-6-(3-(trifluoromethyl)bicyclo[1.1.1]pentan-1-yl)pyrimidine-4,5-diamine (A124) as a mixture (115 mg, 0.26 mmol, 56% yield). A123: ESI-MS (m/z)+: 445.3 (obs). A124: ESI-MS (m/z)+: 449.4 (obs).

### Synthesis of Example 109 and Example 110

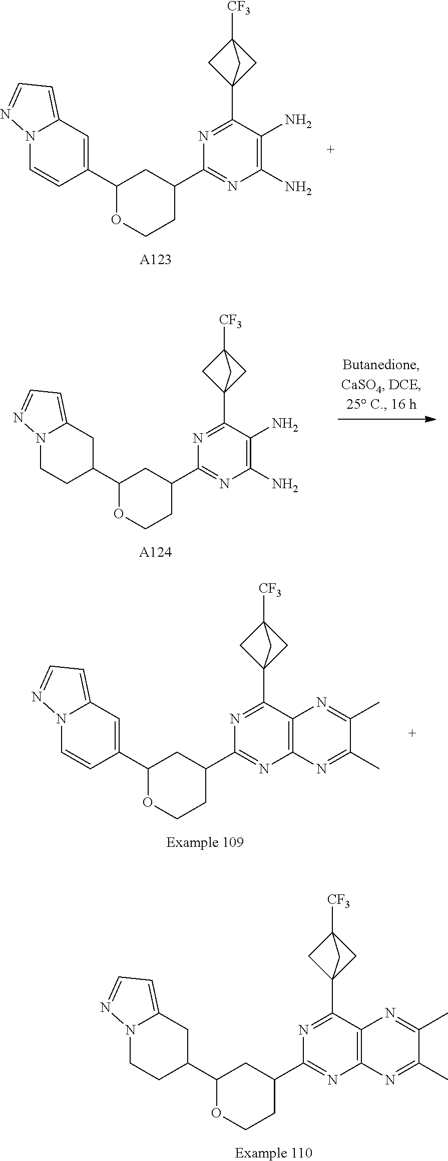

A mixture of 2-(2-pyrazolo[1,5-a]pyridin-5-yltetrahydropyran-4-yl)-6-[3-(trifluoromethyl)-1-bicyclo[1.1.1]pentanyl]pyrimidine-4,5-diamine and 2-(2-(4,5,6,7-tetrahydropyrazolo[1,5-a]pyridin-5-yl)tetrahydro-2H-pyran-4-yl)-6-(3-(trifluoromethyl)bicyclo[1.1.1]pentan-1-yl)pyrimidine-4,5-diamine (A123 and A124, 1.0 eq, 115 mg, 0.26 mmol) was stirred with butane-2,3-dione (1.0 eq, 0.023 mL, 0.26 mmol) and CaSO₄ (10.0 eq, 352 mg, 2.6 mmol) in DCE (2.6 mL) at room temperature overnight. The reaction was quenched with water and extracted with DCM (×3). The combined organic layers were washed with brine, dried over Na₂SO₄, and filtered. The filtrate was concentrated under reduced pressure and purified by reversed-phase column chromatography (15-75% MeCN in water(0.1% of formic acid)), followed by prep-HPLC (Condition 2, Gradient 5) to afford 6,7-dimethyl-2-(2-pyrazolo[1,5-a]pyridin-5-yltetrahydropyran-4-yl)-4-[3-(trifluoromethyl)-1-bicyclo[1.1.1]pentanyl]pteridine (Example 109, 6.3 mg, 0.013 mmol, 5% yield) and 6,7-dimethyl-2-[2-(4,5,6,7-tetrahydropyrazolo[1,5-a]pyridin-5-yl)tetrahydropyran-4-yl]-4-[3-(trifluoromethyl)-1-bicyclo[1.1.1]pentanyl]pteridine (Example 110, 33 mg, 0.067 mmol, 26% yield) as solids. Example 109: ¹H NMR (CHCl₃-d, 400 MHz): δ_{H} 8.42 (d, J=7.1 Hz, 1H), 7.91 (d, J=1.8 Hz, 1H), 7.56 (s, 1H), 6.81 (dd, J=7.2, 1.5 Hz, 1H), 6.49-6.46 (m, 1H), 4.59-4.55 (m, 1H), 4.38-4.33 (m, 1H), 3.89-3.81 (m, 1H), 3.54-3.46 (m, 1H), 2.83-2.79 (m, 3H), 2.75 (s, 3H), 2.61 (d, J=15.1 Hz, 6H), 2.45-2.39 (m, 1H), 2.21-2.15 (m, 2H), 2.09-1.99 (m, 1H). ¹⁹F-NMR (CHCl₃-d, 376 MHz) δ_{F}−73.0. ESI-MS (m/z)+: 495.3 (obs). Example 110: ¹H NMR (CHCl₃-d, 400 MHz): δ_{H} 7.42 (s, 1H), 5.96 (d, J=6.9 Hz, 1H), 4.35 (s, 1H), 4.19 (d, J=11.4 Hz, 1H), 4.04 (s, 1H), 3.67-3.61 (m, 1H), 3.38 (s, 2H), 2.94 (s, 1H), 2.80 (s, 3H), 2.76 (s, 3H), 2.64 (s, 6H), 2.22 (d, J=12.6 Hz, 1H), 2.11-1.83 (m, 6H). ¹⁹F-NMR (CHCl₃-d, 376 MHz) δ_{F}−73.0. ESI-MS (m/z)+: 499.4 (obs).

### Method 49: Synthesis of Example 111

### Synthesis of A127

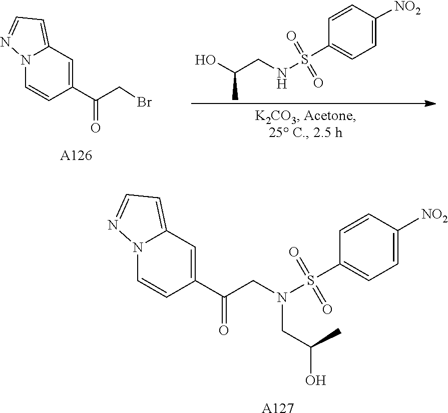

K₂CO₃ (3.0 eq, 1.42 g, 10.3 mmol) was added to a solution of 2-bromo-1-pyrazolo[1,5-a]pyridin-5-yl-ethanone (A126, 1.0 eq, 820 mg, 3.43 mmol) and N-[(2R)-2-hydroxypropyl]-4-nitrobenzene-sulfonamide (1.0 eq, 893 mg, 3.43 mmol) in acetone (8.6 mL), and the reaction was stirred at room temperature for 2.5 h. The reaction was quenched with water and extracted with EtOAc (×3). The combined organic layers were washed with brine, dried over Na₂SO₄, and filtered. The filtrate was concentrated under reduced pressure and purified by normal phase column chromatography (20-60% EtOAc in hexanes) to afford N-[(2R)-2-hydroxypropyl]-4-nitro-N-(2-oxo-2-pyrazolo[1,5-a]pyridin-5-yl-ethyl)benzenesulfonamide (A127, 1.12 g, 2.67 mmol, 78% yield). ESI-MS (m/z)+: 419.2 (obs).

### Synthesis of A128

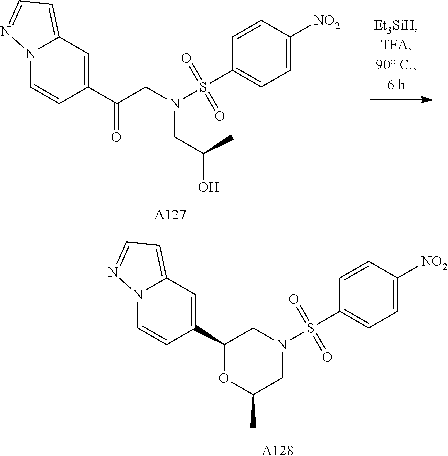

A flame dried vial under argon was charged with 4-nitro-N-(2-oxo-2-pyrazolo[1,5-a]pyridin-5-yl-ethyl)-N-[rac-(2R)-2-hydroxypropyl]benzenesulfonamide (A127, 1.0 eq, 380 mg, 0.91 mmol) and TFA (63.4 eq, 4.4 mL, 57.6 mmol). Triethylsilane (8.0 eq, 1.2 mL, 7.3 mmol) was added dropwise and the solution was stirred at 90° C. overnight. The resulting mixture was concentrated under reduced pressure and purified by normal phase column chromatography (5-60% EtOAc in hexanes) to afford (2R,6S)-2-methyl-4-(4-nitrophenyl)sulfonyl-6-pyrazolo[1,5-a]pyridin-5-yl-morpholine (A128, 290 mg, 0.72 mmol, 79% yield). ESI-MS (m/z)+: 403.2 (obs).

### Synthesis of A129

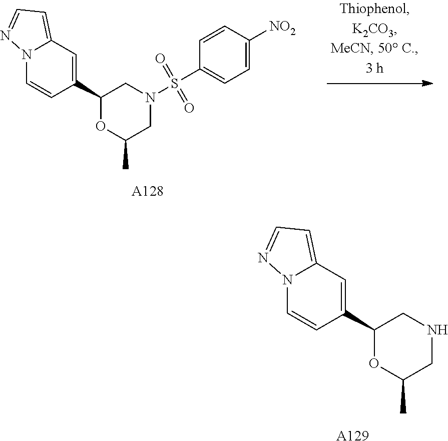

Thiophenol (3.0 eq, 45 μL, 0.44 mmol) was added to a suspension of (2R,6S)-2-methyl-4-(4-nitrophenyl)sulfonyl-6-pyrazolo[1,5-a]pyridin-5-yl-morpholine (A128, 1.0 eq, 59 mg, 0.15 mmol) and K₂CO₃ (5.0 eq, 101 mg, 0.73 mmol) in MeCN (1.5 mL) at 50° C. under argon atmosphere, and the reaction was stirred at 50° C. for 3 h. The reaction was quenched with HCl (10% aq.) and extracted with DCM. The organic layer was discarded, the aqueous layer was basified with NaHCO₃ and extracted with EtOAc (×3). The combined organic layers were dried over Na₂SO₄, filtered, and concentrated under reduced pressure to afford (2R,6S)-2-methyl-6-pyrazolo[1,5-a]pyridin-5-yl-morpholine (A129, 31.8 mg, 0.15 mmol, 99% yield).

### Synthesis of Example 111

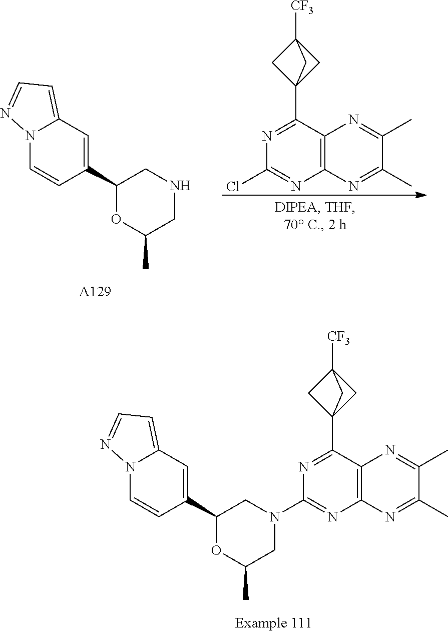

(2R,6S)-2-methyl-6-pyrazolo[1,5-a]pyridin-5-yl-morpholine (A129, 1.0 eq, 62 mg, 0.29 mmol), 2-chloro-6,7-dimethyl-4-[3-(trifluoromethyl)-1-bicyclo[1.1.1]pentanyl]pteridine (1.5 eq, 141 mg, 0.43 mmol) and DIPEA (3.0 eq, 0.15 mL, 0.86 mmol) in THF (3.0 mL) were stirred at 70° C. for 2 h. The resulting mixture was concentrated under reduced pressure and purified by normal phase column chromatography (0-3% MeOH in DCM) to afford (2R,6S)-4-[6,7-dimethyl-4-[3-(trifluoromethyl)-1-bicyclo[1.1.1]pentanyl]pteridin-2-yl]-2-methyl-6-pyrazolo[1,5-a]pyridin-5-yl-morpholine (Example 111, 34 mg, 0.067 mmol, 23% yield) as a solid. ESI-MS (m/z)+: 510.3 (obs). ¹H-NMR (CHCl₃-d, 400 MHz): δ_{H} 8.42-8.51 (1H, m), 7.93-7.99 (1H, m), 7.61-7.67 (1H, m), 6.80-6.89 (1H, m), 6.46-6.57 (1H, m), 5.07-5.20 (1H, m), 4.88-5.03 (1H, m), 4.57-4.66 (1H, m), 3.80-3.92 (1H, m), 2.88-2.97 (1H, m), 2.77-2.86 (1H, m), 2.66-2.70 (3H, m), 2.62-2.64 (3H, m), 2.56-2.60 (6H, m), 1.34-1.42 (3H, m). ¹⁹F-NMR (CHCl₃-d, 376 MHz) δ_{F}−72.9 (s, 3F).

### Method 50: Synthesis of Example 117

### Synthesis of A130

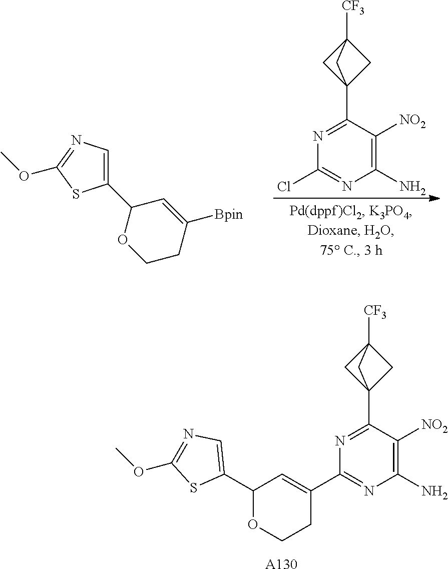

To a reaction vial equipped with a Teflon-coated magnetic stirring bar was added 2-methoxy-5-[4-(4,4,5,5-tetramethyl-1,3,2-dioxaborolan-2-yl)-3,6-dihydro-2H-pyran-6-yl]thiazole (1.0 eq, 98 mg, 0.27 mmol), 2-chloro-5-nitro-6-[3-(trifluoromethyl)-1-bicyclo[1.1.1]pentanyl]pyrimidin-4-amine (1.05 eq, 86 mg, 0.28 mmol), K₃PO₄ (2.5 eq, 140 mg, 0.66 mmol), Pd(dppf)Cl₂ DCM (0.1 eq, 22 mg, 0.027 mmol), 1,4-dioxane (1.6 mL) and water (0.16 mL). The vial was sealed, degassed under argon and stirred at 75° C. for 3 h. The reaction mixture was cooled to RT and filtered over celite, rinsing with EtOAc. The filtrate was concentrated under reduced pressure and purified by silica gel column chromatography (5-60% EtOAc in Hexanes) to yield 2-[6-(2-methoxythiazol-5-yl)-3,6-dihydro-2H-pyran-4-yl]-5-nitro-6-[3-(trifluoromethyl)-1-bicyclo[1.1.1]pentanyl]pyrimidin-4-amine (A130, 64 mg, 0.14 mmol, 51% yield) as a solid. ESI-MS (m/z)+: 470.3 (obs). ¹H NMR (CHCl₃-d, 400 MHz): δ_{H} 7.36 (d, J=2.5 Hz, 1H), 7.03 (d, J=0.7 Hz, 1H), 6.54 (s, 2H), 5.47 (q, J=2.5 Hz, 1H), 4.05 (s, 3H), 4.03-3.99 (m, 1H), 3.88-3.83 (m, 1H), 2.42 (s, 6H).

### Synthesis of A131

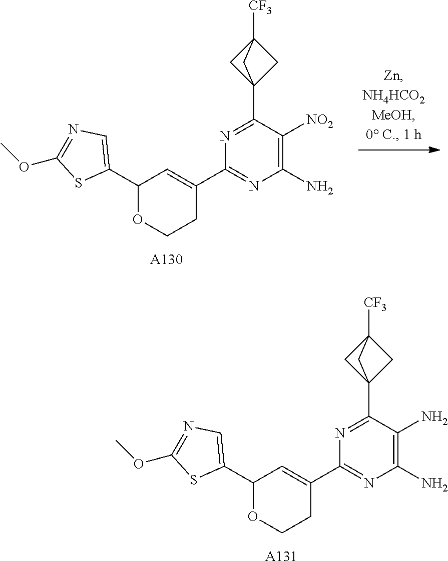

To a suspension of 2-[6-(2-methoxythiazol-5-yl)-3,6-dihydro-2H-pyran-4-yl]-5-nitro-6-[3-(trifluoromethyl)-1-bicyclo[1.1.1]pentanyl]pyrimidin-4-amine (A130, 1.0 eq, 60 mg, 0.13 mmol) in MeOH (2 mL) at 0° C. was added ammonium formate (8.0 eq, 64 mg, 1.0 mmol), followed by addition of Zn powder (8.0 eq, 67 mg, 1.0 mmol), and the reaction was stirred for 1 h at 0° C. The resulting mixture was filtered through celite rinsing with MeOH, and concentrated under reduced pressure to afford 2-[6-(2-methoxythiazol-5-yl)-3,6-dihydro-2H-pyran-4-yl]-6-[3-(trifluoromethyl)-1-bicyclo[1.1.1]pentanyl]-pyrimidine-4,5-diamine (A131, 56 mg, 0.13 mmol, 99% yield) as a solid. ESI-MS (m/z)+: 440.3 (obs)

### Synthesis of A132

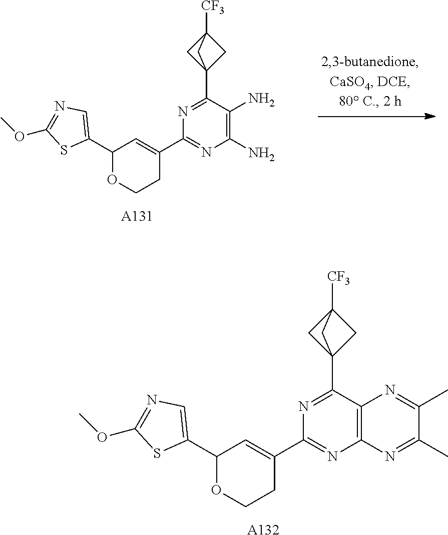

A suspension of 2-[6-(2-methoxythiazol-5-yl)-3,6-dihydro-2H-pyran-4-yl]-6-[3-(trifluoromethyl)-1-bicyclo[1.1.1]pentanyl]pyrimidine-4,5-diamine (A131, 1.0 eq, 56 mg, 0.13 mmol) and CaSO₄ (10 eq, 173 mg, 1.3 mmol) in DCE (2 mL) was stirred overnight at 80° C. The reaction mixture was taken up in DCM and filtered over celite, rinsing with DCM. The filtrate was concentrated under reduced pressure and purified by silica gel flash column chromatography (50-100% of EtOAc in Hexanes) to afford 5-[4-[6,7-dimethyl-4-[3-(trifluoromethyl)-1-bicyclo[1.1.1]pentanyl]pteridin-2-yl]-3,6-dihydro-2H-pyran-6-yl]-2-methoxy-thiazole (A132, 28 mg, 0.06 mmol, 45% yield) as an oil. ESI-MS (m/z)+: 490.3 (obs).

### Synthesis of Example 117

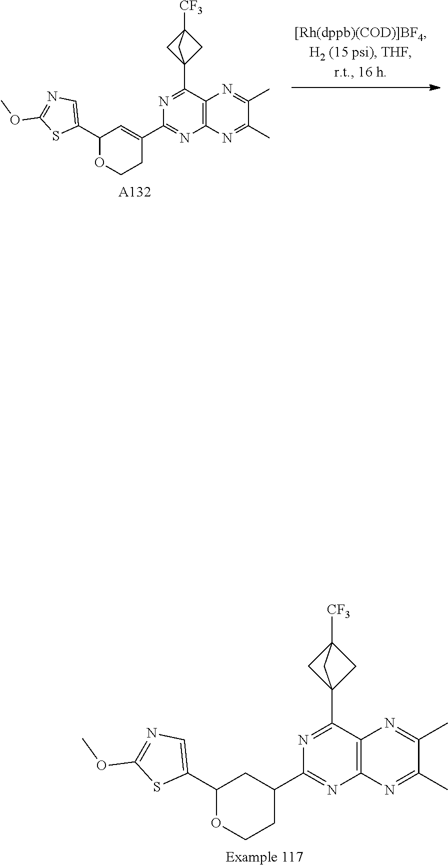

A round-bottomed flask equipped with a Teflon-coated magnetic stirring bar was charged with [Rh(dppb)(COD)] BF₄ (0.27 eq, 10 mg, 0.014 mmol). The flask was purged with argon and a solution of 5-[4-[6,7-dimethyl-4-[3-(trifluoromethyl)-1-bicyclo[1.1.1]pentanyl]pteridin-2-yl]-3,6-dihydro-2H-pyran-6-yl]-2-methoxy-thiazole (A132, 1.0 eq, 26 mg, 0.053 mmol) in THF (1 mL) was added. The heterogeneous reaction mixture was then purged with H₂ and stirred at 55° C. under H₂ atmosphere (15 psi) for 16 h. The reaction mixture was cooled to RT and filtered over a pad of celite, rinsing with DCM. The filtrate was concentrated under reduced pressure and purified by silica gel flash column chromatography (0-50% EtOAc:EtOH (3:1) in Hexanes) to afford 2-methoxy-5-[rac-(2R,4S)-4-[6,7-dimethyl-4-[3-(trifluoromethyl)-1-bicyclo[1.1.1]pentanyl]pteridin-2-yl]tetrahydropyran-2-yl]thiazole ((rac)-Example 117, 2.5 mg, 5.1 μmol, 10% yield) as a solid. ¹H NMR (CHCl₃-d, 400 MHz): δ_{H} 7.05 (d, J=0.7 Hz, 1H), 4.67 (dd, J=11.3, 1.5 Hz, 1H), 4.29-4.24 (m, 1H), 4.05 (d, J=5.0 Hz, 3H), 3.83-3.77 (m, 1H), 3.47-3.40 (m, 1H), 2.81 (s, 3H), 2.77 (s, 3H), 2.64 (s, 6H), 2.46-2.43 (m, 1H), 2.22-2.11 (m, 3H). ¹⁹F NMR (CHCl₃-d, 376 MHz): δ_{F}−73.0 (s, 3F). ESI-MS (m/z)+: 492.3 (obs).

### Method 51: Synthesis of Example 119

### Synthesis of A133

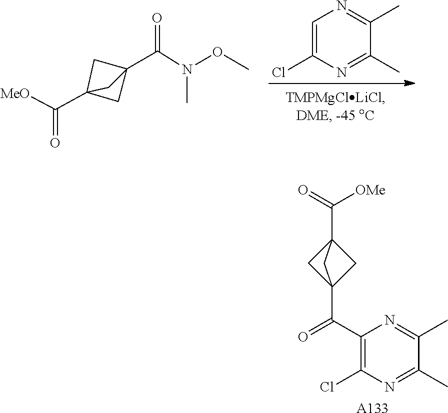

To a stirring solution of methyl 3-[methoxy(methyl)carbamoyl]bicyclo[1.1.1]pentane-1-carboxylate (1.0 eq, 2.67 g, 10.0 mmol), and 5-chloro-2,3-dimethyl-pyrazine (1.2 eq, 1.4 mL, 12.0 mmol) in anhydrous DME (20 mL), cooled to −45° C., was added chloro-(2,2,6,6-tetramethyl-1-piperidyl)-magnesium (1.3 eq, 13 mL, 13.0 mmol) dropwise over 2 min. The reaction mixture was stirred at −45° C. for 2 h. The reaction mixture was quenched with sat. NH₄Cl (aq) (10 mL) and warmed to RT. EtOAc (150 mL) and H₂O (150 mL) were added. The layers were separated, and the organic layer was washed with brine. The organic layer was dried over anhydrous Na₂SO₄, filtered, concentrated under reduced pressure, and purified by silica gel flash column chromatography (5-30% EtOAc in hexanes) to afford methyl 3-(3-chloro-5,6-dimethylpyrazine-2-carbonyl)bicyclo[1.1.1]pentane-1-carboxylate (A133, 2.22 g, 7.53 mmol, 75% yield) as a solid. ESI-MS (m/z⁺): 295.1 (obs). ¹H-NMR (CHCl₃-d, 400 MHz): δ_{H} 3.69 (s, 3H), 2.57 (s, 3H), 2.55 (s, 3H), 2.50 (s, 6H).

### Synthesis of A134

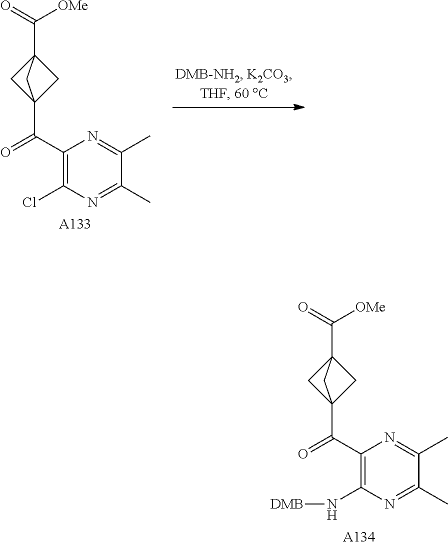

To a stirring solution of methyl 3-(3-chloro-5,6-dimethylpyrazine-2-carbonyl)-bicyclo[1.1.1]pentane-1-carboxylate (A133, 1.0 eq, 1.74 g, 5.9 mmol) in anhydrous THF (40 mL) was added K₂CO₃ (2.0 eq, 1.63 g, 11.8 mmol) and (2,4-dimethoxyphenyl)methanamine (2.1 eq, 1.9 mL, 12.4 mmol), and the reaction was stirred under reflux at 60° C. for 2 days. The reaction mixture was cooled to RT, diluted with EtOAc (100 mL), and the resulting solution was washed with sat. NH₄Cl (aq) and brine. The organic layer was dried over anhydrous Na₂SO₄, filtered, concentrated under reduced pressure, and purified by silica gel flash column chromatography (2-20% EtOAc in Hexanes) to afford methyl 3-(3-((3,4-dimethylbenzyl)amino)-5,6-dimethylpyrazine-2-carbonyl)bicyclo[1.1.1]pentane-1-carboxylate (A134, 2.45 g, 5.29 mmol, 90% yield) as a solid. ESI-MS (m/z⁺): 426.3 (obs). ¹H-NMR (CHCl₃-d, 400 MHz): δ_{H} 8.75 (t, J=5.4 Hz, 1H), 7.21 (d, J=8.2 Hz, 1H), 6.44 (s, 1H), 6.39 (dd, J=8.2, 1.1 Hz, 1H), 4.64 (d, J=5.7 Hz, 2H), 3.84 (s, 3H), 3.77 (s, 3H), 3.69 (s, 3H), 2.52 (s, 6H), 2.42 (s, 3H), 2.36 (s, 3H).

### Synthesis of A135

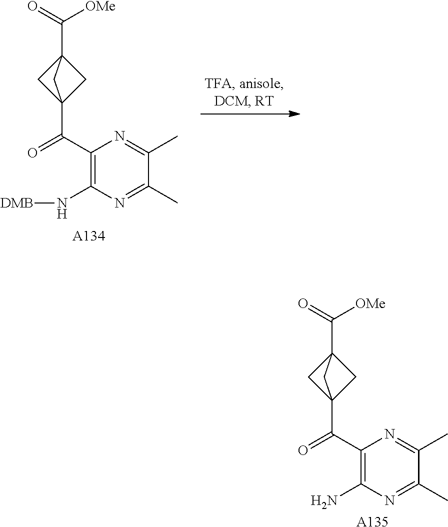

To a solution of methyl 3-[3-[(2,4-dimethoxyphenyl)methylamino]-5,6-dimethylpyrazine-2-carbonyl]bicyclo[1.1.1]pentane-1-carboxylate (A134, 1.0 eq, 2.44 g, 5.73 mmol) in anhydrous DCM (40 mL), was added anisole (1.0 eq, 0.62 mL, 5.73 mmol) and TFA (20.0 eq, 8.78 mL, 115 mmol), and the reaction was stirred at RT for 1 h. The resulting mixture was concentrated under reduced pressure, diluted with EtOAc (50 mL), and washed with sat. NaHCO₃(aq) and brine. The organic layer was dried over anhydrous Na₂SO₄, filtered, concentrated under reduced pressure, and purified by silica gel flash column chromatography (10-45% EtOAc in Hexanes) to afford methyl 3-(3-amino-5,6-dimethyl-pyrazine-2-carbonyl)bicyclo[1.1.1]pentane-1-carboxylate (A135, 1.39 g, 5.05 mmol, 88% yield) as a solid. ESI-MS (m/z⁺): 276.3 (obs). ¹H-NMR (CHCl₃-d, 400 MHz): δ_{H} 3.69 (d, J=8.9 Hz, 3H), 2.57-2.55 (m, 6H), 2.42 (s, 3H), 2.41 (s, 3H).

### Synthesis of A136

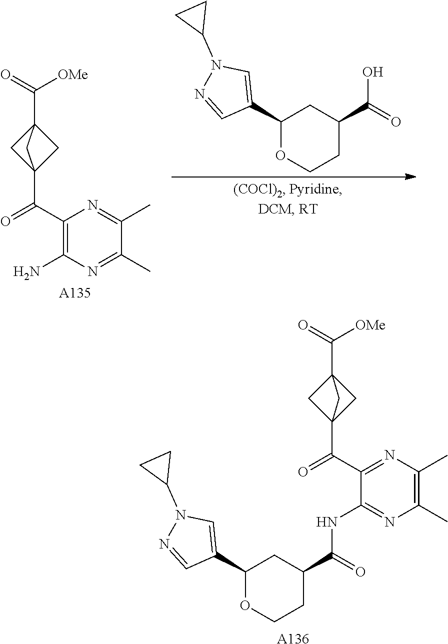

To a stirring solution of (2R,4S)-2-(1-cyclopropylpyrazol-4-yl)tetrahydropyran-4-carboxylic acid (1.1 eq, 217 mg, 0.919 mmol) in anhydrous DCM (10 mL), was added DMF (0.05 mL) and oxalyl chloride (1.2 eq, 0.088 mL, 1.00 mmol) at 0° C. The resulting mixture was warmed to RT and stirred for 1 h. The reaction mixture was cooled to −10° C. and a solution of pyridine (3.0 eq, 0.20 mL, 2.51 mmol) and methyl 3-(3-amino-5,6-dimethyl-pyrazine-2-carbonyl)bicyclo[1.1.1]pentane-1-carboxylate (A135, 1.00 eq, 230 mg, 0.835 mmol) in DCM (5.0 mL) was added dropwise, and the resulting mixture was stirred at RT for 16 h. The resulting mixture was concentrated under reduced pressure, diluted with EtOAc (100 mL), and washed with 5% citric acid (aq), sat. NaHCO₃(aq) and brine. The organic layer was dried over anhydrous Na₂SO₄, filtered, concentrated under reduced pressure, and purified by silica gel flash column chromatography (30-100% (3:1 EtOAc:EtOH) in hexanes) to afford methyl 3-(3-((2R,4S)-2-(1-cyclopropyl-1H-pyrazol-4-yl)tetrahydro-2H-pyran-4-carboxamido)-5,6-dimethylpyrazine-2-carbonyl)bicyclo[1.1.1]pentane-1-carboxylate (A136, 306 mg, 0.62 mmol, 74% yield) as a foam, that was used directly in the next step. ESI-MS (m/z⁺): 494.4 (obs).

### Synthesis of A137

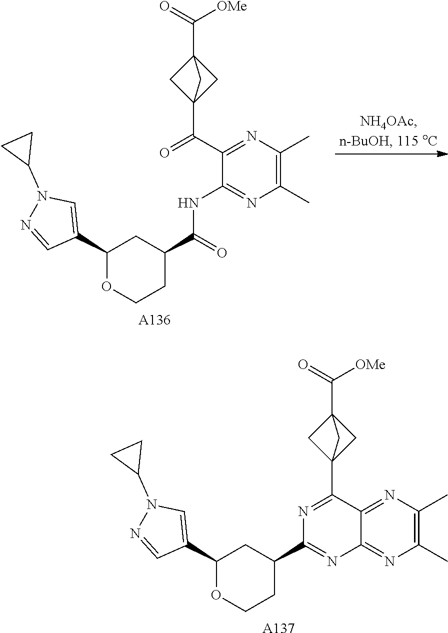

To a stirring solution of methyl 3-(3-((2R,4S)-2-(1-cyclopropyl-1H-pyrazol-4-yl)tetrahydro-2H-pyran-4-carboxamido)-5,6-dimethylpyrazine-2-carbonyl)bicyclo[1.1.1]pentane-1-carboxylate (A136, 1.0 eq, 248 mg, 0.502 mmol) in 1-butanol (5.0 mL) was added ammonium acetate (10.0 eq, 387 mg, 5.02 mmol) at RT, and the reaction was stirred at 115° C. for 1 h. The reaction mixture was cooled to RT and diluted with EtOAc (50 mL). The resulting suspension was washed with 5% citric acid (aq), sat. NaHCO₃(aq) and brine. The organic layer was dried over anhydrous Na₂SO₄ and filtered. The filtrate was concentrated under reduced pressure and purified by silica gel flash column chromatography (10-50% acetone in Hexanes) to afford methyl 3-(2-((2R,4S)-2-(1-cyclopropyl-1H-pyrazol-4-yl)tetrahydro-2H-pyran-4-yl)-6,7-dimethylpteridin-4-yl)bicyclo[1.1.1]pentane-1-carboxylate (A137, 174 mg, 0.37 mmol, 73% yield) as a foam. ESI-MS (m/z⁺): 475.5 (obs). ¹H-NMR (MeOH-d₄, 400 MHz): δ_{H} 7.66 (s, 1H), 7.46 (s, 1H), 4.56 (dd, J=11.4, 1.8 Hz, 1H), 4.19-4.15 (m, 1H), 3.82-3.75 (m, 1H), 3.70 (d, J=3.2 Hz, 4H), 3.61-3.56 (m, 1H), 3.41 (t, J=3.8 Hz, 1H), 2.77 (s, 3H), 2.76 (s, 3H), 2.68 (d, J=4.1 Hz, 6H), 2.27 (d, J=13.3 Hz, 1H), 2.09-2.00 (m, 3H), 1.05-0.98 (m, 4H).

### Synthesis of A138

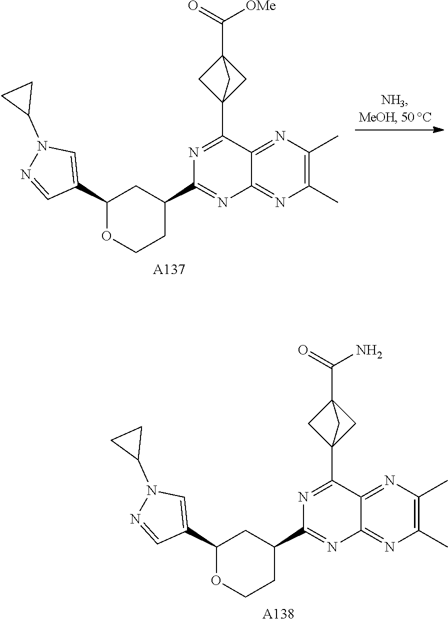

To a stirring solution of methyl 3-(2-((2R,4S)-2-(1-cyclopropyl-1H-pyrazol-4-yl)tetrahydro-2H-pyran-4-yl)-6,7-dimethylpteridin-4-yl)bicyclo[1.1.1]pentane-1-carboxylate (A137, 1.0 eq, 48 mg, 0.10 mmol) in anhydrous MeOH (1.0 mL) was added 7N ammonia in MeOH (25.0 eq, 0.36 mL, 2.50 mmol). The reaction mixture was stirred at 50° C. in a closed system for 2 days. The reaction mixture was cooled to RT and concentrated under reduced pressure to afford 3-(2-((2R,4S)-2-(1-cyclopropyl-1H-pyrazol-4-yl)tetrahydro-2H-pyran-4-yl)-6,7-dimethylpteridin-4-yl)bicyclo[1.1.1]pentane-1-carboxamide (A138, 46 mg, 0.099 mmol, 99% yield) as a film, that was used directly in the next step without further purification. ESI-MS (m/z⁺): 460.4 (obs).

### Synthesis of Example 119

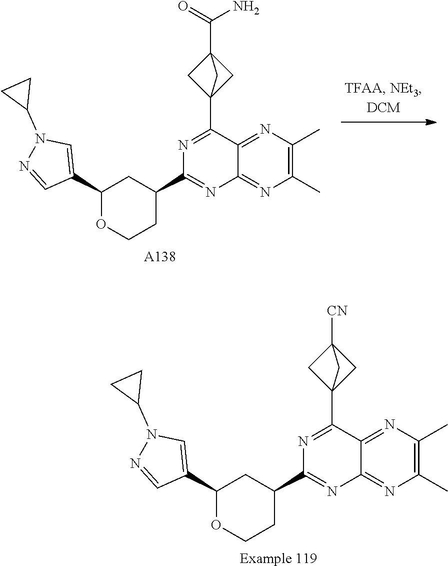

To a solution of 3-(2-((2R,4S)-2-(1-cyclopropyl-1H-pyrazol-4-yl)tetrahydro-2H-pyran-4-yl)-6,7-dimethylpteridin-4-yl)bicyclo[1.1.1]pentane-1-carboxamide (A138, 1.0 eq, 45 mg, 0.098 mmol) and pyridine (2.0 eq, 0.016 mL, 0.196 mmol) in anhydrous DCM (1.0 mL) was added trifluoroacetic anhydride (2.0 eq, 0.028 mL, 0.196 mmol) dropwise at RT, and the reaction was stirred at RT for 20 min. The reaction mixture was diluted with EtOAc (50 mL) and washed with 5% citric acid (aq), sat. NaHCO₃(aq) and brine. The organic layer was dried over anhydrous Na₂SO₄ and filtered. The filtrate was concentrated under reduced pressure and purified by silica gel flash column chromatography (10-40% acetone in hexanes) to afford 3-(2-((2R,4S)-2-(1-cyclopropyl-1H-pyrazol-4-yl)tetrahydro-2H-pyran-4-yl)-6,7-dimethylpteridin-4-yl)bicyclo[1.1.1]pentane-1-carbonitrile (Example 119, 33 mg, 0.074 mmol, 76% yield) as a solid. LC/MS (ESI, m/z⁺): [M+H]=442.2. ¹H-NMR (MeOH-d₄, 400 MHz): δ_{H} 7.66 (s, 1H), 7.45 (s, 1H), 4.56 (dd, J=11.4, 2.1 Hz, 1H), 4.19-4.14 (m, 1H), 3.82-3.75 (m, 1H), 3.61-3.56 (m, 1H), 3.45-3.37 (m, 1H), 2.92 (d, J=11.7 Hz, 6H), 2.77 (d, J=1.1 Hz, 6H), 2.27 (d, J=13.3 Hz, 1H), 2.08-1.99 (m, 3H), 1.05-0.96 (m, 4H).

### Method 52: Synthesis of Example 123

### Synthesis of A142

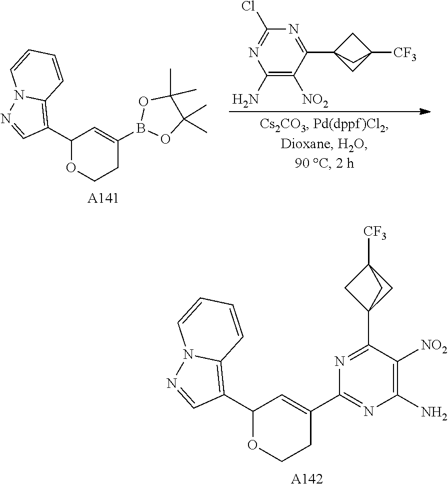

To a glass vial equipped with a Teflon-coated magnetic stirring bar was added 3-[4-(4,4,5,5-tetramethyl-1,3,2-dioxaborolan-2-yl)-3,6-dihydro-2H-pyran-6-yl]pyrazolo[1,5-a]pyridine (A141, 1.0 eq, 90 mg, 0.28 mmol), 2-chloro-5-nitro-6-[3-(trifluoromethyl)-1-bicyclo[1.1.1]pentanyl]pyrimidin-4-amine (1.1 eq, 94 mg, 0.30 mmol), Pd(dppf)Cl₂ DCM (0.125 eq, 28 mg, 0.035 mmol), cesium carbonate (2.5 eq, 225 mg, 0.69 mmol), 1,4-dioxane (2.5 mL) and water (0.25 mL). The vial was sealed, degassed under argon and stirred at 90° C. for 2 h. The reaction was quenched with water and extracted with DCM (×3). The combined organic layers were washed with brine, dried over Na₂SO₄, and filtered. The filtrate was concentrated under reduced pressure and purified by normal phase column chromatography (0-100% EtOAc in hexanes) to afford 5-nitro-2-(6-pyrazolo[1,5-a]pyridin-3-yl-3,6-dihydro-2H-pyran-4-yl)-6-[3-(trifluoromethyl)-1-bicyclo[1.1.1]pentanyl]pyrimidin-4-amine (A142, 83 mg, 0.18 mmol, 64% yield) as an oil. ¹H NMR (400 MHz, CHCl₃-d): δ_{H} 8.02 (d, J=2.3 Hz, 1H), 7.60 (d, J=2.3 Hz, 1H), 7.52 (d, J=8.0 Hz, 1H), 7.10 (dd, J=8.8, 7.0 Hz, 1H), 6.88 (d, J=6.9 Hz, 1H), 6.58 (d, J=2.3 Hz, 2H), 6.32 (d, J=2.5 Hz, 1H), 4.18 (d, J=11.4 Hz, 1H), 4.00-3.97 (m, 1H), 2.73 (s, 2H), 2.38 (s, 6H). ESI-MS (m/z)+: 473.4 (obs).

### Synthesis of A143

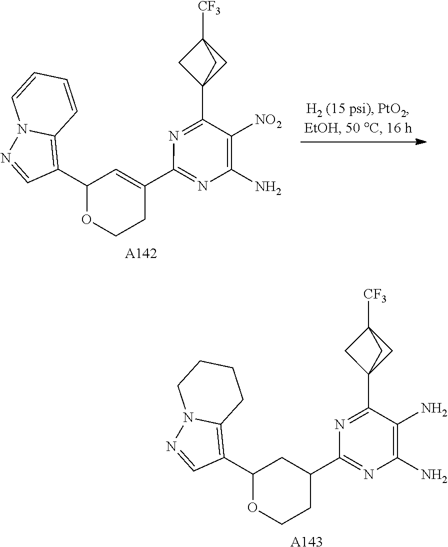

5-nitro-2-(6-pyrazolo[1,5-a]pyridin-3-yl-3,6-dihydro-2H-pyran-4-yl)-6-[3-(trifluoromethyl)-1-bicyclo[1.1.1]pentanyl]pyrimidin-4-amine (A142, 1.0 eq, 45 mg, 0.095 mmol) was dissolved in EtOH (2 mL), and the mixture was purged with argon. PtO₂ (5.0 eq, 108 mg, 0.48 mmol) was then added and the reaction was purged with hydrogen and stirred under hydrogen atmosphere overnight. The resulting mixture was filtered through a celite pad and concentrated under reduced pressure to afford crude 2-[2-(4,5,6,7-tetrahydropyrazolo[1,5-a]pyridin-3-yl)tetrahydropyran-4-yl]-6-[3-(trifluoromethyl)-1-bicyclo[1.1.1]pentanyl]pyrimidine-4,5-diamine (A143, 42 mg, 0.094 mmol, 98% yield), which was used in the next step without further purification. ESI-MS (m/z)+: 449.3 (obs).

### Synthesis of Example 123

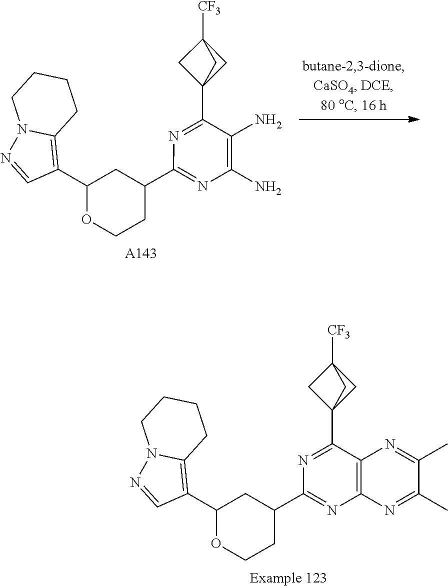

2-[2-(4,5,6,7-tetrahydropyrazolo[1,5-a]pyridin-3-yl)tetrahydropyran-4-yl]-6-[3-(trifluoromethyl)-1-bicyclo[1.1.1]pentanyl]pyrimidine-4,5-diamine (A143, 1.0 eq, 48 mg, 0.11 mmol) was stirred overnight at 80° C. with butane-2,3-dione (1.5 eq, 14 μL, 0.16 mmol) and CaSO₄ (10.0 eq, 0.15 g, 1.07 mmol) in DCE (1.1 mL). The reaction mixture was diluted with DCM and filtered over celite. The filtrate was concentrated under reduced pressure and purified by normal phase column chromatography (50-100% EtOAc in hexanes) to afford 6,7-dimethyl-2-[2-(4,5,6,7-tetrahydropyrazolo[1,5-a]pyridin-3-yl)tetrahydropyran-4-yl]-4-[3-(trifluoromethyl)-1-bicyclo[1.1.1]pentanyl]pteridine (Example 123, 2.0 mg, 4.0 μmol, 4% yield) as a solid. ESI-MS (m/z)+: 499.4 (obs). ¹H NMR (CHCl₃-d, 400 MHz): δ_{H} 7.47 (d, J=1.8 Hz, 1H), 5.97 (s, 1H), 4.51-4.47 (m, 1H), 4.17-4.11 (m, 2H), 3.63 (td, J=11.8, 2.7 Hz, 1H), 3.40 (tt, J=11.9, 4.0 Hz, 1H), 2.81 (s, 3H), 2.77 (s, 3H), 2.65 (s, 6H), 2.29-2.19 (m, 2H), 2.10-1.95 (m, 5H), 1.73-1.64 (m, 2H). ¹⁹F NMR (CHCl₃-d, 376 MHz): δ_{F}−72.9 (s, 3F).

### Method 53: Synthesis of Example 124 and Example 125

### Synthesis of A149

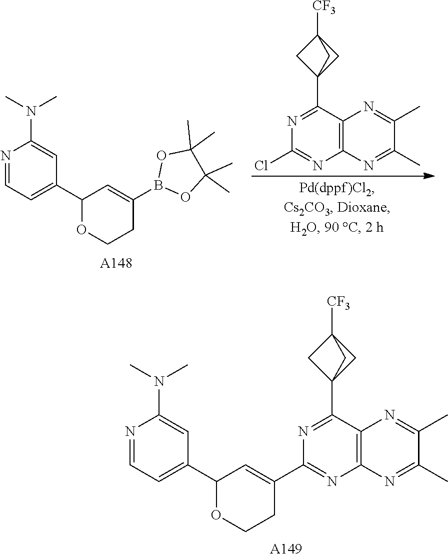

A mixture of 2-chloro-6,7-dimethyl-4-[3-(trifluoromethyl)-1-bicyclo[1.1.1]pentanyl]pteridine (1.1 eq, 127 mg, 0.39 mmol), N,N-dimethyl-4-[4-(4,4,5,5-tetramethyl-1,3,2-dioxaborolan-2-yl)-3,6-dihydro-2H-pyran-6-yl]pyridin-2-amine (A148, 1.0 eq, 116 mg, 0.35 mmol), Cs₂CO₃ (2.5 eq, 285 mg, 0.88 mmol) and Pd(dppf)Cl₂ (0.1 eq, 26 mg, 0.035 mmol) was stirred at 90° C. for 2 h under argon. The reaction was quenched with NaHCO₃ and extracted with EtOAc (×3). The combined organic layers were washed with brine, dried over Na₂SO₄, and filtered. The filtrate was concentrated under reduced pressure and purified by normal phase column chromatography (40-100% EtOAc in hexanes) to afford 4-[4-[6,7-dimethyl-4-[3-(trifluoromethyl)-1-bicyclo[1.1.1]pentanyl]pteridin-2-yl]-3,6-dihydro-2H-pyran-6-yl]-N,N-dimethyl-pyridin-2-amine (A149, 45 mg, 0.091 mmol, 26% yield). ESI-MS (m/z)+: 497.4 (obs).

### Synthesis of Example 124 and Example 125

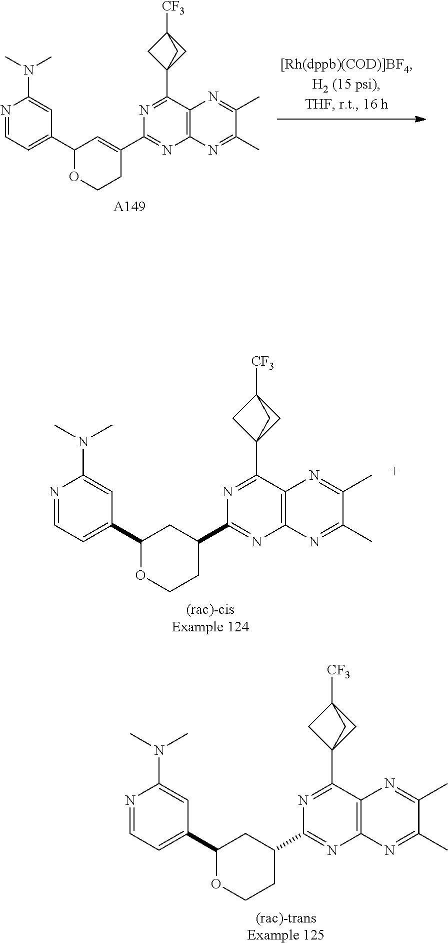

A solution of 4-[4-[6,7-dimethyl-4-[3-(trifluoromethyl)-1-bicyclo[1.1.1]pentanyl]pteridin-2-yl]-3,6-dihydro-2H-pyran-6-yl]-N,N-dimethyl-pyridin-2-amine (A149, 1.0 eq, 45 mg, 0.091 mmol) and [Rh(dppb)(COD)] BF₄ (0.6 eq, 39 mg, 0.054 mmol) in THF (2.0 mL) was purged with a hydrogen balloon and stirred overnight under 15 psi of hydrogen. The reaction was flushed with argon and concentrated under reduced pressure to afford a residue. The residue was purified by reversed-phase column chromatography (15-70% MeCN in water (0.1% formic acid)), and the mixture of diastereoisomers was separated by prep-HPLC (65-100% MeOH in 10 mM ammonium bicarbonate pH=10.0) to afford N,N-dimethyl-4-[rac-4-[6,7-dimethyl-4-[3-(trifluoromethyl)-1-bicyclo[1.1.1]pentanyl]pteridin-2-yl]tetrahydropyran-2-yl]pyridin-2-amine ((rac)-cis-Example 124) (6.0 mg, 0.012 mmol, 13% yield) and N,N-dimethyl-4-[rac-4-[6,7-dimethyl-4-[3-(trifluoromethyl)-1-bicyclo[1.1.1]pentanyl]pteridin-2-yl]tetrahydropyran-2-yl]pyridin-2-amine ((rac)-trans-Example 125) (2.0 mg, 0.004 mmol, 4% yield) as solids. Example 124: ¹H NMR (400 MHz, CHCl₃-d) δ_{H} 8.09 (d, J=5.3 Hz, 1H), 6.58 (s, 1H), 6.53 (d, J=5.3 Hz, 1H), 4.47-4.44 (m, 1H), 4.33 (dd, J=11.5, 2.9 Hz, 1H), 3.79 (td, J=11.8, 2.7 Hz, 1H), 3.47 (tt, J=11.9, 3.8 Hz, 1H), 3.08 (s, 6H), 2.80 (d, J=14.6 Hz, 3H), 2.75 (s, 3H), 2.60 (d, J=14.0 Hz, 6H), 2.34 (d, J=13.3 Hz, 1H), 2.24-2.10 (m, 2H), 2.01-1.92 (m, 1H). ESI-MS (m/z)+: 499.4 (obs). Example 125: ¹H NMR (400 MHz, CHCl₃-d) δ_{H} 8.12 (d, J=5.3 Hz, 1H), 6.59 (s, 1H), 6.54 (d, J=5.3 Hz, 1H), 4.66 (dd, J=9.6, 2.5 Hz, 1H), 4.01-3.97 (m, 1H), 3.86 (dd, J=11.1, 2.4 Hz, 1H), 3.61 (t, J=4.5 Hz, 1H), 3.09 (s, 6H), 2.82 (s, 3H), 2.78 (s, 3H), 2.64 (d, J=13.5 Hz, 6H), 2.54 (d, J=17.2 Hz, 1H), 2.23-2.13 (m, 2H). ESI-MS (m/z)+: 499.4 (obs).

### Method 54: Synthesis of Example 127

### Synthesis of A150

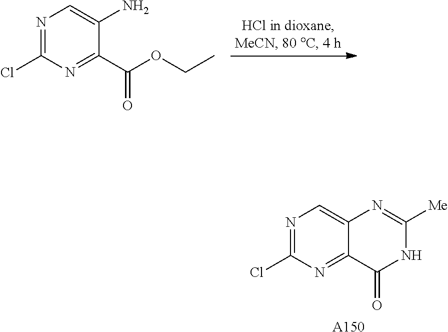

A pressured vial was charged with a solution of ethyl 5-amino-2-chloro-pyrimidine-4-carboxylate (1.0 eq, 2.0 g, 9.9 mmol) in 4 M HCl in 1,4-dioxane (6.1 eq, 15 mL, 60.0 mmol). Acetonitrile (6.0 eq, 3.1 mL, 59.4 mmol) was added, the vial was sealed and stirred at 80° C. for 4 h. The vial was cooled down to RT, the solids were filtered using a Buchner funnel, rinsed with acetonitrile, collected, and dried under high vacuum to yield 6-chloro-2-methyl-1H-pyrimido[5,4-d]pyrimidin-4-one hydrochloride (A150, 2.35 g, 10.1 mmol, 100% yield) as a solid. ESI-MS (m/z)+: 197.0 (obs).

### Synthesis of A151

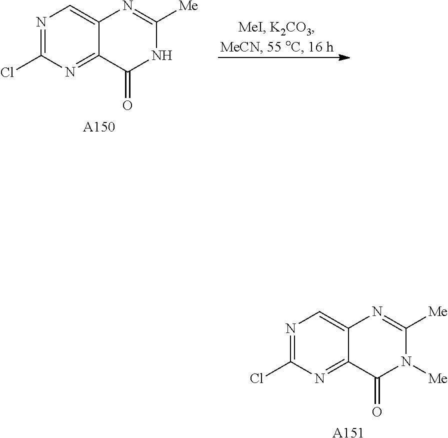

To a suspension of 6-chloro-2-methyl-1H-pyrimido[5,4-d]pyrimidin-4-one hydrochloride (A150, 1.0 eq, 2.35 g, 7.9 mmol) in MeCN (50 mL) was added potassium carbonate (2.51 eq, 2.73 g, 19.8 mmol) and Mel (1.67 eq, 820 μL, 13.1 mmol). The reaction mixture was sealed and stirred at 55° C. for 16 h. The reaction mixture was filtered over a pad of Celite and rinsed with ACN. The resulting mixture was concentrated under reduced pressure and purified by silica gel flash column chromatography (10-70% EtOAc:EtOH (3:1) in Hexanes) to yield 6-chloro-2,3-dimethyl-pyrimido[5,4-d]pyrimidin-4-one (A151, 1.37 g, 6.5 mmol, 83% yield) as a powder. ¹H NMR (CHCl₃-d, 400 MHz): δ_{H} 9.12 (s, 1H), 3.69 (s, 3H), 2.68 (s, 3H). ESI-MS (m/z)+: 211.1 (obs).

### Synthesis of A152

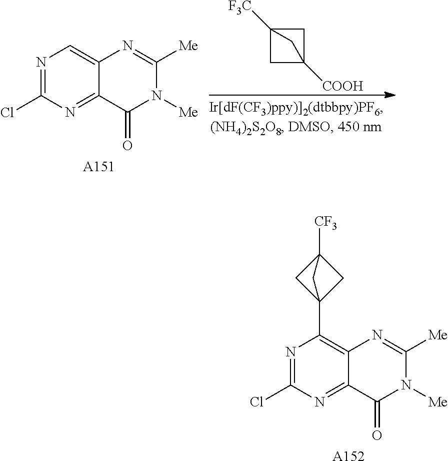

A reaction vial equipped with a Teflon-coated magnetic stirring bar was charged with Ir[dF(CF₃)ppy)]₂(dtbbpy)PF₆ (0.005 eq, 28 mg, 0.026 mmol), ammonium persulfate (2.0 eq, 2.32 g, 10.2 mmol), 6-chloro-2,3-dimethyl-pyrimido[5,4-d]pyrimidin-4-one (A151, 1.0 eq, 1.07 g, 5.1 mmol) and 3-(trifluoromethyl)bicyclo[1.1.1]pentane-1-carboxylic acid (5.0 eq, 4.58 g, 25.4 mmol). The vial was sealed, purged with argon (×3), and anhydrous DMSO (28 mL) was added. The reaction was placed in a Penn PhD photoreactor M2 and irradiated at 450 nm for 2 h. The reaction mixture was diluted with DCM, washed with sat. NaHCO₃(aq.) and the aqueous phase was extracted with DCM (×2). The combined organic phases were washed with brine, dried over anhydrous Na₂SO₄, and filtered. The filtrate was concentrated under reduced pressure and purified by silica gel flash column chromatography (2-65% EtOAc:EtOH (3:1) in Hexanes) to afford 6-chloro-2,3-dimethyl-8-[3-(trifluoromethyl)-1-bicyclo[1.1.1]pentanyl]pyrimido[5,4-d]pyrimidin-4-one (A152, 373 mg, 1.1 mmol, 21% yield) as a solid. ESI-MS (m/z)+: 345.3 (obs).

### Synthesis of Example 127

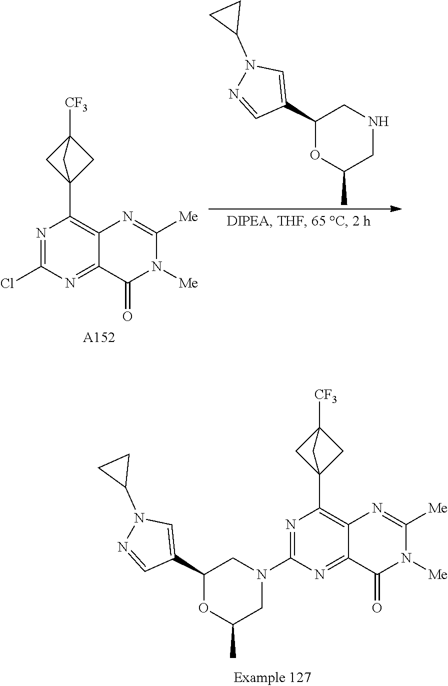

A vial equipped with a Teflon-coated magnetic stirring bar was charged with (2S,6R)-2-(1-cyclopropylpyrazol-4-yl)-6-methyl-morpholine (1.5 eq, 27 mg, 0.13 mmol), 6-chloro-2,3-dimethyl-8-[3-(trifluoromethyl)-1-bicyclo[1.1.1]pentanyl]pyrimido[5,4-d]pyrimidin-4-one (A152, 1.0 eq, 30 mg, 0.087 mmol) and THF (1 mL). DIPEA (3.0 eq, 45 μL, 0.26 mmol) was added and the vial was capped and stirred at 70° C. for 3 h. The resulting mixture was concentrated under reduced pressure and purified by silica gel flash column chromatography (0-100% EtOAc in Hexanes) to yield 6-[(2S,6R)-2-(1-cyclopropylpyrazol-4-yl)-6-methyl-morpholin-4-yl]-2,3-dimethyl-8-[3-(trifluoromethyl)-1-bicyclo[1.1.1]pentanyl]pyrimido[5,4-d]pyrimidin-4-one (Example 127, 42 mg, 0.08 mmol, 93% yield) as a solid. ESI-MS (m/z)+: 516.4 (obs). ¹H NMR (CHCl₃-d, 400 MHz): δ_{H} 7.54 (d, J=1.4 Hz, 2H), 4.85 (dd, J=38.5, 13.2 Hz, 2H), 4.55 (dd, J=11.0, 2.5 Hz, 1H), 3.82-3.74 (m, 1H), 3.60-3.55 (m, 4H), 2.99 (dd, J=13.3, 11.0 Hz, 1H), 2.75 (dd, J=13.3, 10.5 Hz, 1H), 2.57 (s, 3H), 2.51 (s, 6H), 1.31 (d, J=6.2 Hz, 3H), 1.14-1.00 (m, 4H). ¹⁹F NMR (CHCl₃-d, 376 MHz): δ_{F}−73.0 (s, 3F).

### Method 55: Synthesis of Example 129, Example 130, and Example 132

### Synthesis of A153

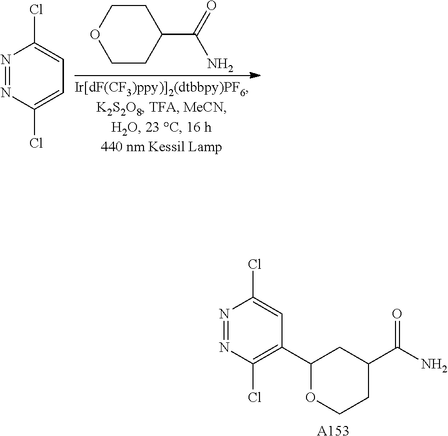

In a round bottom flask was charged Ir[dF(CF₃)ppy)]₂(dtbbpy)PF₆ (2 mol %, 151 mg, 0.134 mmol), potassium persulfate (2.0 eq, 3.63 g, 13.4 mmol), tetrahydropyran-4-carboxamide (15.0 eq, 13.0 g, 101 mmol), MeCN (33 mL), 3,6-dichloropyridazine (1.0 eq, 1.0 g, 6.71 mmol), trifluoroacetic acid (1.0 eq, 0.52 mL, 6.71 mmol) and water (33 mL). The reaction mixture was degassed under nitrogen for 15 min with an outlet needle. The mixture was stirred rapidly and irradiated with one 440 nm Kessil lamp (100% intensity) at room temperature overnight. Water was added and the aqueous layer was extracted with EtOAc (×3). The combined organic layers were washed with brine, dried over Na₂SO₄ and filtered. The filtrate was concentrated under reduced pressure and purified by normal phase column chromatography (20-60% EtOAc in hexanes), followed by reversed-phase column chromatography (5-70% MeCN in water (0.1% formic acid)) to afford 2-(3,6-dichloropyridazin-4-yl)tetrahydropyran-4-carboxamide (A153, 750 mg, 2.72 mmol, 40% yield). ESI-MS (m/z⁺): 276.2 [M+1]+ (obs).

### Synthesis of A154

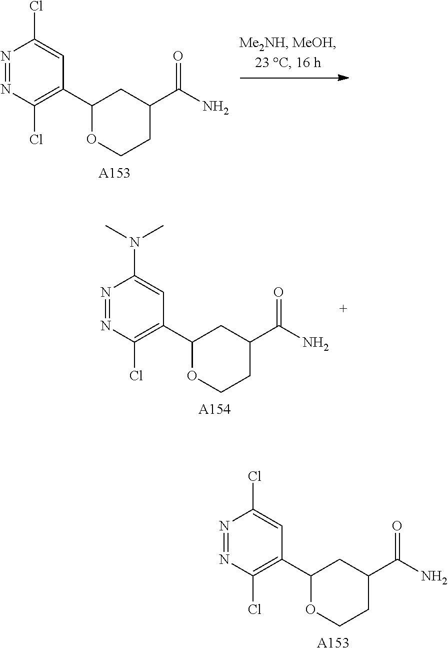

2-(3,6-dichloropyridazin-4-yl)tetrahydropyran-4-carboxamide (A153, 1.0 eq, 350 mg, 1.27 mmol) was stirred overnight in a solution of dimethylamine (2.0 M in MeOH, 3.0 eq, 1.9 mL, 3.8 mmol) in MeOH (2.5 mL). The resulting mixture was concentrated under reduced pressure to afford 2-[3-chloro-6-(dimethylamino)pyridazin-4-yl]tetrahydropyran-4-carboxamide (A154, 360 mg, 1.26 mmol, 99% yield) as a 6:4 mixture of regioisomers. ESI-MS (m/z)+: 285.2 (obs).

### Synthesis of A155 and A156

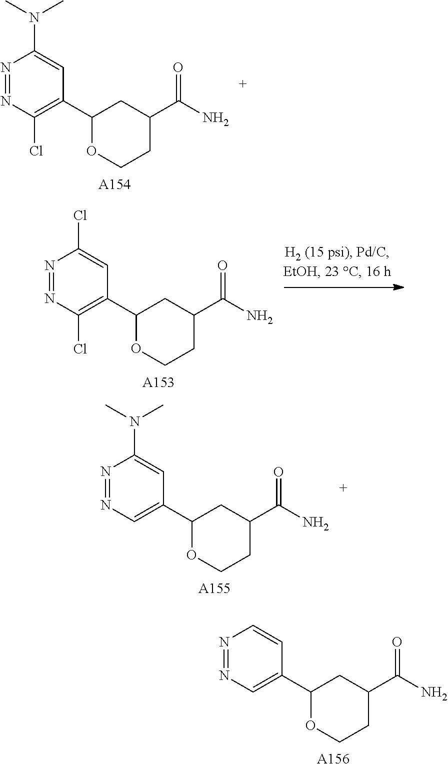

A round-bottom flask was charged with 2-[3-chloro-6-(dimethylamino)pyridazin-4-yl]tetrahydropyran-4-carboxamide (A154, 1.0 eq, 400 mg, 1.40 mmol) (containing traces of A153), TEA (3.0 eq, 0.59 mL, 4.21 mmol), and EtOH (16 mL). The reaction mixture was purged with argon then 10% Pd/C (0.5 eq, 8.0 mg, 0.076 mmol) was added. The resulting mixture was purged with hydrogen (×3) and stirred overnight at room temperature under 1 atm of hydrogen. The reaction was purged with argon and was filtered through a celite pad to afford 2-[6-(dimethylamino)-pyridazin-4-yl]tetrahydropyran-4-carboxamide (A155, 139 mg, 0.56 mmol, 40% yield) contaminated with traces of 2-[6-(dimethylamino)-pyridazin-4-yl]tetrahydropyran-4-carboxamide (A156) as an oil. ESI-MS (m/z)+: 251.2 (obs).

### Synthesis of A157 and A158

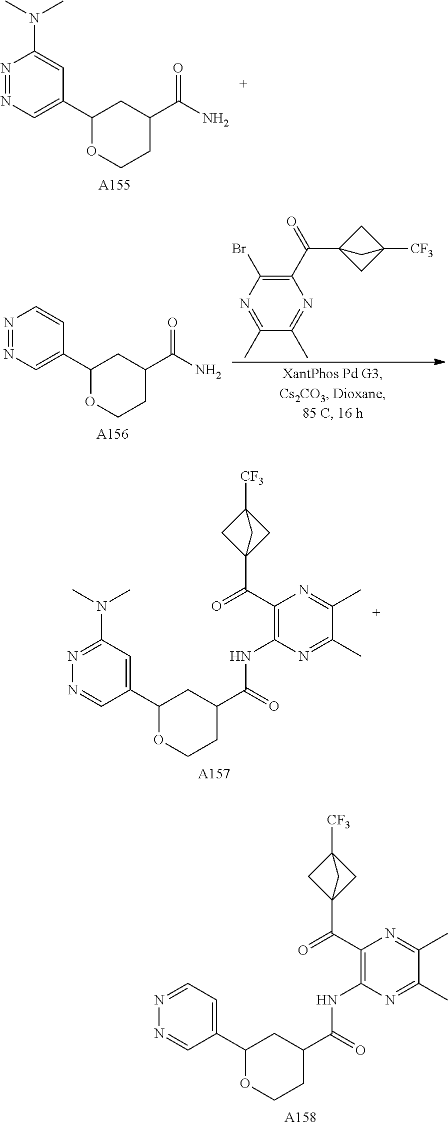

A flame-dried vial was charged with 2-[6-(dimethylamino)pyridazin-4-yl]tetrahydropyran-4-carboxamide (A155, 1.0 eq, 60 mg, 0.24 mmol) contaminated by traces of A156, (3-bromo-5,6-dimethyl-pyrazin-2-yl)-[3-(trifluoromethyl)-1-bicyclo[1.1.1]pentanyl]methanone (1.6 eq, 134 mg, 0.38 mmol), cesium carbonate (3.0 eq, 99 mg, 0.72 mmol) and 1,4-dioxane (2 mL). The reaction mixture was degassed with argon for 5 min then XantPhos Pd G3 (0.1 eq, 23 mg, 0.024 mmol) was added. The reaction mixture was degassed with argon for 5 min, sealed, plunged into a pre-heated oil bath at 85° C. and stirred at this temperature for 3 h. The reaction was quenched with NaHCO₃ and extracted with EtOAc (×3). The combined organic layers were washed with brine, dried over Na₂SO₄, and filtered. The filtrate was concentrated under reduced pressure and purified by normal phase column chromatography (40-100% EtOAc in hexanes) to afford 2-[6-(dimethylamino)pyridazin-4-yl]-N-[5,6-dimethyl-3-[3-(trifluoromethyl)bicyclo[1.1.1]pentane-1-carbonyl]pyrazin-2-yl]tetrahydropyran-4-carboxamide (A157, 107 mg, 0.21 mmol, 86% yield) contaminated by traces of 2-(pyridazin-4-yl)-N-[5,6-dimethyl-3-[3-(trifluoromethyl)bicyclo[1.1.1]pentane-1-carbonyl]pyrazin-2-yl]tetrahydropyran-4-carboxamide (A158). ESI-MS (m/z)+: 519.5 (obs).

### Synthesis of Example 129, Example 130, and Example 132

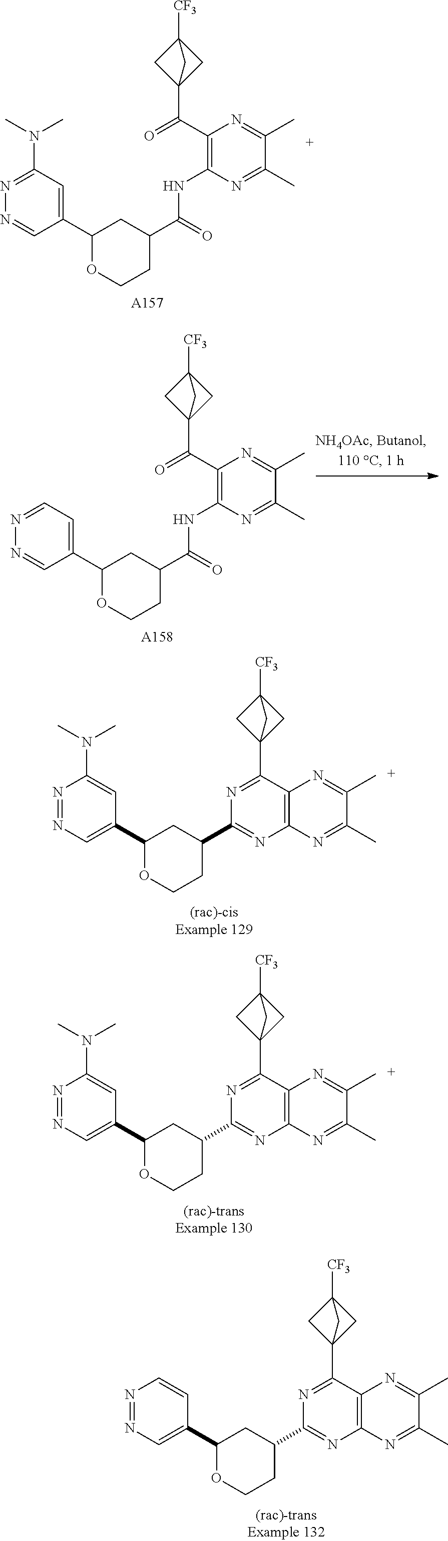

A mixture of 2-[6-(dimethylamino)pyridazin-4-yl]-N-[5,6-dimethyl-3-[3-(trifluoromethyl)-bicyclo[1.1.1]pentane-1-carbonyl]pyrazin-2-yl]tetrahydropyran-4-carboxamide (A157, 1.0 eq, 107 mg, 0.21 mmol) contaminated by traces of 2-(pyridazin-4-yl)-N-[5,6-dimethyl-3-[3-(trifluoromethyl)-bicyclo[1.1.1]pentane-1-carbonyl]pyrazin-2-yl]tetrahydropyran-4-carboxamide (A158) and NH4OAc (20.0 eq, 318 mg, 4.13 mmol) in 1-butanol (10 mL) was stirred at 110° C. for 1 h. The reaction was quenched with NaHCO₃ and extracted with EtOAc (×3). The combined organic layers were washed with brine, dried over Na₂SO₄, and filtered. The filtrate was concentrated under reduced pressure and purified by normal phase column chromatography (40-100% EtOAc in hexanes), followed by prep-HPLC to afford N,N-dimethyl-5-[rac-cis-4-[6,7-dimethyl-4-[3-(trifluoromethyl)-1-bicyclo[1.1.1]pentanyl]pteridin-2-yl]tetrahydropyran-2-yl]pyridazin-3-amine (Example 129, 2.0 mg, 0.004 mmol, 2% yield), N,N-dimethyl-5-[rac-trans-4-[6,7-dimethyl-4-[3-(trifluoromethyl)-1-bicyclo[1.1.1]pentanyl]pteridin-2-yl]tetrahydropyran-2-yl]pyridazin-3-amine (Example 130, 8.9 mg, 0.016 mmol, 8% yield) and [rac-trans-4-[6,7-dimethyl-4-[3-(trifluoromethyl)-1-bicyclo[1.1.1]pentanyl]pteridin-2-yl]tetrahydropyran-2-yl]pyridazin-3-amine (Example 132, 9.5 mg, 0.019 mmol, 9% yield). Example 129: ¹H NMR (400 MHz, CHCl₃-d) δ_{H} 8.49 (s, 1H), 6.86 (s, 1H), 4.49 (d, J=10.1 Hz, 1H), 4.37-4.33 (m, 1H), 3.83-3.77 (m, 1H), 3.50 (s, 1H), 3.18 (s, 6H), 2.79 (s, 3H), 2.76 (s, 3H), 2.63 (s, 6H), 2.38 (d, J=13.3 Hz, 1H), 2.19-2.13 (m, 2H), 1.94 (dd, J=25.2, 11.9 Hz, 1H). ¹⁹F NMR (376 MHz, CHCl₃-d) δ_{F}−73.0 (s, 3F). ESI-MS (m/z)+: 500.4 (obs). Example 130: ¹H NMR (400 MHz, CHCl₃-d) δ_{H} 8.50 (s, 1H), 6.85 (s, 1H), 4.72 (dd, J=10.2, 2.4 Hz, 1H), 4.02-3.98 (m, 1H), 3.88-3.82 (m, 1H), 3.63 (t, J=4.1 Hz, 1H), 3.18 (s, 6H), 2.84 (d, J=16.5 Hz, 4H), 2.78 (s, 3H), 2.66 (s, 6H), 2.56 (d, J=12.8 Hz, 1H), 2.23 (d, J=11.2 Hz, 1H), 2.11-2.03 (m, 1H). ¹⁹F NMR (376 MHz, CHCl₃-d)⁶F −72.9 (s, 3F). ESI-MS (m/z)+: 500.4 (obs). Example 132: ¹H NMR (400 MHz, CHCl₃-d) δ_{H} 9.23 (s, 1H), 9.14 (d, J=5.3 Hz, 1H), 7.54-7.52 (m, 1H), 4.87 (dd, J=10.3, 2.5 Hz, 1H), 4.01-3.97 (m, 1H), 3.83 (td, J=11.5, 2.4 Hz, 1H), 3.64 (t, J=4.1 Hz, 1H), 2.88 (d, J=13.5 Hz, 1H), 2.82 (d, J=4.8 Hz, 3H), 2.78 (d, J=7.3 Hz, 3H), 2.65 (d, J=7.5 Hz, 6H), 2.26-2.20 (m, 1H), 2.12-2.05 (m, 1H). ¹⁹F-NMR (376 MHz, CHCl₃-d) δ_{F}−72.9 (s, 3F). ESI-MS (m/z)+: 457.4 (obs).

### Method 56: Synthesis of Example 140

### Synthesis of A160

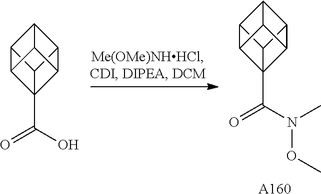

To a stirring solution of cubane-1-carboxylic acid (1.0 eq, 280 mg, 1.91 mmol) in anhydrous DCM (4.0 mL), at RT, was added N,N′-carbonyldiimidazole (1.41 eq, 440 mg, 2.7 mmol) portion-wise, over 2 min. The resulting solution was stirred, under Ar, for 1 h at RT. A solution of N,O-dimethylhydroxylamine hydrochloride (1.8 eq, 340 mg, 3.44 mmol) and N,N-diisopropylethylamine (1.8 eq, 0.6 mL, 3.4 mmol) in anhydrous DCM (3.0 mL) was added, and the reaction was stirred at RT for 18 h. The resulting mixture was concentrated under reduced pressure and taken up in EtOAc (100 mL). The resulting solution was washed with 1.0 M HCl (aq) (×2), sat. NaHCO₃(aq) and brine. The organic layer was dried over anhydrous Na₂SO₄, filtered and concentrated under reduced pressure to afford N-methoxy-N-methylcubane-1-carboxamide (A160, 0.36 g, 1.87 mmol, 98% yield) as a crystalline solid. ESI-MS (m/z⁺): 192.2 (obs). ¹H-NMR (400 MHz, CHCl₃-d): δ_{H} 4.28-4.25 (m, 3H), 4.00-3.97 (m, 4H), 3.70 (s, 3H), 3.18 (s, 3H).

### Synthesis of A161

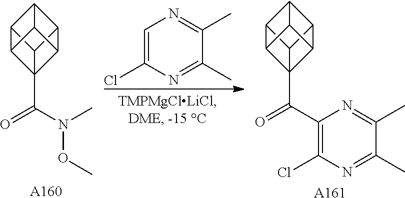

To a stirring solution of 2-chloro-5-methyl-pyrazine (1.5 eq, 0.32 mL, 2.63 mmol), N-methoxy-N-methylcubane-1-carboxamide (A160, 1.0 eq, 335 mg, 1.75 mmol) in anhydrous DME (3.5 mL) at −15° C., was added (2,2,6,6-tetramethyl-1-piperidyl)magnesium chloride (1.0 M in THF/toluene as a 1:1 complex with LiCl) (1.5 eq, 2.6 mL, 2.63 mmol) dropwise, such that the internal temperature of the reaction solution did not exceed −13° C. The reaction mixture was stirred at −15° C. for 90 min. The reaction mixture was quenched with sat. NH₄Cl (aq) (2.0 mL) and warmed to RT. EtOAc (100 mL) and H₂O (50 mL) were added, and the layers were separated. The organic layer was washed with brine, dried over anhydrous Na₂SO₄, and filtered. The filtrate was concentrated under reduced pressure and purified by silica gel flash-column chromatography (5-20% EtOAc in Hexanes) to afford (3-chloro-5,6-dimethyl-pyrazin-2-yl)(cuban-1-yl)methanone (A161, 317 mg, 1.16 mmol, 66% yield) as a crystalline solid. ESI-MS (m/z⁺): 273.2 (obs).

### Synthesis of A162

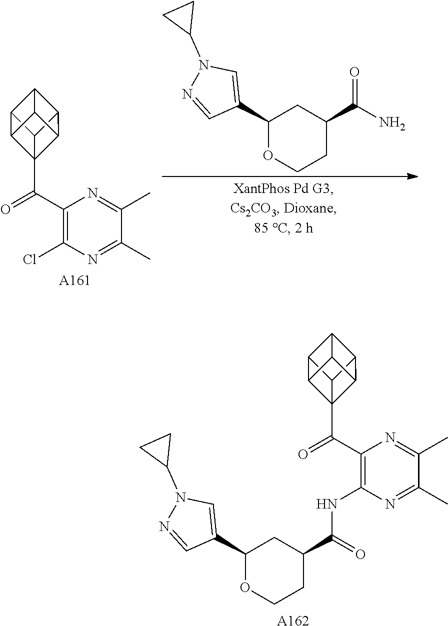

To a microwave vial equipped with a Teflon-coated magnetic stirring bar was added Cs₂CO₃ (1.0 eq, 114 mg, 0.30 mmol), (3-chloro-5,6-dimethylpyrazin-2-yl)(cuban-1-yl)methanone (A161, 1.0 eq, 95 mg, 0.3 mmol), (2R,4S)-2-(1-cyclopropylpyrazol-4-yl)tetrahydropyran-4-carboxamide (1.5 eq, 124 mg, 0.53 mmol), XantPhos Pd G3 (0.07 eq, 23 mg, 0.024 mmol) and anhydrous 1,4-dioxane (3.0 mL). The vial was sealed and purged/degassed under Ar, and the reaction was stirred at 85° C. for 2 h. The reaction mixture was cooled to RT and diluted with EtOAc (50 mL). The resulting solution was washed with water and brine. The organic layer was dried over anhydrous Na₂SO₄, filtered over a short silica plug and concentrated under reduced pressure. The residue was purified by silica-gel flash column chromatography (10-40% Acetone in Hexanes) to afford (2R,4S)-N-(3-(cubane-1-carbonyl)-5,6-dimethylpyrazin-2-yl)-2-(1-cyclopropyl-1H-pyrazol-4-yl)tetrahydro-2H-pyran-4-carboxamide (A162, 93 mg, 0.2 mmol, 56% yield) as a solid. ESI-MS (m/z⁺): 472.4 (obs). ¹H-NMR (400 MHz, CHCl₃-d): δ_{H} 11.39 (s, 1H), 7.52 (s, 1H), 7.49 (s, 1H), 4.43-4.39 (m, 4H), 4.20-4.16 (m, 1H), 4.10 (q, J=4.8 Hz, 3H), 4.05-4.00 (m, 1H), 3.68-3.57 (m, 2H), 2.88 (t, J=3.7 Hz, 1H), 2.59 (s, 3H), 2.49 (s, 3H), 2.22 (d, J=13.3 Hz, 1H), 1.99-1.87 (m, 3H), 1.14-1.08 (m, 2H), 1.07-1.01 (m, 2H).

### Synthesis of Example 140

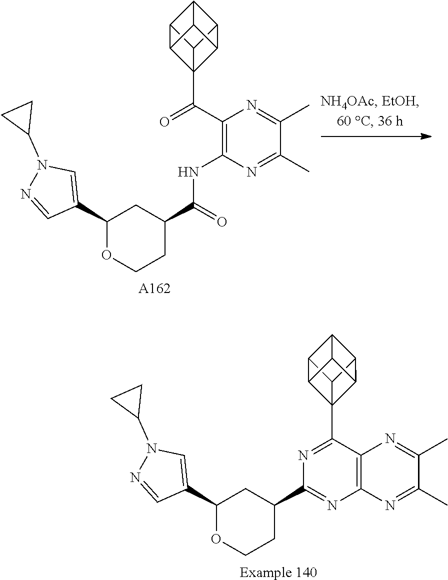

To a stirring solution of (2R,4S)-N-(3-(cubane-1-carbonyl)-5,6-dimethylpyrazin-2-yl)-2-(1-cyclopropyl-1H-pyrazol-4-yl)tetrahydro-2H-pyran-4-carboxamide (A162, 1.0 eq, 900 mg, 0.19 mmol) in EtOH (3.0 mL) at RT, was added ammonium acetate (10.0 eq, 147 g, 1.91 mmol), and the reaction was stirred at 60° C. for 36 h. The reaction was cooled to RT and diluted with EtOAc (50 mL). The resulting suspension was washed with 5% citric acid (aq), sat. NaHCO₃ (aq) and brine. The organic layer was dried over anhydrous Na₂SO₄, filtered, and concentrated under reduced pressure. The residue was purified by silica gel flash column chromatography (1-8% MeOH in DCM) to afford 2-((2R,4S)-2-(1-cyclopropyl-1H-pyrazol-4-yl)tetrahydro-2H-pyran-4-yl)-6,7-dimethyl-4-(cuban-1-yl)pteridine (Example 140, 70 mg, 0.15 mmol, 76% yield) as a solid. ESI-MS (m/z⁺): 453.4 (obs). ¹H-NMR (400 MHz, DMSO-d₆): δ_{H} 7.69 (s, 1H), 7.35 (s, 1H), 4.48-4.43 (m, 4H), 4.16-4.12 (m, 3H), 4.10-4.03 (m, 2H), 3.68-3.59 (m, 2H), 3.35-3.32 (m, 1H), 2.70 (s, 3H), 2.66 (s, 3H), 2.22 (d, J=13.0 Hz, 1H), 1.97 (d, J=13.0 Hz, 1H), 1.88-1.79 (m, 2H), 0.98-0.93 (m, 2H), 0.91-0.85 (m, 2H).

An analogous method was followed to obtain the following compound.

| Ex # | Compound | Starting Material | Characterization |
|---|---|---|---|
| 143 | 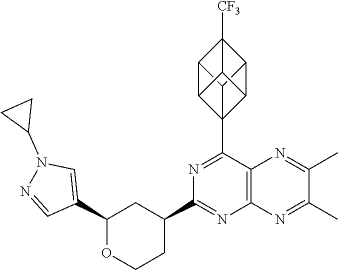 [SMILES: Cc1nc2nc([C@H]3CCO[C@@H](c4cnn(C5CC5)c4)C3)nc(C34C5C6C3C3C4C5C63C(F)(F)F)c2nc1C] | 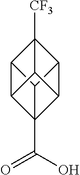 [SMILES: O=C(O)C12C3C4C1C1C2C3C41C(F)(F)F] | The residue was purified by silica gel flash column chromatography (1-8% MeOH in DCM) to afford 2-((2R,4S)-2- (1-cyclopropyl-1H-pyrazol-4- yl)tetrahydro-2H-pyran-4-yl)-6,7- dimethyl-4-(4-(trifluoromethyl)-cuban- 1-yl)pteridine (Example 143, 114 mg, 0.21 mmol, 86% yield) as a solid. ESI- MS (m/z+): 521.3 (obs). ¹H-NMR (400 MHz, CHCl₃-d): δ_{H} 7.49 (s, 1H), 7.48 (s, 1H), 4.54-4.51 (m, 4H), 4.36 (t, J = 4.7 Hz, 3H), 4.26-4.21 (m, 1H), 3.82-3.75 (m, 1H), 3.57-3.51 (m, 1H), 3.48-3.38 (m, 1H), 2.79 (s, 3H), 2.72 (s, 3H), 2.38 (d, J = 13.3 Hz, 1H), 2.20- 2.10 (m, 3H), 1.10-1.03 (m, 2H), 1.02- 0.94 (m, 2H). ¹⁹F-NMR (376 MHz, |
| | Example 143 | 4- | CHCl₃-d): δ_{F} −77.2 (s, 3F). |
| | | (trifluoromethyl)- | |
| | | cubane-1- | |
| | | carboxylic acid | |

### Method 57: Synthesis of Example 142

### Synthesis of A167

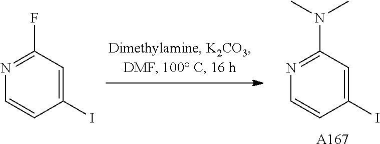

A mixture of 2-fluoro-4-iodo-pyridine (1.0 eq, 4.0 g, 17.9 mmol), dimethylamine (2.0 M in THF, 3.0 eq, 27 mL, 53.8 mmol) and K₂CO₃ (2.0 eq, 4.95 g, 35.9 mmol) was stirred overnight at 100° C. in a sealed reactor. The reaction was quenched with water and extracted with EtOAc (×3). The combined organic layers were washed with brine, dried over Na₂SO₄, and filtered. The filtrate was concentrated under reduced pressure and purified by normal phase column chromatography (0-40% EtOAc in hexanes) to afford 4-iodo-NN-dimethyl-pyridin-2-amine (A167, 3.4 g, 13.7 mmol, 76% yield) as an oil. ESI-MS (m/z)+: 249.1 (obs). ¹H-NMR (400 MHz, CHCl₃-d) δ_{H} 7.79 (d, J=5.0 Hz, 1H), 6.86-6.84 (m, 2H), 3.03 (s, 6H).

### Synthesis of A168

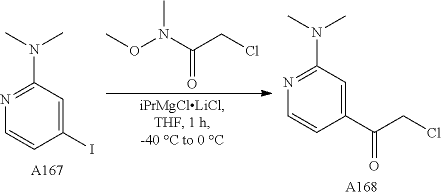

A 1.3 M solution of iPrMgCl LiCl (1.1 eq, 9.9 mL, 12.9 mmol) in THF was added dropwise to a −40° C. solution of 4-iodo-N,N-dimethyl-pyridin-2-amine (A167, 1.0 eq, 2.9 g, 11.7 mmol) in THF (58 mL), and the reaction was stirred for 30 min. 2-chloro-N-methoxy-N-methylacetamide (1.3 eq, 2.09 g, 15.2 mmol) was added, and the reaction was stirred for 1 h at 0° C. The reaction was quenched with sat. NaHCO₃(aq.) and extracted with EtOAc (×3). The combined organic layers were washed with brine, dried over Na₂SO₄, and filtered. The filtrate was concentrated under reduced pressure and purified by normal phase column chromatography (10-50% EtOAc in hexanes) to afford 2-chloro-1-[2-(dimethylamino)-4-pyridyl]ethanone (A168, 2.0 g, 10.1 mmol, 86% yield). ESI-MS (m/z)+: 199.1 (obs).

¹H NMR (CHCl₃-d, 400 MHz): δ_{H} 8.30 (dd, J=5.1, 0.8 Hz, 1H), 6.93 (t, J=1.0 Hz, 1H), 6.86 (dd, J=5.3, 1.4 Hz, 1H), 4.65 (s, 2H), 3.13 (s, 6H).

### Synthesis of A169

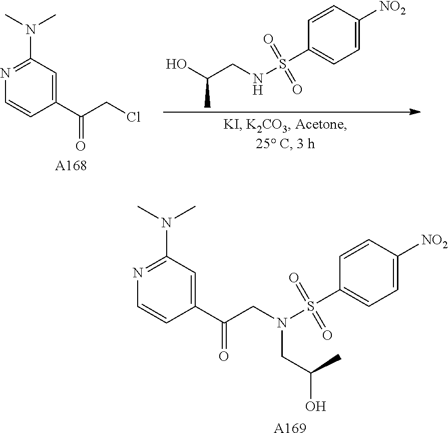

2-chloro-1-[2-(dimethylamino)-4-pyridyl]ethanone (A168, 1.0 eq, 1.0 g, 5.03 mmol) and potassium iodide (1.0 eq, 836 mg, 5.03 mmol) were stirred in acetone (25 mL), and the reaction was stirred for 1 h at room temperature. 4-nitro-N-[rac-(2R)-2-hydroxypropyl]benzenesulfonamide (1.2 eq, 1.57 g, 6.04 mmol) and K₂CO₃ (3.0 eq, 2.09 g, 15.1 mmol) were added to the mixture and the reaction was stirred at room temperature for 3 h. The reaction was quenched with water and extracted with EtOAc (×3). The combined organic layers were washed with brine, dried over Na₂SO₄, and filtered. The filtrate was concentrated under reduced pressure and purified by normal phase column chromatography (0-4% MeOH in DCM), followed by reversed-phase column chromatography (5-70% MeCN in water with 0.1% formic acid) to afford N-[2-[2-(dimethylamino)-4-pyridyl]-2-oxo-ethyl]-4-nitro-N-[rac-(2R)-2-hydroxypropyl]-benzenesulfonamide (A169, 518 mg, 1.23 mmol, 24% yield) as an oil. ESI-MS (m/z)+: 423.3 (obs). ¹H NMR (MeOH-d, 400 MHz): δ_{H} 8.39-8.37 (m, 2H), 8.13 (s, 1H), 8.02-7.99 (m, 2H), 7.93 (d, J=5.7 Hz, 1H), 6.90 (s, 1H), 6.79 (dd, J=5.8, 1.3 Hz, 1H), 4.34 (dd, J=10.6, 3.8 Hz, 1H), 3.77-3.71 (m, 2H), 3.09 (s, 6H), 2.40 (d, J=11.9 Hz, 1H), 2.31 (t, J=11.4 Hz, 1H), 1.19 (d, J=6.4 Hz, 3H).

### Synthesis of A170

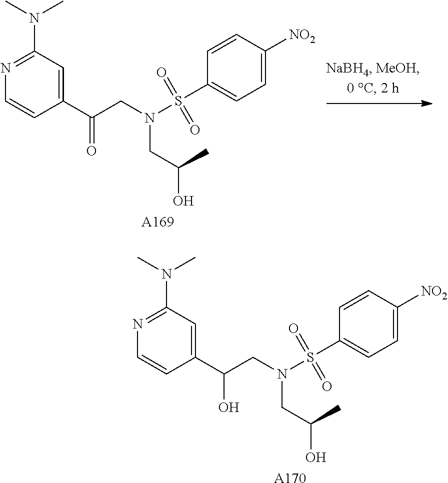

NaBH₄ (3.0 eq, 161 mg, 4.26 mmol) was added portion wise to a 0° C. solution of N-[2-[2-(dimethylamino)-4-pyridyl]-2-oxo-ethyl]-4-nitro-N-[rac-(2R)-2-hydroxypropyl]benzenesulfonamide (A169, 1.0 eq, 600 mg, 1.42 mmol) in MeOH (14 mL), and the reaction was stirred at room temperature for 30 min. The reaction was quenched with sat. NaHCO₃(aq.) and extracted with DCM (×3). The combined organic layers were washed with brine, dried over Na₂SO₄, and filtered. The filtrate was concentrated under reduced pressure and purified by normal phase column chromatography (20-80% EtOAc in hexanes) to afford N-[2-[2-(dimethylamino)-4-pyridyl]-2-hydroxy-ethyl]-4-nitro-N-[rac-(2R)-2-hydroxypropyl]benzenesulfonamide (A170, 150 mg, 0.35 mmol, 25% yield). ESI-MS (m/z)+: 425.3 (obs).

### Synthesis of A171 and A172

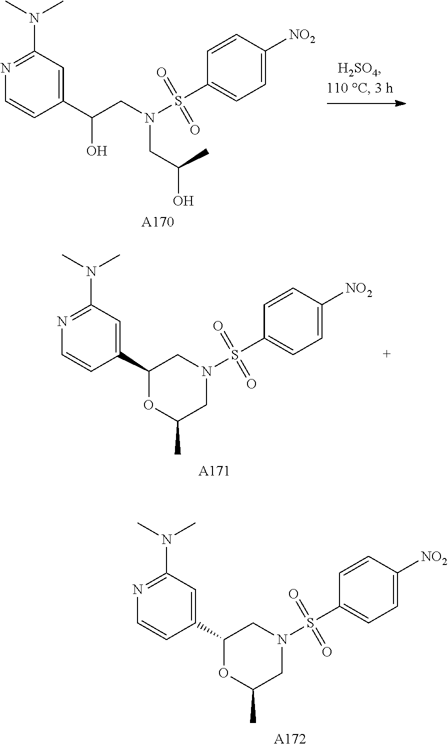

In a sealed tube was added N-[2-[2-(dimethylamino)-4-pyridyl]-2-hydroxy-ethyl]-4-nitro-N-[rac-(2R)-2-hydroxypropyl]benzenesulfonamide (A170, 1.0 eq, 150 mg, 0.35 mmol) and 70% aqueous H₂SO₄ (5 mL, 0.07 M). The vial was sealed, and the resulting mixture was stirred at 110° C. for 3 h. The reaction was cooled down to 0° C. and the acid was quenched with 50% NaOH (aq.) in an ice bath. The aqueous solution was extracted with EtOAc (×3) and the combined organic layers were washed with brine, dried over Na₂SO₄, and filtered. The filtrate was concentrated under reduced pressure and purified by normal phase column chromatography (5-40% EtOAc in hexanes) to afford N,N-dimethyl-4-[rac-(cis)-6-methyl-4-(4-nitrophenyl)sulfonyl-morpholin-2-yl]pyridin-2-amine (A171) and (A172) (20 mg, 0.049 mmol, 14% yield) as a solid. ¹H NMR (CHCL₃-d, 400 MHz): δ_{H} 8.36 (dt, J=9.0, 2.1 Hz, 2H), 8.09 (d, J=5.3 Hz, 1H), 7.93-7.88 (m, 2H), 6.43 (d, J=5.3 Hz, 1H), 6.41 (s, 1H), 4.56 (dd, J=10.3, 2.5 Hz, 1H), 3.89-3.85 (m, 1H), 3.79 (dt, J=11.4, 2.1 Hz, 1H), 3.69 (dt, J=11.3, 1.9 Hz, 1H), 3.06 (d, J=11.7 Hz, 7H), 2.16-2.09 (m, 2H), 1.25 (d, J=8.5 Hz, 3H). ESI-MS (m/z)+: 407.3 (obs).

### Synthesis of A173

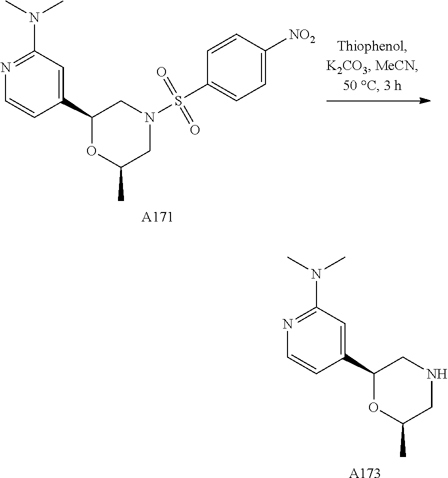

A mixture of N,N-dimethyl-4-[rac-(2S,6R)-6-methyl-4-(4-nitrophenyl)sulfonyl-morpholin-2-yl]pyridin-2-amine (A171, 1.0 eq, 73 mg, 0.18 mmol), K₂CO₃ (5.0 eq, 124 mg, 0.89 mmol) and thiophenol (3.0 eq, 0.055 mL, 0.54 mmol) in MeCN (2.0 mL) was stirred at 50° C. overnight. The reaction was diluted with HCl (1.0 M) and extracted with EtOAc. The organic phase was set aside, and the acidic aqueous layer was basified with sat. NaHCO₃(aq.) to neutral pH and extracted with EtOAc (×3). The combined organic layers were washed with brine, dried over Na₂SO₄, filtered and concentrated under reduced pressure to afford N,N-dimethyl-4-[rac-(2S,6R)-6-methylmorpholin-2-yl]pyridin-2-amine (A173, 30 mg, 0.14 mmol, 75% yield). ESI-MS (m/z)+: 222.2 (obs).

### Synthesis of Example 142

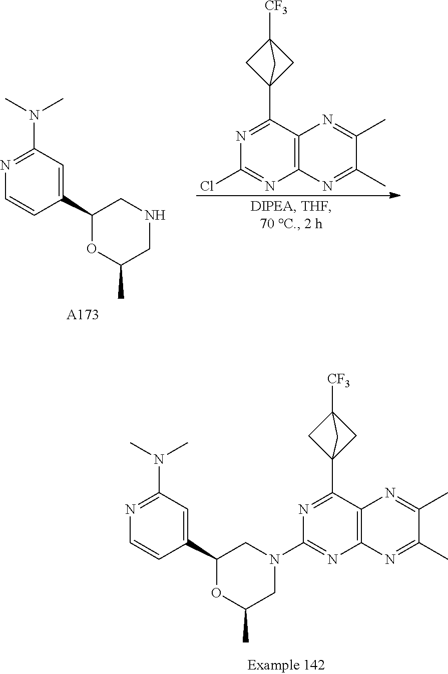 [SMILES: C[C@@H]1CNC[C@H](c2ccnc(N(C)C)c2)O1 ; Cc1nc2nc(Cl)nc(C34CC(C(F)(F)F)(C3)C4)c2nc1C ; "A173" ; Cc1nc2nc(N3C[C@@H](C)O[C@@H](c4ccnc(N(C)C)c4)C3)nc(C34CC(C(F)(F)F)(C3)C4)c2nc1C]

A mixture of 2-chloro-6,7-dimethyl-4-[3-(trifluoromethyl)-1-bicyclo[1.1.1]pentanyl]pteridine (1.5 eq, 74 mg, 0.22 mmol), N,N-dimethyl-4-[rac-(2S,6R)-6-methylmorpholin-2-yl]pyridin-2-amine (A173, 1.0 eq, 33 mg, 0.15 mmol) and DIPEA (3.0 eq, 0.078 mL, 0.45 mmol) in THF (2 mL) was stirred at 70° C. for 2 h. The reaction was concentrated under reduced pressure and purified by normal phase column chromatography (40-100% EtOAc in hexanes) to afford N,N-dimethyl-4-[rac-(2S,6R)-4-[6,7-dimethyl-4-[3-(trifluoromethyl)-1-bicyclo[1.1.1]pentanyl]pteridin-2-yl]-6-methyl-morpholin-2-yl]pyridin-2-amine (Example 142, 16 mg, 0.031 mmol, 21% yield) as a solid. ESI-MS (m/z)+: 514.5 (obs). ¹H NMR (400 MHz, CHCl₃-d) δ_{H} 8.15 (d, 1H, J=5.0 Hz), 6.59 (s, 2H), 5.05 (d, 1H, J=13.0 Hz), 4.94 (d, 1H, J=13.0 Hz), 4.49 (dd, 1H, J=10.7, 2.5 Hz), 3.82-3.78 (m, 1H), 3.10 (s, 6H), 2.89 (dd, 1H, J=13.4, 10.9 Hz), 2.80 (dd, 1H, J=13.3, 10.7 Hz), 2.66 (s, 3H), 2.61 (s, 3H), 2.57 (s, 6H), 1.35 (d, 3H, J=6.2 Hz). ¹⁹F NMR (376 MHz, CHCl₃-d) δ_{F}−72.9 (s, 3F).

### Example A3: In Vitro Assay Data

In vitro Measurement of Triggering Receptor Expressed on Myeloid Cells 2 activity using cellular phosphorylation of Spleen Tyrosine Kinase (“Syk”) Assays

Measurement of TREM2 agonist potency was done using a HEK cell line expressing human TREM2 and DAP12 (HEK293T-hTREM2 cells). Binding of small molecules to, and activation of, TREM2 increases the phosphorylation of Syk. The resultant levels of Syk phosphorylation are measured using a commercial AlphaLisa reagent kit. To perform the assay, HEK-hTREM2 cells were plated at 14,000 cells per well in a 384 well plate, in 25 μL of complete growth media and incubated at 37° C., 5% CO₂ for 20-24 hours.

Prior to the assay, test compounds were diluted in the 384 well plates in assay buffer and allowed to equilibrate for 30 minutes. Growth media was removed from cell plates by inversion on blotting paper, and 25 μL of test articles in assay buffer was added to cells. Cells were incubated for 45 minutes at room temperature. After 45 minutes, assay buffer was removed and 10 μL of lysis buffer was added. Plates were shaken for 20 minutes at 350 RPM at room temperature. After complete lysis, AlphaLisa reagents were added to the lysate, and fluourescence intensity was measured using a Perkin Elmer Envision plate reader. Intensities were used to generate a standard curve, and % activation was calculated. Curve fitting was performed using Prism v9 software, log(agonist) vs response—variable slope (four parameters), and EC50s were calculated from the curve fit.

The results presented in Table B have been generated with the in vitro assay described above. This assay may be used to test any of the compounds described herein to assess and characterize a compound's ability to act as an agonist of TREM2.

Compounds designated as “A” demonstrated an EC₅₀ of ≤100 nM. Compounds designated as “B” demonstrated an EC₅₀>100 nM and ≤500 nM. Compounds designated as “C” demonstrated an EC₅₀>500 nM.

TABLE B
hTREM2 EC50 Data (HEK293 Cells)

| | hTREM2 |
|---|---|
| Ex # | EC50 (nM) |
| 1 | A |
| 2 | C |
| 3 | C |
| 4 | A |
| 5 | A |
| 6 | A |
| 7 | A |
| 8 | A |
| 9 | C |
| 10 | A |
| 11 | A |
| 12 | A |
| 13 | A |
| 15 | C |
| 16 | A |
| 18 | A |
| 19 | A |
| 21 | A |
| 22 | A |
| 23 | A |
| 24 | A |
| 25 | A |
| 26 | A |
| 27 | B |
| 28 | C |
| 29 | A |
| 31 | A |
| 32 | A |
| 33 | A |
| 35 | A |
| 42 | B |
| 44 | B |
| 45 | B |
| 46 | A |
| 65 | A |
| 66 | A |
| 67 | B |
| 68 | A |
| 69 | B |
| 70 | A |
| 71 | A |
| 72 | A |
| 73 | A |
| 74 | A |
| 81 | A |
| 84 | A |
| 86 | C |
| 87 | B |
| 90 | B |
| 91 | A |
| 93 | A |
| 94 | A |
| 95 | A |
| 96 | B |
| 98 | A |
| 99 | A |
| 100 | A |
| 102 | A |
| 104 | C |
| 106 | A |
| 107 | A |
| 108 | A |
| 109 | A |
| 110 | A |
| 111 | A |
| 113 | A |
| 114 | A |
| 115 | A |
| 116 | A |
| 117 | A |
| 119 | B |
| 121 | B |
| 123 | C |
| 124 | A |
| 125 | B |
| 127 | A |
| 129 | A |
| 130 | B |
| 132 | B |
| 134 | B |
| 136 | A |
| 138 | C |
| 140 | A |
| 142 | B |
| 143 | A |

All references, for example, a scientific publication or patent application publication, cited herein are incorporated herein by reference in their entirety and for all purposes to the same extent as if each reference was specifically and individually indicated to be incorporated by reference in its entirety for all purposes.

What is claimed is:

1. A compound of Formula (I): 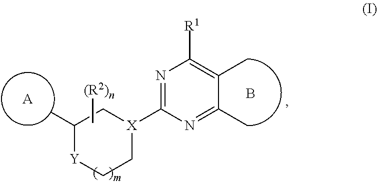
or a pharmaceutically acceptable salt, solvate, stereoisomer, or tautomer thereof, or a pharmaceutically acceptable salt and/or solvate of said compound, stereoisomer, or tautomer, wherein:
Ring A is selected from cycloalkyl, heterocyclyl, aryl, or 5- to 9-membered heteroaryl, wherein each cycloalkyl, heterocyclyl, aryl, and heteroaryl is optionally substituted with one or more R³; wherein when ring A is a 6-membered heteroaryl, the 6-membered heteroaryl is 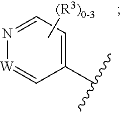
Ring B together with the 6-membered ring system to which it is fused forms a bicyclic ring system selected from 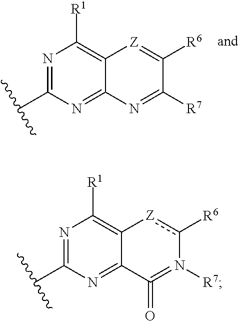
W is C(R⁵) or N;
X is C(R⁵) or N;
Y is C(R¹¹)(R¹²) or O;
Z is C(R¹³), C(R¹³)(R¹⁴), or N, wherein the dashed bond comprises a single or double bond depending on valency;
R¹ is cycloalkyl, heterocyclyl, aryl, or heteroaryl, wherein each cycloalkyl, heterocyclyl, aryl, and heteroaryl is optionally substituted with one or more R⁴;
each R² is independently C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), —N(R^{B})(R^{C}), wherein each alkyl, alkenyl, alkynyl, heteroalkyl, haloalkyl, cycloalkyl, and heterocyclyl is optionally substituted with one or more R⁵; or
two R², taken together with the carbon atom to which they are attached, form an oxo group;
each R³ and R⁴ is independently deuterium, C₁₋₆ alkyl, C₂₋₆ alkenyl, C₂₋₆ alkynyl, C₁₋₆ heteroalkyl, C₁₋₆haloalkyl, cycloalkyl, heterocyclyl, halogen, cyano, oxo, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R⁸;
R⁵ is hydrogen, C₁₋₆ alkyl, C₂₋₆ alkenyl, C₂₋₆ alkynyl, C₁₋₆ heteroalkyl, C₁₋₆ haloalkyl, halogen, cyano, —O(R^{A}), or —N(R^{B})(R^{C}), wherein each alkyl, alkenyl, alkynyl, heteroalkyl, and haloalkyl is optionally substituted with one or more R⁸;
R⁶ and R⁹ are each independently hydrogen, C₁₋₆ alkyl, C₂₋₆ alkenyl, C₂₋₆ alkynyl, C₁₋₆ heteroalkyl, C₁₋₆haloalkyl, cycloalkyl, heterocyclyl, aryl, heteroaryl, halogen, cyano, —O(R^{A}), —C(O)(R^{A}), —C(O)O(R^{A}), —C(O)N(R^{B})(R^{C}), —C(═N-OR^{A})(R^{C}), —N(R^{B})(R^{C}), —N(O)(R^{A}), or —S(O)ₓ(R^{C}), wherein alkyl, alkenyl, alkynyl, heteroalkyl, haloalkyl, cycloalkyl, heterocyclyl, aryl, and heteroaryl is optionally substituted with one or more R⁹;
each R⁸ and R⁹ is independently deuterium, C₁₋₆ alkyl, C₂₋₆ alkenyl, C₂₋₆ alkynyl, C₁₋₆ heteroalkyl, C₁₋₆haloalkyl, cycloalkyl, heterocyclyl, halogen, cyano, —C(O)O(R^{A}), —O(R^{A}), or —N(R^{B})(R^{C}), wherein each alkyl, alkenyl, alkynyl, heteroalkyl, haloalkyl, cycloalkyl, and heterocyclyl is optionally substituted with one or more R¹⁰;
each R^{A} is independently hydrogen, deuterium, C₁₋₆ alkyl, C₁₋₆ heteroalkyl, C₁₋₆ haloalkyl, cycloalkyl, heterocyclyl, or —N(R^{B})(R^{C}), wherein each alkyl, heteroalkyl, haloalkyl, cycloalkyl, and heterocyclyl is optionally substituted with one or more R¹⁷;
each R^{B} and R^{C} is independently hydrogen, C₁₋₆ alkyl, C₁₋₆ heteroalkyl, C₁₋₆ haloalkyl, cycloalkyl, heterocyclyl, or —C(O)-alkyl, wherein each alkyl, heteroalkyl, haloalkyl, cycloalkyl, and heterocyclyl is optionally substituted with one or more R¹⁷;
each R¹⁰ is deuterium, C₁₋₆ alkyl, C₁₋₆ heteroalkyl, C₁₋₆ haloalkyl, cycloalkyl, halogen, or cyano;
each R¹¹ and R¹² is independently hydrogen, C₁₋₆ alkyl, C₂₋₆ alkenyl, C₂₋₆ alkynyl, C₁₋₆ heteroalkyl, C₁₋₆haloalkyl, halogen, cyano, —O(R^{A}), or —N(R^{B})(R^{C}), wherein each alkyl, alkenyl, alkynyl, heteroalkyl, and haloalkyl is optionally substituted with one or more R¹⁷;
each R¹⁷ is deuterium, C₁₋₆ alkyl, C₁₋₆ heteroalkyl, C₁₋₆ haloalkyl, cycloalkyl, halogen, or cyano;
m is 0, 1, or 2;
n is 0, 1, 2, 3, 4, 5, or 6; and
x is 0, 1, or 2.

2. The compound of claim 1, or a pharmaceutically acceptable salt, solvate, stereoisomer, or tautomer thereof, or a pharmaceutically acceptable salt and/or solvate of said compound, stereoisomer, or tautomer, wherein R¹ is selected from cycloalkyl, heterocyclyl, or heteroaryl, each of which is optionally substituted with one or more R⁴.

3. The compound of any one of claims 1-2, or a pharmaceutically acceptable salt, solvate, stereoisomer, or tautomer thereof, or a pharmaceutically acceptable salt and/or solvate of said compound, stereoisomer, or tautomer, wherein R¹ is cycloalkyl optionally substituted with one or more R⁴.

4. The compound of any one of claims 1-3, or a pharmaceutically acceptable salt, solvate, stereoisomer, or tautomer thereof, or a pharmaceutically acceptable salt and/or solvate of said compound, stereoisomer, or tautomer, wherein R¹ is a 4-membered, 5-membered, or 6-membered cycloalkyl, each of which is optionally substituted with one or more R⁴.

5. The compound of any one of claims 1-5, or a pharmaceutically acceptable salt, solvate, stereoisomer, or tautomer thereof, or a pharmaceutically acceptable salt and/or solvate of said compound, stereoisomer, or tautomer, wherein R¹ is selected from cyclopropyl, cyclopentyl, and cyclohexyl, each of which is optionally substituted with one or more R⁴.

6. The compound of any one of the preceding claims, or a pharmaceutically acceptable salt, solvate, stereoisomer, or tautomer thereof, or a pharmaceutically acceptable salt and/or solvate of said compound, stereoisomer, or tautomer, wherein R¹ is cyclohexyl, cyclopentenyl, cyclobutyl, 3-(trifluoromethyl)-1-bicyclo[1.1.1]pentanyl, 3-(difluoromethyl)bicyclo[1.1.1]pentan-1-yl), bicyclo[1.1.1]pentane-1-carbonitrile, 4-(trifluoromethyl)-cuban-1-yl, cubanyl, 1-bicyclo[1.1.1]pentanyl, or 3-(difluoromethyl)bicyclo[1.1.1]pentan-1-yl.

7. The compound of any one of the preceding claims, or a pharmaceutically acceptable salt, solvate, stereoisomer, or tautomer thereof, or a pharmaceutically acceptable salt and/or solvate of said compound, stereoisomer, or tautomer, wherein R¹ is selected from

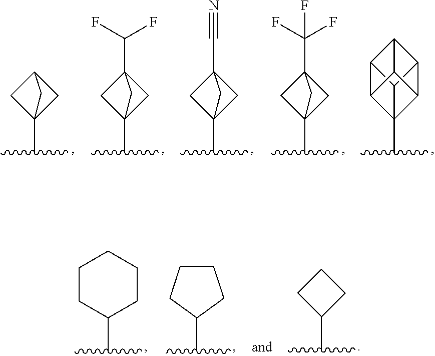

8. The compound of any one of claims 1-2, or a pharmaceutically acceptable salt, solvate, stereoisomer, or tautomer thereof, or a pharmaceutically acceptable salt and/or solvate of said compound, stereoisomer, or tautomer, wherein R¹ is aryl or heteroaryl, each of which is optionally substituted with one or more R⁴.

9. The compound of claim 8, or a pharmaceutically acceptable salt, solvate, stereoisomer, or tautomer thereof, or a pharmaceutically acceptable salt and/or solvate of said compound, stereoisomer, or tautomer, wherein R¹ is phenyl or thiazolyl, each of which is optionally substituted with one or more R⁴.

10. The compound of any one of the preceding claims, or a pharmaceutically acceptable salt, solvate, stereoisomer, or tautomer thereof, or a pharmaceutically acceptable salt and/or solvate of said compound, stereoisomer, or tautomer, wherein R¹ is selected from

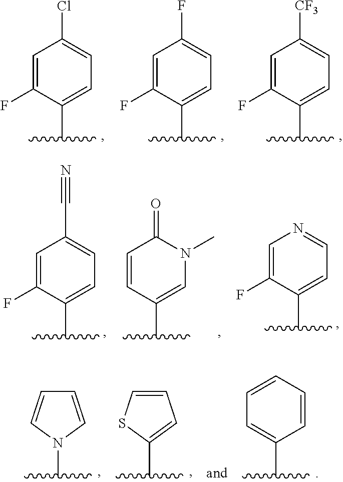

11. The compound of any one of claims 1-10, or a pharmaceutically acceptable salt, solvate, stereoisomer, or tautomer thereof, or a pharmaceutically acceptable salt and/or solvate of said compound, stereoisomer, or tautomer, wherein R⁴ is deuterium, C₁₋₆ alkyl, C₁₋₆ heteroalkyl, C₁₋₆ haloalkyl, or halogen.

12. The compound of any one of claims 1-11, or a pharmaceutically acceptable salt, solvate, stereoisomer, or tautomer thereof, or a pharmaceutically acceptable salt and/or solvate of said compound, stereoisomer, or tautomer, wherein Ring A is heteroaryl substituted with one or more R.

13. The compound of any one of claims 1-12, or a pharmaceutically acceptable salt, solvate, stereoisomer, or tautomer thereof, or a pharmaceutically acceptable salt and/or solvate of said compound, stereoisomer, or tautomer, wherein Ring A is pyrazolyl substituted with one or more R³.

14. The compound of any one of the preceding claims, or a pharmaceutically acceptable salt, solvate, stereoisomer, or tautomer thereof, or a pharmaceutically acceptable salt and/or solvate of said compound, stereoisomer, or tautomer, wherein Ring A is selected from

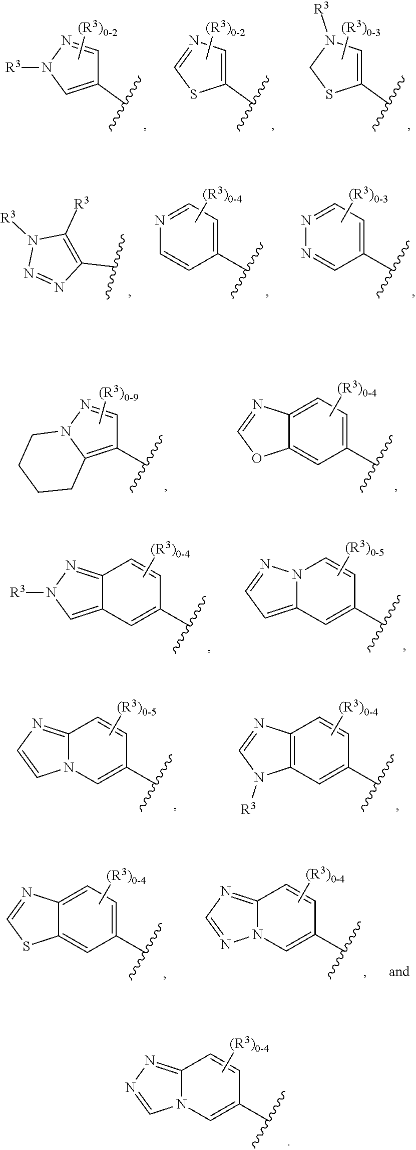

15. The compound of any one of the preceding compounds, or a pharmaceutically acceptable salt, solvate, stereoisomer, or tautomer thereof, or a pharmaceutically acceptable salt and/or solvate of said compound, stereoisomer, or tautomer, wherein Ring A is selected from:

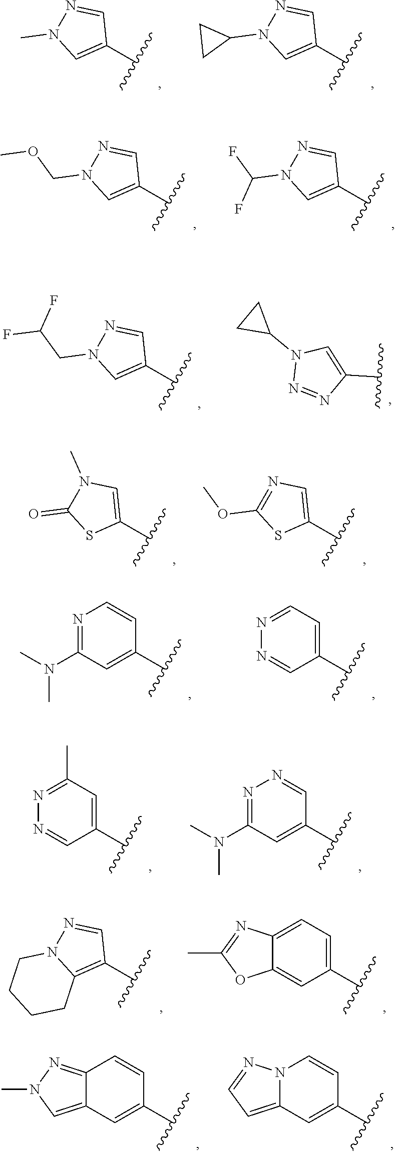

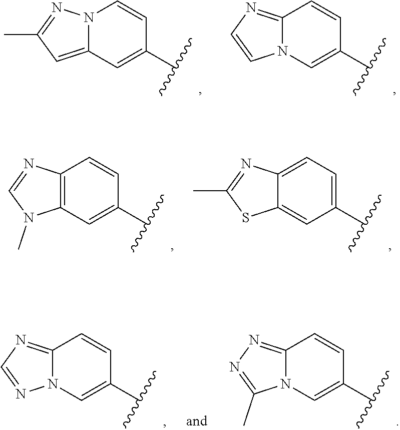 [SMILES: *c1ccn2nc(C)cc2c1 ; *c1ccc2nccn2c1 ; *c1ccc2ncn(C)c2c1 ; *c1ccc2nc(C)sc2c1 ; *c1ccc2ncnn2c1 ; *c1ccc2nnc(C)n2c1]

16. The compound of any one of claims 1-11, or a pharmaceutically acceptable salt, solvate, stereoisomer, or tautomer thereof, or a pharmaceutically acceptable salt and/or solvate of said compound, stereoisomer, or tautomer, wherein Ring A is heterocyclyl substituted with one or more R³.

17. The compound of any one of claims 1-16, or a pharmaceutically acceptable salt, solvate, stereoisomer, or tautomer thereof, or a pharmaceutically acceptable salt and/or solvate of said compound, stereoisomer, or tautomer, wherein R³ is deuterium, C₁₋₆ alkyl, C₁₋₆ heteroalkyl, C₁₋₆haloalkyl, cycloalkyl, halogen, oxo, —O(R^{A}), or —N(R^{B})(R^{C}).

18. The compound of any one of claims 1-17, or a pharmaceutically acceptable salt, solvate, stereoisomer, or tautomer thereof, or a pharmaceutically acceptable salt and/or solvate of said compound, stereoisomer, or tautomer, wherein R³ is cycloalkyl.

19. The compound of any one of the preceding claims, or a pharmaceutically acceptable salt, solvate, stereoisomer, or tautomer thereof, or a pharmaceutically acceptable salt and/or solvate of said compound, stereoisomer, or tautomer, wherein Ring B together with the 6-membered ring system to which it is fused forms a bicyclic ring system selected from

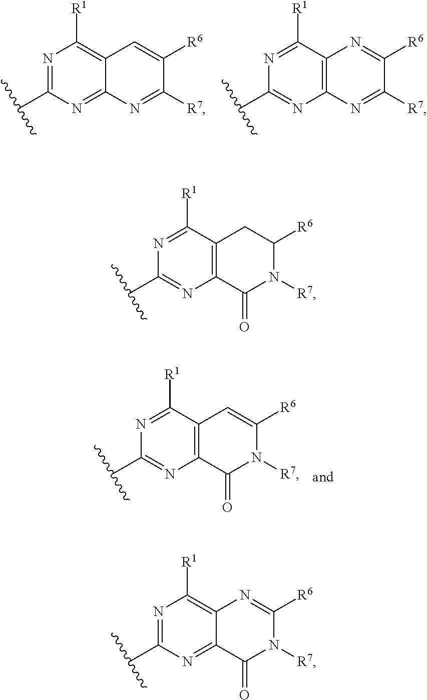

20. The compound of any one of claims 1-19, or a pharmaceutically acceptable salt, solvate, stereoisomer, or tautomer thereof, or a pharmaceutically acceptable salt and/or solvate of said compound, stereoisomer, or tautomer, wherein R⁶ and R⁹ are each independently hydrogen, C₁₋₆ alkyl, C₁₋₆ heteroalkyl, C₁₋₆ haloalkyl, cycloalkyl, heteroaryl, heterocyclyl, halogen, cyano, —O(R^{A}), —C(O)(R^{A}), —C(O)O(R^{A}), —C(O)N(R^{B})(R^{C}), —C(═N—OR^{A})(R^{C}), —N(R^{B})(R^{C}), —N(O)(R^{A}), or —S(O)ₓ(R^{C}), wherein alkyl, heteroalkyl, haloalkyl, cycloalkyl, and heterocyclyl is optionally substituted with one or more R⁹.

21. The compound of any one of claims 1-20, or a pharmaceutically acceptable salt, solvate, stereoisomer, or tautomer thereof, or a pharmaceutically acceptable salt and/or solvate of said compound, stereoisomer, or tautomer, wherein R⁶ is C₁₋₆ alkyl, heterocyclyl, heteroaryl, halogen, haloalkyl, cyano, —O(R^{A}), —C(O)(R^{A}), —C(O)O(R^{A}), —N(R^{B})(R^{C}), —C(═N—OR^{A})(R^{C}), or —S(O)ₓ(R^{C}), and R⁷ is hydrogen or C₁₋₆ alkyl.

22. The compound of any one of claims 1-21, or a pharmaceutically acceptable salt, solvate, stereoisomer, or tautomer thereof, or a pharmaceutically acceptable salt and/or solvate of said compound, stereoisomer, or tautomer, wherein R⁶ is heterocyclyl, heteroaryl, halogen, cyano, —O(R^{A}), —C(O)(R^{A}), —C(O)O(R^{A}), —N(R^{B})(R^{C}), —N(O)(R^{A}), or —S(O)ₓ(R^{C}), and R⁷ is hydrogen or C₁₋₆ alkyl.

23. The compound of any one of claims 1-22, or a pharmaceutically acceptable salt, solvate, stereoisomer, or tautomer thereof, or a pharmaceutically acceptable salt and/or solvate of said compound, stereoisomer, or tautomer, wherein R⁶ is —O(R^{A}), —C(O)(R^{A}), —C(O)O(R^{A}), —N(O)(R^{A}), or —S(O)ₓ(R^{C}), and R⁷ is hydrogen or C₁₋₆ alkyl.

24. The compound of any one of claims 1-23, or a pharmaceutically acceptable salt, solvate, stereoisomer, or tautomer thereof, or a pharmaceutically acceptable salt and/or solvate of said compound, stereoisomer, or tautomer, wherein R⁶ is heterocyclyl, heteroaryl, halogen, or cyano and R⁹ is hydrogen or C₁₋₆ alkyl.

25. The compound of any one of claims 1-24, or a pharmaceutically acceptable salt, solvate, stereoisomer, or tautomer thereof, or a pharmaceutically acceptable salt and/or solvate of said compound, stereoisomer, or tautomer, wherein R⁶ is heterocyclyl or heteroaryl, selected from:

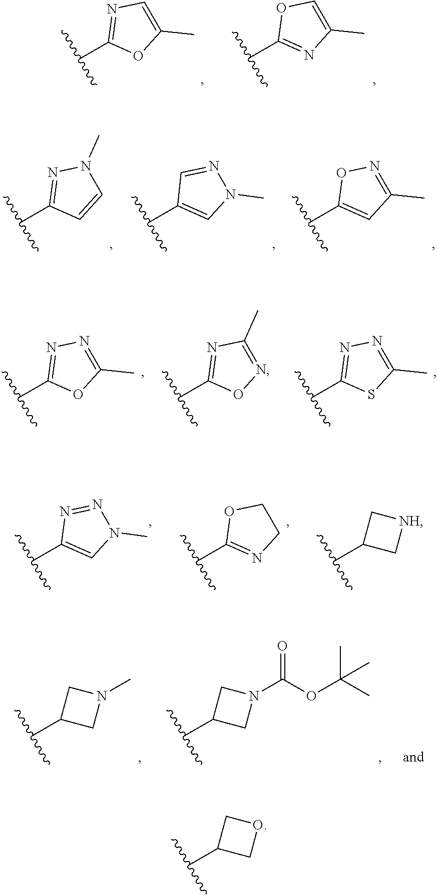

26. The compound of any one of claims 1-24, or a pharmaceutically acceptable salt, solvate, stereoisomer, or tautomer thereof, or a pharmaceutically acceptable salt and/or solvate of said compound, stereoisomer, or tautomer, wherein R⁶ is halogen or cyano and R⁷ is hydrogen or C₁₋₆ alkyl.

27. The compound of any one of claims 1-24, or a pharmaceutically acceptable salt, solvate, stereoisomer, or tautomer thereof, or a pharmaceutically acceptable salt and/or solvate of said compound, stereoisomer, or tautomer, wherein R⁶ and R⁹ are each independently C₁₋₆ alkyl.

28. The compound of any one of claims 1-27, or a pharmaceutically acceptable salt, solvate, stereoisomer, or tautomer thereof, or a pharmaceutically acceptable salt and/or solvate of said compound, stereoisomer, or tautomer, wherein R⁹ is CH₃.

29. The compound of any one of claims 1-28, or a pharmaceutically acceptable salt, solvate, stereoisomer, or tautomer thereof, or a pharmaceutically acceptable salt and/or solvate of said compound, stereoisomer, or tautomer, wherein X is C(R⁵).

30. The compound of any one of claims 1-28, or a pharmaceutically acceptable salt, solvate, stereoisomer, or tautomer thereof, or a pharmaceutically acceptable salt and/or solvate of said compound, stereoisomer, or tautomer, wherein X is N.

31. The compound of any one of the preceding claims, or a pharmaceutically acceptable salt, solvate, stereoisomer, or tautomer thereof, or a pharmaceutically acceptable salt and/or solvate of said compound, stereoisomer, or tautomer, wherein Y is O.

32. The compound of any one of claims 1-30, or a pharmaceutically acceptable salt, solvate, stereoisomer, or tautomer thereof, or a pharmaceutically acceptable salt and/or solvate of said compound, stereoisomer, or tautomer, wherein Y is C(R¹¹)(R¹²) (e.g., CH₂).

33. The compound of any one of claims 1-30, or a pharmaceutically acceptable salt, solvate, stereoisomer, or tautomer thereof, or a pharmaceutically acceptable salt and/or solvate of said compound, stereoisomer, or tautomer, wherein Y is CH₂.

34. The compound of any one of claims 1-33, or a pharmaceutically acceptable salt, solvate, stereoisomer, or tautomer thereof, or a pharmaceutically acceptable salt and/or solvate of said compound, stereoisomer, or tautomer, wherein W is N.

35. The compound of any one of claims 1-33, or a pharmaceutically acceptable salt, solvate, stereoisomer, or tautomer thereof, or a pharmaceutically acceptable salt and/or solvate of said compound, stereoisomer, or tautomer, wherein W is C(R⁵) (e.g., CH).

36. The compound of any one of claims 1-35, or a pharmaceutically acceptable salt, solvate, stereoisomer, or tautomer thereof, or a pharmaceutically acceptable salt and/or solvate of said compound, stereoisomer, or tautomer, wherein Z is selected from C(R¹³) (e.g., CH) or N.

37. The compound of any one of claims 1-36, or a pharmaceutically acceptable salt, solvate, stereoisomer, or tautomer thereof, or a pharmaceutically acceptable salt and/or solvate of said compound, stereoisomer, or tautomer, wherein Z is C(R¹³).

38. The compound of any one of claims 1-36, or a pharmaceutically acceptable salt, solvate, stereoisomer, or tautomer thereof, or a pharmaceutically acceptable salt and/or solvate of said compound, stereoisomer, or tautomer, wherein Z is N.

39. The compound of any one of claims 1-38, or a pharmaceutically acceptable salt, solvate, stereoisomer, or tautomer thereof, or a pharmaceutically acceptable salt and/or solvate of said compound, stereoisomer, or tautomer, wherein n is 0 or 1.

40. The compound of any one of the preceding claims, or a pharmaceutically acceptable salt, solvate, stereoisomer, or tautomer thereof, or a pharmaceutically acceptable salt and/or solvate of said compound, stereoisomer, or tautomer, wherein m is 0 or 1.

41. The compound of any one of the preceding claims, or a pharmaceutically acceptable salt, solvate, stereoisomer, or tautomer thereof, or a pharmaceutically acceptable salt and/or solvate of said compound, stereoisomer, or tautomer, wherein m is 1.

42. The compound of any one of the preceding claims, or a pharmaceutically acceptable salt, solvate, stereoisomer, or tautomer thereof, or a pharmaceutically acceptable salt and/or solvate of said compound, stereoisomer, or tautomer, wherein the compound of Formula (I) is a compound of Formula (I-a): 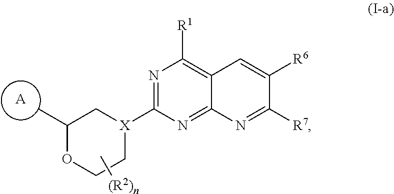
or a pharmaceutically acceptable salt, solvate, stereoisomer, or tautomer thereof, or a pharmaceutically acceptable salt and/or solvate of said compound, stereoisomer, or tautomer, wherein each of Ring A, R¹, R², R⁶, R⁷, n and subvariables thereof are defined as for Formula (I).

43. The compound of any one of the preceding claims, wherein the compound of Formula (I) is a compound of Formula (I-b): 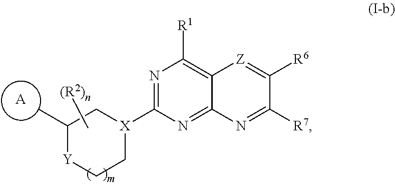
or a pharmaceutically acceptable salt, solvate, stereoisomer, or tautomer thereof, or a pharmaceutically acceptable salt and/or solvate of said compound, stereoisomer, or tautomer, wherein each of Ring A, R¹, R², R⁶, R⁷, X, Y, Z, m, n, and subvariables thereof are defined as for Formula (I).

44. The compound of any one of the preceding claims, wherein the compound of Formula (I) is a compound of Formula (I-c): 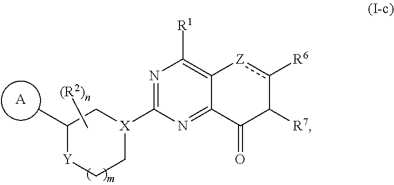
or a pharmaceutically acceptable salt, solvate, stereoisomer, or tautomer thereof, or a pharmaceutically acceptable salt and/or solvate of said compound, stereoisomer, or tautomer, wherein each of Ring A, R¹, R², R⁶, R⁷, X, Y, Z, m, n, and subvariables thereof are defined as for Formula (I).

45. The compound of any one of the preceding claims, wherein the compound of Formula (I) is a compound of Formula (I-d): 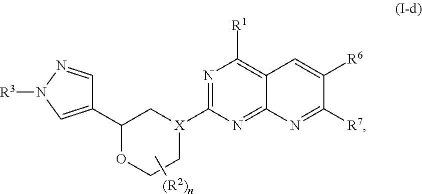
or a pharmaceutically acceptable salt, solvate, stereoisomer, or tautomer thereof, or a pharmaceutically acceptable salt and/or solvate of said compound, stereoisomer, or tautomer, wherein each of R¹, R², R³, R⁶, R⁷, n and subvariables thereof are defined as for Formula (I).

46. The compound of any one of the preceding claims, wherein the compound of Formula (I) is a compound of Formula (I-e): 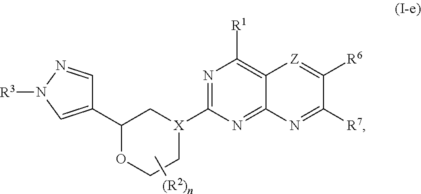
or a pharmaceutically acceptable salt, solvate, stereoisomer, or tautomer thereof, or a pharmaceutically acceptable salt and/or solvate of said compound, stereoisomer, or tautomer, wherein each of R¹, R², R³, R⁶, R⁷, Z, n and subvariables thereof are as defined for Formula (I).

47. The compound of any one of the preceding claims, wherein the compound of Formula (I) is a compound of Formula (I-f) 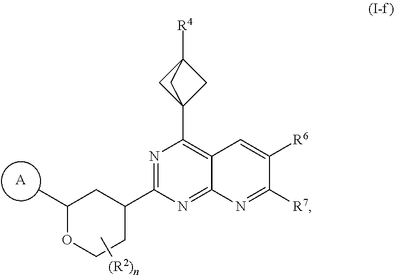
or a pharmaceutically acceptable salt, solvate, stereoisomer, or tautomer thereof, or a pharmaceutically acceptable salt and/or solvate of said compound, stereoisomer, or tautomer, wherein each of R², R⁴, R⁶, R⁷, n and subvariables thereof are defined as for Formula (I).

48. The compound of any one of the preceding claims, wherein the compound of Formula (I) is a compound of Formula (I-g): 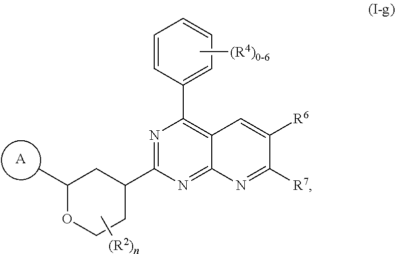
or a pharmaceutically acceptable salt, solvate, stereoisomer, or tautomer thereof, or a pharmaceutically acceptable salt and/or solvate of said compound, stereoisomer, or tautomer, wherein each of R², R⁴, R⁶, R⁷, n and subvariables thereof are defined as for Formula (I).

49. The compound of any one of the preceding claims, wherein the compound of Formula (I) is a compound of Formula (I-h): 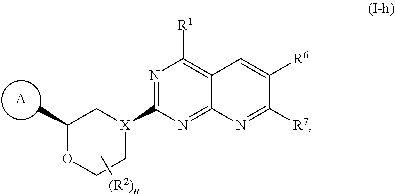
or a pharmaceutically acceptable salt, solvate, stereoisomer, or tautomer thereof, or a pharmaceutically acceptable salt and/or solvate of said compound, stereoisomer, or tautomer, wherein each of R¹, R², R⁶, R⁷, n and subvariables thereof are defined as for Formula (I).

50. The compound of any one of the preceding claims, wherein the compound of Formula (I) is a compound of Formula (I-i): 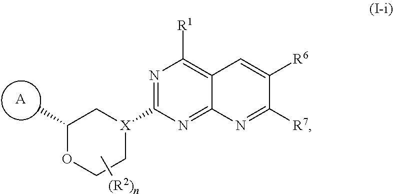
or a pharmaceutically acceptable salt, solvate, stereoisomer, or tautomer thereof, or a pharmaceutically acceptable salt and/or solvate of said compound, stereoisomer, or tautomer, wherein each of R², R¹³, n and subvariables thereof are defined as for Formula (I).

51. The compound of any one of the preceding claims, wherein the compound of Formula (I) is a compound of Formula (I-j): 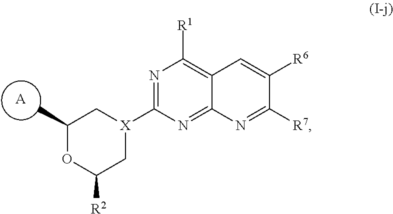
or a pharmaceutically acceptable salt, solvate, stereoisomer, or tautomer thereof, or a pharmaceutically acceptable salt and/or solvate of said compound, stereoisomer, or tautomer, wherein each of R¹, R², R⁶, R⁷, n and subvariables thereof are defined as for Formula (I).

52. The compound of any one of claims 1-51, or a pharmaceutically acceptable salt, solvate, stereoisomer, or tautomer thereof, or a pharmaceutically acceptable salt and/or solvate of said compound, stereoisomer, or tautomer, wherein the compound is a compound provided in Table A.

53. A pharmaceutical composition comprising the compound according to any one of claims 1-52, or a pharmaceutically acceptable salt, solvate, stereoisomer, or tautomer thereof, or a pharmaceutically acceptable salt and/or solvate of said compound, stereoisomer, or tautomer, and a pharmaceutically acceptable excipient.

54. A compound according to any one of claims 1-52, or a pharmaceutically acceptable salt, solvate, stereoisomer, or tautomer thereof, or a pharmaceutically acceptable salt and/or solvate of said compound, stereoisomer, or tautomer, or the pharmaceutical composition of claim 53, for use in treating or preventing a condition associated with a loss of function of human TREM2.

55. A method of treating or preventing a condition associated with a loss of function of human TREM2 in a subject in need thereof, the method comprising administering to the subject a therapeutically effective amount of the compound according to any one of claims 1-52, or a pharmaceutically acceptable salt, solvate, stereoisomer, or tautomer thereof, or a pharmaceutically acceptable salt and/or solvate of said compound, stereoisomer, or tautomer, or the pharmaceutical composition of claim 53.
